(19) United States
(12) Patent Application Publication
(10) Pub. No.: US 2014/0086976 A1
(43) Pub. Date: Mar. 27, 2014

(54) Compositions containing protein polymers and vaccinia virus, and methods of use thereof

(71) Applicants: Aladar A. Szalay, Highland, CA (US); Nanhai George Chen, San Diego, CA (US)

(72) Inventors: Aladar A. Szalay, Highland, CA (US); Nanhai George Chen, San Diego, CA (US)

(21) Appl. No.: 13/987,688

(22) Filed: Aug. 20, 2013

Related U.S. Application Data
(60) Provisional application No. 61/742,895, filed on Aug. 20, 2012.

(51) Int. Cl. A61K 47/42; A61K 35/76

(52) U.S. Cl. CPC A61K 47/42; A61K 35/76
USPC 424/445; 424/93.6; 424/93.2; 424/277.1; 424/174.1; 424/447

(57) ABSTRACT

Provided herein are compositions containing a vaccinia virus and a protein polymer, and articles of manufacture thereof. Also provided are diagnostic and therapeutic methods using the compositions or articles of manufacture.

### RELATED APPLICATIONS

Benefit of priority is claimed to U.S. Provisional Application No. 61/742,895, filed Aug. 20, 2012, entitled “Compositions Containing Protein Polymers and Vaccinia Virus, and Methods of Use Thereof.”

This application is related to International PCT Application Serial No. (Attorney Docket No. 33316.04842.WO02/4842PC), filed the same day herewith, entitled “Compositions Containing Protein Polymers and Vaccinia Virus, and Methods of Use Thereof,” which claims priority to U.S. Provisional Application No. 61/742,895.

The subject matter of each of the above-noted related applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Aug. 20, 2013, is identical, 4.24 megabytes in size, and titled 4842SEQ.001.txt.

### FIELD OF THE INVENTION

Provided herein are compositions containing a vaccinia virus and a protein polymer, and articles of manufacture thereof. Also provided are diagnostic and therapeutic methods using the compositions or articles of manufacture.

### BACKGROUND

Vaccinia is an oncolytic virus and accumulates in wounds and tumors. Oncolytic viral therapy is effected by administering a virus that accumulates in tumor cells and replicates in the tumor cells. By virtue of replication in the cells, and optional delivery of therapeutic agents, tumor cells are lysed, and the tumor shrinks and can be eliminated. Vaccinia viruses are typically administered systemically or locally. There still exists a need for improved or alternative methods of administering vaccinia viruses for various therapeutic and diagnostic applications. Accordingly, it is among the objects herein, to provide virus compositions that can be employed for diagnostic and/or therapeutic methods.

### SUMMARY

Provided herein are vaccinia virus in protein polymer compositions. For example, provided herein are vaccinia virus in silk-elastin like protein polymer compositions containing a vaccinia virus and a silk-elastin like protein polymer (SELP). In such examples, the SELP can contain alternating blocks of at least two units each of a silk-like amino acid sequence and an elastin-like amino acid sequence set forth by the formula {[S]ₘ[E]ₙ}_{O}, wherein: S is the silk-like amino acid sequence; m is the number of silk-like amino acid units; E is the elastin-like amino acid sequence; n is the number of elastin-like amino acid units; and o is the number of monomer repeats. In examples of the compositions, m is 2 to 16, 2 to 10, 2 to 8, 4 to 16, 4 to 10 or 4 to 8. In examples of the compositions, n is 1 to 40, 1 to 16, 2 to 12 or 4 to 8. In examples of the compositions o is 2 to 100, 4 to 50, 6 to 25 or 2 to 20. In particular examples, the choice of repeating units and polymer length is such that the SELP has a molecular weight of at least 15 kD. For example, the SELP has a molecular weight of 15 kD to 100 kD, 40 kD to 90 kD or 60 kD to 85 kD.

The compositions provided herein can be liquid or non-liquid. For example, the compositions are hydrogels. In examples where the composition is a liquid, it is typically a composition that is a precursor hydrogel composition, and that will form a non-liquid or hydrogel form upon time or incubation at physiologic temperature (e.g. about or approximately 37° C.). For example, in examples herein, the SELP is selected from among SELPs that transition from a liquid to a non-liquid form in from about 30 seconds to about 500 minutes at 22° C. to 25° C. In other examples, the SELP is selected from among SELPs that transition from a liquid formulation to a hydrogel at or about 37° C. Any of the compositions herein can further contain an agent that inhibits or decreases hydrogen bonding. For example, the further agent can be urea, guanidine hydrochloride, dimethyl formamide, colloidal gold sol, aqueous lithium bromide or formic acid.

In examples of the compositions provided herein, the alternating units of silk-like amino acid sequences is GAGAGS (SEQ ID NO:26) or SGAGAG (SEQ ID NO:27), or is a variant thereof that is capable of effecting formation of hydrogen bonds; and/or the elastin-like amino acid sequence is VPGG (SEQ ID NO:30), APGVGV (SEQ ID NO:31), VPGVG (SEQ ID NO:32), or GVGVP (SEQ ID NO:29), or is a variant thereof that confers aqueous solublility. In one example, the elastin-like sequence is a variant sequence having an amino acid sequence GXGVP (SEQ ID NO:35) or VPGXG (SEQ ID NO:36), whereby X is defined as set forth in the sequence listing. For example, the elastin-like sequence is VPGKG (SEQ ID NO:37) or GKGVP (SEQ ID NO:38). In other examples, the sequence is a variant that contains a conservative substitution of any of SEQ ID NOS: 26, 27, 29-32, or 37-38. The conservative substitution is replacement of serine with threonine or replacement of glycine with alanine.

In examples of the compositions provided herein, the SELP contains a sequence of amino acids having a structural formula and amino acid sequence that is [(VPGVG)₈(GAGAGS)₂]₁₈ (SEQ ID NO:39); [(GVGVP)₄(GAGAGS)₉]₁₃ (SEQ ID NO:40); [(VPGVG)₈(GAGAGS)₄]₁₂ (SEQ ID NO:41); [(VPGVG)₈(GAGAGS)₆]₁₂ (SEQ ID NO:42); [(VPGVG)₈(GAGAGS)₈]₁₁ (SEQ ID NO:43); [(VPGVG)₁₂(GAGAGS)₈]₈ (SEQ ID NO:44); [(VPGVG)₁₆(GAGAGS)₈]₇ (SEQ ID NO:45); [(VPGVG)₃₂(GAGAGS)₈]₅ (SEQ ID NO:46); (GAGAGS)₁₂ GAAVTGRGDSPASAAGY (GAGAGS)₅(GVGVGP)₈]₆ (SEQ ID NO:47); [(GAGAGS)₂ (GVGVP)₄ GKGVP (GVGVP)₃]₆ (SEQ ID NO:48); [(GAGAGS)₂(GVGVP)₄ GKGVP (GVGVP)₃]₁₂ (SEQ ID NO:49); [(GAGAGS)₂(GVGVP)₄ GKGVP (GVGVP)₃]₁₈ (SEQ ID NO:50); [(GAGAGS)₂ (GVGVP)₄ GKGVP (GVGVP)₃]₁₇ GAGAGS)₂ (SEQ ID NO:51); [(GAGAGS)₂-(GVGVP)₄-(GKGVP)-(GVGVP)₃-(GAGAGS)₂]₁₃ (SEQ ID NO:52); [GAGAGS (GVGVP)₄ GKGVP (GVGVP)₃(GAGAGS)₂]₁₂ (SEQ ID NO:53); [(GVGVP)₄GKGVP(GVGVP)₁₁(GAGAGS)₄]₅(GVGVP)₄GKGVP(GVGVP)₁₁(GAG AGS)₂(SEQ ID NO:54); [(GVGVP)₄(GKGVP)(GVGVP)₁₁(GAGAGS)₄]₇(GVGVP)₄GKGVP(GVGVP)₁₁(GA GAGS)₂ (SEQ ID NO:55); [(GVGVP)₄GKGVP(GVGVP)₁₁(GAGAGS)₄]₉(GVGVP)₄GKGVP(GVGVP)₁₁(GAG AGS)₂ (SEQ ID NO:56); [GAGS(GAGAGS)₂(GVGVP)₄GKGVP(GVGVP)₁₁(GAGAGS)₅GA]₆ (SEQ ID NO:57); [(GAGAGS)₂ (GVGVP)₁ LGPLGP (GVGVP)₃ GKGVP (GVGVP)₃]₁₅ (GAGAGS)₂ (SEQ ID NO:73); and [(GAGAGS)₂ (GVGVP)₁ GFFVRARR (GVGVP)₃ GKGVP (GVGVP)₃)₁₅(GAGAGS)₂ (SEQ ID NO:74). For example, the SELP is a polymer that is [(GAGAGS)₂(GVGVP)₄GKGVP (GVGVP)₃]₁₇ GAGAGS)₂ (SEQ ID NO:51); [(GAGAGS)₂-(GVGVP)₄-(GKGVP)-(GVGVP)₃-(GAGAGS)₂]₁₃ (SEQ ID NO:52); or [GAGS(GAGAGS)₂(GVGVP)₄GKGVP(GVGVP)₁₁(GAGAGS)₅GA]₆ (SEQ ID NO:57).

In any of the examples of compositions herein, the SELP can further contain an N-terminal head sequence and/or a C-terminal tail sequence. In one example, the N-terminal head sequence has the sequence of amino acids set forth as MDPVVLQRRDWENPGVTQLNRLAAHPPFASDPM (set forth in SEQ ID NO:58). In another example, the C-terminal tail sequence has the sequence of amino acids set forth as GAGAMDPGRYQDLRSHHHHHH (SEQ ID NO:59) or GAMDPGRYQDLRSHHHHHH (SEQ ID NO:60).

In particular examples of the compositions provided herein, the vaccinia virus is in a SELP that is SELP-27K (SEQ ID NOS: 61), SELP-47K (SEQ ID NO:62) or SELP-815K (SEQ ID NO:63).

In the compositions provided herein, the SELP is present in the composition at a weight percentage (wt %) of the composition of from or from about 2% (w/w) to about 50% (w/w), from about 2% (w/w) to about 35% (w/w), from about 2% (w/w) to about 20% (w/w), from about 2% (w/w) to about 12% (w/w), from about 4% (w/w) to about 50% (w/w), from about 4% (w/w) to about 35% w/w, from about 4% (w/w) to about 12%, from about 4% (w/w) to about 8% (w/w), from about 5% (w/w) to about 50% (w/w), from about 10% (w/w) to about 50% (w/w) or from about 20% (w/w) to about 35% (w/w). For example, the SELP is present in the composition at a weight percentage (wt %) of the composition of from at least about or about 4%, 5%, 6%, 7%, 8%, 9%, 10%, 11%, 12%, 13%, 14%, 15% or 20% (w/w).

In any of the compositions provided herein, the vaccinia virus in the composition can be a Lister, Western Reserve (WR), Copenhagen (Cop), Bern, Paris, Tashkent, Tian Tan, Wyeth (DRYVAX), 1′-ID-J, IHD-W, Brighton, Ankara, CVA382, Modified Vaccinia Ankara (MVA), Dairen I, LC16 m8, LC16M0, LIVP, ACAM2000, WR 65-16, Connaught, New York City Board of Health (NYCBH), EM-63 or a NYVAC strain. In particular example, the vaccinia virus is a Lister strain virus. For example, the vaccinia virus is an LIVP virus, a clonal strain of an LIVP virus, or a modified form thereof containing nucleic acid encoding a heterologous gene product. In one example, the LIVP virus or modified form thereof has a sequence of nucleotides set forth in SEQ ID NO:1, or a sequence of nucleotides that has at least 95% sequence identity to SEQ ID NO:1. In another example, the LIVP virus is a clonal strain of LIVP or a modified form thereof that has a sequence of nucleotides selected from: a) nucleotides 10,073-180,095 of SEQ ID NO:2, nucleotides 11,243-182,721 of SEQ ID NO:3, nucleotides 6,264-181,390 of SEQ ID NO:4, nucleotides 7,044-181,820 of SEQ ID NO:5, nucleotides 6,674-181,409 of SEQ ID NO:6, nucleotides 6,716-181,367 of SEQ ID NO:7 or nucleotides 6,899-181,870 of SEQ ID NO:8; orb) a sequence of nucleotides that has at least 97% sequence identity to a sequence of nucleotides 10,073-180,095 of SEQ ID NO:2, nucleotides 11,243-182,721 of SEQ ID NO:3, nucleotides 6,264-181,390 of SEQ ID NO:4, nucleotides 7,044-181,820 of SEQ ID NO:5, nucleotides 6,674-181,409 of SEQ ID NO:6, nucleotides 6,716-181,367 of SEQ ID NO:7 or nucleotides 6,899-181,870 of SEQ ID NO:8. In any of the examples of a vaccinia virus, including an LIVP virus or a clonal strain of an LIVP virus, the virus strain can contain a left and/or right inverted terminal repeat. In examples of the compositions herein, the vaccinia virus or modified form thereof contains a sequence of nucleotides set forth in SEQ ID NOS: 2-8, or a sequence of nucleotides that has at least 97% sequence identity to a sequence of nucleotides set forth in SEQ ID NO: 2-8.

For example, in any of the examples of compositions provided herein, the vaccinia virus is a modified form, such as a modified form of an LIVP virus or a clonal strain of an LIVP virus. The modified form is one where nucleic acid encoding a heterologous gene product is inserted into or in place of a non-essential gene or region in the genome of the virus. For example, the nucleic acid encoding the heterologous gene product is inserted at the hemagglutinin (HA), thymidine kinase (TIC), F14.5L, vaccinia growth factor (VGF), A35R, N1L, E2L/E3L, K1L/K2L, superoxide dismutase locus, 7.5K, C7-K1L, B13R+B14R, A26L or 14L gene loci in the genome of the virus. In such examples, the heterologous gene product is a therapeutic or reporter gene product. For example, the heterologous gene product is one that encodes an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging. In one example, the heterologous gene product is an antimetastatic agent and the antimetastatic agent inhibits metastatic colonization or inhibits cell invasion in an in vitro cell invasion assay. For example, the heterologous gene product is an antiangiogenic agent and the antiangiogenic agent inhibits blood vessel formation in a tumor.

In examples of the compositions provided herein, the vaccinia virus is modified by insertion of nucleotides that encode a heterologous gene product that is a therapeutic agent or protein that is a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor. For example, the heterologous gene product can be a therapeutic protein that is a granulocyte macrophage colony stimulating factor (GM-CSF), monocyte chemotactic protein-1 (MCP-1), interleukin-6 (IL-6), interleukin-24 (IL-24), interferon gamma-induced protein 10 (IP-10), lymphotoxin inducible expression competes with HSV glycoprotein D for HVEM a receptor expressed on T-lymphocytes (LIGHT), p60 superantigen, OspF, OspG, signal transducer and activator of transcription protein (STATlalpha), STAT1beta, plasminogen k5 domain (hK5), pigment epithelium-differentiation factor (PEDF), single chain anti-VEGF antibody, single chain anti-DLL4 antibody, single chain anti-fibroblast activation protein (FAP), NM23, cadherin 1 (ECAD or cdhl), relaxin 1 (RLN1), matrix metallopeptidase 9 (MMP9), erythropoietin (EPO), microRNAl26 (miR-126), microRNA 181, microRNA 335, manganese superoxide dismutase (MnSOD), E3 ubiquitin protein ligase 1 (HACE1), natriuretic peptide precursor A (nppa1), carboxypeptidase G2 (CPG2), alcohol dehydrogenase (ADH), CDC6, or bone morphogenetic protein 4 (BMP4).

In other examples of the compositions provided herein, the vaccinia virus is modified by insertion of nucleotides that encode a heterologous gene product that is a reporter gene product. The reporter gene product can be a fluorescent protein, a bioluminescent protein, an enzyme, or a cell surface protein that is capable of detection. For example, the cell surface protein is a receptor, transporter or ligand that binds to a detectable moiety or a moiety that is capable of detection. In such examples, the detectable moiety is selected from among a radiolabel, a chromogen, or a fluorescent moiety. In another example, the receptor or transporter protein is an iron receptor, an iron transporter, a copper uptake transporter or an ion transporter protein. For example, the receptor or transporter protein is an ion transporter protein that is a sodium ion transporter, such as a sodium ion transporter that is a norepinephrine transporter (NET) or the sodium iodide symporter (NIS). In other examples, the reporter is a fluorescent protein that is a green fluorescent protein, an enhanced green fluorescent protein, a blue fluorescent protein, a cyan fluorescent protein, a yellow fluorescent protein, a red fluorescent protein, or a far-red fluorescent protein. For example, the fluorescent protein is TurboFP635. In further examples, the reporter is an enzme, such as a luciferase, β-glucuronidase, β-galactosidase, chloramphenicol acetyl tranferase (CAT), alkaline phosphatase, or horseradish peroxidase. In some examples, the enzyme is one that can be detected by reaction of the enzyme with a substrate. For example, provided herein are compositions containing a vaccinia virus, such as an LIVP virus, that is modified by insertion of nucleotides that encode a heterologous gene product that is a green click beetle luciferase, a lux operon, an infrared fluorescent protein, a flavin reductase protein, mNeptune far-red fluorescent protein, green fluorescent protein (GFP), red fluorescent protein (RFP), coelenterazine-binding protein (CBP), human epinephrine receptor (hNET), a sodium iodide symporter (NIS) protein, a cytochrome p450 family enzyme, allostatin A receptor (AlstR), Pep1 Receptor (PEPR-1), LAT-4, sterol 14 alpha-demethylase (Cyp51), transferrin receptor (TR), ferritin, divalent metal transporter (DMT), Magnetotactic A (MagA), cisplatin influx transporter (CTRL), newt AG (nAG), Oct4, NANOG, Ngn3, Pdx1 or Mafa.

In examples of the compositions provided herein, the vaccinia virus is modified by insertion of nucleotides that encodes a heterologous gene product, wherein the nucleic acid encoding the heterologous gene product is operably linked to a promoter. The promoter can be a mammalian promoter or a viral promoter. For example, the promoter is selected from among P_{7.5k}, P₁₁ₖ, P_{SE}, P_{SEL}, P_{SL}, H5R, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b or K1 promoters.

Among the vaccinia viruses contained in compositions provided herein are vaccinia viruses that have a sequence of nucleotides selected from among any of SEQ ID NOS:9, 18-23 and 25, or a sequence of nucleotides that exhibits at least 97% sequence identity to any of SEQ ID NOS: 9, 18-23 and 25. For example, the vaccina virus can contain a sequence of nucleotides that exhibits at least 98% or at least 99% sequence identity to any of SEQ ID NOS: 9, 18-23 and 25.

In any of the compositions provided herein, the vaccinia virus is present in the composition in an amount that is from or from about 1×10⁵ to 1×10¹² pfu, 1×10⁶ to 1×10¹⁰ pfu or 1×10⁷ to 1×10¹⁰ pfu. For example, the vaccinia virus can be present in the composition in an amount that is at least or about at least or 1×10⁶, 1×10⁷, 1×10⁸, 1×10⁹, 2×10⁹, 3×10⁹, 4×10⁹, or 5×10⁹ pfu.

In examples of any of the compositions provided herein, the volume of the compositions can be from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL or 0.5 mL to 5 mL. For example, the volume of the composition is at least or about at least or 0.05 mL, 0.5 mL, 1 mL, 2 mL, 3 mL, 4 mL, 5 mL, 6 mL, 7 mL, 8 mL, 9 mL or 10 mL. In examples herein, the compositions can be formulated for direct administration.

Any of the compositions provided herein can further contain a pharmaceutically acceptable carrier. The compositions can be formulated for use in therapy as pharmaceutical compositions. The compositions provided herein can be formulated for local or systemic injection. For example, the compositions can be formulated for intravenous administration. In other examples, the compositions are formulated for topical administration.

Provided herein are combinations containing a composition containing a vaccinia virus in protein polymer, such as any of the compositions provided herein, and an anti-cancer agent. The anticancer agent can be a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent or a combination of any of the preceding thereof.

Provided herein are methods of treating a disease or condition in a subject that is any disease or condition that is or can be treated by a vaccinia virus by administering to a subject a composition containing a vaccinia virus in a protein polymer, such as any of the compositions provided herein. Medical uses of the compositions provided herein include use of any of the compositions provided herein for treating a disease or condition that is or can be treated by a vaccinia virus. Hence, any of the compositions provided herein can be formulated as a medicament for treating a disease or condition in a subject that is any disease or condition that is or can be treated by a vaccinia virus. For example, in any of the methods, uses or compositions provided herein, the disease or condition is a proliferative disorder or condition. The proliferative disorder or condition can be a cancer or a wound, in particular a wound healing disorder or condition. The wound can be an internal or an external wound. In some examples, the wound is a dermal wound (e.g. keloid and hypertrophic scars and other similar wounds). Hence, examples of methods, uses and compositions provided herein are any for treating a cancer in a subject. Other examples of methods, uses or compositions provided herein are any for treating a wound disorder or condition, such as a wound healing disorder or condition, in a subject.

In any of the methods provided herein, the composition is administered locally or systemically. For example, the composition is administered intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally. In some examples, the composition is administered locally inside a body cavity.

In examples of the methods, uses and compositions provided herein, the proliferative disease is cancer. The cancer can be a carcinoma, sarcoma, lymphoma or leukemia. For example, the cancer is a cancer of the tongue, mouth, throat, stomach, cecum, colon, rectum, breast, ovary, uterus, thyroid, adrenal cortex, lung, kidney, prostate or pancreas. In examples herein, the proliferative disorder is a tumor or a metastasis. For example, the tumor is a solid tumor. In particular examples of methods provided herein for treating a cancer, such as a tumor or solid tumor, the composition is administered intravenously.

In other examples of the methods, uses or compositions provided herein, the proliferative disease is a surface or skin wound. In one example of the methods, uses, or compositions provided herein, the proliferative disease is a cancer that is a tumor, and in particular is a surgically resected tumor. The surgically resected tumor can be any tumor where the volume of the residual tumor is 10 mm³ to 300 m³, 10 mm³ to 100 mm³, 25 mm³ to 100 mm³, 50 mm³ to 100 mm³, 50 mm³ to 250 mm³ or 100 mm³ to 200 mm³. For example, the volume of the residual tumor is less than 100 mm³, 90 mm³, 80 mm³, 70 mm³, 60 mm³, 50 mm³, 40 mm³, 30 mm³ or 20 mm³. In other examples of methods, uses or compositions provided herein, the proliferative disease is a cancer that is a skin cancer. For example, the skin cancer is a melanoma, a basal cell carcinoma of the skin or a squamous cell carcinoma. In particular examples of the methods provided herein for treating a proliferative disease that is a surface or skin wound, for example a surgically resected tumor or skin cancer, the composition is administered topically.

In any of the examples of the methods provided herein, the subject is a human or non-human animal. For example, the subject is a non-human animal that is a horse, cat, dog, cow, pig, sheep, goat, mouse, rabbit, chicken, rat, and guinea pig.

In any of the methods provided herein, the composition is administered to deliver at least or about 1×10⁵ pfu of virus. For example, the composition is administered to deliver an amount of virus that is at least or about or is 1×10⁶ pfu, 1×10⁷ pfu, 1×10⁸ pfu, 1×10⁹ pfu, 1×10¹⁰ pfu, 1×10¹¹ pfu, 1×10¹² pfu, 1×10¹³ pfu, or 1×10¹⁴ pfu. In examples of the methods provided herein, any of the compositions provided herein can be administered two times, three times, four times, five times, six times or seven times.

In any of the methods provided herein, the method can further include administering a second therapeutic agent or treatment for the treatment of the proliferative disorder. In one example, the other treatment can be surgery, radiation therapy, immunosuppressive therapy or administration of an anticancer agent. For example, the anticancer agent can be a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent or a combination of any of the preceding thereof. For example, the anticancer agent is cisplatin, carboplatin, gemcitabine, irinotecan, an anti-EGFR antibody or an anti-VEGF antibody. In such examples, the composition and the other treatment or therapeutic agent, for example anticancer agent, are administered sequentially, simultaneously, or intermittently.

Also provided herein is a device containing a composition containing a vaccinia virus in protein polymer, such as any of the compositions provided herein. In such examples, the composition can be coated on a surface of the device. The device can be any device that is capable of being applied to a surface of the body of a subject. For example, the device is a patch, bandage, wrap, dressing, suture, film or mesh. In particular examples, the device is a wound dressing or bandage.

Provided herein are method of treating a skin lesion in a subject by applying any of the devices provided herein to the surface of the skin of a subject to cover the skin lesion. Also provided are any of the devices provided herein for use in treating a skin lesion in a subject. The skin lesion is a wound or other proliferative skin lesion. The proliferative skin lesion can be one that is benign, premalignant or malignant. For example, the proliferative skin lesion is a skin cancer. The skin cancer can be a melanoma, a basal cell carcinoma of the skin or a squamous cell carcinoma. In other example, the wound is a traumatic wound or a post-surgical wound. For example, the wound is a traumatic wound that is a burn, scrape or cut. For example, the wound is a post-surgical wound that is a surgically resected tumor. In any of the methods provided herein for treating a skin lesion in a subject, the subject is a human or non-human animal. For example, the subject is a non-human animal that is a horse, cat, dog, cow, pig, sheep, goat, mouse, rabbit, chicken, rat, or guinea pig.

### DETAILED DESCRIPTION

### Outline

A. Definitions

B. Virus Polymer Compositions and Methods of Viral Delivery and Treatment
1. Vaccinia Viruses
2. Delivery of Vaccinia Viruses
3. SELP Compositions

C. Vaccinia Viruses and LIVP
1. Lister and LIVP Strains
2. Heterologous Nucleic Acid and Modified Viruses
   a. Exemplary Modifications
      i. Diagnositc or Reporter Gene Products
      ii. Therapeutic Gene Products
      iii. Modifications to alter attenuation of the viruses
   b. Exemplary Modified or Recombinant Viruses
   c. Control of Heterologous Gene Expression
   d. Methods of Generating Modified Viruses
3. Methods of Producing Viruses
   a. Host cells for Propagation
   b. Concentration Determination
   c. Storage Methods
   d. Preparation of Virus

D. Protein Polymers
1. Silk-elastin Like Polymers (SELP)
2. Methods of Preparing and Generating Polymers

E. Vaccinia Virus-Protein Polymer (VV-Polymer) Compositions
1. Methods of Making Compositions
2. Exemplary VV-SELP Compositions
3. Dosage Forms, Carriers and Excipients
4. Combinations
5. Kits

F. Devices and Articles of Manufacture

G. Assays to Assess Virus Activity or Composition Properties
1. Characterization of Hydrogel Compositions
2. Evaluation of Virus Diffusion or Release
3. Evaluation of Viral Integrity
4. Anti-Tumorigenicity and Efficacy
   a. Tumor-Associated Replication Indicator
   b. Cytotoxicity
   c. Tumor Growth
5. Toxicity/Safety

H. Therapeutic, Diagnositic and Monitoring Methods
1. Therapeutic or Diagnostic Methods
   a. Systemic Delivery to Treat or Detect Proliferative or Inflammatory Cells or Tissues (e.g. Tumors)
   b. Delivery to Treat or Detect Wounds of Hyperproliferative Surface Lesions
      Surgically Resected Tumor
2. Dosages and Dosage Regime
3. Combination Therapy
   a. Oncolytic or Therapeutic Virus
   b. Therapeutic Compounds
4. Monitoring
   a. Monitoring viral gene expression
   b. Monitoring tumor size
   c. Monitoring antibody titer
   d. Monitoring general health diagnostics
   e. Monitoring coordinated with treatment

I. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the interne can come and go, but equivalent information can be found by searching the internet. Reference thereto evidences the availability and public dissemination of such information.

As used herein, a matrix is a surrounding substance within which something else is contained. For purposes herein, a matrix refers to the structural properties or architecture of a solid or semi-solid (including a hydrogel) in which other components can be cast, mixed, dispersed or dissolved. For example, a matrix can contain atherapeutic product or virus.

As used herein, a liquid or fluid refers to a composition that flows freely.

As used herein, a hydrogel matrix or hydrogel refers to a semisolid composition constituting a substantial amount of water. A hydrogel can be formed from a network of polymer chains in which polymers or mixtures thereof are dissolved or dispersed. Hydrogels are composed of polymers that will swell without dissolving when placed in water or other biological fluids. A hydrogel is significantly more viscous than water or other similar liquid. Hence, for purposes herein, a hydrogel is generally a non-liquid form.

As used herein, viscous refers to a composition that exhibits a resistance to flow compared to water, and therefore exhibits a higher viscosity than water. For example, a viscous substance or composition is one that is has a thick consistency, between a solid and a liquid.

As used herein, viscosity refers to a measure of the resistance of fluid to flow. Viscosity can be measured in centipoise (cp), whereby water is the standard at 1 cps. Viscosity can be measured using a tube viscosimeter, a rotational viscometer (e.g. a cone plate type viscometer), a Gilmont viscometer, cannon capillary viscometer and other similar devices or apparatuses well known to one of skill in the art. Method and techniques for measuring or assessing viscosity of a sample are well known to one of skill in the art.

As used herein, a polymer refers to a molecule composed of a number of repeat units.

As used herein, a protein polymer refers to a polymer made up of repeating amino acid sequence units, wherein the repeating units are derived from a natural or synthetic protein. For example, in some embodiments, the repeating sequence units are derived from natural supporting structure materials such as silk, elastin, collagen and keratin. In alternative embodiments, the repeating sequence units are derived from synthetic structures. The polymer can be a polypeptide having an amino acid sequence made up of repeating units of smaller, identical monomer units linked together. Polymers can have high molecular weights of 5 kD to 200 kD, generally at least 15 kD, such as at least 20 kD, 30 kD, 40 kD, 50 kD, 60 kD, 70 kD, 80 kD, 90 kD, 100 kD or more. Exemplary protein polymers herein are any that are capable of irreversibly transitioning from liquid solution to a hydrogel gel (sol-to-gel transition). The transition generally can occur spontaneously as a function of time, temperature, concentration of polymer, and other factors. In particular, protein polymers herein are capable of sol-to-gel transition at physiologic temperatures.

As used herein, a silk-elastinlike polymer (SELP) refers to a protein polymer containing alternating units of silk-like units and elastin-like units and that is capable of transitioning from a liquid to a hydrogel. The transition generally can occur spontaneously as a function of time, temperature, concentration of polymer, and other factors. In particular, SELPs herein are capable of sol-to-gel transition at physiologic temperatures. Silk provides cross linking capability and renders mechanical strength, while elastin enhances aqueous solubility. A SELP typically has the formula {[S]ₘ[E]ₙ}_{O}, whereby S is the silk-like amino acid sequence; m is the number of silk-like amino acid units; E is the elastin-like amino acid sequence; n is the number of elastin-like amino acid units; and o is the number of monomer repeats. The particular amino acid sequence of the silk-like or elastin-like unit and the number of units and repeats of monomer units can be empirically determined as described herein or known to one of skill in the art. SELPs also can contain intervening sequences between the silk-like and elastin-like units. SELPs are known in the art (see e.g. U.S. Pat. No. 5,606,019; U.S. Pat. No. 5,723,588; U.S. Pat. No. 5,770,697; U.S. Pat. No. 6,380,154; U.S. Pat. No. 6,423,333; Megeed et al. (2002) Advanced Drug Delivery Reviews, 54:1075-1091; Cappello et al. (1998) Journal of Controlled Release, 53:105-117; Gustafson et al. (2010) Advanced Drug Delivery Reviews, 62:1509-1523; Haider et al. (2004) Molecular Pharmaceutics, 2:139-150; Hatefi et al. (2007) Pharmaceutical Research, 24:773). Exemplary SELPs are set forth in Table 6 and contain repeating sequences set forth in any of SEQ ID NOS: 39-57, 73 and 74. In particular, reference to SELPs herein include SELP-27K (SEQ ID NO: 61); SELP-47K (SEQ ID NO:62) and SELP-815K (SEQ ID NO:63).

As used herein, a silk-like unit refers to a sequence of amino acids found naturally in silk fibroid protein and that promote protein crystallization by permitting formation of hydrogen bonds. Exemplary of such sequences are GAGAGS (SEQ ID NO:26) or SGAGAG (SEQ ID NO:27). Reference to silk-like units also include variants thereof that effect or influence hydrogen bond formation, and hence gelation, of the protein polymer.

As used herein, an elastin-like unit refers to a sequence of amino acids found in naturally occurring elastin and that influence water solublility. Exemplary of such sequences are GVGVP (SEQ ID NO.29), VPGG (SEQ ID NO:30), APGVGV (SEQ ID NO:31), or VPGVG (SEQ ID NO:32). Reference to elastin-like units also include variants thereof that confer or influence aqueous solubility of the protein polymer. Exemplary of such variants are GXGVP (SEQ ID NO:35) or VPGXG (SEQ ID NO:36), such as VPGKG (SEQ ID NO:37) or GKGVP (SEQ ID NO:38).

A “variant” with reference to a silk-like unit or elastin-like unit refers to a silk-like unit or elastin-like unit that has an amino acid sequence that is altered by one or more amino acids. Typically, a unit sequence is altered by 1, 2 or 3 amino acids. The variant can have an amino acid replacement(s), deletions or insertions. For example, the variant can have “conservative” changes, wherein a substituted amino acid has similar structural or chemical properties (e.g. replacement of leucine with isoleucine). Exemplary conservative amino acid substitutions are set forth in Table 1. In some cases, a variant can have “nonconservative” changes (e.g., replacement of a glycine with a tryptophan). Similar minor variations can also include amino acid deletions or insertions, or both. In addition to the teaching herein, guidance in determining which amino acid residues can be substituted, inserted, or deleted without abolishing bioactivity can be found using computer programs well known in the art, for example, DNASTAR software.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in TABLE 1 as follows:

TABLE 1

| Original residue | Exemplary conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, reference to physiologic temperature refers to the body temperature of a subject, and generally is or is about 37.0° C.±0.5° C., such as or about 37.0° C.

As used herein, a “vaccinia in protein polymer composition” or “VV-protein polymer” refers to a vaccinia virus that is contained in a protein polymer hydrogel matrix when formed. For purposes herein, since liquid forms are capable of transitioning to a hydrogel form, reference to a VV-protein polymer refers to both liquid and non-liquid forms of the composition.

As used herein, a “vaccinia in SELP composition” or “VV-SELP” or “LIVP-SELP” refers to a vaccinia virus, such as an LIVP, that is contained in a SELP hydrogel matrix when formed. For purposes herein, since liquid forms are capable of transitioning to a hydrogel form, reference to a VV-SELP or LIVP-SELP refers to both liquid and non-liquid forms of the composition.

As used herein, “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

As used herein, plaque forming unit (pfu) or infectious unit (IU) refers to the number of infectious or live viruses. It thus reflects the amount of active virus in the preparation. The pfu can be determined using a plaque formation assay or an end-point dilution assay, which are standard assays known to one of skill in the art.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein the term “vaccinia virus” or “VACV” or “VV” denotes a large, complex, enveloped virus belonging to the poxvirus family. It has a linear, double-stranded DNA genome approximately 190 kbp in length, and which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health vaccinia virus strains.

As used herein, Lister Strain of the Institute of Viral Preparations (LIVP) or LIVP virus strain refers to a virus strain that is the attenuated Lister strain (ATCC Catalog No. VR-1549) that was produced by adaption to calf skin at the Institute of Viral Preparations, Moscow, Russia (Al'tshtein et al. (1985) Dokl. Akad. Nauk USSR 285:696-699). The LIVP strain can be obtained, for example, from the Institute of Viral Preparations, Moscow, Russia (see. e.g., Kutinova et al. (1995) Vaccine 13:487-493); the Microorganism Collection of FSRI SRC VB Vector (Kozlova et al. (2010) Environ. Sci. Technol. 44:5121-5126); or can be obtained from the Moscow Ivanovsky Institute of Virology (C0355 K0602; Agranovski et al. (2006) Atmospheric Environment 40:3924-3929). It also is well known to those of skill in the art; it was the vaccine strain used for vaccination in the USSR and throughout Asia and India. The strain now is used by researchers and is well known (see e.g., Altshteyn et al. (1985) Dokl. Akad. Nauk USSR 285:696-699; Kutinova et al. (1994) Arch. Virol. 134:1-9; Kutinova et al. (1995) Vaccine 13:487-493; Shchelkunov et al. (1993) Virus Research 28:273-283; Sroller et al. (1998) Archives Virology 143:1311-1320; Zinoviev et al., (1994) Gene 147:209-214; and Chkheidze et al. (1993) FEBS 336:340-342). Among the LIVP strains is one that contains a genome having a sequence of nucleotides set forth in SEQ ID NO:1, or a sequence that is at least or at least about 97%, 98% or 99% identical to the sequence of nucleotides set forth in SEQ ID NO:1. An LIVP virus strain encompasses any virus strain or virus preparation that is obtained by propagation of LIVP through repeat passage in cell lines.

As used herein, an LIVP clonal strain or LIVP clonal isolate refers to a virus that is derived from the LIVP virus strain by plaque isolation, or other method in which a single clone is propagated, and that has a genome that is homogenous in sequence. Hence, an LIVP clonal strain includes a virus whose genome is present in a virus preparation propagated from LIVP. An LIVP clonal strain does not include a recombinant LIVP virus that is genetically engineered by recombinant means using recombinant DNA methods to introduce heterologous nucleic acid. In particular, an LIVP clonal strain has a genome that does not contain heterologous nucleic acid that contains an open reading frame encoding a heterologous protein. For example, an LIVP clonal strain has a genome that does not contain non-viral heterologous nucleic acid that contains an open reading frame encoding a non-viral heterologous protein. As described herein, however, it is understood that any of the LIVP clonal strains provided herein can be modified in its genome by recombinant means to generate a recombinant virus. For example, an LIVP clonal strain can be modified to generate a recombinant LIVP virus that contains insertion of nucleotides that contain an open reading frame encoding a heterologous protein.

As used herein, LIVP 1.1.1 is an LIVP clonal strain that has a genome having a sequence of nucleotides set forth in SEQ ID NO:2, or a genome having a sequence of nucleotides that has at least 97%, 98%, or 99% sequence identity to the sequence of nucleotides set forth in SEQ ID NO:2.

As used herein, LIVP 2.1.1 is an LIVP clonal strain that has a genome having a sequence of nucleotides set forth in SEQ ID NO:3, or a genome having a sequence of nucleotides that has at least 97%, 98%, or 99% sequence identity to the sequence of nucleotides set forth in SEQ ID NO:3.

As used herein, LIVP 4.1.1 is an LIVP clonal strain that has a genome having a sequence of nucleotides set forth in SEQ ID NO:4, or a genome having a sequence of nucleotides that has at least 97%, 98% or 99% sequence identity to the sequence of nucleotides set forth in SEQ ID NO:4.

As used herein, LIVP 5.1.1 is an LIVP clonal strain that has a genome having a sequence of nucleotides set forth in SEQ ID NO:5, or a genome having a sequence of nucleotides that has at least 97%, 98% or 99% sequence identity to the sequence of nucleotides set forth in SEQ ID NO:5.

As used herein, LIVP 6.1.1 is an LIVP clonal strain that has a genome having a sequence of nucleotides set forth in SEQ ID NO:6, or a genome having a sequence of nucleotides that has at least 97%, 98% or 99% sequence identity to the sequence of nucleotides set forth in SEQ ID NO:6.

As used herein, LIVP 7.1.1 is an LIVP clonal strain that has a genome having a sequence of nucleotides set forth in SEQ ID NO:7, or a genome having a sequence of nucleotides that has at least 97%, 98% or 99% sequence identity to the sequence of nucleotides set forth in SEQ ID NO:7.

As used herein, LIVP 8.1.1 is an LIVP clonal strain that has a genome having a sequence of nucleotides set forth in SEQ ID NO:8, or a genome having a sequence of nucleotides that has at least 97%, 98% or 99% sequence identity to the sequence of nucleotides set forth in SEQ ID NO:8.

As used herein, the term “modified virus” refers to a virus that is altered compared to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one or more heterologous nucleic acid sequences in the form of a gene expression cassette for the expression of a heterologous gene.

As used herein, a modified LIVP virus strain refers to an LIVP virus that has a genome that is not contained in LIVP, but is a virus that is produced by modification of a genome of a strain derived from LIVP. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein. For example, modified viruses herein can contain one or more heterologous nucleic acid sequences in the form of a gene expression cassette for the expression of a heterologous gene.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, “production by recombinant methods” or “methods using recombinant DNA methods” or variations thereof refers to the use of the well known methods of molecular biology for expressing proteins encoded by cloned DNA.

As used herein a “gene expression cassette” or “expression cassette” is a nucleic acid construct, containing nucleic acid elements that are capable of effecting expression of a gene in hosts that are compatible with such sequences. Expression cassettes include at least promoters and optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acid include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, a heterologous protein or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter also can be a hybrid promoter composed of different elements derived from different native promoters.

As used herein, the term, “therapeutic gene product” or “therapeutic polypeptide” or “therapeutic agent” refers to any heterologous protein expressed by the therapeutic virus that ameliorates the symptoms of a disease or disorder or ameliorates the disease or disorder. Therapeutic agents include, but are not limited to, moieties that inhibit cell growth or promote cell death, that can be activated to inhibit cell growth or promote cell death, or that activate another agent to inhibit cell growth or promote cell death. Optionally, the therapeutic agent can exhibit or manifest additional properties, such as, properties that permit its use as an imaging agent, as described elsewhere herein. Exemplary therapeutic agents include, for example, cytokines, growth factors, photosensitizing agents, radionuclides, toxins, anti-metabolites, signaling modulators, anti-cancer antibiotics, anti-cancer antibodies, angiogenesis inhibitors, chemotherapeutic compounds or a combination thereof.

As used herein, a “reporter gene” is a gene that encodes a reporter molecule that can be detected when expressed by a virus provided herein or encodes a molecule that modulates expression of a detectable molecule, such as a nucleic acid molecule or a protein, or modulates an activity or event that is detectable. Hence reporter molecules include, nucleic acid molecules, such as expressed RNA molecules, and proteins.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured. Detectable labels can be used to image one or more of any of the viruses provided herein. Detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition. Exemplary detectable labels include, for example, metals such as colloidal gold, iron, gadolinium, and gallium-67, fluorescent moieties, and radionuclides. Exemplary fluorescent moieties and radionuclides are provided elsewhere herein.

As used herein, LIVP GLV-1h68 is an LIVP virus that contains-ruc-gfp (a luciferase and green fluorescent protein fusion gene (see e.g. U.S. Pat. No. 5,976,796), beta-galactosidase (LacZ) and beta-glucuronidase (gusA) reporter genes inserted into the F14.5L, J2R (thymidine kinase) and A56R (hemagglutinin) loci, respectively. The genome of GLV-1h68 has a sequence of nucleotides set forth in SEQ ID NO:9, or a sequence of nucleotides that has at least 97%, 98% or 99% sequence identity to the sequence of nucleotides set forth in SEQ ID NO:9.

As used herein, a virus preparation or virus composition, for example an LIVP virus preparation, refers to a virus composition obtained by propagation of a virus strain, for example an LIVP virus strain, an LIVP clonal strain or a modified or recombinant virus strain, in vivo or in vitro in a culture system. For example, an LIVP virus preparation refers to a viral composition obtained by propagation of a virus strain in host cells, typically upon purification from the culture system using standard methods known in the art. A virus preparation generally is made up of a number of virus particles or virions. If desired, the number of virus particles in the sample or preparation can be determined using a plaque assay to calculate the number of plaque forming units per sample unit volume (pfu/mL), assuming that each plaque formed is representative of one infective virus particle. Each virus particle or virion in a preparation can have the same genomic sequence compared to other virus particles (i.e. the preparation is homogenous in sequence) or can have different genomic sequences (i.e. the preparation is heterogenous in sequence). It is understood to those of skill in the art that, in the absence of clonal isolation, heterogeneity or diversity in the genome of a virus can occur as the virus resproduces, such as by homologous recombination events that occur in the natural selection processes of virus strains (Plotkin & Orenstein (eds) “Recombinant Vaccinia Virus Vaccines” in Vaccines, 3^{rd} edition (1999)).

As used herein, a nanoparticle refers to a colloidal particle for delivery of a molecule or agent that is microscopic in size of between or about between 1 and 1000 nanometers (nm), such as 1 and 100 nm, and that behave as a whole unit in terms of transport and properties. Nanoparticles include those that are uniform in size. Nanoparticles include those that contain a targeting molecule attached to the outside.

As used herein, “targeting molecule” or “targeting ligand” refers to any molecular signal directing localization to specific cells, tissues or organs. Examples of targeting ligands include, but are not limited to, protein, polypeptide or portions thereof that bind to cell surface molecules, including, but not limited to, proteins, carbohydrates, lipids or other such moiety. For example, targeting ligands include proteins or portions thereof that bind to cell surface receptors or antibodies directed to antigens expressed selectively on a target cell. Targeting ligands include, but are not limited to growth factors, cytokines, adhesion molecules, neuropeptides, protein hormones and single-chain antibodies (scFv).

As used herein, a delivery vehicle for administration refers to a lipid-based or other polymer-based composition, such as liposome, micelle or reverse micelle, that associates with an agent, such as a virus provided herein, for delivery into a host subject.

As used herein, accumulation of a virus in a particular tissue refers to the distribution or colonization of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s), the immunocompetence of the host and dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect.

As used herein, activity refers to the in vitro or in vivo activities of a compound or virus provided herein. For example, in vivo activities refer to physiological responses that result following in vivo administration thereof (or of a composition or other mixture). Activity, thus, encompasses resulting therapeutic effects and pharmaceutical activity of such compounds, compositions and mixtures. Activities can be observed in in vitro and/or in vivo systems designed to test or use such activities.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, a “parameter indicative of anti-tumor activity or anti-tumorigenic activity” refers to a property mediated by a virus that is associated with anti-tumor activity. Parameters indicative of anti-tumor activity can be assessed in vitro or in vivo upon administration to a subject. Exemplary parameters indicative of anti-tumor activity include, but are not limited to, infectivity of tumor cells, accumulation of virus in tumor tissues, viral nucleic acid replication in tumor cells, virus production in tumor cells, viral gene expression in tumor cells, cytotoxicity of tumor cells, tumor cell selectivity, tumor cell type selectivity, decreased tumor size, increased tumor volume, decreased tumor weight, and initiation of specific and nonspecific anti-tumor immune responses. Assays that assess any of the above parameters or other anti-tumorigenic properties are known to one of skill in the art. Exemplary assays are described herein. Hence, a virus that exhibits any one or more of the above activities or properties exhibits anti-tumor activity.

As used herein, “toxicity” (also referred to as virulence or pathogenicity herein) with reference to a virus refers to the deleterious or toxic effects to a host upon administration of the virus. For an oncolytic virus, such as LIVP, the toxicity of a virus is associated with its accumulation in non-tumorous organs or tissues, which can impact the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on weight.

As used herein, a “parameter indicative of toxicity” refers to a property mediated by a virus that is associated with its toxicity, virulence or pathogenicity. Parameters indicative of toxicity generally are assessed in vivo upon administration to a subject. Exemplary parameters indicative of toxicity include, but are not limited to, decreased survival of the subject, decreased body weight, fever, rash, allergy, fatigue, abdominal pain, induction of an immune response in the subject and pock formation. Assays or measures that assess any of the above parameters or other toxic properties known to one of skill in the art are described herein or are known to one of skill in the art. Hence, a virus that mediates any one or more of the above activities or properties in a host exhibits some degree of toxicity.

As used herein, nucleic acids include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem. 243:3557-3559 (1968), and adopted 37 C.F.R. §§1.821-1.822, abbreviations for amino acid residues are shown in Table 1:

TABLE 2
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 2) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, recitation that nucleotides or amino acids “correspond to” nucleotides or amino acids in a disclosed sequence, such as set forth in the Sequence Listing, refers to nucleotides or amino acids identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g. terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences (or any two polypeptides have amino acid sequences) that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wild/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Generally, when comparing nucleotide sequences herein, an alignment with no penalty for end gaps (e.g. terminal gaps are not penalized) is used.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues and other tissues and cells involved in other proliferative disorders, wounds and other tissues involved in inflammatory responses.

As used herein, a wound or lesion refers to any damage to any tissue in a living organism. The tissue can be an internal tissue, such as the stomach lining or a bone, or an external tissue, such as the skin. As such, a wound or lesion can include, but is not limited to, a gastrointestinal tract ulcer, a broken bone, a neoplasia, and cut or abraded skin. A wound or lesion can be in a soft tissue, such as the spleen, or in a hard tissue, such as bone. The wound or lesion can have been caused by any agent, including traumatic injury, infection or surgical intervention.

As used herein, a skin lesion refers to a lesion on the surface of the skin. The skin lesion can be have been caused by a traumatic injury, infection, surgical intervention or an environmental factor. Exemplary of skin lesions include, but are not limited to, precancerous lesion (e.g. actinic keratosis of the skin), a cancerous lesion (e.g. skin cancer), a traumatic wound (e.g. burn or scar) or a post-surgical wound (e.g. surgically resected tumor). In particular, the lesion is a skin cancer lesion such as basal cell carcinoma or squamous cell carcinoma.

As used herein, a tumor, also known as a neoplasm, is an abnormal mass of tissue that results when cells proliferate at an abnormally high rate. Tumors can show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and blastic tumors.

As used herein, a resected tumor refers to a tumor in which a significant portion of the tumor has been excised. The excision can be effected by surgery (i.e. surgically resected tumor). The resection can be partial or complete.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, proliferative disorders or hyperproliferative disorders include any disorders involving abnormal proliferation of cells. Such disorders include, but are not limited to, neoplastic diseases, inflammatory responses and disorders, e.g. including wound healing responses, psoriasis, restenosis, macular degeneration, diabetic retinopathies, endometriosis, benign prostatic hypertrophy, hypertrophic scarring, cirrhosis, proliferative vitreoretinopathy, retinopathy of prematurity, and immunoproliferative diseases or disorders, e.g. inflammatory bowel disease, rheumatoid arthritis, systemic lupus erythematosus (SLE) and vascular hyperproliferation secondary to retinal hypoxia or vasculitis.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenström's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, an anticancer agent or compound (used interchangeably with “antitumor or antineoplastic agent”) refers to any agents, or compounds, used in anticancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Anticancer agents include antimetastatic agents. Exemplary anticancer agents include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. As used herein, reference to an anticancer or chemotherapeutic agent includes combinations or a plurality of anticancer or chemotherapeutic agents unless otherwise indicated.

As used herein, a “chemosensitizing agent” is an agent which modulates, attenuates, reverses, or affects a cell's or organism's resistance to a given chemotherapeutic drug or compound. The terms “modulator”, “modulating agent”, “attenuator”, “attenuating agent”, or “chemosensitizer” can be used interchangeably to mean “chemosensitizing agent.” In some examples, a chemosensitizing agent can also be a chemotherapeutic agent. Examples of chemosensitizing agents include, but are not limited to, radiation, calcium channel blockers (e.g., verapamil), calmodulin inhibitors (e.g., trifluoperazine), indole alkaloids (e.g., reserpine), quinolines (e.g., quinine), lysosomotropic agents (e.g., chloroquine), steroids (e.g., progesterone), triparanol analogs (e.g., tamoxifen), detergents (e.g., Cremophor EL), texaphyrins, and cyclic antibiotics (e.g., cyclosporine).

As used herein, a subject includes any organism, including an animal for whom diagnosis, screening, monitoring or treatment is contemplated. Animals include mammals such as primates and domesticated animals. An exemplary primate is human. A patient refers to a subject, such as a mammal, primate, human, or livestock subject afflicted with a disease condition or for which a disease condition is to be determined or risk of a disease condition is to be determined.

As used herein, a patient refers to a human subject exhibiting symptoms of a disease or disorder.

As used herein, treatment of a subject that has a condition, disorder or disease means any manner of treatment in which the symptoms of the condition, disorder or disease are ameliorated or otherwise beneficially altered. Treatment encompasses any pharmaceutical use of the viruses described and provided herein.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, treatment of a wound refers to any manner of treatment in which the signs or symptoms of having a wound are ameliorated or otherwise beneficially altered. Typically, treatment encompasses alleviation of the wound, shrinkage of the wound, reduction in the size of the wound or other similar result that is associated with wound healing.

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, efficacy means that upon administration of a virus or virus composition, the virus will colonize proliferating or immunoprivileged cells, such as tumor cells, and replicate. Colonization and replication in tumor cells is indicative that the treatment is or will be an effective treatment.

As used herein, effective treatment with a virus is one that can increase survival compared to the absence of treatment therewith. For example, a virus is an effective treatment if it stabilizes disease, causes tumor regression, decreases severity of disease or slows down or reduces metastasizing of the tumor.

As used herein, a composition refers to any mixture. It can be a solution, suspension, liquid, gel, powder, paste, aqueous, non-aqueous or any combination thereof.

As used herein, a formulation refers to a composition containing at least one active pharmaceutical or therapeutic agent and one or more excipients.

As used herein, a co-formulation refers to a composition containing two or more active or pharmaceutical or therapeutic agents and one or more excipients.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, direct administration refers to administration of a composition without dilution.

As used herein, a kit is a packaged combination, optionally, including instructions for use of the combination and/or other reactions and components for such use.

As used herein, an “article of manufacture” is a product that is made and sold. As used throughout this application, the term is intended to encompass articles containing a vaccinia virus and protein polymer (e.g. SELP) contained in the same or separate articles of packaging.

As used herein, a device refers to a thing made or adapted for a particular task. Exemplary of devices herein are devices that cover or coat or are capable of contacting the epidermis or surface of the skin. Examples of such devices include, but are not limited to, a wrap, bandage, bind, dress, suture, patch, gauze or dressing.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Hence, “about 5 milliliters” means “about 5 milliliters” and also “5 milliliters.” Generally “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow.

### B. VIRUS POLYMER COMPOSITIONS AND METHODS OF VIRAL DELIVERY AND TREATMENT

Provided herein are compositions containing an oncolytic vaccinia virus in protein polymer (VV-protein polymer), such as in SELP polymer (e.g. VV-SELP), and in particular LIVP-SELP compositions. The VV-protein polymer or VV-SELP compositions, for example LIVP-SELP compositions, are stable at physiologic temperatures (e.g. 34° C. to 37° C.) for at least one week and up to four weeks or more, and are stable at room temperature for even longer. By virtue of the increased stability of the oncolytic virus, the stable VV-SELP compositions can be used for topical delivery to mucosal surfaces for the treatment of wounds, tumors or resected tumors or other hyperproliferative lesions. In addition, the VV-SELP compositions, including LIVP-SELP compositions, also can be used in methods of administration of virus, for example by intravenous administration, to increase virus delivery to proliferating cells or tissues, including immunoprivileged cells and tissues, for examples tumors, wounds or other proliferating cells or tissues.

1. Vaccinia Viruses

Vaccinia viruses are oncolytic viruses that possess a variety of features that make them particularly suitable for use in wound and cancer gene therapy. For example, vaccinia is a cytoplasmic virus, thus, it does not insert its genome into the host genome during its life cycle. Unlike many other viruses that require the host's transcription machinery, vaccinia virus can support its own gene expression in the host cell cytoplasm using enzymes encoded in the viral genome. Vaccinia viruses also have a broad host and cell type range. In particular vaccinia viruses can accumulate in immunoprivileged cells or immunoprivileged tissues, including tumors and/or metastases, and also including wounded tissues and cells. Yet, unlike other oncolytic viruses, vaccinia virus can typically be cleared from the subject to whom the viruses are administered by activity of the subject's immune system, and hence are less toxic than other viruses such as adenoviruses. Thus, while the viruses can typically be cleared from the subject to whom the viruses are administered by activity of the subject's immune system, viruses can nevertheless accumulate, survive and proliferate in immunoprivileged cells and tissues such as tumors because such immunoprivileged areas are sequestered from the host's immune system.

Vaccinia viruses also can be easily modified by insertion of heterologous genes. This can result in the attenuation of the virus and/or permit delivery of therapeutic proteins. For example, the vaccinia virus genome has a large carrying capacity for foreign genes, where up to 25 kb of exogenous DNA fragments (approximately 12% of the vaccinia genome size) can be inserted. The genomes of several of the vaccinia strains have been completely sequenced, and many essential and nonessential genes identified. Due to high sequence homology among different strains, genomic information from one vaccinia strain can be used for designing and generating modified viruses in other strains. Finally, the techniques for production of modified vaccinia strains by genetic engineering are well established (Moss, Curr. Opin. Genet. Dev. 3 (1993), 86-90; Broder and Earl, Mol. Biotechnol. 13 (1999), 223-245; Timiryasova et al., Biotechniques 31 (2001), 534-540).

Various vaccina viruses have been demonstrated to exhibit antitumor activities. In one study, for example, nude mice bearing nonmetastatic colon adenocarcinoma cells were systemically injected with a WR strain of vaccinia virus modified by having a vaccinia growth factor deletion and an enhanced green fluorescence protein inserted into the thymidine kinase locus. The virus was observed to have antitumor effect, including one complete response, despite a lack of exogenous therapeutic genes in the modified virus (McCart et al. (2001) Cancer Res 1:8751-8757). In another study, vaccinia melanoma oncolysate (VMO) was injected into sites near melanoma positive lymph nodes in a Phase III clinical trial of melanoma patients. As a control, New York City Board of Health strain vaccinia virus (VV) was administered to melanoma patients. The melanoma patients treated with VMO had a survival rate better than that for untreated patients, but similar to patients treated with the VV control (Kim et al. (2001) Surgical Oncol 10:53-59). LIVP strains of vaccinia virus also have been used for the diagnosis and therapy of tumors, and for the treatment of wounded and inflamed tissues and cells (see e.g. Zhang et al. (2007) Surgery, 142:976-983; Lin et al. (2008) J. Clin. Endocrinol., Metab., 93:4403-7; Kelly et al. (2008) Hum gene There., 19:774-782; Yu et al. (2009) Mol Cancer Ther., 8:141-151; Yu et al. (2009) Mol Cancer, 8:45; U.S. Pat. No. 7,588,767; U.S. Pat. No. 8,052,968; and U.S. Published application No. US20040234455). For example, when intravenously administered, LIVP strains have been demonstrated to accumulate in internal tumors at various loci in vivo, and have been demonstrated to effectively treat human tumors of various tissue origin, including, but not limited to, breast tumors, thyroid tumors, pancreatic tumors, metastatic tumors of pleural mesothelioma, squamous cell carcinoma, lung carcinoma and ovarian tumors. LIVP strains of vaccinia, including attenuated forms thereof, exhibit less toxicity than WR strains of vaccinia virus, and results in increased and longer survival of treated tumor-bearing animal models (see e.g. U.S. Published Patent Appl. No. US20110293527).

2. Delivery of Vaccinia Viruses

For the treatment of immunoprivileged cells or immunoprivileged tissues, including tumors and/or metastases, and wounded tissues and cells, vaccinia viruses are typically administered by direct intratumoral injection, intraperitoneal injection or by intravenous injection. In particular, due to the lower toxicity of vaccinia virus compared to other oncolytic viruses, such as adenovirus, vaccinia virus strains can be administered intravenously. This is advantageous, since intravenous administration permits a bolus of virus to be injected into the bloodstream for rapid dissemination throughout the subject to the circulatory system. The virus is able to access and accumulate in the immunoprivileged cells or tissues, including to tumor metastases. Since the treatment is not localized to direct injection of a tumor or wounded or inflamed tissue, intravenous administration generally is a more potent route of administration than other injection routes.

3. SELP Compositions

Silk-elastinlike polymers (SELPs) are hydrogel polymers that exhibit pore size and gelation properties in vivo that permits the distribution of and controls release of bioactive agents contained therein. SELPs also have been used for the intratumoral delivery of adenovirus (see e.g. Gustafson et al. (2010) Mol Pharm, 7:1050-1056). Unlike other polymer delivery systems that alter surface functionalization of virus, SELPs do not interfere with viral cell transduction.

For delivery of virus, SELPs have been used to limit the systemic exposure of the virus to the immune system (Gustafson et al. (2010) Mol Pharm, 7:1050-1056). For example, Gustafason et al. shows that a problem even with direct intratumoral injection of adenvovirus, is that there is still some unwanted systemic exposure. The study demonstrated that administration of the virus in a SELP matrix decreased systemic exposure of virus when delivered intratumorally, controlled release of the virus at the injection site, and thereby led to localized, prolonged and increased overall gene expression levels at the site of interest.

Unlike other viruses that are limited by systemic delivery, as noted above vaccinia virus can be delivered systemically. While SELPs have been previously used to limit systemic exposure of virus, it is found herein that SELPs can increase systemic viral delivery upon intravenous administration. This is advantageous for delivery of vaccinia virus, which is a virus that exhibits little toxicity when delivered systemically. For example, while intravenous delivery of vaccinia virus, such as LIVP strains, is effective for treating tumors, it is found herein that intravenous delivery of vaccinia virus can be improved in the presence of SELP. As shown herein, this results in increased delivery of the virus to tumors, including increased tumor cell infectivity and replication efficiency. As shown herein, this result is not achieved by administration of VV-SELP by intratumoral direct injection. The VV-SELP compositions, such as LIVP-SELP compositions, can be delivered intravenously to effect a more potent and robust treatment of immunoprivileged cells or immunoprivileged tissues, including tumors and/or metastases, and wounded tissues and cells, than achieved by intravenous delivery of virus alone. Accordingly, VV-SELP compositions formulated for intravenous administration can be administered at lower dosages and/or at a lesser frequency than virus alone, which can further limit any toxicity issues of the already safe viruses. The increased delivery and infectivity of target cells and tissues achieved by SELPs also can result in an increased survival or therapeutic efficacy than an equivalent dosage of virus alone.

Besides exhibiting properties that increase delivery and infectivity of virus upon systemic delivery, it is also found herein that SELPs increase the stability of virus. For example, it is shown herein that an exemplary vaccinia virus LIVP strain exhibits a rapid decline of infectious particles over time at physiologic temperature of 37° C. For example, exposure of the virus to 37° C. resulted in an almost 70% decrease in the number of infectious plaque forming units within 12 hours, to less than 1% within one week, and no detectable infectious particles present after more than one week. In contrast, exposure of the same LIVP strain in a SELP matrix to 37° C. dramatically increased stability of the virus, such that a significant percentage of viral particles were viable at one week and remained viable for up to 4 weeks at 37° C.

The increased stability of vaccinia virus afforded by SELP has applications for stable storage of vaccinia virus (e.g. a LIVP) in polymer (e.g. SELP) compositions. In addition, increased stability of vaccinia virus in polymer also permits particular delivery applications that are not achievable with non-polymer conjugated virus. For example, stable VV-SELP compositions can be used for topical applications. This includes, for example, topical delivery to treat wounds, a hyperproliferative lesion, such as a carcinoma, or a resected tumors. The VV-SELP compositions can be applied to wounds, lesions or tumors directly or can be applied in a bandage, films, strips or patches.

For example, one of the problems with intratumoral or direct injection of virus is the difficulty of the virus to adequately distribute throughout the tumor mass. It is found herein that topical delivery of virus to a tumor bed immediately following surgical resection is a method to achieve delivery of virus to low, rather than high, volume tumors. This can overcome problems associated with inadequate distribution of virus in tumors. For example, it is demonstrated herein that intraoperative, direct application of VV-SELP to a resected tumor that represents a low volume residual disease optimizes viral delivery and tumor penetration. Thus, such a method can be used in conjunction with methods where a tumor is removed by surgical resection removing the majority of disease, but where residual disease remains.

The following sections describe exemplary vaccinia viruses (e.g. LIVP and strains thereof) and protein polymers (e.g. SELPs) for preparation of the VV-polymer compositions provided herein, in particular VV-SELP compositions. Exemplary articles of manufacture and methods using the VV-polymer, such as VV-SELP, compositions also are described.

### C. VACCINIA VIRUSES AND LIVP

Provided herein are compositions containing a vaccinia virus in a protein polymer. Vaccinia is a cytoplasmic virus, thus, it does not insert its genome into the host genome during its life cycle. Vaccinia virus has a linear, double-stranded DNA genome of approximately 180,000 base pairs in length that is made up of a single continuous polynucleotide chain (Baroudy et al. (1982) Cell, 28:315-324). The structure is due to the presence of 10,000 base pair inverted terminal repeats (ITRs). The ITRs are involved in genome replication. Genome replication is believed to involve self-priming, leading to the formation of high molecular weight concatemers (isolated from infected cells) which are subsequently cleaved and repaired to make virus genomes. See, e.g., Traktman, P., Chapter 27, Poxvirus DNA Replication, pp. 775-798, in DNA Replication in Eukaryotic Cells, Cold Spring Harbor Laboratory Press (1996). The genome encodes for approximately 250 genes. In general, the nonsegmented, noninfectious genome is arranged such that centrally located genes are essential for virus replication (and are thus conserved), while genes near the two termini effect more peripheral functions such as host range and virulence. Vaccinia viruses practice differential gene expression by utilizing open reading frames (ORFs) arranged in sets that, as a general principle, do not overlap.

Vaccinia virus possesses a variety of features for use in cancer gene therapy and vaccination including broad host and cell type range, and low toxicity. For example, while most oncolytic viruses are natural pathogens, vaccinia virus has a unique history in its widespread application as a smallpox vaccine that has resulted in an established track record of safety in humans. Toxicities related to vaccinia administration occur in less than 0.1% of cases, and can be effectively addressed with immunoglobulin administration. In addition, vaccinia virus possesses a large carrying capacity for foreign genes (up to 25 kb of exogenous DNA fragments (approximately 12% of the vaccinia genome size) can be inserted into the vaccinia genome), high sequence homology among different strains for designing and generating modified viruses in other strains, and techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). Vaccinia virus strains have been shown to specifically colonize solid tumors, while not infecting other organs (see, e.g., Zhang et al. (2007) Cancer Res 67:10038-10046; Yu et al., (2004) Nat Biotech 22:313-320; Heo et al., (2011) Mol Ther 19:1170-1179; Liu et al. (2008) Mol Ther 16:1637-1642; Park et al., (2008) Lancet Oncol, 9:533-542). A variety of vaccinia virus strains are available for the compositions herein, including Western Reserve (WR) (SEQ ID NO: 10), Copenhagen (SEQ ID NO: 11), Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary of known viruses are set forth in Table 3A. Exemplary of vaccinia viruses for use in the methods provided herein include, but are not limited to, Lister strain or LIVP strain of vaccinia viruses or modified forms thereof. LIVP exhibits less virulence than the WR strain. Also, for example, a recombinant derivative of LIVP, designated GLV-1h68 (set forth in SEQ ID NO:9; GenBank Acc. No. EU410304) and GLV-1h64 (set forth in SEQ ID NO:18) exhibit tumor targeting properties and an improved safety profile compared to its parental LIVP strain (set forth in SEQ ID NO:1) and the WR strain (Zhang et al. (2009) Mol. Genet. Genomics, 282:417-435).

TABLE 3A

| | | Reference (e.g. GenBank |
|---|---|---|
| Name | Abbreviations | Accession No.) |
| Vaccinia virus strain Western | WR | AY243312 |
| Reserve | | |
| Vaccinia virus strain | COP | M35027 |
| Copenhagen | | |
| Vaccinia Lister major strain | LIST | AY678276 |
| Vaccinia Lister isolate | LC | AY678277 |
| LC16MO | | |
| Vaccinia Lister clone | VACV107 | DQ121394 |
| VACV107 | | |
| Vaccinia virus strain | ACAM | AY313847 |
| ACAM2000 | | |
| Vaccinia virus strain DUKE | DUKE | DQ439815; Li et al. |
| | | (2006) Virology J, 3: 88 |
| Vaccinia virus strain Ankara | MVA | U94848 |
| Vaccinia virus Clone3 | CLONE3 | AY138848 |

1. Lister and LIVP Strains

Exemplary vaccinia viruses are Lister or LIVP vaccinia viruses. Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression.

The vaccinia virus in the compositions provided herein can be based on modifications to the Lister strain of vaccinia virus. LIVP is a vaccinia strain derived from Lister (ATCC Catalog No. VR-1549). As described elsewhere herein, the LIVP strain can be obtained from the Lister Institute of Viral Preparations, Moscow, Russia; the Microorganism Collection of FSRI SRC VB Vector; or can be obtained from the Moscow Ivanovsky Institute of Virology (C0355 K0602). The LIVP strain was used for vaccination throughout the world, particularly in India and Russia, and is widely available. LIVP and its production are described, for example, in U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917, 2011/0064650; Zhang et al. (2009) Mol. Genet. Genomics, 282:417-435). A sequence of a parental genome of LIVP is set forth in SEQ ID NO:1.

LIVP strains in the compositions provided herein also include clonal strains that are derived from LIVP and that can be present in a virus preparation propagated from LIVP. The LIVP clonal strains have a genome that differs from the parental sequence set forth in SEQ ID NO:1. The clonal strains provided herein exhibit greater anti-tumorigenicity and/or reduced toxicity compared to the recombinant or modified virus strain designated GLV-1h68 (having a genome set forth in SEQ ID NO:9).

The LIVP and clonal strains have a sequence of nucleotides that have at least 70%, such as at least 75%, 80%, 85% or 90% sequence identity to SEQ ID NO: 1. For example, the clonal strains have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9% or 100% sequence identity to SEQ ID NO: 1. Such LIVP clonal viruses include viruses that differ in one or more open reading frames (ORF) compared to the parental LIVP strain that has a sequence of amino acids set forth in SEQ ID NO: 1. The LIVP clonal virus strains provided herein can contain a nucleotide deletion or mutation in any one or more nucleotides in any ORF compared to SEQ ID NO: 1, or can contain an addition or insertion of viral DNA compared to SEQ NO: 1.

LIVP strains in the compositions provided herein include those that have a nucleotide sequence corresponding to nucleotides 10,073-180,095 of SEQ ID NO:2, nucleotides 11,243-182,721 of SEQ ID NO:3, nucleotides 6,264-181,390 of SEQ ID NO:4, nucleotides 7,044-181,820 of SEQ ID NO:5, nucleotides 6,674-181,409 of SEQ ID NO:6, nucleotides 6,716-181,367 of SEQ ID NO:7 or nucleotides 6,899-181,870 of SEQ ID NO:8, or to a complement thereof. In some examples, the LIVP strain for use in the methods is a clonal strain of LIVP or a modified form thereof containing a sequence of nucleotides that has at least 97%, 98%, 99% or more sequence identity to a sequence of nucleotides 10,073-180,095 of SEQ ID NO:2, nucleotides 11,243-182,721 of SEQ ID NO:3, nucleotides 6,264-181,390 of SEQ ID NO:4, nucleotides 7,044-181,820 of SEQ ID NO:5, nucleotides 6,674-181,409 of SEQ ID NO:6, nucleotides 6,716-181,367 of SEQ ID NO:7 or nucleotides 6,899-181,870 of SEQ ID NO:8. LIVP clonal strains provided herein generally also include terminal nucleotides corresponding to a left and/or right inverted terminal repeat (ITR). Exemplary LIVP strains include but are not limited to virus strains designated LIVP 1.1.1 having a genome containing a sequence of nucleotides set forth in SEQ ID NO: 2 or a sequence of nucleotides that exhibits at least 97% sequence identity to SEQ ID NO:2; a virus strain designated LIVP 2.1.1 having a genome containing a sequence of nucleotides set forth in SEQ ID NO: 3 or a sequence of nucleotides that exhibits at least 97%, 98%, 99% or more sequence identity to SEQ ID NO:3; a virus strain designated LIVP 4.1.1 having a genome containing a sequence of nucleotides set forth in SEQ ID NO: 4 or a sequence of nucleotides that exhibits at least 97%, 98%, 99% or more sequence identity to SEQ ID NO:4; a virus strain designated LIVP 5.1.1 having a genome containing a sequence of nucleotides set forth in SEQ ID NO: 5 or a sequence of nucleotides that exhibits at least 97%, 98%, 99% or more sequence identity to SEQ ID NO:5; a virus strain designated LIVP 6.1.1 having a sequence of nucleotides set forth in SEQ ID NO: 6 or a sequence of nucleotide that exhibits at least 97%, 98%, 99% or more sequence identity to SEQ ID NO:6; a virus strain designated LIVP 7.1.1 having a genome containing a sequence of nucleotides set forth in SEQ ID NO: 7 or a sequence of nucleotides that exhibits at least 97%, 98%, 99% or more sequence identity to SEQ ID NO:7; or a virus strain designated LIVP 8.1.1 having a genome containing a sequence of nucleotides set forth in SEQ ID NO: 8 or a sequence of nucleotides that exhibits at least 97%, 98%, 99% or more sequence identity to SEQ ID NO:8.

2. Heterologous Nucleic Acid and Modified Viruses

The large genome size of poxviruses, such as the vaccinia viruses in the compositions provided herein, allows large inserts of heterologous DNA and/or multiple inserts of heterologous DNA to be incorporated into the genome (Smith and Moss (1983) Gene 25(1):21-28). The vaccinia viruses in the compositions provided herein can be modified by insertion of 1, 2, 3, 4, 5, 6, 7, 8, 9, 10 or more heterologous DNA molecules. Generally, the one or more heterologous DNA molecules are inserted into a non-essential region of the virus genome. For example, the one or more heterologous DNA molecules are inserted into a locus of the virus genome that is non-essential for replication in proliferating cells, such as tumor cells. Exemplary insertion sites are provided herein below and are known in the art.

In some examples, the virus can be modified to express an exogenous or heterologous gene. Exemplary exogenous gene products include proteins and RNA molecules. The modified viruses can express a therapeutic gene product, a detectable gene product, a gene product for manufacturing or harvesting, an antigenic gene product for antibody harvesting, or a viral gene product. The characteristics of such gene products are described herein and elsewhere.

In some examples, the viruses can be modified to express two or more gene products, such as 2, 3, 4, 5, 6, 7, 8, 9, 10 or more gene products, where any combination of the two or more gene products can be one or more detectable gene products, therapeutic gene products, gene products for manufacturing or harvesting or antigenic gene products for antibody harvesting or a viral gene product. In one example, a virus can be modified to express an anticancer gene product. In another example, a virus can be modified to express two or more gene products for detection or two or more therapeutic gene products. In some examples, one or more proteins involved in biosynthesis of a luciferase substrate can be expressed along with luciferase. When two or more exogenous genes are introduced, the genes can be regulated under the same or different regulatory sequences, and the genes can be inserted in the same or different regions of the viral genome, in a single or a plurality of genetic manipulation steps. In some examples, one gene, such as a gene encoding a detectable gene product, can be under the control of a constitutive promoter, while a second gene, such as a gene encoding a therapeutic gene product, can be under the control of an inducible promoter. Methods for inserting two or more genes into a virus are known in the art and can be readily performed for a wide variety of viruses using a wide variety of exogenous genes, regulatory sequences, and/or other nucleic acid sequences.

The heterologous DNA can be an exemplary gene, including any from the list of human genes and genetic disorders authored and edited by Dr. Victor A. McKusick and his colleagues at Johns Hopkins University and elsewhere, and developed for the World Wide Web by NCBI, the National Center for Biotechnology Information; online, Mendelian Inheritance in Man, OMIM™ Center for Medical Genetics, Johns Hopkins University (Baltimore, Md.), and National Center for Biotechnology Information, National Library of Medicine (Bethesda, Md.), 1999; and those available in public databases, such as PubMed and GenBank (see, e.g., (ncbi.nlm.nih.gov/entrez/query.fcgi?db=OMIM).

In particular, viruses provided herein can be modified to express an anti-tumor antibody, an anti-metastatic gene or metastasis suppressor genes; cell matrix degradative genes; hormones; growth factors; immune modulatory molecules, including a cytokine, such as interleukins or interferons, a chemokine, including CXC chemokines, costimulatory molecules; ribozymes; transporter protein; antibody or fragment thereof; antisense RNA; siRNA; microRNAs; protein ligands; a mitosis inhibitor protein; an antimiotic oligopeptide; an anti-cancer polypeptide; anti-cancer antibiotics; angiogenesis inhibitors; anti-angiogenic factors; tissue factors; a prodrug converting enzyme; genes for tissue regeneration and reprogramming human somatic cells to pluripotency; enzymes that modify a substrate to produce a detectable product or signal or are detectable by antibodies; a viral attenuation factors; a superantigen; proteins that can bind a contrasting agent, chromophore, or a compound of ligand that can be detected; tumor suppressors; cytotoxic protein; cytostatic protein; genes for optical imaging or detection including luciferase, a fluorescent protein such as a green fluorescent protein (GFP) or GFP-like protein, a red fluorescent protein (RFP), a far-red fluorescent protein, a near-infrared fluorescent protein, a yellow fluorescent protein (YFP), an orange fluorescent protein (OFP), a cerulean fluorescent proein (CFP), or a blue fluorescent protein (BFP), and phycobiliproteins from certain cyanobacteria and eukaryotic algae, including phycoerythrins (red) and the phycocyanins (blue); genes for PET imaging; genes for MRI imaging; or genes to alter attenuation of the viruses.

a. Exemplary Modifications

Exemplary heterologous genes for modification of viruses herein are known in the art (see e.g. U.S. Pub. Nos. US2003-0059400, US2003-0228261, US2009-0117034, US2009-0098529, US2009-0053244, US2009-0081639 and US2009-0136917; U.S. Pat. Nos. 7,588,767 and 7,763,420; and International Pub. No. WO 2009/139921). A non-limiting description of exemplary genes encoding heterologous proteins for modification of virus strains is set forth in the following table. The sequence of the gene and encoded proteins are known to one of skill in the art from the literature. Hence, provided herein are virus strains, including any of the clonal viruses provided herein, that contain nucleotides encoding any of the heterologous proteins listed in Table 3B.

TABLE 3B
Exemplary Genes and Gene Products

```
Detectable gene products
  Optical Imaging
  Luciferase
```

bacterial luciferase
luciferase (from Vibrio harveyi or Vibrio fischerii)

luxA
luxB
luxC
luxD
luxE
luxAB
luxCD
luxABCDE

firefly luciferase
Renilla luciferase from Renilla renformis
Gaussia luciferase
luciferases found among marine arthropods
luciferases that catalyze the oxidation of Cypridina (Vargula) luciferin
luciferases that catalyze the oxidation of Coleoptera luciferin

luciferase photoproteins

aequorin photoprotein to which luciferin is non-covalently bound

click beetle luciferase

CBG99
CBG99-mRFP1

Fusion Proteins

Ruc-GFP

Fluorescent Proteins
GFP

aequorin from Aequorea victoria
GFP from Aequorea victoria
GFP from Aequorea coerulescens
GFP from the anthozoan coelenterates Renilla reniformis and Renilla kollikeri
(sea pansies)
Emerald (Initrogen, Carlsbad, CA)
EGFP (Clontech, Palo Alto, CA)
Azami-Green (MBL International, Woburn, MA)
Kaede (MBL International, Woburn, MA)
ZsGreen1 (Clontech, Palo Alto, CA)
CopGFP (Evrogen/Axxora, LLC, San Diego, CA)
Anthozoa reef coral
Anemonia sea anemone
Renilla sea pansy
Galaxea coral
Acropora brown coral
Trachyphyllia stony coral
Pectiniidae stony coral
GFP-like proteins

RFP

RFP from the corallimorph Discosoma (DsRed) (Matz et al. (1999) Nature
Biotechnology 17: 969-973)
Heteractis reef coral, Actinia or Entacmaea sea anemone
RFPs from Discosoma variants

mRFP1 (Wang et al. (2004) Proc. Natl. Acad. Sci. U.S.A.101: 16745-9)
mCherry (Wang et al. (2004) PNAS USA.101(48): 16745-9)
tdTomato (Wang et al. (2004) PNAS USA.101(48): 16745-9)
mStrawberry (Wang et al. (2004) PNAS USA.101(48): 16745-9)
mTangerine (Wang et al. (2004) PNAS USA.101(48): 16745-9)
DsRed2 (Clontech, Palo Alto, CA)
DsRed-T1 (Bevis and Glick (2002) Nat. Biotechnol. 20: 83-87)
Anthomedusa J-Red (Evrogen)
Anemonia AsRed2 (Clontech, Palo Alto, CA)

far-red fluorescent protein

TurboFP635
mNeptune monomeric far-red fluorescent protein
Actinia AQ143 (Shkrob et al. (2005) Biochem J. 392(Pt 3): 649-54)
Entacmaea eqFP611 (Wiedenmann et al. (2002) PNAS USA. 99(18): 11646-51)
Discosoma variants

mPlum (Wang et al. (2004) PNAS USA.101(48): 16745-9)
mRasberry (Wang et al. (2004) PNAS USA.101(48): 16745-9)
Heteractis HcRed1 and t-HcRed (Clontech, Palo Alto, CA)

IFP (infrared fluorescent protein)
near-infrared fluorescent protein
YFP

EYFP (Clontech, Palo Alto, CA)
YPet (Nguyen and Daugherty (2005) Nat Biotechnol. 23(3): 355-60)
Venus (Nagai et al. (2002) Nat. Biotechnol. 20(1): 87-90)
ZsYellow (Clontech, Palo Alto, CA)
mCitrine (Wang et al. (2004) PNAS USA.101(48): 16745-9)

OFP

cOFP (Stratagene, La Jolla, CA)
mKO (MBL International, Woburn, MA)
mOrange (Wang et al.. (2004) PNAS USA.101(48): 16745-9)

CFP

Cerulean (Rizzo (2004) Nat Biotechnol. 22(4): 445-9)
mCFP (Wang et al. (2004) PNAS USA.101(48): 16745-9)
AmCyan1 (Clontech, Palo Alto, CA)
MiCy (MBL International, Woburn, MA)
CyPet (Nguyen and Daugherty (2005) Nat Biotechnol. 23(3): 355-60)

BFP

EBFP (Clontech, Palo Alto, CA);
phycobiliproteins from certain cyanobacteria and eukaryotic algae, phycoerythrins
(red) and the phycocyanins (blue)

R-Phycoerythrin (R-PE)
B-Phycoerythrin (B-PE)
Y-Phycoerythrin (Y-PE
C-Phycocyanin (P-PC)
R-Phycocyanin (R-PC)
Phycoerythrin 566 (PE 566)
Phycoerythrocyanin (PEC)
Allophycocyanin (APC)
frp Flavin Reductase
CBP Coelenterazine-binding protein 1
PET imaging

Cyp11B1 transcript variant 1
Cyp11B1 transcript variant 2
Cyp11B2
AlstR
PEPR-1
LAT-4 (SLC43A2)
Cyp51 transcript variant 1
Cyp51 transcript variant 2

Transporter proteins
Solute carrier transporter protein families (SLC)

SLC1 solute carrier 1 transporter protein family
SLC1A1, SLC1A2, SLC1A3, SLC1A4, SLC1A5, SLC1A6, SLC1A7
SLC2 solute carrier 2 transporter protein family
SLC2A1, SLC2A2, SLC2A3, SLC2A4, SLC2A5, SLC2A6, SLC2A7, SLC2A8,
SLC2A9, SLC2A10, SLC2A11, SLC2A12, SLC2A13, SLC2A14)
SLC3 solute carrier 3 transporter protein family
SLC3A1, SLC3A2
SLC 4 solute carrier 4 transporter protein family
SLC4A1, SLC4A2, SLC4A3, SLC4A4, SLC4A5, SLC4A6, SLC4A7, SLC4A8,
SLC4A9, SLC4A10, SLC4A11
SLC5 solute carrier 5 transporter protein family
SLC5A1 sodium/glucose cotransporter 1
SLC5A2 sodium/glucose cotransporter 2
SLC5A3 sodium/myo-inositol cotransporter
SLC5A4 low affinity sodium-glucose cotransporter
SLC5A5 sodium/iodide cotransporter
SLC5A6 sodium-dependent multivitamin transporter
SLC5A7 high affinity choline transporter 1
SLC5A8 sodium-coupled monocarboxylate transporter 1
SLC5A9 sodium/glucose cotransporter 4
SLC5A10 sodium/glucose cotransporter 5, isoform 1
sodium/glucose cotransporter 5, isoform 2
sodium/glucose cotransporter 5, isoform 3
sodium/glucose cotransporter 5, isoform 4
SLC5A11 sodium/myo-inositol cotransporter 2, isoform 1
sodium/myo-inositol cotransporter 2, isoform 2
sodium/myo-inositol cotransporter 2, isoform 3
sodium/myo-inositol cotransporter 2, isoform 4
SLC5A12 sodium-coupled monocarboxylate transporter 2, isoform 1
sodium-coupled monocarboxylate transporter 2, isoform 2

Sodium Iodide Symporter (NIS)
hNIS (NM_000453)
hNIS (BC105049)
hNIS (BC105047)
hNIS (non-functional hNIS variant containing an additional 11 aa)

SLC6 solute carrier 6 transporter protein family
SLC6A1 sodium- and chloride-dependent GABA transporter 1
SLC6A2 norepinephrine transporter (sodium-dependent noradrenaline transporter)
SLC6A3 sodium-dependent dopamine transporter
SLC6A4 sodium-dependent serotonin transporter
SLC6A5 sodium- and chloride-dependent glycine transporter 1
SLC6A6 sodium-and chloride-dependent taurine transporter
SLC6A7 sodium-dependent proline transporter
SLC6A8 sodium- and chloride-dependent creatine transporter
SLC6A9 sodium- and chloride-dependent glycine transporter 1, isoform 1
sodium- and chloride-dependent glycine transporter 1, isoform 2
sodium- and chloride-dependent glycine transporter 1, isoform 3
SLC6A10 sodium- and chloride-dependent creatine transporter 2
SLC6A11 sodium- and chloride-dependent GABA transporter 3
SLC6A12 sodium- and chloride-dependent betaine transporter
SLC6A13 sodium- and chloride-dependent GABA transporter 2
SLC6A14 Sodium- and chloride-dependent neutral and basic amino acid transporter
B(0+)
SLC6A15 Orphan sodium- and chloride-dependent neurotransmitter transporter
NTT73
SLC6A16 Orphan sodium- and chloride-dependent neurotransmitter transporter
NTT5
SLC6A17 Orphan sodium- and chloride-dependent neurotransmitter transporter
NTT4
Sodium SLC6A18 Sodium- and chloride-dependent transporter XTRP2
SLC6A19 Sodium-dependent neutral amino acid transporter B(0)
SLC6A20 Sodium- and chloride-dependent transporter XTRP3
Norepinephrine Transporter (NET)

Human Net (hNET) transcript variant 1 (NM_001172504)
Human Net (hNET) transcript variant 2 (NM_001172501)
Human Net (hNET) transcript variant 3 (NM_001043)
Human Net (hNET) transcript variant 4 (NM_001172502)
Non-Human Net

SLC7 solute carrier 7 transporter protein family
SLC7A1, SLC7A2, SLC7A3, SLC7A4, SLC7A5, SLC7A6, SLC7A7, SLC7A8,
SLC7A9, SLC7A10, SLC7A11, SLC7A13, SLC7A14
SLC8 solute carrier 8 transporter protein family
SLC8A1, SLC8A2, SLC8A3
SLC9 solute carrier 9 transporter protein family
SLC9A1, SLC9A2, SLC9A3, SLC9A4, SLC9A5, SLC9A6, SLC9A7, SLC9A8,
SLC9A9, SLC9A10, SLC9A11
SLC10 solute carrier 10 transporter protein family
SLC10A1, SLC10A2, SLC10A3, SLC10A4, SLC10A5, SLC10A6, SLC10A7
SLC11 solute carrier 11 transporter protein family
SLC11A1
SCL11A2 or hDMT

SLC11A2 transcript variant 4
SLC11A2 transcript variant 1
SLC11A2 transcript variant 2
SLC11A2 transcript variant 3
SLC11A2 transcript variant 5
SLC11A2 transcript variant 6
SLC11A2 transcript variant 7

SLC12 solute carrier 12 transporter protein family
SLC12A1, SLC12A1, SLC12A2, SLC12A3, SLC12A4, SLC12A5, SLC12A6,
SLC12A7, SLC12A8, SLC12A9
SLC13 solute carrier 13 transporter protein family
SLC13A1, SLC13A2, SLC13A3, SLC13A4, SLC13A5
SLC14 solute carrier 14 transporter protein family
SLC14A1, SLC14A2
SLC15 solute carrier 15 transporter protein family
SLC15A1, SLC15A2, SLC15A3, SLC15A4
SLC16 solute carrier 16 transporter protein family
SLC16A1, SLC16A2, SLC16A3, SLC16A4, SLC16A5, SLC16A6, SLC16A7,
SLC16A8, SLC16A9, SLC16A10, SLC16A11, SLC16A12, SLC16A13,
SLC16A14
SLC17 solute carrier 17 transporter protein family
SLC17A1, SLC17A2, SLC17A3, SLC17A4, SLC17A5, SLC17A6, SLC17A7,
SLC17A8
SLC18 solute carrier 18 transporter protein family
SLC18A1, SLC18A2, SLC18A3
SLC19 solute carrier 19 transporter protein family
SLC19A1, SLC19A2, SLC19A3
SLC20 solute carrier 20 transporter protein family
SLC20A1, SLC20A2
SLC21 solute carrier 21 transporter protein family

subfamily 1; SLCO1A2, SLCO1B1, SLCO1B3, SLCO1B4,
SLCO1C1
subfamily 2; SLCO2A1, SLCO2B1
subfamily 3; SLCO3A1
subfamily 4; SLCO4A1, SLCO4C1
subfamily 5; SLCO5A1

SLC22 solute carrier 22 transporter protein family
SLC22A1, SLC22A2, SLC22A3, SLC22A4, SLC22A5, SLC22A6, SLC22A7,
SLC22A8, SLC22A9, SLC22A10, SLC22A11, SLC22A12, SLC22A13,
SLC22A14, SLC22A15, SLC22A16, SLC22A17, SLC22A18, SLC22A19,
SLC22A20
SLC23 solute carrier 23 transporter protein family
SLC23A1, SLC23A2, SLC23A3, SLC23A4
SLC24 solute carrier 24 transporter protein family
SLC24A1, SLC24A2, SLC24A3, SLC24A4, SLC24A5, SLC24A6
SLC25 solute carrier 25 transporter protein family
SLC25A1, SLC25A2, SLC25A3, SLC25A4, SLC25A5, SLC25A6, SLC25A7,
SLC25A8, SLC25A9, SLC25A10, SLC25A11, SLC25A12, SLC25A13,
SLC25A14, SLC25A15, SLC25A16, SLC25A17, SLC25A18, SLC25A19,
SLC25A20, SLC25A21, SLC25A22, SLC25A23, SLC25A24, SLC25A25,
SLC25A26, SLC25A27, SLC25A28, SLC25A29, SLC25A30, SLC25A31,
SLC25A32, SLC25A33, SLC25A34, SLC25A35, SLC25A36, SLC25A37,
SLC25A38, SLC25A39, SLC25A40, SLC25A41, SLC25A42, SLC25A43,
SLC25A44, SLC25A45, SLC25A46
SLC26 solute carrier 26 transporter protein family
SLC26A1, SLC26A2, SLC26A3, SLC26A4, SLC26A5, SLC26A6, SLC26A7,
SLC26A8, SLC26A9, SLC26A10, SLC26A11
SLC27 solute carrier 27 transporter protein family
SLC27A1, SLC27A2, SLC27A3, SLC27A4, SLC27A5, SLC27A6
SLC28 solute carrier 28 transporter protein family
SLC28A1, SLC28A2, SLC28A3
SLC29 solute carrier 29 transporter protein family
SLC29A1, SLC29A2, SLC29A3, SLC29A4
SLC30 solute carrier 30 transporter protein family
SLC30A1, SLC30A2, SLC30A3, SLC30A4, SLC30A5, SLC30A6, SLC30A7,
SLC30A8, SLC30A9, SLC30A10
SLC31 solute carrier 31 transporter protein family
SLC31A1
SLC32 solute carrier 32 transporter protein family
SLC32A1
SLC33 solute carrier 33 transporter protein family
SLC33A1
SLC34 solute carrier 34 transporter protein family
SLC34A1, SLC34A2, SLC34A3
SLC35 solute carrier 35 transporter protein family

subfamily A; SLC35A1, SLC35A2, SLC35A3, SLC35A4, SLC35A5
subfamily B; SLC35B1, SLC35B2, SLC35B3, SLC35B4
subfamily C; SLC35C1, SLC35C2
subfamily D; SLC35D1, SLC35D2, SLC35D3
subfamily E; SLC35E1, SLC35E2, SLC35E3, SLC35E4

SLC36 solute carrier 36 transporter protein family
SLC36A1, SLC36A2, SLC36A3, SLC36A4
SLC37 solute carrier 37 transporter protein family
SLC37A1, SLC37A2, SLC37A3, SLC37A4
SLC38 solute carrier 38 transporter protein family
SLC38A1, SLC38A2, SLC38A3, SLC38A4, SLC38A5, SLC38A6
SLC39 solute carrier 39 transporter protein family
SLC39A1, SLC39A2, SLC39A3, SLC39A4, SLC39A5, SLC39A6, SLC39A7,
SLC39A8, SLC39A9, SLC39A10, SLC39A11, SLC39A12, SLC39A13,
SLC39A14
SLC40 solute carrier 40 transporter protein family
SLC40A1
SLC41 solute carrier 41 transporter protein family
SLC41A1, SLC41A2, SLC41A3
SLC42 solute carrier 42 transporter protein family
RHAG, RhBG, RhCG
SLC43 solute carrier 43 transporter protein family
SLC43A1
SLC43A2
SLC43A3
SLC44 solute carrier 44 transporter protein family
SLC44A1, SLC44A2, SLC44A3, SLC44A4, SLC44A5
SLC45 solute carrier 45 transporter protein family
SLC45A1, SLC45A2, SLC54A3, SLC45A4
SLC46 solute carrier 46 transporter protein family
SLC46A1, SLC46A2
SLC47 solute carrier 47 transporter protein family
SLC47A1, SLC47A2

MRI Imaging

Human transferrin receptor
Human transferrin receptor
Mouse transferrin receptor
Human ferritin light chain (FTL)
Human ferritin heavy chain
FTL 498-199InsTC, a mutated form of the ferritin light chain
Bacterial ferritin

E. coli
E. coli strain K12
S. aureus strain MRSA252
S. aureus strain NCTC 8325
H. pylori B8

bacterioferritin
codon optimized bacterioferritin
MagA

Enzymes that modify a substrate to produce a detectable product or signal, or are
detectable by antibodies

alpha-amylase
alkaline phosphatase
secreted alkaline phosphatase
peroxidase
T4 lysozyme
oxidoreductase
pyrophosphatase

Therapeutic genes
therapeutic gene product
antigens

tumor specific antigens
tumor-associated antigens
tissue-specific antigens
bacterial antigens
viral antigens
yeast antigens
fungal antigens
protozoan antigens
parasite antigens
mitogens

an antibody or fragment thereof

virus-specific antibodies

antisense RNA
siRNA

siRNA directed against expression of a tumor-promoting gene

an oncogene
growth factor
angiogenesis promoting gene
a receptor

siRNA molecule directed against expression of any gene essential for cell growth,
cell replication or cell survival.
siRNA molecule directed against expression of any gene that stabilizes the cell
membrane or otherwise limits the number of tumor cell antigens released from the
tumor cell.

protein ligands
an antitumor oligopeptide
an antimitotic peptide

tubulysin,
phomopsin
hemiasterlin
taltobulin (HTI-286, 3)
cryptophycin

a mitosis inhibitor protein
an antimitotic oligopeptide
an anti-cancer polypeptide antibiotic
anti-cancer antibiotics

tissue factors

Tissue Factor (TF)
αvβ3-integrin RGD fusion protein

Immune modulatory molecules

GM-CSF
MCP-1 or CCL2 (Monocyte Chemoattractant Protein-1) Human
MCP-1 murine
IP-10 or Chemokine ligand 10 (CXCL10)
LIGHT
P60 or SEQSTM1 (Sequestosome 1 transcript variant 1)
P60 or SEQSTM1 (Sequestosome 1 transcript variant 3)
P60 or SEQSTM1 (Sequestosome 1 transcript variant 2)
OspF
OspG
STAT1alpha
STAT1beta

Interleukins

IL-18 (Interleukin-18)
IL-11 (Interleukin-11)
IL-6 (Interleukin-6)
sIL-6R-IL-6
interleukin-12
interleukin-1
interleukin-2
IL-24 (Interleukin-24)
IL-24 transcript variant 1
IL-24 transcript variant 4
IL-24 transcript variant 5
IL-4
IL-8
IL-10

chemokines

IP-10 (CXCL)
Thrombopoetin
members of the C—X—C and C-C chemokine families
RANTES
MIP1-alpha
MIP1-beta
MIP-2

CXC chemokines

GROα
GROβ (MIP-2)
GROγ
ENA-78
LDGF-PPBP
GCP-2
PF4
Mig
IP-10
SDF-1α/β
BUNZO/STRC33
I-TAC
BLC/BCA-1
MDC
TECK
TARC
HCC-1
HCC-4
DC-CK1
MIP-3α
MIP-3β
MCP-2
MCP-3 (Monocyte Chemoattractant Protein-3, CCL7)
MCP-4
MCP-5 (Monocyte Chemoattractant Protein-5; CCL12)
Eotaxin (CCL11)
Eotaxin-2/MPIF-2
I-309
MIP-5/HCC-2
MPIF-1
6Ckine
CTACK
MEC
lymphotactin
fractalkine

Immunoglobulin superfamily of cytokines

B7.1
B7.2.

Anti-angiogenic genes/angiogenesis inhibitors

Human plasminogen k5 domain (hK5)
PEDF (SERPINF1) (Human)
PEDF (mouse)
anti-VEGF single chain antibody (G6)
anti-DLL4 s.c. antibody GLAF-3
tTF-RGD (truncated human tissue factor protein fused to an RGD peptide)

viral attenuation factors

Interferons

IFN-γ
IFN-α
IFN-β

Antibody or scFv

Therapeutic antibodies (i.e. anticancer antibodies)

Rituximab (RITUXAN)
ADEPT
Trastuzumab (Herceptin)
Tositumomab (Bexxar)
Cetuximab (Erbitux)
Ibritumomab (90Y-Ibritumomab tiuexetan; Zevalin)
Alemtuzumab (Campath-1H)
Epratuzumab (Lymphocide)
Gemtuzumab ozogamicin (Mylotarg)
Bevacimab (Avastin) and Edrecolomab (Panorex)
Infliximab

Metastasis suppressor genes

NM23 or NME1 Isoform a
NM23 or NME1 Isoform b

Anti-metastatic genes

E-Cad
Gelsolin
LKB1 (STK11)
RASSF1
RASSF2
RASSF3
RASSF4
RASSF5
RASSF6
RASSF7
RASSF8
Syk
TIMP-1 (Tissue Inhibitor of Metalloproteinase Type-1)
TIMP-2 (Tissue Inhibitor of Metalloproteinase Type-2)
TIMP-3 (Tissue Inhibitor of Metalloproteinase Type-3)
TIMP-4 (Tissue Inhibitor of Metalloproteinase Type-4)
BRMS-1
CRMP-1
CRSP3
CTGF
DRG1
KAI1
KiSS1 (kisspeptin)
kisspeptin fragments

kisspeptin-10
kisspeptin-13
kisspeptin-14
kisspeptin-54

Mkk4
Mkk6
Mkk7
RKIP
RHOGDI2
SSECKS
TXNIP/VDUP1

Cell matrix-degradative genes

Relaxin 1
hMMP9

Hormones

Human Erythropoietin (EPO)

MicroRNAs

pre-miRNA 181a (sequence inserted into viral genome)
miRNA 181a
mmu-miR-181a MIMAT0000210 mature miRNA 181a
pre-miRNA 126 (sequence inserted into the vial genome)
miRNA 126
hsa-miR-126 MI000471
hsa-miR-126 MIMAT0000445
pre-miRNA 335 (sequence inserted into the viral genome)
miRNA 335
hsa-miR-335 MI0000816
hsa-miR-335 MIMAT0000765

Genes for tissue regeneration and reprogramming Human somatic cells to pluripotency

nAG
Oct4
NANOG
Ngn (Neogenin 1) transcript variant 1
Ngn (Neogenin 1) transcript variant 2
Ngn (Neogenin 1) transcript variant 3
Ngn3
Pdx1
Mafa

Additional Genes

Myc-CTR1
FCU1
mMnSOD
HACE1
nppa1
GCP-2 (Granulocyte Chemotactic Protein-2, CXCL6)
hADH
Wildtype CDC6
Mut CDC6
GLAF-3 anti-DLL4 scFv
GLAF-4 anti-FAP (Fibroblast Activation Protein) scFv (Brocks et al., (2001) Mol.
Medicine 7(7): 461-469)
GLAF-5 anti-FAP scFv
BMP4
wildtype F14.5L

Other Proteins

WT1
p53
pseudomonas exotoxin
diphtheria toxin
Arf or p16
Bax
Herpes simplex virus thymidine kinase
E. coli purine nucleoside phosphorylase
angiostatin
endostatin
Rb
BRCA1
cystic fibrosis transmembrane regulator (CFTR)
Factor VIII
low density lipoprotein receptor
alpha-galactosidase
beta-glucocerebrosidase
insulin
parathyroid hormone
alpha-1-antitrypsin
rsCD40L
Fas-ligand
TRAIL
TNF
microcin E492
xanthineguanine phosphoribosyltransferase (XGPRT)
E. coli guanine phosphoribosyltransferase (gpt)
hyperforin
endothelin-1 (ET-1)
connective tissue growth factor (CTGF)
vascular endothelial growth factor (VEGF)
cyclooxygenase
COX-2
cyclooxygenase-2 inhibitor
MPO (Myeloperoxidase)
Apo A1 (Apolipoprotein A1)
CRP (C Reactive Protein)
Fibrinogen
SAP (Serum Amyloid P)
FGF-basic (Fibroblast Growth Factor-basic)
PPAR-agonist
PE37/TGF-alpha fusion protein

Replacement of the A34R gene with another A34R gene from a different strain in order
to increase the EEV form of the virus

A34R from VACV IHD-J
A34R with a mutation at codon 151 (Lys 151 to Asp)
A34R with a mutation at codon 151 (Lys 151 to Glu)

Non-coding Sequence

Non-proteins
Non-coding nucleic acid

Ribozymes

Group I introns
Group II introns
RNaseP
hairpin ribozymes
hammerhead ribozymes

Prodrug converting enzymes

varicella zoster thymidine kinase
cytosine deaminase
purine nucleoside phosphorylase (e.g., from E. coli)
beta lactamase
carboxypeptidase G2
carboxypeptidase A
cytochrome P450

cytochrome P450-2B1
cytochrome P450-4B1

horseradish peroxidase
nitroreductase
rabbit carboxylesterase
mushroom tyrosinase
beta galactosidase (lacZ) (i.e., from E. coli)
beta glucuronidase (gusA)
thymidine phosphorylase
deoxycytidine kinase
linamerase

Proteins detectable by antibodies

chloramphenicol acetyl transferase
hGH

Viral attenuation factors

virus-specific antibodies

mucins
thrombospondin

tumor necrosis factors (TNFs)

TNFα

Superantigens
Toxins

diphtheria toxin
Pseudomonas exotoxin
Escherichia coli Shiga toxin
Shigella toxin
Escherichia coli Verotoxin 1
Toxic Shock Syndrome Toxin 1
Exfoliating Toxins (EXft)
Streptococcal Pyrogenic Exotoxin (SPE) A, B and C
Clostridial Perfringens Enterotoxin (CPET)
staphylococcal enterotoxins

SEA, SEB, SEC1, SEC2, SED, SEE and SEH

Mouse Mammary Tumor Virus proteins (MMTV)
Streptococcal M proteins
Listeria monocytogenes antigen p60
mycoplasma arthritis superantigens

Proteins that can bind a contrasting agent, chromophore, or a compound or ligand that
can be detected

siderophores

enterobactin
salmochelin
yersiniabactin
aerobactin

Growth Factors

platelet-derived growth factor (PDG-F)
keratinocyte growth factor (KGF)
insulin-like growth factor-1 (IGF-1)
insulin-like growth factor-binding proteins (IGFBPs)
transforming growth factor (TGF-alpha)
Growth factors for blood cells

Granulocyte Colony Stimulating Factor (G-CSF)

growth factors that can boost platelets

Other Groups

BAC (Bacterial Artificial Chromosome) encoding several or all proteins of a
specific pathway, e.g. woundhealing-pathway
MAC (Mammalian Artificial Chromosome) encoding several or all proteins of a

specific pathway, e.g. woundhealing-pathway

tumor antigen
RNAi
ligand binding proteins
proteins that can induce a signal detectable by MRI
angiogenins
photosensitizing agents
anti-metabolites
signaling modulators
chemotherapeutic compounds
lipases
proteases
pro-apoptotic factors
anti-cancer vaccine

antigen vaccines
whole cell vaccines (i.e., dendritic cell vaccines)
DNA vaccines
anti-idiotype vaccines

tumor suppressors
cytotoxic protein
cytostatic proteins
costimulatory molecules

cytokines and chemokines

cancer growth inhibitors
gene therapy
BCG vaccine for bladder cancer

Proteins that interact with host cell proteins

i. Diagnostic or Reporter Gene Products

In some examples, the viruses provided herein can express one or more additional genes whose products are detectable or whose products are capable of inducing a detectable signal. In some examples, the viruses provided herein contain nucleic acid that encodes a detectable protein or a protein capable of inducing a detectable signal. Expression of such proteins allows detection of the virus in vitro and in vivo. A variety of detectable gene products, such as detectable proteins are known in the art, and can be used with the viruses provided herein.

Exemplary of such proteins are enzymes that can catalyze a detectable reaction or catalyze formation of a detectable product, such as, for example, luciferases, such as a click beetle luciferase, a Renilla luciferase, a firefly luciferase or beta-glucoronidase (GusA). Also exemplary of such proteins are proteins that emit a detectable signal, including fluorescent proteins, such as a green fluorescent protein (GFP) or a red fluorescent protein (RFP). A variety of DNA sequences encoding proteins that can emit a detectable signal or that can catalyze a detectable reaction, such as luminescent or fluorescent proteins, are known and can be used in the viruses and methods provided herein. Transformation and expression of these genes in viruses can permit detection of viral infection, for example, using a low light and/or fluorescence imaging camera.

Exemplary genes encoding light-emitting proteins include, for example, genes from bacterial luciferase from Vibrio harveyi (Belas et al., Science 218 (1982), 791-793), bacterial luciferase from Vibrio fischerii (Foran and Brown, Nucleic acids Res. 16 (1988), 177), firefly luciferase (de Wet et al., Mol. Cell. Biol. 7 (1987), 725-737), aequorin from Aequorea victoria (Prasher et al., Biochem. 26 (1987), 1326-1332), Renilla luciferase from Renilla renformis (Lorenz et al, PNAS USA 88 (1991), 4438-4442). The luxA and luxB genes of bacterial luciferase can be fused to produce the fusion gene (Fab₂), which can be expressed to produce a fully functional luciferase protein (Escher et al., PNAS 86: 6528-6532 (1989)). In some examples, luciferases expressed by viruses can require exogenously added substrates such as decanal or coelenterazine for light emission. In other examples, viruses can express a complete lux operon, which can include proteins that can provide luciferase substrates such as decanal. For example, viruses containing the complete lux operon sequence, when injected intraperitoneally, intramuscularly, or intravenously, allowed the visualization and localization of microorganisms in live mice indicating that the luciferase light emission can penetrate the tissues and can be detected externally (Contag et al., (1995) Mol. Microbiol. 18: 593-603).

Exemplary fluorescent proteins include green fluorescent protein from Aequorea victoria (Prasher et al., Gene 111: 229-233 (1987), and GFP variants and variants of GFP-like proteins. Such fluorescent proteins include monomeric, dimeric and tetrameric fluorescent proteins. Exemplary monomeric fluorescent proteins include, but are not limited to: violet fluorescent proteins, such as for example, Sirius; blue fluorescent proteins, such as for example, Azurite, EBFP, SBFP2, EBFP2, TagBFP; cyan fluorescent proteins, such as for example, mTurquoise, eCFP, Cerulean, SCFP, TagCFP, mTFP1; green fluorescent proteins, such as for example, GFP, mUkG1, aAG1, AcGFP1, TagGFP2, EGFP, mWasabi, EmGFP (Emerald); yellow fluorescent proteins, such as for example; TagYFP, EYFP, Topaz, SYFP2, YPet, Venus, Citrine; orange fluorescent proteins, such as for example, mKO, mKO2, mOrange, mOrange2, red fluorescent proteins, such as for example; TagRFP, TagRFPt, mStrawberry, mRuby, mCherry; far red fluorescent proteins, such as for example; mRasberry, mKate2, mPlum, and mNeptune; and fluorescent proteins having an increased Stokes shift (i.e. >100 nm distance between excitation and emission spectra), such as for example, Sapphire, T-Sapphire, mAmetrine, and mKeima. Exemplary dimeric and tetrameric fluorescent proteins include, but are not limited to: AmCyan1, Midori-Ishi Cyan, copGFP (ppluGFP2), TurboGFP. ZsGreen, TurboYFP, ZsYellow1, TurboRFP, dTomato, DsRed2, DsRed-Express, DsRed-Express2, DsRed-Max, AsRed2, TurboFP602, RFP611, Katushka (TurboFP635), Katushka2, and AQ143. Excitation and emission spectra for exemplary fluorescent proteins are well-known in the art (see also e.g. Chudakov et al. (2010) Physiol Rev 90, 1102-1163).

Exemplary detectable proteins also include proteins that can bind a contrasting agent, chromophore, or a compound or ligand that can be detected, such as a transferrin receptor or a ferritin; and reporter proteins, such as E. coli β-galactosidase, β-glucuronidase, xanthine-guanine phosphoribosyltransferase (gpt).

Also exemplary of detectable proteins are gene products that can specifically bind a detectable compound, including, but not limited to receptors, metal binding proteins (e.g., siderophores, ferritins, transferrin receptors), ligand binding proteins, and antibodies. Also exemplary of detectable proteins are transporter proteins that can bind to and transport detectable molecules. Such molecules can be used for detection of the virus, such as for applications involving imaging. Any of a variety of detectable compounds can be used, and can be imaged by any of a variety of known imaging methods. Exemplary compounds include receptor ligands and antigens for antibodies. The ligand can be labeled according to the imaging method to be used. Exemplary imaging methods include, but are not limited to, X-rays, magnetic resonance methods, such as magnetic resonance imaging (MRI) and magnetic resonance spectroscopy (MRS), and tomographic methods, including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography and ultrasonic tomography.

Labels appropriate for X-ray imaging are known in the art, and include, for example, Bismuth (III), Gold (III), Lanthanum (III) or Lead (II); a radioactive ion, such as ⁶⁷Copper, ⁶⁷Gallium, ⁶⁸Gallium, ¹¹¹Indium, ¹¹³Indium, ¹²³Iodine, ¹²⁵Iodine, ¹³¹Iodine, ¹⁹⁷Mercury, ²⁰³Mercury, ¹⁸⁶Rhenium, ¹⁸⁸Rhenium, ⁹⁷Rubidium, ¹⁰³Rubidium, ⁹⁹Technetium or ⁹⁰Yttrium; a nuclear magnetic spin-resonance isotope, such as Cobalt (II), Copper (II), Chromium (III), Dysprosium (III), Erbium (III), Gadolinium (III), Holmium (III), Iron (II), Iron (III), Manganese (II), Neodymium (III), Nickel (II), Samarium (III), Terbium (III), Vanadium (II) or Ytterbium (III); or rhodamine or fluorescein.

Labels appropriate for magnetic resonance imaging are known in the art, and include, for example, gadolinium chelates and iron oxides. Use of chelates in contrast agents is known in the art. Labels appropriate for tomographic imaging methods are known in the art, and include, for example, β-emitters such as ¹¹C, ¹³N, ¹⁵O or ⁶⁴Cu or γ-emitters such as ¹²³I. Other exemplary radionuclides that can, be used, for example, as tracers for PET include ⁵⁵Co, ⁶⁷Ga, ⁶⁸Ga, ⁶⁰Cu(II), ⁶⁷Cu(II), ⁵⁷Ni, ⁵²Fe and ¹⁸F (e.g., ¹⁸F-fluorodeoxyglucose (FDG)). Examples of useful radionuclide-labeled agents are a ⁶⁴Cu-labeled engineered antibody fragment (Wu et al. (2002) PNAS USA 97: 8495-8500), ⁶⁴Cu-labeled somatostatin (Lewis et al. (1999) J. Med. Chem. 42: 1341-1347), ⁶⁴Cu-pyruvaldehyde-bis(N4-methylthiosemicarbazone)(⁶⁴Cu-PTSM) (Adonai et al. (2002) PNAS USA 99: 3030-3035), ⁵²Fe-citrate (Leenders et al. (1994) J. Neural. Transm. Suppl. 43: 123-132), ⁵²Fe/^{52m}Mn-citrate (Calonder et al. (1999) J. Neurochem. 73: 2047-2055) and ⁵²Fe-labeled iron (III) hydroxide-sucrose complex (Beshara et al. (1999) Br. J. Haematol. 104: 288-295, 296-302).

Exemplary of detectable proteins are transporter proteins that can bind to and transport detectable molecules, such as human epinephrine transporter (hNET) or sodium iodide symporter (NIS) that can bind to and transport detectable molecules, such as MIBG and other labeled molecules (e.g., Na¹²⁵I), into the cell.

Other exemplary detectable proteins are proteins encoded by genes for melanin synthesis. Many genes are known to be involved in melanin biosynthesis (see e.g. Simon et al. (2009) Pigment Cell Melanoma Res, 22:563-79). Melanin is a pigment that can be subdivided into the brownish/black eumelanin and the reddish brown pheomelanin. Exemplary of such genes include, but are not limited to, mouse tyrosinase (mTYR), human tyrosinase related protein 1 (tyrp1) and human Dopachrome tautomerase/tyrosinase related protein 2 (DC2). A virus expressing a gene for melanin synthesis can be used to infect hosts or cells to obtain cells with high light absorption rates over the whole visible spectrum. The resulting cells or animals can be imaged using any imaging system capable of detecting high light absorption rates over the whole visible spectrum and/or across different penetration scales. For example, a (multispectral) photo-/optoacoustic tomography—(MS)OAT can be used (see e.g. Ntziachristos (2010) Nature Methods, 7:603-14; Li et al. (2007) J Biomed Optics Letters, 12:1-3).

The viruses can be modified for purposes of using the viruses for imaging, including for the purpose of dual imaging in vitro and/or in vivo to detect two or more detectable gene products, gene products that produce a detectable signal, gene products that can bind a detectable compound, or gene products that can bind other molecules to form a detectable product. In some examples, the two or more gene products are expressed by different viruses, whereas in other examples the two or more gene products are produced by the same virus. For example, a virus can express a gene product that emits a detectable signal and also express a gene product that catalyzes a detectable reaction. In other examples, a virus can express one or more gene products that emit a detectable signal, one or more gene products that catalyze a detectable reaction, one or more gene products that can bind a detectable compound or that can form a detectable product, or any combination thereof. Any combination of such gene products can be expressed by the viruses provided herein and can be used in combination with any of the methods provided herein. Imaging of such gene products can be performed, for example, by various imaging methods as described herein and known in the art (e.g., fluorescence imaging, MRI, PET, among many other methods of detection). Imaging of gene products can also be performed using the same method, whereby gene products are distinguished by their properties, such as by differences in wavelengths of light emitted. For example, a virus can express more than one fluorescent protein that differs in the wavelength of light emitted (e.g., a GFP and an RFP). In another non-limiting example, an RFP can be expressed with a luciferase. In yet other non-limiting examples, a fluorescent gene product can be expressed with a gene product, such as a ferritin or a transferrin receptor, used for magnetic resonance imaging. A virus expressing two or more detectable gene products or two or more viruses expressing two or more detectable gene products can be imaged in vitro or in vivo using such methods. In some examples the two or more gene products are expressed as a single polypeptide, such as a fusion protein. For example a fluorescent protein can be expressed as a fusion protein with a luciferase protein.

Ii. Therapeutic Gene Products

Viruses provided herein also can contain a heterologous nucleic acid molecule that encodes one or more therapeutic gene products. Therapeutic gene products include products that cause cell death or cause an anti-tumor immune response. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound.

Exemplary therapeutic gene products that can be expressed by the viruses provided herein include, but are not limited to, gene products (i.e., proteins and RNAs), including those useful for tumor therapy, such as, but not limited to, an anticancer agent, an antimetastatic agent, or an antiangiogenic agent. For example, exemplary proteins useful for tumor therapy include, but are not limited to, tumor suppressors, cytostatic proteins and costimulatory molecues, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor. For example, a large number of therapeutic proteins that can be expressed for tumor treatment in the viruses and methods provided herein are known in the art, including, but not limited to, a transporter, a cell-surface receptor, a cytokine, a chemokine, an apoptotic protein, a mitosis inhibitor protein, an antimitotic oligopeptide, an antiangiogenic factor (e.g., hk5), angiogenesis inhibitors (e.g., plasminogen kringle 5 domain, anti-vascular endothelial growth factor (VEGF) scAb, tTF-RGD, truncated human tissue factor-αᵥβ₃-integrin RGD peptide fusion protein), anticancer antibodies, such as a single-chain antibody (e.g., an antitumor antibody or an antiangiogenic antibody, such as an anti-VEGF antibody or an anti-epidermal growth factor receptor (EGFR) antibody), a toxin, a tumor antigen, a prodrug converting enzyme, a ribozyme, RNAi, and siRNA.

Additional therapeutic gene products that can be expressed by the oncolytic reporter viruses include, but are not limited to, cell matrix degradative genes, such as but not limited to, relaxin-1 and MMP9, and genes for tissue regeneration and reprogramming human somatic cells to pluripotency, such as but not limited to, nAG, Oct4, NANOS, Neogenin-1, Ngn3, Pdx1 and Mafa.

Costimulatory molecules for the methods provided herein include any molecules which are capable of enhancing immune responses to an antigen/pathogen in vivo and/or in vitro. Costimulatory molecules also encompass any molecules which promote the activation, proliferation, differentiation, maturation or maintenance of lymphocytes and/or other cells whose function is important or essential for immune responses.

An exemplary, non-limiting list of therapeutic proteins includes tumor growth suppressors such as IL-24, WT1, p53, diphtheria toxin, Arf, Bax, HSV TK, E. coli purine nucleoside phosphorylase, angiostatin and endostatin, p16, Rb, BRCA1, cystic fibrosis transmembrane regulator (CFTR), Factor VIII, low density lipoprotein receptor, beta-galactosidase, alpha-galactosidase, beta-glucocerebrosidase, insulin, parathyroid hormone, alpha-1-antitrypsin, rsCD40L, Fas-ligand, TRAIL, TNF, antibodies, microcin E492, diphtheria toxin, Pseudomonas exotoxin, Escherichia coli Shiga toxin, Escherichia coli Verotoxin 1, and hyperforin. Exemplary cytokines include, but are not limited to, chemokines and classical cytokines, such as the interleukins, including for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factor (GM-CSF), erythropoietin and exemplary chemokines including, but not limited to CXC chemokines such as IL-8 GROα, GROβ, GROγ, ENA-78, LDGF-PBP, GCP-2, PF4, Mig, IP-10, SDF-1α/β, BUNZO/STRC33, I-TAC, BLC/BCA-1; CC chemokines such as MIP-1α, MIP-1β, MDC, TECK, TARC, RANTES, HCC-1, HCC-4, DC-CK1, MIP-3α, MIP-3β, MCP-1, MCP-2, MCP-3, MCP-4, Eotaxin, Eotaxin-2/MPIF-2, I-309, MIP-5/HCC-2, MPIF-1, 6Ckine, CTACK, MEC; lymphotactin; and fractalkine. Exemplary other costimulatory molecules include immunoglobulin superfamily of cytokines, such as B7.1, B7.2.

Exemplary therapeutic proteins that can be expressed by the viruses provided herein and used in the methods provided herein include, but are not limited to, erythropoietin (e.g., SEQ ID NO: 12), an anti-VEGF single chain antibody (e.g., SEQ ID NO: 13), a plasminogen K5 domain (e.g., SEQ ID NO: 14), a human tissue factor-αvβ3-integrin RGD fusion protein (e.g., SEQ ID NO: 15), interleukin-24 (e.g., SEQ ID NO: 16), or immune stimulators, such as SIL-6-SIL-6 receptor fusion protein (e.g., SEQ ID NO: 17).

In some examples, the viruses provided herein can express one or more therapeutic gene products that are proteins that convert a less active compound into a compound that causes tumor cell death. Exemplary methods of conversion of such a prodrug compound include enzymatic conversion and photolytic conversion. A large variety of protein/compound pairs are known in the art, and include, but are not limited to, Herpes simplex virus thymidine kinase/ganciclovir, Herpes simplex virus thymidine kinase/(E)-5-(2-bromovinyl)-2′-deoxyuridine (BVDU), varicella zoster thymidine kinase/ganciclovir, varicella zoster thymidine kinase/BVDU, varicella zoster thymidine kinase/(E)-5-(2-bromovinyl)-1-beta-D-arabinofuranosyluracil (BVaraU), cytosine deaminase/5-fluorouracil, cytosine deaminase/5-fluorocytosine, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid (CMDA), carboxypeptidase A/methotrexate-phenylamine, cytochrome P450/acetominophen, cytochrome P450-2B1/cyclophosphamide, cytochrome P450-4B1/2-aminoanthracene, 4-ipomeanol, horseradish peroxidase/indole-3-acetic acid, nitroreductase/CB1954, rabbit carboxylesterase/7-ethyl-10-[4-(1-piperidino)-1-piperidino]carbonyloxy-camptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin glucuronide, thymidine phosphorylase/5′-deoxy-5-fluorouridine, deoxycytidine kinase/cytosine arabinoside, and linamerase/linamarin.

Other therapeutic gene products that can be expressed by the viruses provided herein include siRNA and microRNA molecules. The siRNA and/or microRNA molecule can be directed against expression of a tumor-promoting gene, such as, but not limited to, an oncogene, growth factor, angiogenesis promoting gene, or a receptor. The siRNA and/or microRNA molecule also can be directed against expression of any gene essential for cell growth, cell replication or cell survival. The siRNA and/or microRNA molecule also can be directed against expression of any gene that stabilizes the cell membrane or otherwise limits the number of tumor cell antigens released from the tumor cell. Design of an siRNA or microRNA can be readily determined according to the selected target of the siRNA; methods of siRNA and microRNA design and down-regulation of genes are known in the art, as exemplified in U.S. Pat. Pub. Nos. 2003-0198627 and 2007-0044164, and Zeng et al., Molecular Cell 9:1327-1333 (2002).

Therapeutic gene products include viral attenuation factors, such as antiviral proteins. Antiviral proteins or peptides can be expressed by the viruses provided herein. Expression of antiviral proteins or peptides can control viral pathogenicity. Exemplary viral attenuation factors include, but are not limited to, virus-specific antibodies, mucins, thrombospondin, and soluble proteins such as cytokines, including, but not limited to TNFα, interferons (for example IFNα, IFNβ, or IFNγ) and interleukins (for example IL-1, IL-12 or IL-18).

Another exemplary therapeutic gene product that can be expressed by the viruses provided herein is a protein ligand, such as antitumor oligopeptide. Antitumor oligopeptides are short protein peptides with high affinity and specificity to tumors. Such oligopeptides could be enriched and identified using tumor-associated phage libraries (Akita et al. (2006) Cancer Sci. 97(10):1075-1081). These oligopeptides have been shown to enhance chemotherapy (U.S. Pat. No. 4,912,199). The oligopeptides can be expressed by the viruses provided herein. Expression of the oligopeptides can elicit anticancer activities on their own or in combination with other chemotherapeutic agents. An exemplary group of antitumor oligopeptides is antimitotic peptides, including, but not limited to, tubulysin (Khalil et al. (2006) Chembiochem. 7(4):678-683), phomopsin, hemiasterlin, taltobulin (HTI-286, 3), and cryptophycin. Tubulysin is from myxobacteria and can induce depletion of cell microtubules and trigger the apoptotic process. The antimitotic peptides can be expressed by the viruses provide herein and elicit anticancer activities on their own or in combination with other therapeutic modalities.

Another exemplary therapeutic gene product that can be expressed by the viruses provided herein is an anti-metastatic agent that inhibits one or more steps of the metastatic cascade. The encoded anti-metastatic agents include agents that inhibit invasion of local tissue, inhibit intravasation into the bloodstream or lymphatics, inhibit cell survival and transport through the bloodstream or lymphatics as emboli or potentially single cells, inhibit cell lodging in microvasculature at the secondary site, inhibit growth into microscopic lesions and subsequently into overt metastatic lesions, and/or inhibit metastasis formation and growth within the primary tumor, where the inhibition of metastasis formation is not a consequence of inhibition of primary tumor growth.

Exemplary anti-metastatic agents expressed by the viruses provided herein can directly or indirectly inhibit one or more steps of the metastatic cascade. Exemplary anti-metastatic agents include, but are not limited to, the following: BRMS-1 (Breast Cancer Metastasis Suppressor 1), CRMP-1 (Collapsin Response Mediator Protein-1), CRSP-3 (Cofactor Required for Sp1 transcriptional activation subunit 3), CTGF (Connective Tissue Growth Factor), DRG-1 (Developmentally-regulated GTP-binding protein 1), E-Cad (E-cadherin), gelsolin, KAI1, KiSS1 (Kisspeptin 1/Metastin), kispeptin-10, kispeptin-13, kispeptin-14, kispeptin-54, LKB 1 (STK11 (serine/threonine kinase 11)), JNKK1/MKK4 (c-Jun-NH2-Kinase Kinase/Mitogen activated Kinase Kinase 4), MKK6 (mitogen activated kinase kinase 6), MKK7 (mitogen activated kinase kinase 7), Nm23 (NDP Kinase A), RASSF1-8 (Ras association (RalGDS/AF-6) domain family members), RKIP (Raf kinase inhibitor protein), RhoGDI2 (Rho GDP dissociation inhibitor 2), SSECKS (src-suppressed C-kinase substrate), Syk, TIMP-1 (Tissue inhibitor of metalloproteinase-1), TIMP-2 (Tissue inhibitor of metalloproteinase-2), TIMP-3 (Tissue inhibitor of metalloproteinase-3), TIMP-4 (Tissue inhibitor of metalloproteinase-4), TXNIP/VDUP1 (Thioredoxin-interacting protein). Such list of anti-metastatic agents is not meant to be limiting. Any gene product that can suppress metastasis formation via a mechanism that is independent of inhibition of growth within the primary tumor is encompassed by the designation of an anti-metastatic agent or metastasis suppressor and can be expressed by a virus as provided herein. One of skill in the art can identify anti-metastatic genes and can construct a virus expressing one or more anti-metastatic genes for therapy.

Another exemplary therapeutic gene product that can be expressed by the viruses provided herein is a protein that sequesters molecules or nutrients needed for tumor growth. For example, the virus can express one or more proteins that bind iron, transport iron, or store iron, or a combination thereof. Increased iron uptake and/or storage by expression of such proteins not only, increases contrast for visualization and detection of a tumor or tissue in which the virus accumulates, but also depletes iron from the tumor environment. Iron depletion from the tumor environment removes a vital nutrient from the tumors, thereby deregulating iron hemostasis in tumor cells and delaying tumor progression and/or killing the tumor.

Additionally, iron, or other labeled metals, can be administered to a tumor-bearing subject, either alone, or in a conjugated form. An iron conjugate can include, for example, iron conjugated to an imaging moiety or a therapeutic agent. In some cases, the imaging moiety and therapeutic agent are the same, e.g., a radionuclide. Internalization of iron in the tumor, wound, area of inflammation or infection allows the internalization of iron alone, a supplemental imaging moiety, or a therapeutic agent (which can deliver cytotoxicity specifically to tumor cells or deliver the therapeutic agent for treatment of the wound, area of inflammation or infection). These methods can be combined with any of the other methods provided herein.

The administered virus also can be modified to stimulate humoral and/or cellular immune response in the subject, such as the induction of cytotoxic T lymphocytes responses. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

For example, the viruses provided herein can be modified to express one or more antigens. Sustained release of the antigen can result in an immune response by the viral-infected host, in which the host can develop antibodies against the antigen and/or the host can develop an immune response against cells expressing the antigen. Exemplary antigens include, but are not limited to, tumor specific antigens, tumor-associated antigens, tissue-specific antigens, bacterial antigens, viral antigens, yeast antigens, fungal antigens, protozoan antigens, parasite antigens and mitogens. Superantigens are antigens that can activate a large immune response, often brought about by a large response of T cells. A variety of superantigens are known in the art including, but not limited to, diphtheria toxin, staphylococcal enterotoxins (SEA, SEB, SEC1, SEC2, SED, SEE and SEH), Toxic Shock Syndrome Toxin 1, Exfoliating Toxins (EXft), Streptococcal Pyrogenic Exotoxin A, B and C(SPE A, B and C), Mouse Mammary Tumor Virus proteins (MMTV), Streptococcal M proteins, Clostridial Perfringens Enterotoxin (CPET), Listeria monocytogenes antigen p60, and mycoplasma arthritis superantigens.

Since many superantigens also are toxins, if expression of a virus of reduced toxicity is desired, the superantigen can be modified to retain at least some of its superantigenicity while reducing its toxicity, resulting in a compound such as a toxoid. A variety of recombinant superantigens and toxoids of superantigens are known in the art, and can readily be expressed in the viruses provided herein. Exemplary toxoids include toxoids of diphtheria toxin, as exemplified in U.S. Pat. No. 6,455,673 and toxoids of Staphylococcal enterotoxins, as exemplified in U.S. Pat. Pub. No. 2003-0009015.

III. Modifications to Alter Attenuation of the Viruses

Viruses provided herein can be further attenuated by addition, deletion and/or modification of nucleic acid in the viral genome. In one example, the virus is attenuated by addition of heterologous nucleic acid that contains an open reading frame that encodes one or more gene products (e.g. a diagnostic gene product or a therapeutic gene product as described above). In another example, the virus is attenuated by modification of heterologous nucleic acid that contains an open reading frame that encodes one or more gene products. In a further example, the heterologous nucleic acid is modified by increasing the length of the open reading frame, removal of all or part of the open reading frame or replacement of all or part of the open reading frame. Such modifications can affect viral toxicity by disruption of one or more viral genes or by increasing or decreasing the transcriptional and/or translational load on the virus (see, e.g., International Patent Publication No. WO 2008/100292).

In another example, the virus can be attenuated by modification or replacement of one or more promoters contained in the virus. Such promoters can be replaced by stronger or weaker promoters, where replacement results in a change in the attenuation of the virus. In one example, a promoter of a virus provided herein is replaced with a natural promoter. In one example, a promoter of a virus provided herein is replaced with a synthetic promoter. Exemplary promoters that can replace a promoter contained in a virus can be a viral promoter, such as a vaccinia viral promoter, and can include a vaccinia early, intermediate, early/late or late promoter. Additional exemplary viral promoters are provided herein and known in the art and can be used to replace a promoter contained in a virus.

In another example, the virus can be attenuated by removal or all or a portion of a heterologous nucleic acid molecule contained in the virus. The portion of the heterologous nucleic acid that is removed can be 1, 2, 3, 4, 5 or more, 10 or more, 15 or more, 20 or more, 50 or more, 100 or more, 1000 or more, 5000 or more nucleotide bases. In another example, the virus is attenuated by modification of a heterologous nucleic acid contained in the virus by removal or all or a portion of a first heterologous nucleic acid molecule and replacement by a second heterologous nucleic acid molecule, where replacement changes the level of attenuation of the virus. The second heterologous nucleic acid molecule can contain a sequence of nucleotides that encodes a protein or can be a non-coding nucleic acid molecule. In some examples, the second heterologous nucleic acid molecule contains an open reading frame operably linked to a promoter. The second heterologous nucleic acid molecule can contain one or more open reading frames or one or more promoters. Further, the one or more promoters of the second heterologous nucleic acid molecule can be one or more stronger promoters or one or more weaker promoters, or can be a combination or both.

Attenuated vaccinia viruses are known in the art and are described, for example, in U.S. Patent Pub. Nos. US 2005-0031643 now U.S. Pat. Nos. 7,588,767, 7,588,771 and 7,662,398, US 2008-0193373, US 2009-0098529, US 2009-0053244, US 2009-0155287, US 2009-0081639, US 2009-0117034 and US 2009-0136917, and International Patent Pub. Nos. WO 2005/047458, WO 2008/100292 and WO 2008/150496.

Viruses provided herein also can contain a modification that alters its infectivity or resistance to neutralizing antibodies. In one non-limiting example deletion of the A35R gene in an vaccinia LIVP strain can decrease the infectivity of the virus. In some examples, the viruses provided herein can be modified to contain a deletion of the A35R gene. Exemplary methods for generating such viruses are described in PCT Publication No. WO2008/100292, which describes vaccinia LIVP viruses GLV-1j87, GLV-1j88 and GLV-1j89, which contain deletion of the A35R gene.

In another non-limiting example, replacement of viral coat proteins (e.g., A34R, which encodes a viral coat glycoprotein) with coat proteins from either more virulent or less virulent virus strains can increase or decrease the clearance of the virus from the subject. In one example, the A34R gene in a vaccinia LIVP strain can be replaced with the A34R gene from vaccinia IHD-J strain. Such replacement can increase the extracellular enveloped virus (EEV) form of vaccinia virus and can increase the resistance of the virus to neutralizing antibodies.

b. Exemplary Modified or Recombinant Viruses

Exemplary modified vaccinia viruses provided herein are those derived from the Lister strain, and in particular the attenuated Lister strain LIVP. Recombinant LIVP viruses have been generated and are known in the art. The modified LIVP viruses can be modified by insertion, deletion or amino acid replacement of heterologous nucleic acid compared to an LIVP strain having a genome set forth in any one of SEQ ID NOS: 1-8, or having a genome that exhibits at least 97%, 98%, 99% or more sequence identity to any of SEQ ID NOS: 1-8. Table 4 sets forth exemplary viruses, the reference or partental LIVP (e.g. LIVP set forth in SEQ ID NO:1 or GLV-1h68 set forth in SEQ ID NO:9) and the resulting genotype. The exemplary modifications of the Lister strain can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health).

TABLE 4
Recombinant Viruses

| Virus | Parent | Genotype | | | | |
|---|---|---|---|---|---|---|
| Name | Virus | F14.5L | J2R | A56R | A34R | A35R |
| GLV-1h68 | LIVP | (PSE/L)Ruc- | (PSE/L)rTrfR- | (P11)gusA | wt | wt |
| | | GFP | (P7.5)lacZ | | | |
| GLV-1i69 | GLV-1h68 | (PSE/L)Ruc- | (PSE/L)rTrfR- | (P11)gusA | A34R | wt |
| | | GFP | (P7.5)lacZ | | from | |
| | | | | | IHD-J | |
| GLV-1h70 | GLV-1h68 | (PSE/L)Ruc- | (PSE/L)rTrfR- | ko | wt | wt |
| | | GFP | (P7.5)lacZ | | | |
| GLV-1h71 | GLV-1h68 | ko | (PSE/L)rTrfR- | (P11)gusA | wt | wt |
| | | | (P7.5)lacZ | | | |
| GLV-1h72 | GLV-1h68 | (PSE/L)Ruc- | ko | (P11)gusA | wt | wt |
| | | GFP | | | | |
| GLV-1h73 | GLV-1h70 | ko | (PSE/L)rTrfR- | ko | wt | wt |
| | | | (P7.5)lacZ | | | |
| GLV-1h74 | GLV-1h73 | ko | ko | ko | wt | wt |
| GLV-1h76 | GLV-1h68 | (PSE/L)Ruc- | (PSE)GM-CSF | (P11)gusA | wt | wt |
| | | GFP | | | | |
| GLV-1h77 | GLV-1h68 | (PSE/L)Ruc- | (PSE/L)GM-CSF | (P11)gusA | wt | wt |
| | | GFP | | | | |
| GLV-1h78 | GLV-1h68 | (PSE/L)Ruc- | (PSL)GM-CSF | (P11)gusA | wt | wt |
| | | GFP | | | | |
| GLV-1h79 | GLV-1h68 | (PSE/L)Ruc- | (PSE/L)mMCP-1 | (P11)gusA | wt | wt |
| | | GFP | | | | |
| GLV-1h80 | GLV-1h68 | (PSE/L)Ruc- | (PSL)mMCP-1 | (P11)gusA | wt | wt |
| | | GFP | | | | |
| GLV-1h81 | GLV-1h68 | (PSE/L)Ruc- | (PSE/L)rTrfR- | (PSE/L)hk5 | wt | wt |
| | | GFP | (P7.5)lacZ | | | |
| GLV-1h82 | GLV-1h22 | (PSE/L)Ruc- | (PSE/L)TrfR- | (PSE/L)ftn | wt | wt |
| | | GFP | (P7.5)lacZ | | | |
| GLV-1h83 | GLV-1h68 | (PSE/L)Ruc- | (PSE/L)rTrfR- | (PSE/L)ftn | wt | wt |
| | | GFP | (P7.5)lacZ | | | |
| GLV-1h84 | GLV-1h68 | ko | (PSE/L)CBG99- | ko | wt | wt |
| | | | mRFP1 | | | |
| GLV-1h85 | GLV-1h72 | ko | ko | (P11)gusA | wt | wt |
| GLV-1h86 | GLV-1h72 | (PSE/L)Ruc- | ko | ko | wt | wt |
| | | GFP | | | | |
| GLV-1j87 | GLV-1h68 | (PSE/L)Ruc- | (PSE/L)rTrfR- | (P11)gusA | wt | ko |
| | | GFP | (P7.5)lacZ | | | |
| GLV-1j88 | GLV-1h73 | ko | (PSE/L)rTrfR- | ko | wt | ko |
| | | | (P7.5)lacZ | | | |
| GLV-1j89 | GLV-1h74 | ko | ko | ko | wt | ko |
| GLV-1h90 | GLV-1h68 | (PSE/L)Ruc- | (PSE/L)rTrfR- | (PSE)sIL- | wt | wt |
| | | GFP | (P7.5)lacZ | 6R/IL-6 | | |
| GLV-1h91 | GLV-1h68 | (PSE/L)Ruc- | (PSE/L)rTrfR- | (PSE/L)sIL- | wt | wt |
| | | GFP | (P7.5)lacZ | 6R/IL-6 | | |
| GLV-1h92 | GLV-1h68 | (PSE/L)Ruc- | (PSE/L)rTrfR- | (PSL)sIL- | wt | wt |
| | | GFP | (P7.5)lacZ | 6R/IL-6 | | |
| GLV-1h93 | GLV-1h68 | (PSE/L)Ruc- | (PSE/L)rTrfR- | (PSE)FCU1 | wt | wt |
| | | GFP | (P7.5)lacZ | | | |
| GLV-1h94 | GLV-1h68 | (PSE/L)Ruc- | (PSE/L)rTrfR- | (PSEL)FCU1 | wt | wt |
| | | GFP | (P7.5)lacZ | | | |
| GLV-1h95 | GLV-1h68 | (PSE/L)Ruc- | (PSE/L)rTrfR- | (PSL)FCU1 | wt | wt |
| | | GFP | (P7.5)lacZ | | | |
| GLV-1h96 | GLV-1h68 | (PSE)IL-24 | (PSE/L)rTrfR- | (P11)gusA | wt | wt |
| | | | (P7.5)lacZ | | | |
| GLV-1h97 | GLV-1h68 | (PSEL)IL-24 | (PSE/L)rTrfR- | (P11)gusA | wt | wt |
| | | | (P7.5)lacZ | | | |
| GLV-1h98 | GLV-1h68 | (PSL)IL-24 | (PSE/L)rTrfR- | (P11)gusA | wt | wt |
| | | | (P7.5)lacZ | | | |
| GLV-1h99 | GLV-1h68 | (PSE)hNET | (PSE/L)rTrfR- | (P11)gusA | wt | wt |
| | | | (P7.5)lacZ | | | |
| GLV- | GLV-1h68 | (PSE/L)Ruc- | (PSE)hNET | (P11)gusA | wt | wt |
| 1h100 | | GFP | | | | |
| GLV- | GLV-1h68 | (PSE/L)Ruc- | (PSL)hNET | (P11)gusA | wt | wt |
| 1h101 | | GFP | | | | |
| GLV- | GLV-1h68 | (PSE/L)Ruc- | (PSE/L)rTrfR- | (PSE)hDMT | wt | wt |
| 1h102 | | GFP | (P7.5)lacZ | | | |
| GLV- | GLV-1h68 | (PSE/L)Ruc- | (PSL)hMCP1 | (P11)gusA | wt | wt |
| 1h103 | | GFP | | | | |
| GLV- | GLV-1h68 | (PSE/L)Ruc- | (PSE)tTF-RGD | (P11)gusA | wt | wt |
| 1h104 | | GFP | | | | |
| GLV- | GLV-1h68 | (PSE/L)Ruc- | (PSE/L)tTF- | (P11)gusA | wt | wt |
| 1h105 | | GFP | RGD | | | |
| GLV- | GLV-1h68 | (PSE/L)Ruc- | (PSL)tTF-RGD | (P11)gusA | wt | wt |
| 1h106 | | GFP | | | | |
| GLV- | GLV-1h68 | (PSE/L)Ruc- | (PSE)G6-FLAG | (P11)gusA | wt | wt |
| 1h107 | | GFP | | | | |
| GLV- | GLV-1h68 | (PSE/L)Ruc- | (PSE/L)G6- | (P11)gusA | wt | wt |
| 1h108 | | GFP | FLAG | | | |
| GLV- | GLV-1h68 | (PSE/L)Ruc- | (PSL)G6-FLAG | (P11)gusA | wt | wt |
| 1h109 | | GFP | | | | |
| GLV- | GLV-1h68 | (PSE/L)Ruc- | (PSE/L)rTrfR- | (PSE)bfr | wt | wt |
| 1h110 | | GFP | (P7.5)lacZ | | | |
| GLV- | GLV-1h68 | (PSE/L)Ruc- | (PSE/L)rTrfR- | (PSE/L)bfr | wt | wt |
| 1h111 | | GFP | (P7.5)lacZ | | | |
| GLV- | GLV-1h68 | (PSE/L)Ruc- | (PSE/L)rTrfR- | (PSL)bfr | wt | wt |
| 1h112 | | GFP | (P7.5)lacZ | | | |
| GLV- | GLV-1h68 | (PSE/L)Ruc- | (PSE/L)rTrfR- | (PSE/L)bfrₒₚₜ | wt | wt |
| 1h113 | | GFP | (P7.5)lacZ | | | |
| GLV- | GLV-1h68 | (PSE/L)Ruc- | (PSE/L)rTrfR- | (PSE)mtr | wt | wt |
| 1h114 | | GFP | (P7.5)lacZ | | | |
| GLV- | GLV-1h68 | (PSE/L)Ruc- | (PSE/L)rTrfR- | (PSE/L)mtr | wt | wt |
| 1h115 | | GFP | (P7.5)lacZ | | | |
| GLV- | GLV-1h68 | (PSE/L)Ruc- | (PSE)mMnSOD | (P11)gusA | wt | wt |
| 1h116 | | GFP | | | | |
| GLV- | GLV-1h68 | (PSE/L)Ruc- | (PSE/L)mMnSOD | (P11)gusA | wt | wt |
| 1h117 | | GFP | | | | |
| GLV- | GLV-1h68 | (PSE/L)Ruc- | (PSL)mMnSOD | (P11)gusA | wt | wt |
| 1h118 | | GFP | | | | |
| GLV- | GLV-1h68 | (PSE/L)Ruc- | (PSE)mIP-10 | (P11)gusA | wt | wt |
| 1h119 | | GFP | | | | |
| GLV- | GLV-1h68 | (PSE/L)Ruc- | (PSE/L)mIP-10 | (P11)gusA | wt | wt |
| 1h120 | | GFP | | | | |
| GLV- | GLV-1h68 | (PSE/L)Ruc- | (PSL)mIP-10 | (P11)gusA | wt | wt |
| 1h121 | | GFP | | | | |
| GLV- | GLV-1h68 | (PSE/L)Ruc- | (PSE)mLIGHT | (P11)gusA | wt | wt |
| 1h122 | | GFP | | | | |
| GLV- | GLV-1h68 | (PSE/L)Ruc- | (PSE/L)mLIGHT | (P11)gusA | wt | wt |
| 1h123 | | GFP | | | | |
| GLV- | GLV-1h68 | (PSE/L)Ruc- | (PSL)mLIGHT | (P11)gusA | wt | wt |
| 1h124 | | GFP | | | | |
| GLV- | GLV-1h68 | (PSE/L)Ruc- | (PSE)CBP | (P11)gusA | wt | wt |
| 1h125 | | GFP | | | | |
| GLV- | GLV-1h68 | (PSE/L)Ruc- | (PSE/L)CBP | (P11)gusA | wt | wt |
| 1h126 | | GFP | | | | |
| GLV- | GLV-1h68 | (PSE/L)Ruc- | (PSL)CBP | (P11)gusA | wt | wt |
| 1h127 | | GFP | | | | |
| GLV- | GLV-1h68 | (PSE/L)Ruc- | (PSE)P60 | (P11)gusA | wt | wt |
| 1h128 | | GFP | | | | |
| GLV-h129 | GLV-1h68 | (PSE/L)Ruc- | (PSE/L)P60 | (P11)gusA | wt | wt |
| | | GFP | | | | |
| GLV- | GLV-1h68 | (PSE/L)Ruc- | (PSL)P60 | (P11)gusA | wt | wt |
| 1h130 | | GFP | | | | |
| GLV- | GLV-1h68 | (PSE/L)Ruc- | (PSE)hFLH | (P11)gusA | wt | wt |
| 1h131 | | GFP | | | | |
| GLV- | GLV-1h68 | (PSE/L)Ruc- | (PSE/L)hFLH | (P11)gusA | wt | wt |
| 1h132 | | GFP | | | | |
| GLV- | GLV-1h68 | (PSE/L)Ruc- | (PSL)hFLH | (P11)gusA | wt | wt |
| 1h133 | | GFP | | | | |
| GLV- | GLV-1h68 | (PSE/L)CBG9 | (PSE/L)rTrfR- | (P11)gusA | wt | wt |
| 1h134 | | 9-mRFP1 | (P7.5)lacZ | | | |
| GLV- | GLV-1h68 | wt | (PSE/L)rTrfR- | (P11)gusA | wt | wt |
| 1e135 | | | (P7.5)lacZ | | | |
| GLV- | GLV-1h68 | (PSE/L)Ruc- | (PSE)PEDF | (P11)gusA | wt | wt |
| 1h136 | | GFP | | | | |
| GLV- | GLV-1h68 | (PSE/L)Ruc- | (PSE/L)PEDF | (P11)gusA | wt | wt |
| 1h137 | | GFP | | | | |
| GLV- | GLV-1h68 | (PSE/L)Ruc- | (PSL)PEDF | (P11)gusA | wt | wt |
| 1h138 | | GFP | | | | |
| GLV- | GLV-1h68 | (PSE/L)Ruc- | (PSE/L)rTrfR- | (PSE)hNET | wt | wt |
| 1h139 | | GFP | (P7.5)lacZ | | | |
| GLV- | GLV-1h68 | (PSE/L)Ruc- | (PSE)CYP11B1 | (P11)gusA | wt | wt |
| 1h140 | | GFP | | | | |
| GLV- | GLV-1h68 | (PSE/L)Ruc- | (PSE/L)CYP11B1 | (P11)gusA | wt | wt |
| 1h141 | | GFP | | | | |
| GLV- | GLV-1h68 | (PSE/L)Ruc- | (PSL)CYP11B1 | (P11)gusA | wt | wt |
| 1h142 | | GFP | | | | |
| GLV- | GLV-1h68 | (PSE/L)Ruc- | (PSE)CYP11B2 | (P11)gusA | wt | wt |
| 1h143 | | GFP | | | | |
| GLV- | GLV-1h68 | (PSE/L)Ruc- | (PSE/L)CYP11B2 | (P11)gusA | wt | wt |
| 1h144 | | GFP | | | | |
| GLV- | GLV-1h68 | (PSE/L)Ruc- | (PSL)CYP11B2 | (P11)gusA | wt | wt |
| 1h145 | | GFP | | | | |
| GLV- | GLV- | (PSE/L)Ruc- | (PSE)hNET | (PSE)IL-24 | wt | wt |
| 1h146 | 1h100 | GFP | | | | |
| GLV- | GLV-1h68 | (PSE/L)Ruc- | (PSE)HACE1 | (P11)gusA | wt | wt |
| 1h147 | | GFP | | | | |
| GLV- | GLV-1h68 | (PSE/L)Ruc- | (PSE/L)HACE1 | (P11)gusA | wt | wt |
| 1h148 | | GFP | | | | |
| GLV- | GLV-1h68 | (PSE/L)Ruc- | (PSL)HACE1 | (P11)gusA | wt | wt |
| 1h149 | | GFP | | | | |
| GLV- | GLV- | (PSE/L)Ruc- | (PSL)hNET | (PSE)IL-24 | wt | wt |
| 1h150 | 1h101 | GFP | | | | |
| GLV- | GLV-1h68 | (PSE/L) Ruc- | (PSE/L)TfR- | (PSE)hNIS | wt | wt |
| 1h151 | | GFP | (P7.5)lacZ | | | |
| GLV- | GLV-1h68 | (PSE/L) Ruc- | (PSE/L)TfR- | (PSE)hNISa | wt | wt |
| 1h153 | | GFP | (P7.5)lacZ | | | |
| GLV- | GLV-1h22 | (P_{SE/L}) Ruc- | (P_{SE/L})TfR- | (P_{SE/L})bfrₒₚₜ. | wt | wt |
| 1h154 | | GFP | (P_{7.5})lacZ | | | |
| GLV- | GLV-1h22 | (PSE/L) Ruc- | (PSE/L)TfR- | (PSE/L)hFH | wt | wt |
| 1h155 | | GFP | (P7.5)lacZ | | | |
| GLV- | GLV- | (PSE/L) Ruc- | (PSE/L)mtr | (PSE/L)bfrₒₚₜ | wt | wt |
| 1h156 | 1h113 | GFP | | | | |
| GLV- | GLV-1h68 | (PSE/L) Ruc- | (PSE/L)mtr | (PSE/L)hFH | wt | wt |
| 1h157 | | GFP | | | | |
| GLV- | GLV-1h68 | (PSE/L) Ruc- | (PSE/L)TfR- | (PSE/L)G6- | wt | wt |
| 1h158 | | GFP | (P7.5)lacZ | scAb | | |
| GLV- | GLV-1h68 | (PSE/L) Ruc- | (PSE/L)TfR- | (PSL)G6-scAb | wt | wt |
| 1h159 | | GFP | (P7.5)lacZ | | | |
| GLV- | GLV-1h68 | (PSE/L)Ruc- | (PSEL)luxAB | (P11)gusA | wt | wt |
| 1h160 | | GFP | | | | |
| GLV- | GLV-1h68 | (PSE/L)Ruc- | (PSE/L)TfR- | (PSEL)luxCD | wt | wt |
| 1h161 | | GFP | (P7.5)lacZ | | | |
| GLV- | GLV-1h68 | (PSEL)luxE | (PSE/L)rTrfR- | (P11)gusA | wt | wt |
| 1h162 | | | (P7.5)lacZ | | | |
| GLV- | GLV- | (PSE/L)Ruc- | (PSE)hNET | (PSE/L)G6- | wt | wt |
| 1h163 | 1h100 | GFP | | scAb | | |
| GLV- | GLV- | (PSE/L)Ruc- | (PSE)hNET | (PSL)G6-scAb | wt | wt |
| 1h164 | 1h100 | GFP | | | | |
| GLV- | GLV-1h68 | (PSE/L)Ruc- | (PSE)nAG | (P11)gusA | wt | wt |
| 1h165 | | GFP | | | | |
| GLV- | GLV-1h68 | (PSE/L)Ruc- | (PSEL)NAG | (P11)gusA | wt | wt |
| 1h166 | | GFP | | | | |
| GLV- | GLV-1h68 | (PSE/L)Ruc- | (PSL)nAG | (P11)gusA | wt | wt |
| 1h167 | | GFP | | | | |
| GLV- | GLV-1h68 | (PSE/L)Ruc- | (PSE)RLN | (P11)gusA | wt | wt |
| 1h168 | | GFP | | | | |
| GLV- | GLV-1h68 | (PSE/L)Ruc- | (PSEL)RLN | (P11)gusA | wt | wt |
| 1h169 | | GFP | | | | |
| GLV- | GLV-1h68 | (PSE/L)Ruc- | (PSL)RLN | (P11)gusA | wt | wt |
| 1h170 | | GFP | | | | |
| GLV- | GLV-1h68 | (PSE/L)Ruc- | (PSE)NM23A | (P11)gusA | wt | wt |
| 1h171 | | GFP | | | | |
| GLV- | GLV-1h68 | (PSE/L)Ruc- | (PSEL)NM23A | (P11)gusA | wt | wt |
| 1h172 | | GFP | | | | |
| GLV- | GLV-1h68 | (PSE/L)Ruc- | (PSL)NM23 | (P11)gusA | wt | wt |
| 1h173 | | GFP | | | | |
| GLV- | GLV-1h68 | (PSE/L)Ruc- | (PSE)NPPA1 | (P11)gusA | wt | wt |
| 1h174 | | GFP | | | | |
| GLV- | GLV-1h68 | (PSE/L)Ruc- | (PSEL)NPPA1 | (P11)gusA | wt | wt |
| 1h175 | | GFP | | | | |
| GLV- | GLV-1h68 | (PSE/L)Ruc- | (PSL)NPPA1 | (P11)gusA | wt | wt |
| 1h176 | | GFP | | | | |
| GLV- | GLV-1h68 | (PSE/L)Ruc- | (PSE)STAT1α | (P11)gusA | wt | wt |
| 1h177 | | GFP | | | | |
| GLV- | GLV-1h68 | (PSE/L)Ruc- | (PSEL)STAT1α | (P11)gusA | wt | wt |
| 1h178 | | GFP | | | | |
| GLV- | GLV-1h68 | (PSE/L)Ruc- | (PSL)STAT1α | (P11)gusA | wt | wt |
| 1h179 | | GFP | | | | |
| GLV- | GLV-1h68 | (PSE/L)Ruc- | (PSE)CPG2 | (P11)gusA | wt | wt |
| 1h180 | | GFP | | | | |
| GLV- | GLV-1h68 | (PSE/L)Ruc- | (PSEL)CPG2 | (P11)gusA | wt | wt |
| 1h181 | | GFP | | | | |
| GLV- | GLV-1h68 | (PSE/L)Ruc- | (PSL)CPG2 | (P11)gusA | wt | wt |
| 1h182 | | GFP | | | | |
| GLV- | GLV-1h68 | (PSE/L)Ruc- | (PSE)Ecad | (P11)gusA | wt | wt |
| 1h183 | | GFP | | | | |
| GLV- | GLV-1h68 | (PSE/L) Ruc- | (PSE/L)TfR- | (PSE)magA | wt | wt |
| 1h184 | | GFP | (P7.5)lacZ | | | |
| GLV- | GLV-1h68 | (PSE/L)Ruc- | (PSL)Ecad | (P11)gusA | wt | wt |
| 1h185 | | GFP | | | | |
| GLV- | GLV-1h68 | (PSE/L) Ruc- | (PSE/L)TfR- | (PSEL)FTL | wt | wt |
| 1h186 | | GFP | (P7.5)lacZ | 498-499InsTC | | |
| GLV- | GLV-1h68 | (PSE/L) Ruc- | (PSE/L)TfR- | (PSEL)FTL | wt | wt |
| 1h187 | | GFP | (P7.5)lacZ | | | |
| GLV- | GLV-1h68 | (PSE/L) Ruc- | (PSE/L)TfR- | (PSE)FUKW | wt | wt |
| 1h188 | | GFP | (P7.5)lacZ | | | |
| GLV- | GLV-1h68 | (PSE/L) Ruc- | (PSE/L)TfR- | (PSEL)FUKW | wt | wt |
| 1h189 | | GFP | (P7.5)lacZ | | | |
| GLV- | GLV-1h68 | (PSE/L) Ruc- | (PSE/L)TfR- | (PSL)FUKW | wt | wt |
| 1h190 | | GFP | (P7.5)lacZ | | | |
| GLV- | GLV-1h68 | (PSE/L)Ruc- | (PSE)STAT1β | (P11)gusA | wt | wt |
| 1h191 | | GFP | | | | |
| GLV- | GLV-1h68 | (PSE/L)Ruc- | (PSEL)STAT1β | (P11)gusA | wt | wt |
| 1h192 | | GFP | | | | |
| GLV- | GLV-1h68 | (PSE/L)Ruc- | (PSL)STAT1β | (P11)gusA | wt | wt |
| 1h193 | | GFP | | | | |
| GLV- | GLV- | (PSE)luxE | (PSE/L)TfR- | (PSEL)luxCD | wt | wt |
| 1h194 | 1h161 | | (P7.5)lacZ | | | |
| GLV- | GLV- | (PSE/L)Ruc- | (PSE)luxAB | (PSEL)luxCD | wt | wt |
| 1h195 | 1h161 | GFP | | | | |
| GLV- | GLV-1h68 | (PSE/L)Ruc- | (PSE)181a | (P11)gusA | wt | wt |
| 1h196 | | GFP | | | | |
| GLV- | GLV-1h68 | | | | | |
| 1h197 | | | | | | |
| GLV- | GLV-1h68 | (PSE/L)Ruc- | (PSL)181a | (P11)gusA | wt | wt |
| 1h198 | | GFP | | | | |
| GLV- | GLV-1h68 | (PSE/L)Ruc- | (PSE)335 | (P11)gusA | wt | wt |
| 1h199 | | GFP | | | | |
| GLV- | GLV-1h68 | (PSE/L)Ruc- | (PSL)335 | (P11)gusA | wt | wt |
| 1h201 | | GFP | | | | |
| GLV- | GLV-1h68 | | | | | |
| 1h202 | | | | | | |
| GLV- | GLV-1h68 | (PSE/L)Ruc- | (PSEL)126 | (P11)gusA | wt | wt |
| 1h203 | | GFP | | | | |
| GLV- | GLV-1h68 | | | | | |
| 1h204 | | | | | | |
| GLV- | GLV-1h68 | (PSE/L) Ruc- | (PSE/L)TfR- | (PSE)NANOG | wt | wt |
| 1h205 | | GFP | (P7.5)lacZ | | | |
| GLV- | GLV-1h68 | (PSE/L) Ruc- | (PSE/L)TfR- | (PSE)Oct4 | wt | wt |
| 1h208 | | GFP | (P7.5)lacZ | | | |
| GLV- | GLV-1h68 | (PSE/L)Ruc- | (P7.5E)hEPO | (P11)gusA | wt | wt |
| 1h210 | | GFP | | | | |
| GLV- | GLV-1h68 | (PSE/L)Ruc- | (PSE)hEPO | (P11)gusA | wt | wt |
| 1h211 | | GFP | | | | |
| GLV- | GLV-1h68 | (PSE/L)Ruc- | (PSEL)hEPO | (P11)gusA | wt | wt |
| 1h212 | | GFP | | | | |
| GLV- | GLV-1h68 | (PSE/L)Ruc- | (PSL)hEPO | (P11)gusA | wt | wt |
| 1h213 | | GFP | | | | |
| GLV- | GLV-1h68 | (PSE/L)Ruc- | (PSE)OspF | (P11)gusA | wt | wt |
| 1h214 | | GFP | | | | |
| GLV- | GLV-1h68 | (PSE/L)Ruc- | (PSE)OspG | (P11)gusA | wt | wt |
| 1h215 | | GFP | | | | |
| GLV- | GLV-1h68 | (PSE/L)Ruc- | (PSEL)OspG | (P11)gusA | wt | wt |
| 1h216 | | GFP | | | | |
| GLV- | GLV-1h68 | (PSE/L)Ruc- | (PSL)OspG | (P11)gusA | wt | wt |
| 1h217 | | GFP | | | | |
| GLV- | GLV-1h84 | ko | (PSE/L)CBG99- | (PSE)RLN | wt | wt |
| 1h218 | | | mRFP1 | | | |
| GLV- | GLV-1h84 | ko | (PSE/L)CBG99- | (PSEL)RLN | wt | wt |
| 1h219 | | | mRFP1 | | | |
| GLV- | GLV-1h84 | ko | (PSE/L)CBG99- | (PSL)RLN | wt | wt |
| 1h220 | | | mRFP1 | | | |
| GLV- | GLV- | (PSE)luxE | (PSEL)luxAB | (P11)gusA | wt | wt |
| 1h221 | 1h160 | | | | | |
| GLV- | GLV-1h68 | (PSE/L)Ruc- | (PSE)Ngn3 | (P11)gusA | wt | wt |
| 1h222 | | GFP | | | | |
| GLV- | GLV-1h68 | (PSE/L)Ruc- | (PSEL)Ngn3 | (P11)gusA | wt | wt |
| 1h223 | | GFP | | | | |
| GLV- | GLV-1h68 | (PSE/L)Ruc- | (PSL)Ngn3 | (P11)gusA | wt | wt |
| 1h224 | | GFP | | | | |
| GLV- | GLV-1h68 | (PSE/L)Ruc- | (PSE)hADH | (P11)gusA | wt | wt |
| 1h225 | | GFP | | | | |
| GLV- | GLV-1h68 | (PSE/L)Ruc- | (PSEL)hADH | (P11)gusA | wt | wt |
| 1h226 | | GFP | | | | |
| GLV- | GLV-1h68 | (PSE/L)Ruc- | (PSL)hADH | (P11)gusA | wt | wt |
| 1h227 | | GFP | | | | |
| GLV- | GLV- | (PSE)luxE | (PSE)luxAB | (PSEL)luxCD | wt | wt |
| 1h228 | 1h194 | | | | | |
| GLV- | GLV- | (PSEL)luxE | (PSE)luxAB | (PSEL)luxCD | wt | wt |
| 1h229 | 1h195 | | | | | |
| GLV- | GLV-1h68 | (PSE/L)Ruc- | (PSE)Myc-CTR1 | (P11)gusA | wt | wt |
| 1h230 | | GFP | | | | |
| GLV- | GLV-1h68 | (PSE/L)Ruc- | (PSL)Myc-CTR1 | (P11)gusA | wt | wt |
| 1h231 | | GFP | | | | |
| GLV- | GLV-1h68 | (PSE/L)Ruc- | (PSE)CTR1 | (P11)gusA | wt | wt |
| 1h232 | | GFP | | | | |
| GLV- | GLV-1h68 | (PSE/L)Ruc- | (PSE)mPEDF | (P11)gusA | wt | wt |
| 1h233 | | GFP | | | | |
| GLV- | GLV-1h68 | (PSE/L)Ruc- | (PSEL)mPEDF | (P11)gusA | wt | wt |
| 1h234 | | GFP | | | | |
| GLV- | GLV-1h68 | (PSE/L)Ruc- | (PSL)mPEDF | (P11)gusA | wt | wt |
| 1h235 | | GFP | | | | |
| GLV- | GLV-1h73 | (PSE/L)Ruc- | rtfr(PE/L) | (PSE)WTCDC6 | wt | wt |
| 1h236 | | GFP | (P7.5)lacZ | | | |
| GLV- | GLV-1h73 | (PSE/L)Ruc- | rtfr(PE/L) | (PSE)MutCDC6 | wt | wt |
| 1h237 | | GFP | (P7.5)lacZ | | | |
| GLV- | GLV-1h68 | (PSE/L) Ruc- | (PSE/L)TfR- | (PSL)CBG99- | wt | wt |
| 1h238 | | GFP | (P7.5)lacZ | mRFP1 | | |
| GLV- | GLV-1h68 | (PSE/L)Ruc- | (PSE)GLAF-3 | (P11)gusA | wt | wt |
| 1h239 | | GFP | | | | |
| GLV- | GLV-1h68 | (PSE/L)Ruc- | (PSEL)GLAF-3 | (P11)gusA | wt | wt |
| 1h240 | | GFP | | | | |
| GLV- | GLV-1h68 | (PSE/L)Ruc- | (PSL)GLAF-3 | (P11)gusA | wt | wt |
| 1h241 | | GFP | | | | |
| GLV- | GLV-1h68 | (PSE/L)Ruc- | (PE))luxABCDE | (P11)gusA | wt | wt |
| 1h242 | | GFP | | | | |
| GLV- | GLV- | (PSE/L)Ruc- | (PE))luxABCDE | (PSE)frp | wt | wt |
| 1h243 | 1h242 | GFP | | | | |
| GLV- | GLV- | (PSE/L) Ruc- | (PSE)hNISa | (PSEL)FUKW | wt | wt |
| 1h244 | 1h189 | GFP | | | | |
| GLV- | GLV- | (PSE/L) Ruc- | (PSEL)hNISa | (PSEL)FUKW | wt | wt |
| 1h245 | 1h189 | GFP | | | | |
| GLV- | GLV- | (PSE/L) Ruc- | (PSL)hNISa | (PSEL)FUKW | wt | wt |
| 1h246 | 1h189 | GFP | | | | |
| GLV- | GLV-1h68 | (PSE/L) Ruc- | (PSE/L)TfR- | (PSE)IFP | wt | wt |
| 1h247 | | GFP | (P7.5)lacZ | | | |
| GLV- | GLV-1h68 | (PSE/L) Ruc- | (PSE/L)TfR- | (PSEL)IFP | wt | wt |
| 1h248 | | GFP | (P7.5)lacZ | | | |
| GLV- | GLV-1h68 | (PSE/L) Ruc- | (PSE/L)TfR- | (PSL)IFP | wt | wt |
| 1h249 | | GFP | (P7.5)lacZ | | | |
| GLV- | GLV- | (PSE/L) Ruc- | (PSE/L)TfR- | (PSL)FUKW | wt | wt |
| 1h250 | 1h190 | GFP | (P7.5)lacZ | | | |
| GLV- | GLV-1h68 | (PSE)hNISa | (PSE/L)rTrfR- | (P11)gusA | wt | wt |
| 1h251 | | | (P7.5)lacZ | | | |
| GLV- | GLV-1h68 | (PSE/L)Ruc- | (PSE)hNISa | (P11)gusA | wt | wt |
| 1h252 | | GFP | | | | |
| GLV- | GLV-1h71 | ko | (PSE/L)TfR- | (PSE)FUKW | wt | wt |
| 1h253 | | | (P7.5)lacZ | | | |
| GLV- | GLV-1h71 | ko | (PSE/L)TfR- | (PSL)FUKW | wt | wt |
| 1h254 | | | (P7.5)lacZ | | | |
| GLV- | GLV-1h68 | (PSE/L)Ruc- | (PSE)hMMP9 | (P11)gusA | wt | wt |
| 1h255 | | GFP | | | | |
| GLV- | GLV-1h68 | (PSE/L)Ruc- | (PSL)hMMP9 | (P11)gusA | wt | wt |
| 1h256 | | GFP | | | | |
| GLV- | GLV-1h68 | (PSE/L) Ruc- | (PSE/L)TfR- | (PSE)mNeptune | wt | wt |
| 1h257 | | GFP | (P7.5)lacZ | | | |
| GLV- | GLV-1h68 | (PSE/L) Ruc- | (PSE/L)TfR- | (PSEL)mNeptune | wt | wt |
| 1h258 | | GFP | (P7.5)lacZ | | | |
| GLV- | GLV-1h68 | (PSE/L) Ruc- | (PSE/L)TfR- | (PSL)mNeptune | wt | wt |
| 1h259 | | GFP | (P7.5)lacZ | | | |
| GLV- | GLV-1h68 | (PSE)mNeptune | (PSE/L)rTrfR- | (P11)gusA | wt | wt |
| 1h260 | | | (P7.5)lacZ | | | |
| GLV- | GLV-1h68 | (PSEL)mNeptune | (PSE/L)rTrfR- | (P11)gusA | wt | wt |
| 1h261 | | | (P7.5)lacZ | | | |
| GLV- | GLV-1h68 | (PSL)mNeptune | (PSE/L)rTrfR- | (P11)gusA | wt | wt |
| 1h262 | | | (P7.5)lacZ | | | |
| GLV- | GLV- | (PSE)mNeptune | (PSE)hNET | (PSL)G6-scAb | wt | wt |
| 1h263 | 1h164 | | | | | |
| GLV- | GLV- | (PSEL)mNeptune | (PSE)hNET | (PSL)G6-scAb | wt | wt |
| 1h264 | 1h164 | | | | | |
| GLV- | GLV- | (PSL)mNeptune | (PSE)hNET | (PSL)G6-scAb | wt | wt |
| 1h265 | 1h164 | | | | | |
| GLV- | GLV- | (PSE/L) Ruc- | (PSE)AlstR | (PSEL)FUKW | wt | wt |
| 1h266 | 1h189 | GFP | | | | |
| GLV- | GLV- | (PSE/L) Ruc- | (PSEL)AlstR | (PSEL)FUKW | wt | wt |
| 1h267 | 1h189 | GFP | | | | |
| GLV- | GLV- | (PSE/L) Ruc- | (PSL)AlstR | (PSEL)FUKW | wt | wt |
| 1h268 | 1h189 | GFP | | | | |
| GLV- | GLV- | (PSE/L) Ruc- | (PSE)PEPR1 | (PSEL)FUKW | wt | wt |
| 1h269 | 1h189 | GFP | | | | |
| GLV- | GLV- | (PSE/L) Ruc- | (PSEL)PEPR1 | (PSEL)FUKW | wt | wt |
| 1h270 | 1h189 | GFP | | | | |
| GLV- | GLV- | (PSE/L) Ruc- | (PSL)PEPR1 | (PSEL)FUKW | wt | wt |
| 1h271 | 1h189 | GFP | | | | |
| GLV- | GLV- | (PSE/L) Ruc- | (PSE)LAT4 | (PSEL)FUKW | wt | wt |
| 1h272 | 1h189 | GFP | | | | |
| GLV- | GLV- | (PSE/L) Ruc- | (PSEL)LAT4 | (PSEL)FUKW | wt | wt |
| 1h273 | 1h189 | GFP | | | | |
| GLV- | GLV- | (PSE/L) Ruc- | (PSL)LAT4 | (PSEL)FUKW | wt | wt |
| 1h274 | 1h189 | GFP | | | | |
| GLV- | GLV- | (PSE/L) Ruc- | (PSE)Cyp51 | (PSEL)FUKW | wt | wt |
| 1h275 | 1h189 | GFP | | | | |
| GLV- | GLV- | (PSE/L) Ruc- | (PSEL)Cyp51 | (PSEL)FUKW | wt | wt |
| 1h276 | 1h189 | GFP | | | | |
| GLV- | GLV- | (PSE/L) Ruc- | (PSL)Cyp51 | (PSEL)FUKW | wt | wt |
| 1h277 | 1h189 | GFP | | | | |
| GLV- | GLV- | (PSE/L)Ruc- | (PSE)BMP4 | (PSEL)FUKW | wt | wt |
| 1h284 | 1h189 | GFP | | | | |
| GLV- | GLV- | (PSE/L)Ruc- | (PSEL)BMP4 | (PSEL)FUKW | wt | wt |
| 1h285 | 1h189 | GFP | | | | |
| GLV- | GLV- | (PSE/L)Ruc- | (PSL)BMP4 | (PSEL)FUKW | wt | wt |
| 1h286 | 1h189 | GFP | | | | |

For example, GLV-1h68 (also named RVGL21, SEQ ID NO: 9; described in U.S. Pat. Pub. No. 2005-0031643, now U.S. Pat. Nos. 7,588,767, 7,588,771, 7,662,398) is an attenuated virus of the LIVP strain containing a genome set forth in SEQ ID NO:1 that contains DNA insertions in gene loci F14.5L (also designated in LIVP as F3) gene locus, thymidine kinase (TK) gene locus, and hemagglutinin (HA) gene locus with expression cassettes encoding detectable marker proteins. Specifically, GLV-1h68 contains an expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) inserted into the HA gene locus.

Other recombinant LIVP viruses are derived from GLV-1h68 and contain heterologous DNA that encodes a gene product or products (see e.g. see e.g. U.S. Pub. Nos. US2003-0059400, US2003-0228261, US2007-0202572, US2007-0212727, US2009-0117034, US2009-0098529, US2009-0053244, US2009-0155287, US2009-0081639, US2009-0136917, US2009-0162288, US2010-0062016, US2010-0233078 and US2010-0196325; U.S. Pat. Nos. 7,588,767, 7,588,771, 7,662,398 and 7,754,221 and 7,763,420; and International Pub. No. WO 2009/139921). Exemplary of such recombinant viruses include those set forth in Table 4, including but not limited to, GLV-1h64 (set forth in SEQ ID NO:18); viruses that encode the far-red fluorescent protein TurboFP635 (scientific name “Katushka”; SEQ ID NO:24) from the sea anemone Entacmaea quadricolor, GLV-1h188 (SEQ ID NO: 19), GLV-1h189 (SEQ ID NO: 20), GLV-1h190 (SEQ ID NO: 21), GLV-1h253 (SEQ ID NO: 22), and GLV-1h254 (SEQ ID NO: 23).

Modified vaccinia viruses also include viruses that are modified by introduction of heterologous nucleic acid into an LIVP strain containing a genome set forth in any of SEQ ID NO: 2-8, or a genome that exhibits at least 97%, 98%, 99% or more sequence identity to any of SEQ ID NOS:2-8. For example, exemplary of a modified vaccinia virus is a virus that is modified by insertion, deletion or replacement of heterologous nucleic acid compared to an LIVP strain having a genome set forth in SEQ ID NO:2. Exemplary of such as strain is GLV-2b372, which contains TurboFP635 (Far-red fluorescent protein “katushka”; set forth in SEQ ID NO:24) under the control of the vaccinia synthetic early/late promoter at the TK locus. The genome of GLV-1b372 has the sequence of nucleotides set forth in SEQ ID NO:25.

c. Control of Heterologous Gene Expression

In some examples, the heterologous nucleic acid also can contain one or more regulatory sequences to regulate expression of an open reading frame encoding the heterologous RNA and/or protein. Suitable regulatory sequences which, for example, are functional in a mammalian host cell are well known in the art. Expression can also be influenced by one or more proteins or RNA molecules expressed by the virus. Gene regulatory elements, such as promoters and enhancers, possess cell type specific activities and can be activated by certain induction factors (e.g., hormones, growth factors, cytokines, cytostatic agents, irradiation, heat shock) via responsive elements. A controlled and restricted expression of these genes can be achieved using such regulatory elements as internal promoters to drive the expression of therapeutic genes in viral vector constructs.

For example, the one or more heterologous nucleic acid molecules can be operably linked to a promoter for expression of the heterologous RNA and/or protein. For example, a heterologous nucleic acid that is operably linked to a promoter is also called an expression cassette. Hence, viruses provided herein can have the ability to express one or more heterologous genes. Gene expression can include expression of a protein encoded by a gene and/or expression of an RNA molecule encoded by a gene. In some embodiments, the viruses provided herein can express exogenous genes at levels high enough that permit harvesting products of the exogenous genes from the tumor. Expression of heterologous genes can be controlled by a constitutive promoter, or by an inducible promoter. In other examples, organ or tissue-specific expression can be controlled by regulatory sequences. In order to achieve expression only in the target organ, for example, a tumor to be treated, the foreign nucleotide sequence can be linked to a tissue specific promoter and used for gene therapy. Such promoters are well known to those skilled in the art (see, e.g., Zimmermann et al., Neuron 12: 11-24 (1994); Vidal et al., EMBO J. 9: 833-840 (1990); Mayford et al., Cell 81: 891-904 (1995); and Pinkert et al., Genes & Dev. 1: 268-76 (1987)).

Exemplary promoters for the expression of heterologous genes are known in the art. The heterologous nucleic acid can be operatively linked to a native promoter or a heterologous promoter that is not native to the virus. Any suitable promoters, including synthetic and naturally-occurring and modified promoters, can be used. Exemplary promoters include synthetic promoters, including synthetic viral and animal promoters. Native promoter or heterologous promoters include, but are not limited to, viral promoters, such as vaccinia virus and adenovirus promoters.

In one example, the promoter is a poxvirus promoter, such as, for example, a vaccinia virus promoter. Vaccinia viral promoters for the expression of one or more heterologous genes can be synthetic or natural promoters, and include vaccinia early, intermediate, early/late and late promoters. Exemplary vaccinia viral promoters for controlling heterologous gene expression include, but are not limited to, P_{7.5k}, P₁₁ₖ, P_{SE}, P_{SEL}, P_{SL}, H5R, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b or K1 promoters. Other viral promoters include, but are not limited to, adenovirus late promoter, Cowpox ATI promoter, or T7 promoter. Strong late promoters can be used to achieve high levels of expression of the heterologous genes. Early and intermediate-stage promoters also can be used. In one example, the promoters contain early and late promoter elements, for example, the vaccinia virus early/late promoter vaccinia late promoter P₁₁ₖ, a synthetic early/late vaccinia P_{SEL} promoter (Patel et al., (1988) Proc. Natl. Acad. Sci. USA 85: 9431-9435; Davison and Moss, (1989) J Mol Biol 210: 749-769; Davison et al. (1990) Nucleic Acids Res. 18: 4285-4286; Chakrabarti et al. (1997), BioTechniques 23: 1094-1097). The viruses provided herein can exhibit differences in characteristics, such as attenuation, as a result of using a stronger promoter versus a weaker promoter. For example, in vaccinia, synthetic early/late and late promoters are relatively strong promoters, whereas vaccinia synthetic early, P_{7.5k} early/late, P_{7.5k} early, and P₂₈ late promoters are relatively weaker promoters (see e.g., Chakrabarti et al. (1997) BioTechniques 23(6) 1094-1097). Combinations of different promoters can be used to express different gene products in the same virus or two different viruses.

As is known in the art, regulatory sequences can permit constitutive expression of the exogenous gene or can permit inducible expression of the exogenous gene. Further, the regulatory sequence can permit control of the level of expression of the exogenous gene. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

Hence, expression of heterologous genes can be controlled by a constitutive promoter or by an inducible promoter. Inducible promoters can be used to provide tissue specific expression of the heterologous gene or can be inducible by the addition of a regulatory molecule to provide temporal specific induction of the promoter. In some examples, inducible expression can be under the control of cellular or other factors present in a tumor cell or present in a virus-infected tumor cell. In further examples, inducible expression can be under the control of an administrable substance, including IPTG, RU486 or other known induction compounds. Additional regulatory sequences can be used to control the expression of the one or more heterologous genes inserted the virus. Any of a variety of regulatory sequences are available to one skilled in the art according to known factors and design preferences.

d. Methods of Generating Modified Viruses

The viruses for use in the compositions herein can be modified by insertion, deletion, replacement or mutation as described herein, for example insertion or replacement of heterologous nucleic acid, using standard methodologies well known in the art for modifying viruses. Methods for modification include, for example, in vitro recombination techniques, synthetic methods, direct cloning, and in vivo recombination methods as described, for example, in Sambrook et al., Molecular Cloning: A Laboratory Manual, 2nd edition, Cold Spring Harbor Laboratory Press, cold Spring Harbor N.Y. (1989), and in the Examples disclosed herein.

For example, generation of recombinant viruses, including recombinant vaccinia virus, is well known in the art, and typically involves the generation of gene cassettes or transfer vectors using standard techniques in molecular biology (see, e.g., U.S. Pat. No. 7,588,767 and US2009-0053244-A1, which describe exemplary methods of generating recombinant LIVP vaccinia viruses). Such techniques include various nucleic acid manipulation techniques, nucleic acid transfer protocols, nucleic acid amplification protocols, and other molecular biology techniques known in the art.

For example, point mutations or small insertions or deletions can be introduced into a gene of interest through the use of oligonucleotide mediated site-directed mutagenesis. In another example, homologous recombination can be used to introduce a mutation in the nucleic acid sequence or insertion or deletion of a nucleic acid molecule into a target sequence of interest. In some examples, mutations, insertions or deletions of nucleic acid in a particular gene can be selected for using a positive or negative selection pressure. See, e.g., Current Techniques in Molecular Biology, (Ed. Ausubel, et al.).

Nucleic acid amplification protocols include, but are not limited to, the polymerase chain reaction (PCR), or amplification via viruses or organisms, such as, but not limited to, bacteria, yeast, insect or mammalian cells. Use of nucleic acid tools such as plasmids, vectors, promoters and other regulating sequences, are well known in the art for a large variety of viruses and cellular organisms.

Nucleic acid transfer protocols include calcium chloride transformation/transfection, electroporation, liposome mediated nucleic acid transfer, N-[1-(2,3-dioloyloxy)propyl]-N,N,N-trimethylammonium methylsulfate meditated transformation, and others. Further a large variety of nucleic acid tools are available from many different sources, including various commercial sources. One skilled in the art will be readily able to select the appropriate tools and methods for genetic modifications of any particular virus according to the knowledge in the art and design choice.

Hence, any of a variety of modifications can be readily accomplished using standard molecular biological methods known in the art. The modifications will typically be one or more truncations, deletions, mutations or insertions of the viral genome. In one example, the modification can be specifically directed to a particular sequence in the viral genome. The modifications can be directed to any of a variety of regions of the viral genome, including, but not limited to, a regulatory sequence, a gene-encoding sequence, an intergenic sequence, a sequence without a known role, or a non-essential region of the viral genome. Any of a variety of regions of viral genomes that are available for modification are readily known in the art for many viruses, including LIVP.

Heterologous nucleic acid molecules are typically inserted into the viral genome in an intergenic region or in a locus that encodes a nonessential viral gene product. Insertion of heterologous nucleic acid at such sites generally does not significantly affect viral infection or replication in the target tissue. Exemplary insertion sites are known in the art and include, but are not limited to, J2R (thymidine kinase (TK)), A56R (hemagglutinin (HA)), F14.5L, vaccinia growth factor (VGF), A35R, N1L, E2L/E3L, K1L/K2L, superoxide dismutase locus, 7.5K, C7-K1L (host range gene region), B13R+B14R (hemorrhagic region), A26L (A type inclusion body region (ATI)) or I4L (large subunit, ribonucleotide reductase) gene loci. Insertion sites for the viruses provided herein also include sites that correspond to intragenic regions described in other poxviruses such as Modified Vaccinia Ankara (MVA) virus (exemplary sites set forth in U.S. Pat. No. 7,550,147), NYVAC (exemplary sites set forth in U.S. Pat. No. 5,762,938).

Methods for the generation of recombinant viruses using recombinant DNA techniques are well known in the art (e.g., see U.S. Pat. Nos. 4,769,330; 4,603,112; 4,722,848; 4,215,051; 5,110,587; 5,174,993; 5,922,576; 6,319,703; 5,719,054; 6,429,001; 6,589,531; 6,573,090; 6,800,288; 7,045,313; He et al. (1998) PNAS 95(5): 2509-2514; Racaniello et al., (1981) Science 214: 916-919; and Hruby et al., (1990) Clin Micro Rev. 3:153-170). Methods for the generation of recombinant vaccinia viruses are well known in the art (e.g., see Hruby et al., (1990) Clin Micro Rev. 3:153-170, U.S. Pat. Pub. No. 2005-0031643, now U.S. Pat. Nos. 7,588,767, 7,588,771, 7,662,398 and U.S. Pat. No. 7,045,313).

For example, generating a recombinant vaccinia virus that expresses a heterologous gene product typically includes the use of a recombination plasmid which contains the heterologous nucleic acid, optionally operably linked to a promoter, with vaccinia virus DNA sequences flanking the heterologous nucleic acid to facilitate homologous recombination and insertion of the gene into the viral genome. Generally, the viral DNA flanking the heterologous gene is complementary to a non-essential segment of vaccinia virus DNA, such that the gene is inserted into a nonessential location. The recombination plasmid can be grown in and purified from Escherichia coli and introduced into suitable host cells, such as, for example, but not limited to, CV-1, BSC-40, BSC-1 and TK-143 cells. The transfected cells are then superinfected with vaccinia virus which initiates a replication cycle. The heterologous DNA can be incorporated into the vaccinia viral genome through homologous recombination, and packaged into infection progeny. The recombinant viruses can be identified by methods known in the art, such as by detection of the expression of the heterologous gene product, or by using positive or negative selection methods (U.S. Pat. No. 7,045,313).

In another example, the recombinant vaccinia virus that expresses a heterologous gene product can be generated by direct cloning (see, e.g. U.S. Pat. No. 6,265,183 and Scheiflinger et al. (1992) Proc. Natl. Acad. Sci. USA 89: 9977-9981). In such methods, the heterologous nucleic acid, optionally operably linked to a promoter, is flanked by restriction endonuclease cleavage sites for insertion into a unique restriction endonuclease site in the target virus. The virus DNA is purified using standard techniques and is cleaved with the sequence-specific restriction endonuclease, where the sequence is a unique site in the virus genome. Any unique site in the virus genome can be employed provided that modification at the site does not interfere with viral replication. For example, in vaccinia virus strain LIVP, the NotI restriction site is located in the ORF encoding the F14.5L gene with unknown function (Mikryukov et al., Biotekhnologiya 4: 442-449 (1988)). Table 5 provides a summary of unique restriction sites contained in exemplary LIVP strains and designates the nucleotide position of each. Such LIVP strains can be modified herein by direct cloning and insertion of heterologous DNA into the site or sites. Generally, insertion is in a site that is located in a non-essential region of the virus genome. For example, exemplary modifications herein include insertion of a foreign DNA sequence into the NotI digested virus DNA.

TABLE 5
Unique restriction endonuclease cleavage sites in LIVP clonal isolates

| Restriction Enzyme/ | | | | | | | | | LIVP |
|---|---|---|---|---|---|---|---|---|---|
| Site | | 1.1.1 | 2.1.1 | 4.1.1 | 5.1.1 | 6.1.1 | 7.1.1 | 8.1.1 | Parental |
| Name/ | SEQ | (SEQ ID | (SEQ ID | (SEQ ID | (SEQ ID | (SEQ ID | (SEQ ID | (SEQ ID | (SEQ ID |
| sequence | ID NO | NO: 2) | NO: 3) | NO: 4) | NO: 5) | NO: 6) | NO: 7) | NO: 8) | NO: 1) |
| SbfI | 64 | 40033/ | 40756/ | 39977/ | 40576/ | 40177/ | 40213/ | 40493/ | 38630/ |
| CCTGCAGG | | 40029 | 40752 | 39973 | 40572 | 40173 | 40209 | 40489 | 38626 |
| NotI | 65 | 42989/ | 43712/ | 42933/ | 43532/ | 43133/ | 43169/ | 43449/ | 41586/ |
| GCGGCCGC | | 42998 | 43716 | 42937 | 43536 | 43137 | 43173 | 43453 | 41590 |
| SgrAI | 66 | 114365/ | 115107/ | 114308/ | 114924/ | 114489/ | 114548/ | 114845/ | 112975/ |
| CRCCGGYG | | 114369 | 115111 | 114312 | 114928 | 114493 | 114552 | 114849 | 112979 |
| SmaI | 67 | 159260 | NA | NA | NA | NA | NA | NA | NA |
| CCCGGG | | | | | | | | | |
| TspMI | 68 | 159258/ | NA | NA | NA | NA | NA | NA | NA |
| CCCGGG | | 159262 | | | | | | | |
| XmaI | 69 | 159258/ | NA | NA | NA | NA | NA | NA | NA |
| CCCGGG | | 159262 | | | | | | | |
| ApaI | 70 | 180516/ | NA | 180377/ | 181027/ | 180638/ | 180596/ | 180972/ | NA |
| CCCGGG | | 180512 | | 180373 | 181023 | 180634 | 180592 | 180968 | |
| PspOMI | 71 | 180512/ | NA | 180373/ | 181023/ | 180634/ | 180592/ | 180968/ | NA |
| CCCGGG | | 180516 | | 180377 | 181027 | 180638 | 180596 | 180972 | |

In some examples, the virus genomic DNA is first modified by homologous recombination to introduce one or more unique restriction sites in the virus (see, e.g. Mackett et al. (1984) J. Virol. 857-864). Following cleavage with the restriction endonuclease, the cleaved DNA is optionally treated with a phosphatase to remove a phosphate moiety from an end of the DNA segment that is produced by cleavage with the endonuclease. Typically, a plasmid vector is generated that contains the heterologous DNA for insertion flanked by the restriction sites. Prior to insertion into the virus, the heterologous DNA is excised from the plasmid by cleavage with the sequence specific restriction endonuclease. The heterologous DNA is then ligated to the cleaved viral DNA and is packaged in a permissive cell line by infection of the cells with a helper virus, such as, but not limited to a fowpox virus or a puv-inactivated helper vaccinia virus, and transfection of the ligated DNA into the infected cells.

In some examples, the methods involve homologous recombination and/or use of unique restriction sites in the virus. For example, a recombinant LIVP vaccinia virus with an insertion, for example, in the F14.5L gene (e.g., in the Not I restriction site of an LIVP isolate) can be prepared by the following steps: (a) generating (i) a vaccinia shuttle/transfer plasmid containing the modification (e.g. a gene expression cassette or a modified F14.5L gene) inserted at a restriction site, X (e.g. Not I), where the restriction site in the vector is flanked by parental virus sequences of the target insertion site and (ii) an LIVP virus DNA digested at restriction site X (e.g. Not I) and optionally dephosphorylated; (b) infecting cells with PUV-inactivated helper vaccinia virus and transfecting the infected host cells with a mixture of the constructs of (i) and (ii) of step a; and (c) isolating the recombinant vaccinia viruses from the transfectants. One skilled in the art knows how to perform such methods (see, e.g., Timiryasova et al. (Biotechniques 31: 534-540 (2001)). Typically, the restriction site X is a unique restriction site in the virus as described above.

In one example, the methods include introducing into the viruses one or more genetic modifications, followed by screening the viruses for properties reflective of the modification or for other desired properties. In some examples, the modification can be fully or partially random, whereupon selection of any particular modified virus can be determined according to the desired properties of the modified the virus.

3. Methods of Producing Viruses

Viruses in the compositions provided herein can be produced by methods known to one of skill in the art. Typically, the virus is propagated in host cells, quantified and prepared for storage before finally being prepared in the compositions described herein. The virus can be propagated in suitable host cells to enlarge the stock, the concentration of which is then determined. In some examples, the infectious titer is determined, such as by plaque assay. The total number of viral particles also can be determined. The viruses are stored in conditions that promote stability and integrity of the virus, such that loss of infectivity over time is minimized. In some examples, a large amount of virus is produced and stored in small aliquots of known concentration that can be used for multiple procedures over an extended period of time. Conditions that are most suitable for various viruses will differ, and are known in the art, but typically include freezing or drying, such as by lyophilization. The viruses can be stored at a concentration of 10⁵-10¹⁰ pfu/mL, for example, 10⁷-10⁹ pfu/mL, such as at least or about or 10⁶ pfu/mL, 10⁷ pfu/mL, 10⁸ pfu/mL or 10⁹ pfu/mL. Immediately prior to preparing compositions provided herein, the stored viruses can be reconstituted (if dried for storage) and diluted in an appropriate medium or solution.

The following sections provide exemplary methods that can be used for the production and preparation of viruses for use in preparing viruses in the compositions provided herein.

a. Host Cells for Propagation

Virus strains can be propagated in an appropriate host cell. Such cells can be a group of a single type of cells or a mixture of different types of cells. Host cells can include cultured cell lines, primary cells, and proliferative cells. These host cells can include any of a variety of animal cells, such as mammalian, avian and insect cells and tissues that are susceptible to the virus, such as vaccinia virus, infection, including chicken embryo, rabbit, hamster, and monkey kidney cells. Suitable host cells include, but are not limited to, hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, and cell lines including, for example, CV-1, BSC40, Vero, and BSC-1, and human HeLa cells. Typically, viruses are propagated in cell lines that that can be grown at monolayers or in suspension. For example, exemplary cell lines for the propagation of vaccinia viruses include, but are not limited to, CV-1, BSC40, Vero, BGM, BSC-1 and RK-13 cells. Purification of the cultured strain from the system can be effected using standard methods.

b. Concentration Determination

The concentration of virus in a solution, or virus titer, can be determined by a variety of methods known in the art. In some methods, a determination of the number of infectious virus particles is made (typically termed plaque forming units (PFU)), while in other methods, a determination of the total number of viral particles, either infectious or not, is made. Methods that calculate the number of infectious virions include, but are not limited to, the plaque assay, in which titrations of the virus are grown on cell monolayers and the number of plaques is counted after several days to several weeks, and the endpoint dilution method, which determines the titer within a certain range, such as one log. Methods that determine the total number of viral particles, including infectious and non-infectious, include, but are not limited to, immunohistochemical staining methods that utilize antibodies that recognize a viral antigen and which can be visualized by microscopy or FACS analysis; optical absorbance, such as at 260 nm; and measurement of viral nucleic acid, such as by PCR, RT-PCR, or quantitation by labeling with a fluorescent dye.

c. Storage Methods

Once the virus has been purified (or to a desired purity) and the titer has been determined, the virus can be stored in conditions which optimally maintain its infectious integrity. Typically, viruses are stored in the dark, because light serves to inactivate the viruses over time. Viral stability in storage is usually dependent upon temperatures. Although some viruses are thermostable, most viruses are not stable for more than a day at room temperature, exhibiting reduced viability (Newman et al., (2003) J. Inf. Dis. 187:1319-1322). Vaccinia virus is generally stable at refrigerated temperatures, and can be stored in solution at 4° C., frozen at, for example −20° C., −70° C. or −80° C., or lyophilized with little loss of viability (Newman et al., (2003) J. Inf. Dis. 187:1319-1322, Hruby et al., (1990) Clin. Microb. Rev. 3:153-170). Methods and conditions suitable for the storage of particular viruses are known in the art, and can be used to store the viruses used in the methods presented herein. It is understood that stability of the virus can be increased by providing the viruses in protein polymer (e.g. SELP) compositions as provided herein. This section describes parameters for storage of viruses that are not contained in a protein polymer.

For short-term storage of viruses, for example, 1 day, 2 days, 4 days or 7 days, temperatures of approximately 4° C. are generally recommended. For long-term storage, most viruses can be kept at −20° C., −70° C. or −80° C. When frozen in a simple solution such as PBS or Tris solution (20 mM Tris pH 8.0, 200 NaCl, 2-3% glycerol or sucrose) at these temperatures, the virus can be stable for 6 months to a year, or even longer. Repeated freeze-thaw cycles are generally avoided, however, since it can cause a decrease in viral titer. The virus also can be frozen in media containing other supplements in the storage solution which can further preserve the integrity of the virus. For example, the addition of serum or bovine serum albumin (BSA) to a viral solution stored at −80° C. can help retain virus viability for longer periods of time and through several freeze-thaw cycles.

In other examples, the virus sample is dried for long-term storage at ambient temperatures. Viruses can be dried using various techniques including, but not limited to, freeze-drying, foam-drying, spray-drying and desiccation. Water is a reactant in nearly all of the destructive pathways that degrade viruses in storage. Further, water acts as a plasticizer, which allows unfolding and aggregation of proteins. Since water is a participant in almost all degradation pathways, reduction of the aqueous solution of viruses to a dry powder provides an alternative composition methodology to enhance the stability of such samples. Lyophilization, or freeze-drying, is a drying technique used for storing viruses (see, e.g., Cryole et al., (1998) Pharm. Dev. Technol., 3(3), 973-383). There are three stages to freeze-drying; freezing, primary drying and secondary drying. During these stages, the material is rapidly frozen and dehydrated under high vacuum. Once lyophilized, the dried virus can be stored for long periods of time at ambient temperatures, and reconstituted with an aqueous solution when needed. Various stabilizers can be included in the solution prior to freeze-drying to enhance the preservation of the virus. For example, it is known that high molecular weight structural additives, such as serum, serum albumin or gelatin, aid in preventing viral aggregation during freezing, and provide structural and nutritional support in the lyophilized or dried state. Amino acids such as arginine and glutamate, sugars, such as trehalose, and alcohols such as mannitol, sorbitol and inositol, can enhance the preservation of viral infectivity during lyophilization and in the lyophilized state. When added to the viral solution prior to lyophilization, urea and ascorbic acid can stabilize the hydration state and maintain osmotic balance during the dehydration period. Typically, a relatively constant pH of about 7.0 is maintained throughout lyophilization.

Other methods for the storage of viruses at ambient, refrigerated or freezing temperatures are known in the art, and include, but are not limited to, those described in U.S. Pat. Nos. 5,149,653; 6,165,779; 6,255,289; 6,664,099; 6,872,357; and 7,091,030; and in U.S. Pat. Pub. Nos. 2003-0153065, 2004-003841 and 2005-0032044.

d. Preparation of Virus

Immediately prior to use, the virus can be prepared at an appropriate concentration in suitable media, and can be maintained at a cool temperature, such as on ice, until use. If the virus was lyophilized or otherwise dried for storage, then it can be reconstituted in an appropriate aqueous solution. The aqueous solution in which the virus is prepared is typically the medium used in the assay (e.g., DMEM or RPMI) or one that is compatible, such as a buffered saline solution (e.g., PBS, TBS, Hepes solution). For pharmaceutical applications, the virus can be immediately prepared or reconstituted in a pharmaceutical solution. Numerous pharmaceutically acceptable solutions for use are well known in the art (see e.g. Remington's Pharmaceutical Sciences (18^{th} edition) ed. A. Gennaro, 1990, Mack Publishing Co., Easton, Pa.). In one example, the viruses can be diluted in a physiologically acceptable solution, such as sterile saline or sterile buffered saline, with or without an adjuvant or carrier. In other examples, the pharmaceutical solution can contain a component that provides viscosity (e.g. glycerol) and/or component that has bactericidal properties (e.g. phenol). The virus can be reconstituted or diluted to provide the desired concentration or amount. The particular concentration can be empirically determined by one of skill in the art depending on the particular application.

### D. PROTEIN POLYMERS

The compositions provided herein contain a vaccinia virus in a protein polymer matrix or hydrogel. Hydrogel-forming polymers are polymers that are capable of absorbing a substantial amount of water to form elastic or inelastic gels. Hydrogel-forming polymers offer the flexibility of being maintained or delivered in liquid or gelled form. For example, the vaccinia virus in protein polymer compositions provided herein can be liquid at room temperature and form hydrogels at body temperature. The release of vaccinia virus incorporated into the hydrogel takes place through the gelled matrix via a diffusion mechanism.

Exemplary protein polymers include, but are not limited to, any described in U.S. Pat. Nos. 5,243,038, 5,641,648, 5,760,004, 5,770,697, 5,773,249, 5,830,713, 6,148,348, 6,140,072, 6,034,220, 6,018,030, 6,355,766, 7,546,147,7,662.409 or 6,380,154; U.S. Published Patent Application Nos. US2007/0098702, US2010/0022455, US2009/0093621, US2010/143487 or US2010/0261652; International Published PCT Application No. WO2004/104020; or Capello et al. (1998) J. Controlled Release, 53:105-117, Ghandehari et al. (2009) Polymer, 50:366-374; Haider et al. (2005) Molecular Phamaceutics, 2:139-150; Price et al. (2012) International Journal of Pharmaceutics, 427:97-104; and Megeed et al. (2002) Advanced Drug Delivery Reviews, 54:1075-1091).

A protein polymer for use in preparing the hydrogels compositions herein can be composed of a protein or proteins having multiple repeating amino acid residues.

The protein generally contains repetitive amino acid units of from or about between 3 to 20 amino acids. The protein typically is a natural protein or is derivative, artificial and/or synthetic sequence thereof. In particular, the protein is one that is capable of being degraded and/or safely resorbed upon administration to a subject in vivo.

Exemplary protein polymers are composed of silk-like units, elastin-like units, collagen-like units, keratin-like units, or any combination thereof. For example, silk-like proteins have a repeating unit of GAGAGS (SEQ ID NO:26) or SGAGAG (SEQ ID NO:27), which is a repeating unit that is found naturally in silk fibroin protein represented as GAGAG(SGAGAG)₈SGAAGY (SEQ ID NO:28). Elastin-like proteins have a base repeating unit of GVGVP (SEQ ID NO.29), VPGG (SEQ ID NO:30), APGVGV (SEQ ID NO:31), or VPGVG (SEQ ID NO:32), which are repeating units found in naturally occurring elastin. Collagen-like proteins have repeating triad units of G-x-y (x=any amino acid, often alanine or proline; y=any amino acid, often proline or hydroxy-proline). Typically, x and y are selected such that the proline content in the triads of the polymer is less than about 45% (see e.g. U.S. Pat. No. 5,773,249). A single collagen-like unit can contain at least about 2 and not more than about 100 tandemly repeated triads, more usually not more than about 75, frequently not more than about 50, more frequently not more than about 25. Keratin-like units contain a “heptad” repeat unit made up of a seven amino acid long stretch with two positions separated by two amino acids, usually positions three and six, occupied consistently with hydrophobic, aliphatic or aromatic residues (see e.g., U.S. Pat. No. 5,514,581). Exemplary of such repeating units are AKLKLAE (SEQ ID NO:33) or AKLELAE (SEQ ID NO:34). Other natural proteins containing repetitive amino acids units are known (see e.g. U.S. Pat. No. 6,355,776).

It is understood that variations in amino acids in the repeating unit are permitted, such as the particular order of the amino acids in the sequence and conservative substitutions, such as, but not limited to, replacing serine with threonine and glycine with alanine. For example, amino acid sequence units and elements of the polymers can be modified by amino acid replacement, e.g. conservative substitution, of amino acids at various positions in their sequences. For example, examples of modified elastin-like blocks have been reported (see e.g., Urry et al. (1992) Biopolymers 32:1243-1250). Substitutions of amino acids can impart changes in the chemical nature of the protein within which these blocks reside. For example, the replacement of the first valine in GVGVP (SEQ ID NO:29) with a more hydrophobic amino acid such as phenylalanine will decrease the lower critical solution temperature at which the elastin-like protein polymer is soluble. Replacing this valine with a more hydrophilic amino acid such as lysine will increase the lower critical solution temperature of the polymer solution. While these modified elastin-like blocks can affect certain chemical or physical properties, they can be readily chosen so as not to destroy the ability of protein polymers containing crystallizable silk-like blocks to acquire a non-liquid form, i.e., by gelation or solidification. Exemplary modified elastin-like units also include those depicted by a base repeating unit of GXGVP (SEQ ID NO:35) or VPGXG (SEQ ID NO:36), where X is valine, lysine, histidine, glutamic acid, arginine, aspartic acid, serine, tryptophan, tyrosine, phenylalanine, leucine, glutamine, asparagine, cysteine or methionine, and typically valine or lysine.

Exemplary of alternating units in the polymers herein are amino acid sequences that 1) promote protein crystallization (e.g. silk-like amino acid sequence unit) and 2) influence water solubility (e.g. keratin-like, collagen-like or elastin-like amino acid sequence unit). A protein polymer can have the following formulas: [(C)a(X)b]c or [(X)_{b}(C)a]c, where “X” represents an amino acid sequence element that is an elastin-like, collagen-like or keratin-like unit and “b” represents the number of such units present in the monomer segment, “C” represents an amino acid sequence unit of from about 3 to 30 amino acid which promotes protein crystallization and “a” represents the number of such units present in the monomer segment and “c” represents the number of monomer units which are repeated in the protein polymer. Monomer segments are generally composed of multiple protein crystallization units (e.g. silk-like elastin) followed by multiple elastin-like, collagen-like or keratin-like units or vice versa (as shown in the above formulas), however, insertion of one or more of one unit type within a run of multiple units of the other unit type can also be employed. By selecting the number and order of amino acid motifs or repeats in the monomeric units of each polymer, the hydrophilic properties of the polymer can be modified.

Amino acid sequence units that promote protein crystallization are sequences from about 3 to 30 amino acids in length which, for the most part, possess relatively simple amino acids with relatively low molecular weight side chains including, for example, glycine, alanine, serine, threonine, cysteine and valine. Because these “protein crystallization units” possess, for the most part, relatively small molecular weight amino acids, they are capable of forming extended chain conformations such as β-sheets or β-strands that allow chains of the polypeptide to come into close proximity where hydrogen bonding may occur. These units allow the formation of ordered structures. Different protein crystallization units are known in the art (e.g., Fossey et al., Biopolymers 31(13):1529-1542 (1991)). Exemplary of protein crystallization units for use in protein polymers are “silk-like” units that generally possess the amino acid sequence GAGAGS (SEQ ID NO:26) or SGAGAG (SEQ ID NO:27). The silk-like units can be combined with an elastin-like unit to generate silk-elastin protein polymers (SELPs).

The ratio of the alternating units can be adjusted depending on the desired hydrophilic or other properties of the polymer. For example, in the case of SELPs, the nature of the elastinlike blocks, and their length and position within the monomers influences the water solubility of the SELP. For example, decreasing the length and/or content of the silk-like block domains, while maintaining the length of the elastin-like block domains, increases the water solubility of the polymers. Generally, the ratio of amino acid sequence units that promote protein crystallization (e.g. silk-like amino acid sequence unit) versus that amino acid sequence unit that influence water solubility (e.g. keratin-like, collagen-like or elastin-like amino acid sequence unit) per monomer segment is in the range of about 0.5, usually about 1 to 5. For the most part, there will be at least two protein crystallization units (e.g. silk-like amino acid sequence unit) per monomer segment and not more than about 16, usually not more than about 12, and generally ranging from about 2 to 8 or from about 4 to 8. For the elastin-like, collagen-like or keratin-like amino acid sequence elements, there will usually be at least two per monomer segment, typically at least about four, generally ranging from about 6 to 32, 6 to 18 or 6 to 16.

Typically, the polymers provided herein contain a plurality of monomer segments in order to generate higher molecule weight protein polymers. The polymers in the compositions provided herein contain multiple repeats of monomer units. The protein polymers are at least about 15 kDa and generally not more than about 250 kDa, usually not more than about 175 kDa, more usually not more than 125 kDa, typically ranging from about 15 to 100 kDa and more generally from about 50 to 90 kDa in size. In order to achieve repetitive protein polymers within these molecular weight ranges, the number of repetitive monomer segments incorporated into the polymer will provide for the desired molecular weight. In this regard, the number of monomer segments in the polymer can vary widely, depending upon the size of each individual monomer. Thus, the number of monomers can vary generally from about 2 to 100, usually from about 2 to 40, more usually ranging from about 6 to 20 and generally from about 8 to 13.

The amino acid sequence of protein polymers in the compositions herein also can contain non-repetitive amino acid units at the N- and C-termini of the protein polymer. Such sequences are referred to as the head and tail portions of the amino acid sequence of the polymer. Usually, the terminal sequences will contribute fewer than ten number percent of the total amino acids, more usually fewer than five number percent of the total amino acids present in the polymer. Generally, the terminal amino acid sequences will range from about 0-125 amino acids, more usually from about 0-60 amino acids, where the total number of amino acids will generally not exceed about 100 amino acids, more usually not exceed about 50 amino acids. For example, based upon the method of preparation of the polymer, the N- and/or C-termini can contain additional non-repetitive amino acid sequences. For example, generally, the presence of a non-repetitive N-terminus will be the result of insertion of the gene into a vector in a manner that results in expression of a fusion protein. Any protein which does not interfere with the desired properties of the product can provide the N-terminus. Particularly, endogenous host proteins, e.g. bacterial proteins, can be employed. The choice of protein can depend on the nature of the transcriptional initiation region. The N- and C-terminal sequence can be one that can be, if desired, removed in whole or in part by a protease.

The protein polymers also can be modified to contain intervening amino acid sequences between one or more monomer segments or the alternating block units which make up the monomer segment or by otherwise modifying one or more amino acid residues present in the polymer. Intervening sequences can include from about 1 to 60, usually about 3 to 40 amino acids, and may provide for a wide variety of properties including promotion of polymer chain interactions mediated by hydrogen bonding, salt bridges and/or hydrophobic interactions. For example, by including amino acids that have chemically reactive sidechains, sites can be provided for linking a variety of chemically or physiologically active compounds, for cross-linking, for covalently bonding compounds that can change the rate of resorption, tensile properties, or for altering the rate of release of an incorporated biologically active compound. Thus, amino acids such as cysteine, aspartic acid, glutamic acid, lysine and arginine can be incorporated in these intervening sequences. Alternatively, intervening sequences can provide for sequences that exhibit a physiological property or activity, such as cell binding, specific protein binding, enzyme substrates, or specific receptor binding. In addition, one or more amino acid residues in the polymer can be modified, either chemically or otherwise, to provide for or influence a particular property, such as polymerization rates, tensile strengths, or rates of resorption in vivo. For example, hydroxyalkylation at various amino acid sites can be made.

1. Silk-Elastin Like Polymers (SELP)

Exemplary protein polymers for use in the compositions herein is a silk-elastinlike protein polymer (SELP). SELPs are exemplary polymers that are initially water soluble, but can spontaneously convert to gels. SELPs contain monomer units composed of alternating amino acid sequence units that are identical or similar to those found in natural silks and elastins. The silk units permit formation of hydrogen bonds for the formation of hydrogels, while the elastin units confer aqueous solubility.

For example, the units employed in SELP protein polymers generally have the “silk-like” amino acid sequences GAGAGS (SEQ ID NO:26) or SGAGAG (SEQ ID NO:27) and the “elastin-like” amino acid sequences VPGG (SEQ ID NO:30), APGVGV (SEQ ID NO:31), VPGVG (SEQ ID NO:32), VPGKG (SEQ ID NO:37), GKGVP (SEQ ID NO:38) or GVGVP (SEQ ID NO:29). It is understood that the particular sequence can be varied to alter a property of the polymer, such as is described below, by inclusion of conservative substitutions, such as, but not limited to, replacing serine with threonine and glycine with alanine, or by the particular order of the amino acids in the sequence. Also, in some examples, the particular sequence can be varied by having alternate multimers with the same or different handedness.

At body temperature, SELPS undergo a nonreversible crystallization event resulting in gelation, which can be controlled by adjusting the copolymer structure. For example, the silk and elastin units can be combined in various ratios and sequences to produce polymers with various properties. For example, the addition of elastin units disrupts the crystalline structure in silks and makes the silk-elastin copolymers more water soluble. In contrast, increasing the number of silk units increases the rate of gelation. In addition, the length of the polymer and its molecular weight also influence the degree of cross-linking of formation of hydrogels. Thus, varying the number of monomer repeats in the polymer also can influence the properties of gelation, release and biodegradation of the SELPs. In the compositions herein, the choice of polymer is such that the compositions are liquid at room temperature, and form a hydrogel within minutes upon exposure to body temperature, for example, after injection. For example, the exemplary SELP, SELP-47K undergoes a soluble in aqueous medium to gel transition to form hydrogels (sol-to-gel) that is slow at room temperature (several occurs), but that occurs within minutes at 37° C. It is within the level of one of skill in the art to empirically choose a SELP for use in the composition herein depending on the particular virus, the particular application or treatment, including the particular tumor or the particular route of administration (e.g. topical or intravenous).

Generally, silk-elastinlike polymers (also called ProLastins) can be defined using the general formula {[S]ₘ[E]ₙ}_{O}, where “S” is the silklike block or other similar sequence and “E” is an elastic-like block or other similar sequence (see e.g. Cappello et al. (1998) Journal of Controlled. Release, 53:105-117). Generally, m is from 2 to 16, such as 2 to 8 and n can be any number, but generally is from 1 to 16. In particular, the ratio of silk-like units (m) to elastin-like units (n) can be from 1:20 to 20:1. In other aspects, the ratio is 1:1 to 1:16, such as 1:2 to 1:10, 1:2 to 1:8, 1:2 to 1:6, or 1:2 to 1:4. With respect to length and size of the polymer, o is the number of monomer repeats and generally is from 2 to 100. The protein polymers generally have a molecular weight from 15,000 to 100,000 daltons (Da), for example, 60,000 to 85,000 Da. The number of monomer repeats o can be chosen to yield this molecular weight depending on the chosen units m and n.

Exemplary SELPs are set forth in Table 6. In addition to the repeating units or blocks, all polymers also can contain additional N- and C-terminal sequences (heads and tail sequences). Exemplary of a head sequence is MDPVVLQRRDWENPGVTQLNRLAAHPPFASDPM (set forth in SEQ ID NO:58). Exemplary of a tail sequence is GAGAMDPGRYQDLRSHHHHHH (set forth in SEQ ID NO:59) or GAMDPGRYQDLRSHHHHHH (set forth in SEQ ID NO:60). The head and tail portions are derived from the genetic expression system employed and can be removed by specific chemical cleavage if desired.

TABLE 6
Exemplary SELPs

| | | Repeated Sequence^{a} | |
|---|---|---|---|
| NAME | MW | (Seq ID) | Reference |
| SELP 0 | 80,502 | [(VPGVG)₈(GAGAGS)₂]₁₈ | U.S. Pat. No. 6,380,154; Megeed et al. |
| | | (SEQ ID NO: 39) | (2002) Advanced Drug Delivery |
| | | | Reviews, 54: 1075-1091 |
| SELP1 | 89000 | [(GVGVP)₄(GAGAGS)₉]₁₃ | Megeed et al. (2002) Advanced |
| | | (SEQ ID NO: 40) | Drug Delivery Reviews, 54: 1075- |
| | | | 1091 |
| SELP 8 | 69,934 | [(VPGVG)₈(GAGAGS)₄]₁₂^{b} | U.S. Pat. No. 6,380,154; Megeed et al. |
| | | (SEQ ID NO: 41) | (2002) Advanced Drug Delivery |
| | | | Reviews, 54: 1075-1091 |
| SELP 7 | 80,338 | [(VPGVG)₈(GAGAGS)₆]₁₂^{b} | U.S. Pat. No. 6,380,154; Megeed et al. |
| | | (SEQ ID NO: 42) | (2002) Advanced Drug Delivery |
| | | | Reviews, 54: 1075-1091 |
| SELP3 | 84,267 | [(VPGVG)₈(GAGAGS)₈]₁₁^{b} | U.S. Pat. No. 6,380,154; Megeed et al. |
| | | (SEQ ID NO: 43) | (2002) Advanced Drug Delivery |
| | | | Reviews, 54: 1075-1091 |
| SELP 4 | 79,574 | [(VPGVG)₁₂(GAGAGS)₈]₈^{b} | U.S. Pat. No. 6,380,154; Megeed et al. |
| | | (SEQ ID NO: 44) | (2002) Advanced Drug Delivery |
| | | | Reviews, 54: 1075-1091 |
| SELP 5 | 84,557 | [(VPGVG)₁₆(GAGAGS)₈]₇^{b} | U.S. Pat. No. 6,380,154; Megeed et al. |
| | | (SEQ ID NO: 45) | (2002) Advanced Drug Delivery |
| | | | Reviews, 54: 1075-1091 |
| SELP 6 | | [(VPGVG)₃₂(GAGAGS)₈]₅ | U.S. Pat. No. 6,380,154 |
| | | (SEQ ID NO: 46) | |
| SELP F | 75,957 | (GAGAGS)₁₂GAAVTGRGDSPASA | U.S. Pat. No. 6,380,154; Megeed et al. |
| | | AGY (GAGAGS)₅(GVGVGP)₈]₆^{b} | (2002) Advanced Drug Delivery |
| | | (SEQ ID NO: 47) | Reviews, 54: 1075-1091 |
| SELP 0K | | [(GAGAGS)₂(GVGVP)₄GKGVP | U.S. Pat. No. 6,380,154; Megeed et al. |
| (SELP | | (GVGVP)₃]₆ | (2002) Advanced Drug Delivery |
| 27K, 6- | | (SEQ ID NO: 48) | Reviews, 54: 1075-1091; Cappello |
| mer) | | | et al. (1998) Journal of |
| | | | Controlled Release, 53: 105-117 |
| SELP 0K | | [(GAGAGS)₂(GVGVP)₄GKGVP | U.S. Pat. No. 6,380,154; Megeed et al. |
| (SELP | | (GVGVP)₃]₁₂ | (2002) Advanced Drug Delivery |
| 27K, 12- | | (SEQ ID NO: 49) | Reviews, 54: 1075-1091; Cappello |
| mer) | | | et al. (1998) Journal of |
| | | | Controlled Release, 53: 105-117 |
| SELP 0K | | [(GAGAGS)₂(GVGVP)₄GKGVP | U.S. Pat. No. 6,380,154; Megeed et al. |
| (SELP | | (GVGVP)₃]₁₈ | (2002) Advanced Drug Delivery |
| 27K, 18- | | (SEQ ID NO: 50) | Reviews, 54: 1075-1091; Cappello |
| mer) | | | et al. (1998) Journal of |
| | | | Controlled Release, 53: 105-117 |
| SELP 0K | 76,639 | [(GAGAGS)₂(GVGVP)₄GKGVP | U.S. Pat. No. 6,380,154; |
| (SELP | | (GVGVP)₃]₁₇GAGAGS)₂ | U.S. Pat. No. 6,423,333; |
| 27K, 17- | | (SEQ ID NO: 51) | Megeed et al. (2002) Advanced |
| mer) | | | Drug Delivery Reviews, 54: 1075- |
| (hereinafter | | | 1091; Cappello et al. (1998) |
| SELP27K) | | | Journal of Controlled Release, |
| | | | 53: 105-117; |
| SELP0K- | 76,389 | [(GAGAGS)₂(GVGVP)₁LGPLGP | U.S. Pat. No. 6,423,333 |
| CS1 | | (GVGVP)₃GKGVP(GVGVP)₃]₁₅ | |
| (SELP27K, | | (GAGAGS)₂ | |
| 15-mer) | | (SEQ ID NO: 73) | |
| SELP0K- | 83,218 | [(GAGAGS)₂(GVGVP)₁GFFVRARR | U.S. Pat. No. 6,423,333 |
| CS2 | | (GVGVP)₃GKGVP | |
| | | (GVGVP)₃)₁₅(GAGAGS)₂ | |
| | | (SEQ ID NO: 74) | |
| SELP 8K | 69,814 | [(GAGAGS)₂-(GVGVP)₄-(GKGVP) | U.S. Pat. No. 6,380,154; Megeed et al. |
| (SELP47K) | | (GVGVP)₃-(GAGAGS)₂]₁₃ | (2002) Advanced Drug Delivery |
| | | (SEQ ID NO: 52) | Reviews, 54: 1075-1091; Cappello |
| | | | et al. (1998) Journal of |
| | | | Controlled Release, 53: 105-117; |
| | | | Gustafson et al. (2010) Advanced |
| | | | Drug Delivery Reviews, 62: 1509- |
| | | | 1523; Haider et al. (2004) |
| | | | Molecular Pharmaceutics, 2: 139- |
| | | | 150 |
| SELP 9K | 60,103 | [GAGAGS(GVGVP)₄GKGVP | U.S. Pat. No. 6,380,154; Megeed et al. |
| | | (GVGVP)₃(GAGAGS)₂]₁₂ | (2002) Advanced Drug Delivery |
| | | (SEQ ID NO: 53) | Reviews, 54: 1075-1091 |
| SELP 415K | 65,374 | [GVGVP)₄GKGVP(GVGVP)₁₁ | Haider et al. (2004) Molecular |
| timer | Da | (GAGAGS)₄]₅(GVGVP)₄GKGVP | Pharmaceutics, 2: 139-150; |
| | | (GVGVP)₁₁(GAGAGS)₂ | |
| | | (SEQ ID NO: 54) | |
| SELP 415K | 71,500 | [(GVGVP)₄(GKGVP)(GVGVP)₁₁ | Haider et al. (2004) Molecular |
| 8 mer | Da | (GAGAGS)₄]₇(GVGVP)₄GKGVP | Pharmaceutics, 2: 139-150; |
| | | (GVGVP)₁₁(GAGAGS)₂ | Gustafson et al. (2010) Advanced |
| | | (SEQ ID NO: 55) | Drug Delivery Reviews, 62: 1509- |
| | | | 1523 |
| SELP | 87860 | [GVGVP)₄GKGVP(GVGVP)₁₁ | Haider et al. (2004) Molecular |
| 415K- | | (GAGAGS)₄]₉(GVGVP)₄GKGVP | Pharmaceutics, 2: 139-150; |
| 10 mer | | (GVGVP)₁₁(GAGAGS)₂ | |
| | | (SEQ ID NO: 56) | |
| SELP 815K | 65374 | [GAGS(GAGAGS)₂(GVGVP)₄ | Gustafson et al. (2010) Advanced |
| 6-mer | | GKGVP(GVGVP)₁₁ | Drug Delivery Reviews, 62: 1509- |
| | | (GAGAGS)5GA]₆ | 1523; Gustafson et al. (2010) |
| | | (SEQ ID NO: 57) | Mol. Pharm., 7: 1050-1056 |

^{a}depicted without the head and tail sequences as set forth in SEQ ID NOS: 58 and 59 or 60
^{b}depicted without the split of the first and last block domain of the polymer. Thus, the SELP additionally includes a further repeat of the monomer, whereby a partial segment of monomer exists at the beginning and the remaining segment at the end. This is due to a split of the encoded amino acid sequence within the silk units, such that the first and last block domain of the polymer is split within the silk bocks whereby both parts sum to a whole domain

In one example, exemplary of a SELP in the VV-SELP compositions herein is SELP-27K. SELP-27K contains the repeating structure (S₂E₄E_{K}E₃)₁₇, where S is the silk-like sequence of amino acids GAGAGS (SEQ ID NO:26), E is the elastin-like sequence GVGVP (SEQ ID NO:29), and E_{K} is the elastin like sequence modified with a lysine residue GKGVP (SEQ ID NO:38). The repeating structure is depicted in SEQ ID NO: 51. The multimeric portion of SELP 27K is expressed as a fusion protein between amino-terminus “head” and carboxy-terminus “tail” sequences of 33 and 19 amino acids, respectively. Hence, the polymer further can contain a head and tail (N- and C-terminus) sequence. The head sequence of amino acids is MDPVVLQRRD WENPGVTQLN RLAAHPPFAS DPM (SEQ ID NO:58) and the tail sequence is GAM DPGRYQDLRSHHHHHH (SEQ ID NO:60). The SELP27K has a molecular weight of about 77,000 Daltons. For example, SELP27K is set forth as follows (SEQ ID NO:61):

[MDPVVLQRRDWENPGVTQLNRL AAHPPFASDPM]
[(GAGAGS)₂-(GVGVP)₄-(GKGVP)-(GVGVP)₃]₁₇-(GAGAGS)₂-
[GAMDPGRYQDLRSHHHHHH]

In another example, exemplary of a SELP in the VV-SELP compositions herein is SELP-47K. SELP-47K contains the repeating structure (S₂E₃E_{K}E₄S₂)₁₃, where S is the silk-like sequence of amino acids GAGAGS (SEQ ID NO:26), E is the elastin-like sequence GVGVP (SEQ ID NO:29), and E_{K} is the elastin like sequence modified with a lysine residue GKGVP (SEQ ID NO:38). This repeating structure is depicted in SEQ ID NO:52. The multimeric portion of SELP 47K is expressed as a fusion protein between amino-terminus “head” and carboxy-terminus “tail” sequences of 33 and 19 amino acids, respectively. Hence, the polymer further can contain a head and tail (N- and C-terminus) sequence. The head sequence of amino acids is MDPVVLQRRD WENPGVTQLN RLAAHPPFAS DPM (SEQ ID NO:58) and the tail sequence is GAM DPGRYQDLRSHHHHHH (SEQ ID NO:60). The SELP47K has a molecular weight of about 70,000 Daltons, and a pI of 10.5. For example, SELP47K is set forth as follows (SEQ ID NO:62):

[MDPVVLQRRDWENPGVTQLNRL AAHPPFASDPM]
[(GAGAGS)₂-(GVGVP)₄-(GKGVP)-(GVGVP)₃-(GAGAGS)₂]₁₃-
[GAMDPGRYQDLRSHHHHHH]

In a further example, exemplary of a SELP in the VV-SELP compositions herein is SELP-815K. SELP-815K contains the repeating structure (S₂E₄E_{K}E₁₁S₆)₆, where S is the silk-like sequence of amino acids GAGAGS (SEQ ID NO:26), E is the elastin-like sequence GVGVP (SEQ ID NO:29), and E_{K} is the elastin like sequence modified with a lysine residue GKGVP (SEQ ID NO:38). It is named because it contains a monomer repeat of eight silk units followed by 15 elastin units, with one elastin unit containing a lysine substitution. One of the silk units is split such that the first and last block domain of the polymer is split within the silk bocks whereby both parts sum to a whole domain. This repeating structure is set forth in SEQ ID NO:57. The multimeric portion of SELP 815K is expressed as a fusion protein between amino-terminus “head” and carboxy-terminus “tail” sequences of 33 and 19 amino acids, respectively. Hence, the polymer further can contain a head and tail (N- and C-terminus) sequence. The head sequence of amino acids is MDPVVLQRRD WENPGVTQLN RLAAHPPFAS DPM (SEQ ID NO:58) and the tail sequence is GAM DPGRYQDLRSHHHHHH (SEQ ID NO:60). The SELP815K has a molecular weight of about 65,000 Daltons. For example, SELP815K is set forth as follows (SEQ ID NO:63):

MDPVVLQRRDWENPGVTQLNRLAAHPPFASDPM
[GAGS(GAGAGS)₂(GVGVP)₄GKGVP(GVGVP)₁₁(GAGAGS)₅GA]₆
GASMDPGRYQDLRSHHHHHH

2. Methods of Preparing and Generating Polymers

The oligomers or repeat polymers, including SELP polymers, can be prepared by various methods known to one of skill in the art (Megeed et al. (2002) Advanced Drug Delivery Reviews, 54:1075-1091). For example, the repeat polymers can be synthesized by generally recognized methods of chemical synthesis (for example, L. Andersson et al., Large-scale synthesis of peptides, Biopolymers 55(3), 227-50 (2000)), genetic manipulation (for example, J. Cappello, Genetically Engineered Protein Polymers, Handbook of Biodegradable Polymers, Domb, A. J.; Kost, J.; Wiseman, D. (Eds.), Harvard Academic Publishers, Amsterdam; pages 387-414), and enzymatic synthesis (for example, C H. Wong & K. T. Wang, New Developments in Enzymatic Peptide Synthesis, Experientia 47(11-12), 1123-9 (1991)). For example, the repeat sequence protein polymers can be synthesized using the methods described in U.S. Pat. Nos. 5,243,038 and 6,355,776. In another example, the repeat sequence protein polymers can by synthesized utilizing non-ribosomal peptide synthase (for example, H. V. Dohren, et al., Multifunctional Peptide Synthase, Chem. Rev 97, 2675-2705 (1997).

For example, the protein polymers, including SELP polymers, can be prepared in accordance with the manner described in U.S. Pat. No. 5,243,038. For example, one procedure involves synthesizing small segments of single stranded DNA of from about 15 to 150 nucleotides to provide a plurality of fragments which have cohesive ends, which can be ligated together to form a segment or a plurality of segments. The first dsDNA fragment is cloned to ensure the appropriate sequence, followed by the addition of successive fragments, which are in turn cloned and characterized, to ensure that the integrity of the sequence is retained. The fragments are joined together to form a monomer segment which, as described above, then becomes the major repeating building block of the polymer gene.

For example, two different oligomeric units can be prepared where the termini of the two units are complementary one with the other but the termini of the same unit are unable to bind together. In this way one can build individual oligomeric units and then join them together to form the concatemer, where the intervening linking sequences are defined at least in part by the termini. Alternatively, long single strands can be prepared, cloned and characterized, generally being of at least 100 nucleotides and up to about 300 nucleotides, where the two single strands are hybridized, cloned and characterized and can then serve as the monomer segment. The monomers can then be multimerized, having complementary termini, particularly cohesive ends, so that the polymer will have two or more monomers present. The multimers can then be cloned in an appropriate vector and characterized to determine the number of monomers and the desired size polymer selected.

Depending upon the construct, the 5′ terminus can provide for the initiation codon methionine, or the structural gene can be joined to an adapter that can provide for a unique sequence (optionally cleavable by a specific enzyme) at the 5′ terminus or can be inserted into a portion of gene, usually endogenous to the host, in proper reading frame so as to provide for a fusion product. By providing for appropriate complementary termini between the adapter or truncated gene and the 5′ end of the subject structural gene, the sequences can be joined in proper reading frame to provide for the desired protein. The inclusion of adapters or fusion proteins can provide specific sequences for purposes such as linking, secretion, complex formation with other proteins, or affinity purification.

Expression can be achieved in an expression host using transcriptional regulatory regions functional in the expression host. The expression host can be prokaryotic or eukaryotic, particularly bacterial (e.g. E. coli, B. subtilis); yeast (e.g. Saccharomyces, Neurospora); insect cells, plant cells, mammalian cells. If desired, a signal sequence can be provided for secretion of the polymer. A wide variety of signal sequences are known and have been used extensively for secreting proteins which are not normally secreted by the expression host.

After completion of expression, where the protein is retained in the host, the cells are disrupted and the product extracted from the lysate. Where the product is secreted, the product can be isolated from the supernatant. In either case, various techniques for purifying the products can be employed, depending upon whether the products are soluble or insoluble in the medium. Where insoluble, impurities can be extracted from the polymer, leaving the polymer intact. Where soluble, the polymer can be purified in accordance with conventional methods, such as extraction or chromatography. In particular examples, supernatant or homogenized cell extract can be filtered and protein precipitated therefrom. For example, protein polymer can be precipitated by mixing a filtered solution with ammonium sulfate to 25% saturation. The precipitated product can be reconstituted with a liquid, for example, water. The precipitated polymer can be further filtered and/or purified. The product can be concentrated to a desired concentration, and generally is lyophilized to form a powder. The lyophilized powder can be stored, for example at −70° C.

Due to the ability of protein polymers to gel, protein polymers typically are prepared as powders in lyophilized form. A protein polymer solution, such as a SELP polymer solution, can be formulated into a liquid composition by dissolving a polymer or a mixture thereof in a biocompatible liquid. For example, protein polymer-containing solutions can be prepared in, for example, water, saline, phosphate buffered saline or other buffer or isotonic aqueous solution with or without other additives. Exemplary of other additives include, for example, mannitol, glucose, alcohol, vegetable oil, and the like. Faster gellation or crystallization of a liquid composition containing the protein polymer can be obtained by increasing the concentration of the polymer in the liquid. Generally, polymer compositions have a weight percentage (wt %) of from about 2% (w/w) to about 50% (w/w) of the composition being protein polymer, such as from about 5% (w/w) to about 50% (w/w), about 10% (w/w) to about 50% (w/w), about 20% (w/w) to about 35% (w/w), and generally at least about or about 20% (w/w). The specific concentration can be empirically determined by one of skill in the art and is dependent on factors such as the particular SELP, the proposed use, the time and temperature of storage and use and other factors. For example, SELP 47K has a higher silk:elastin (S:E) ratio and forms hydrogels in the concentration range 4-12 wt %, whereas SELP 415K forms hydrogels above 10 wt % concentration due to its low S:E ratio.

The resulting polymers can be characterized using methods known in the art, including for example, amino acid compositional analysis, microchemical elemental analysis, moisture analysis and characterization of gelation (see e.g. Cappello et al. (1998) Journal of Controlled Release, 53:105-117).

### E. VACCINIA VIRUS-PROTEIN POLYMER (VV-POLYMER) COMPOSITIONS

Provided herein are VV-polymer compositions containing an oncolytic virus in a protein polymer matrix or gel. For example, provided herein are VV-SELP formulations, and in particular LIVP-SELP compositions. The VV-polymer (e.g. VV-SELP) compositions provided herein include those that are liquid compositions or non-liquid compositions (e.g. gel, solid, or other form that substantially lacks the property of flow). Protein polymer compositions, such as SELP polymer-containing compositions, undergo a nonreversible crystallization event resulting in gelation. Hence, it is understood that liquid polymer compositions provided herein containing vaccinia virus (e.g. LIVP) are capable of irreversibly acquiring a non-liquid form with time or under physiological conditions (e.g., at normal body temperature after administration in vivo).

Vaccinia virus (e.g. an LIVP) in protein polymer compositions provided herein, for example SELP compositions is stable and retains viral integrity at 37° C. for at least one week (e.g. 7 days) or greater than one week. The viral integrity can be retained for at least 8 days, 9 days, 10 days, 11 days, 12 days, 13 days, two weeks, three weeks and up to a month at 37° C. The viral integrity of vaccinia virus, such as an LIVP, in a protein polymer composition provided herein is at least or greater than or about or 10%, 11%, 12%, 13%, 14%, 15%, 16%, 17%, 18%, 19%, 20%, 25%, 30%, 35%, 40%, 45%, 50%, 55%, 65%, 70%, 80%, 90%, 95% of the viral integrity of the starting virus stock prior to incubation at 37° C. By virtue of the stability, the compositions provided herein can be used to effect delivery of virus at physiologic temperatures over an extended time period. Thus, the polymers can be used in surface application, such as patch formation or wound healing applications, and also in slow release applications. In addition, the polymers also minimize or reduce antigenicity of administered virus in animals and humans. Hence, the compositions provided herein can provide protection from immune responses.

The compositions provided herein can be used in various methods known to one of skill in the art, and in particular for the therapy of tumors or treatment of wounded and inflamed tissues and cells. The VV-polymer compositions, such as VV-SELP (e.g. LIVP-SELP) compositions, can be administered or introduced to virtually any in vivo site by a number of means. Exemplary of administration techniques of the coated viruses herein include, but are not limited to, injection by syringe into a site of interest, use of trocar or catheter, surgical implantation, placement into open wounds or other cavities, or coating on a bandage, gauze or other wrap for application to a body surface. In addition, the coated viruses also can be used for systemic (e.g. intravenous), oral, intraperitoneal and intratumoral applications.

1. Methods of Making Compositions

Vaccina virus-protein polymer compositions, such as VV-SELP compositions, can be made by methods known to one of skill in the art for generating polymer compositions capable of gelation (see e.g. U.S. Pat. No. 6,380,154;). For example, liquid polymer solutions, such as silk-elastin polymer solutions, can be mixed directly with a vaccinia virus composition or virus stock (e.g. LIVP composition). In particular examples, a silk-elastin polymer, such as any described in Section D is mixed directly with an LIVP composition, such as any described in Section C.

The components can be mixed by any method that is known to one of skill in the art, such that the resulting mixture is a liquid solution containing the components therein. Typically, the components are mixed in a biocompatible solute or liquid such as, but not limited to, water, saline, phosphate buffered saline, tris(hydroxymethyl)methylamine (Tris), minimum essential medium (MEM) or other buffer or isotonic aqueous solution. Typically, mixing occurs at temperatures less than 37° C. where gelation can quickly occur, and generally at temperatures less than 30° C. and most typically at room temperature of about or between about 20° C. to 25° C. Gentle mixing is generally desired. For example, the components can be combined at room temperature and the solution gently swirled or inverted periodically for a sufficient time to mix the components. The mixture or combination can be incubated together for at least 10 seconds, 30 seconds, 1 minute, 5 minutes, 10 minutes, 20 minutes, 30 minutes, 40 minutes, 50 minutes, 60 minutes or longer. Generally, incubation and mixing is effected in a sufficient time before the composition acquires a non-liquid form.

The resulting liquid compositions will, over time, undergo a gelation process to yield a hydrogel composition. The transition from a liquid form to a non-liquid form occurs without the need for chemical crosslinking via chemical reaction or irradiation. In this way, no chemical changes to the protein polymer composition or to any biologically active substance contained in a composition thereof will occur. The rate of gelation, solidification or crystallization can be influenced by such things as the number of protein crystallization units in the polymer (the greater the relative number of protein crystallization units, the greater the rate of acquiring a non-liquid form in vivo), the concentration of the polymer (the greater the concentration of the protein polymer in the liquid composition, the greater the rate of acquiring a non-liquid form in vivo), temperature (the greater the temperature, the greater the rate of acquiring a non-liquid form in vivo) and other solution conditions.

Further, the rate of release of the vaccinia virus from the non-liquid form can depend on the concentration of the virus, its solubility in the polymer matrix, the composition of the polymer, including the relative number of protein crystallization units present therein and the conditions under which release takes place. Polymer compositions can be selected to provide for varying rates of release (i.e., quick release or sustained release over an extended period of time). It is within one of skill in the art to test VV-polymer formualtions (e.g. VV-SELP compositions) to determine the release rate of the virus, and thereby empirically determine the particulars of the composition that are suitable for a desired application or use.

Thus, the particular percentage weight of protein polymer solution that is mixed to generate the VV-polymer compositions can be empirically determined by one of skill in the art and is a function of the particular polymer or SELP, the desired or planned use of the composition, the desired release rate of the virus from the hydrogel, the time period in which gelation is desired, the particular temperature the mixing occurs and other parameters known to one of skill in the art.

In particular examples herein, the polymer compositions are generated so that the resulting VV-polymer compositions, for example VV-SELP compositions (e.g. LIVP-SELP), has a weight percentage (wt %) of from about 2% (w/w) to about 50% (w/w) of the composition being protein polymer, such as from about 2% (w/w) to about 35% (w/w), from about 2% (w/w) to about 20% (w/w), from about 2% (w/w) to about 12% (w/w), from about 4% (w/w) to about 50% (w/w), from about 4% (w/w) to about 35% w/w, from about 4% (w/w) to about 12%, from about 4% (w/w) to about 8% (w/w), from about 5% (w/w) to about 50% (w/w), from about 10% (w/w) to about 50% (w/w), from about 20% (w/w) to about 35% (w/w), and generally at least about or about 4%, 5%, 6%, 7%, 8%, 9%, 10%, 11%, 12%, 13%, 14%, 15% or 20% (w/w). In one example, if the polymer is a SELP815K, the weight percentage of the composition being protein polymer is from about 2% (w/w) to 12% (w/w), such as 4% (w/w) to 12% (w/w), for example 4% (w/w) to 8% (w/w), and generally at least about or about or 4%. In another example, if the polymer is SELP 47K, the weight percentage of the composition being protein polymer is from about 2% (w/w) to 12% (w/w), such as 4% (w/w) to 12% (w/w), for example 4% (w/w) to 8% (w/w), and generally at least about or about or 4%. In other examples, if the polymer is SELP27K, the weight percentage of the composition being protein polymer is from about 2% (w/w) to 12% (w/w), such as 4% (w/w) to 12% (w/w), for example 4% (w/w) to 8% (w/w), and generally at least about or about or 4%. In a further example, if the polymer is SELP415K, the weight percentage of the composition being protein polymer is above 10% (w/w), and generally is about 10% (w/w) to 50% (w/w), such as 20% (w/w) to 35% (w/w).

The oncolytic vaccinia virus in polymer compositions provided herein can be generated to contain a therapeutically effective amount of vaccinia virus. For example, the polymer compositions can be generated from a virus stock solution that is 10⁵-10¹° pfu/mL, for example, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL. Upon mixing with polymer, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. In particular examples, the resulting mixed VV-polymer compositions can contain virus at a concentration of 10⁵-10¹⁰ pfu/mL, for example, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL. For example, the compositions can contain an amount of virus that is or is about 1×10⁵ to 1×10¹² pfu, such as 1×10⁶ to 1×10¹° pfu or 1×10⁷ to 1×10¹⁰ pfu, for example at least or about at least or 1×10⁶, 1×10⁷, 1×10⁸, 1×10⁹, 2×10⁹, 3×10⁹, 4×10⁹, or 5×10⁹ pfu.

Because the transition from the liquid form to a non-liquid form is mediated by hydrogen bonding occurring between protein crystallization units present in the protein polymers, compounds that inhibit hydrogen bonding can be employed to decrease the rate at which the liquid form acquires a non-liquid form. Such compounds include, for example, urea, guanidine hydrochloride, dimethyl formamide, colloidal gold sol, aqueous lithium bromide and formic acid. The concentrations of such compounds can be readily determined by one of skill in the art.

Moreover, additives that increase the rate at which the liquid composition acquires a non-liquid form also can be used when preparing the compositions. Such “nucleating agents” or “accelerators” include, for example, pre-gelled protein polymers such as the SELP or SLP protein polymers described herein or known to one of skill in the art. In particular, such polymers include SLP3 or SLP4, which are described in U.S. Pat. No. 5,243,038. Aqueous solvents also can be used including, for example, ethanol.

Also provided herein are VV-protein polymer compositions, such as VV-SELP compositions (e.g. LIVP-SELP), that are nanoparticles. Methods of generating nanoparticles are known in the art (see e.g. Anumolu et al. (2011) ACS Nano 5:5374-82; International PCT Appl. No. WO2011140024). Such methods include providing a VV-protein polymer (e.g. VV-SELP such as LIVP-SELP) solution in a solvent, forming droplets containing the VV-protein polymer and solvent and removing the solvent to produce nanoparticles. The droplets can be formed by an electrospray aerosol generator or a nebulizer. The nanoparticles generally are uniform in size. For example, on preparation of nanoparticles, nanoparticles can be separated based on size to produce nanoparticles that are substantially uniform in size. Separation can be effected by any method known in the art, for example, using a differential mobility analyzer that separates or purifies nanoparticles based on charge-to-size ratio. The size of the nanoparticles can vary depending upon reaction and solution conditions. In one aspect, the nanoparticles have a diameter from 1 nm to 250 nm, 5 nm to 200 nm, 10 nm to 100 nm, or from 10 nm to 60 nm. The size of the nanoparticles can be selected depending upon the application of the nanoparticles. Additionally, the shape of the nanoparticles can vary as well. For example, the nanoparticles can be spherical or cylindrical. Nanoparticles can be designed to carry a targeting ligand, and in particular a targeting ligand or molecule that targets the nanoparticle to the tumor cells. In one non-limiting example, nanoparticles can be coated with a radionuclide and, optionally, an antibody immunoreactive with a tumor-associated antigen.

2. Exemplary VV-SELP Compositions

Provided herein are compositions of an oncolytic vaccinia virus in SELP polymer (VV-SELP). In particular examples, provided herein are LIVP-SELP compositions containing an LIVP virus in a SELP. The VV-SELP compositions can be liquid or can be non-liquid solid or gel compositions. It is understood that liquid compositions of VV-SELP compositions are precursor hydrogel matrix compositions and are capable of irreversibly acquiring a non-liquid form with time or under physiological conditions (e.g., at normal body temperature after administration in vivo) to generate a hydrogel matrix composition. For example, when a liquid composition is exposed to the physiological temperature of an animal (e.g. a human), it will transform into a non-liquid hydrogel form. The compositions provided herein, in particular the hydrogel compositions, effect sustained release of the virus. Further, the vaccinia virus (e.g. LIVP virus) contained in the polymer (e.g. SELP) hydrogel composition is stable and retains viral integrity for at least one week at 37° C.

Generally, liquid VV-SELP compositions provided herein exhibit sufficient working time as a liquid to allow them to be loaded into a syringe, injected, coated or applied on a bandage or other material or otherwise introduced into the body. For example, the liquid compositions provided herein generally acquire a non-liquid form in from about 30 seconds to about 500 minutes after mixing, such as from about 1 minute to about 250 minutes after mixing, and generally from about 5 minutes to about 125 minutes after mixing. The particular time can be empirically determined and is dependent on factors that include the choice of SELP polymer, the concentration or amount of virus contained therein, the conditions of mixing (e.g. temperature) and other factors that are within the level of one of skill in the art.

Included among the VV-SELP compositions provided herein are compositions containing 10⁵-10¹⁰ pfu/mL of a vaccinia virus contained in a SELP polymer that is from about 2 weight % to about 20 weight % of the composition. The vaccinia virus can be contained in a SELP polymer that is from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The composition can contain 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of vaccinia virus. When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain vaccinia virus in an amount that is from or from about 1×10⁵ to 1×10¹² pfu, such as 1×10⁶ to 1×10¹⁰ pfu or 1×10⁷ to 1×10¹° pfu, for example at least or about at least or 1×10⁶, 1×10⁷, 1×10⁸, 1×10⁹, 2×10⁹, 3×10⁹, 4×10⁹, or 5×10⁹ pfu. The vaccinia virus can be any vaccinia virus known to one of skill in the art, such as any described in Section C above. In particular examples, the vaccinia virus is a Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16 m8, LC16M0, LIVP, ACAM2000, WR 65-16, Connaught, New York City Board of Health (NYCBH), EM-63, or NYVAC vaccinia virus, clonal strain thereof or a modified forms thereof. For example, the vaccinia virus is a modified form that contains one or more heterologous nucleic acid molecules inserted or replaced into the genome of the virus. The SELP polymer can be any polymer known to one of skill in the art, such as any described in Section D above.

Exemplary of compositions herein contain SELP-47K (SEQ ID NO:62), SELP-27K (SEQ ID NO:61) or SELP-815K (SEQ ID NO:63). For example, exemplary of VV-SELP compositions provided herein are compositions containing 10⁵-10¹⁰ pfu/mL of a vaccinia virus contained in a SELP-47K, SELP-27K or SELP-815K polymer that is from about 2 weight % to about 20 weight % of the composition. The vaccinia virus can be contained in a SELP-47K, SELP-27K or SELP-815K polymer that is from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The composition can contain 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of vaccinia virus. When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain vaccinia virus in an amount that is from or from about 1×10⁵ to 1×10¹² pfu, such as 1×10⁶ to 1×10¹⁰ pfu or 1×10⁷ to 1×10¹⁰ pfu, for example at least or about at least or 1×10⁶, 1×10⁷, 1×10⁸, 1×10⁹, 2×10⁹, 3×10⁹, 4×10⁹, or 5×10⁹ pfu. The vaccinia virus can be any vaccinia virus known to one of skill in the art, such as any described in Section C above. In particular examples, the vaccinia virus is a Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16 m8, LC16M0, LIVP, ACAM2000, WR 65-16, Connaught, New York City Board of Health (NYCBH), EM-63, or NYVAC vaccinia virus, clonal strain thereof or a modified forms thereof. For example, the vaccinia virus is a modified form that contains one or more heterologous nucleic acid molecules inserted or replaced into the genome of the virus.

In particular examples, the VV-SELP compositions are LIVP-SELP compositions containing 10⁵-10¹° pfu/mL of an LIVP contained in a SELP polymer that is from about 2 weight % to about 20 weight % of the composition. The LIVP can be contained in a SELP polymer that is from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The LIVP in the compositions provided herein can be any LIVP known to one of skill in the art, including but not limited to, an LIVP containing a genome set forth in any of SEQ ID NOS:1-8, or that exhibits at least 97%, 98%, 99% or more sequence identity to any of SEQ ID NOS:1-8. For example, the LIVP is a modified or recombinant LIVP containing insertion, deletion or replacement of heterologous nucleic acid. In particular, the LIVP is a virus strain containing a modified genome of any of SEQ ID NOS:1-8, including any known in the art or as described herein above in Section C or Table 4. The composition can contain 10⁵-10¹⁰ pfu/mL, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of LIVP, including a clonal strain or modified form thereof in a SELP polymer. When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain an LIVP in an amount that is from or from about 1×10⁵ to 1×10¹² pfu, such as 1×10⁶ to 1×10¹⁰ pfu or 1×10⁷ to 1×10¹° pfu, for example at least or about at least or 1×10⁶, 1×10⁷, 1×10⁸, 1×10⁹, 2×10⁹, 3×10⁹, 4×10⁹, or 5×10⁹ pfu. In particular examples, the LIVP virus can be contained in a SELP polymer that is SELP-47K, SELP-27K or SELP-815K.

For example, provided herein are LIVP-SELP-47K compositions containing 10⁵-10¹⁰ pfu/mL of an LIVP contained in a SELP-47K polymer (set forth in SEQ ID NO:62) that is from about 2 weight % to about 20 weight % of the composition. The SELP-47K in the composition can have a weight percentage of from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The LIVP in the compositions provided herein can be any LIVP known to one of skill in the art, including but not limited to, an LIVP containing a genome set forth in any of SEQ ID NOS:1-8, or that exhibits at least 97%, 98%, 99% or more sequence identity to any of SEQ ID NOS:1-8. For example, the LIVP is a modified or recombinant LIVP containing insertion, deletion or replacement of heterologous nucleic acid. In particular, the LIVP is a virus strain containing a modified genome of any of SEQ ID NOS:1-8, including any known in the art or as described herein above in Section C or Table 4. The composition can contain 10⁵-10¹⁰ pfu/mL, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of LIVP, including a clonal strain or modified form thereof in SELP-47K polymer When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain an LIVP in an amount that is from or from about 1×10⁵ to 1×10¹² pfu, such as 1×10⁶ to 1×10¹⁰ pfu or 1×10⁷ to 1×10¹⁰ pfu, for example at least or about at least or 1×10⁶, 1×10⁷, 1×10⁸, 1×10⁹, 2×10⁹, 3×10⁹, 4×10⁹, or 5×10⁹ pfu.

In another example, provided herein are LIVP-SELP-27K compositions containing 10⁵-10¹⁰ pfu/mL of an LIVP contained in a SELP-27K polymer (set forth in SEQ ID NO:61) that is from about 2 weight % to about 20 weight % of the composition. The SELP-27K in the composition can have a weight percentage of from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The LIVP in the compositions provided herein can be any LIVP known to one of skill in the art, including but not limited to, an LIVP containing a genome set forth in any of SEQ ID NOS:1-8, or that exhibits at least 97%, 98%, 99% or more sequence identity to any of SEQ ID NOS:1-8. For example, the LIVP is a modified or recombinant LIVP containing insertion, deletion or replacement of heterologous nucleic acid. In particular, the LIVP is a virus strain containing a modified genome of any of SEQ ID NOS:1-8, including any known in the art or as described herein above in Section C or Table 4. The composition can contain 10⁵-10¹⁰ pfu/mL, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of LIVP, including a clonal strain or modified form thereof in SELP-27K polymer. When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain an LIVP in an amount that is from or from about 1×10⁵ to 1×10¹² pfu, such as 1×10⁶ to 1×10¹⁰ pfu or 1×10⁷ to 1×10¹⁰ pfu, for example at least or about at least or 1×10⁶, 1×10⁷,1×10⁸, 1×10⁹, 2×10⁹, 3×10⁹, 4×10⁹, or 5×10⁹ pfu.

In a further example, provided herein are LIVP-SELP-815K composition containing 10⁵-10¹⁰ pfu/mL of an LIVP contained in a SELP-815K polymer (set forth in SEQ ID NO:63) that is from about 2 weight % to about 20 weight % of the composition. The SELP-815K in the composition can have a weight percentage of from about 2% (w/w) to about 12% (w/w), from about 4% (w/w) to about 12%, from about 4% (w/w) to about 8% (w/w), and generally at least about or about 4%, 5%, 6%, 7%, 8%, 9%, 10%, 11%, 12%, 13%, 14%, 15% or 20% (w/w). The LIVP in the compositions provided herein can be any LIVP known to one of skill in the art, including but not limited to, an LIVP containing a genome set forth in any of SEQ ID NOS:1-8, or that exhibits at least 97%, 98%, 99% or more sequence identity to any of SEQ ID NOS:1-8. For example, the LIVP is a modified or recombinant LIVP containing insertion, deletion or replacement of heterologous nucleic acid. In particular, the LIVP is a virus strain containing a modified genome of any of SEQ ID NOS:1-8, including any known in the art or as described herein above in Section C or Table 4. The composition can contain 10⁵-10¹° pfu/mL, 5×10⁶ to 5×10⁹ or 10⁷-10⁹ pfu/mL, such as at least or about or 10⁶ pfu/mL, 10⁷ pfu/mL, 10⁸ pfu/mL or 10⁹ pfu/mL of LIVP, including a clonal strain or modified form thereof in SELP-27K polymer. When prepared as a liquid composition as a hydrogel matrix precursor, the liquid composition can have a volume of from or from about 0.01 mL to 100 mL, such as from or from about 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL, 0.5 mL to 5 mL, for example, at least or about at least or 0.05 mL, 0.5 mL or 1 mL. Hence, the resulting composition can contain an LIVP in an amount that is from or from about 1×10⁵ to 1×10¹² pfu, such as 1×10⁶ to 1×10¹⁰ pfu or 1×10⁷ to 1×10¹⁰ pfu, for example at least or about at least or 1×10⁶, 1×10⁷, 1×10⁸, 1×10⁹, 2×10⁹, 3×10⁹, 4×10⁹, or 5×10⁹ pfu.

3. Dosage Forms, Carriers and Excipients

The compositions provided herein can be formulated for administration by any available route known in the art. The composition should suit the mode of administration. Administration can be local, topical or systemic depending upon the locus of treatment. Local administration to an area in need of treatment can be achieved by, for example, but not limited to, local injection (e.g. intratumoral), or by topical application, e.g. in conjunction with a wound dressing after surgery.

The compositions can be provided as a liquid, hydrogel or lyophilized compositions. Typically, VV-SELP compositions are initially formulated and prepared as liquid compositions that are hydrogel matrix precursor, which eventually form hydrogels. As described above, the liquid compositions are prepared by mixing polymer solutions, for example SELP solutions, with virus. The liquid compositions provided herein generally retain aqueous or fluid properties for 30 seconds to 1 hour, and generally 1 minute to 40 minutes or 5 minutes to 30 minutes. Hence, such compositions are typically used immediately shortly after preparation. In some examples, the liquid compositions can be administered to a subject by any injectable route of administration, including but not limited to, intravenous, intratumoral, subcutaneous, intraperitoneal, intradermal or other route. In particular examples herein, the liquid compositions are formulated for intravenous administration. For example, the solutions can be injected through fine gauge hypodermic needles. In other examples, the liquid compositions can be topically applied directly to a wound, tumor or other site of injury or disease, or to a bandage or other similar article. Because the compositions can acquire a non-liquid form with time or under various physiologic conditions, they are useful for releasing the virus incorporated therein to the systemic circulation or to a localized site (e.g. directly to a tumor or wound).

It is understood that liquid or aqueous compositions herein are hydrogel precursor compositions. For example, a hydrogel composition is generated upon delivery, administration or exposure of the liquid composition to the physiologic temperature (e.g. 34° C. to 37° C.) of a body location. Thus, in one example, administration of a composition provided herein formulated as a liquid composition for direct intravenous injection is a hydrogel precursor and will transform to a non-liquid hydrogel upon administration to the systemic environment tof the body. In another example, administration of a composition provided herein formulated as a liquid formation for topical application to a wound or other skin lesion will transform to a non-liquid hydrogel upon exposure to the physiologic temperature of the skin.

Compositions, including liquid preparations, can be prepared by conventional means with pharmaceutically acceptable additives or excipients. Where the compositions are provided in lyophilized form they can be reconstituted just prior to use by an appropriate buffer, for example, a sterile saline solution.

Pharmaceutically acceptable compositions are prepared in view of approvals for a regulatory agency or other agency prepared in accordance with generally recognized pharmacopeia for use in animals and in humans. The compositions can be formulated for single dosage administration or for multiple dosage administration. The compositions can be formulated for direct administration.

For example, any of the compositions provided can contain a physiologically acceptable carrier or excipient. Examples of suitable pharmaceutical carriers are known in the art and include, but are not limited to, water, buffers, saline solutions, phosphate buffered saline solutions, various types of wetting agents, sterile solutions, alcohols, gum arabic, vegetable oils, benzyl alcohols, gelatin, glycerin, carbohydrates, such as lactose, sucrose, dextrose, amylose or starch, sorbitol, mannitol, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preserving agents, analgesic agents, binders, disintegrants, coloring, diluents, excipients, extenders, glidants, solubilizers, stabilizers, tonicity agents, vehicles, viscosity agents, flavoring agents, sweetening agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as, but not limited to, crystalline cellulose, microcrystalline cellulose, citric acid, dextrin, liquid glucose, lactic acid, lactose, magnesium chloride, potassium metaphosphate, starch, among others. Such carriers and/or additives can be formulated by conventional methods and can be administered to the subject at a suitable dose. Stabilizing agents such as lipids, nuclease inhibitors, polymers, and chelating agents can preserve the compositions from degradation within the body. Other suitable compositions for use in a pharmaceutical compositions can be found, for example, in Remington: The Science and Practice of Pharmacy (2005, Twenty-first edition, Gennaro & Gennaro, eds., Lippencott Williams and Wilkins).

Parenteral administration, generally characterized by injection or infusion, either subcutaneously, intramuscularly, intravenous or intradermally is contemplated herein. In particular examples, the compositions are formulated for intravenous administration. Preparations for parenteral administration include sterile solutions ready for injection, sterile dry soluble products, such as lyophilized powders, ready to be combined with a solvent just prior to use, sterile suspensions ready for injection, sterile dry insoluble products ready to be combined with a vehicle just prior to use and sterile emulsions. For example, lyophilized compositions can be prepared. In such examples, the hydrogel composition is dried to generate a lyophilized from. The dried hydrogel matrix can be re-hydrated by resuspension in an aqueous solution.

Injectables can be prepared in conventional forms, either as liquid solutions or suspensions, solid forms suitable for solution or suspension in liquid prior to injection, or as emulsions. The compositions provided herein can be formulated in an aqueous solutions, such as in a physiologically compatible buffer. Exemplary parenteral vehicles or buffers include, but are not limited to, Hanks' solution, Ringer's solution, or physiological saline buffer, phosphate buffered saline (PBS), a sodium chloride solution, Ringer's dextrose, dextrose and sodium chloride, lactated Ringer's or fixed oils. For example, suitable carriers include solutions containing thickening and solubilizing agents, such as glucose, polyethylene glycol, and polypropylene glycol and mixtures thereof. The concentration of the pharmaceutically active compound is adjusted so that an injection or infusion provides an effective amount to produce the desired pharmacological effect.

In other examples, the compositions provided herein are formulated for topical administration. Topical mixtures are prepared as described for local and systemic administration. The resulting mixture can be a solution, suspension or emulsions and are formulated as creams, gels, ointments, emulsions, solutions, elixirs, lotions, suspensions, tinctures, pastes, foams, aerosols, irrigations, sprays, suppositories, bandages, dermal patches or any other compositions suitable for topical administration. The compounds can be formulated for local or topical application, such as for topical application to the skin and mucous membranes, in the form of gels, creams, and lotions. Topical administration is contemplated for transdermal delivery. Compositions suitable for transdermal administration are provided. They can be provided in any suitable format, such as discrete patches, dressings, bandages, wraps, film or other similar article adapted to remain in intimate contact with the epidermis of the recipient for a prolonged period of time. For example, sustained release of a composition provided herein can be performed, for example, using a drug delivering bandage, i.e. putting a thin slab of the liquid, semi-solid or solid polymer gel (e.g., a hydrogel) onto the skin, covered by a suitable bandage for prevention of movement and dehydration, and allowing sustained drug delivery from the matrix and through the skin.

The viscosity of the liquid or non-liquid composition can be adjusted or chosen depending on the particular use or mode of administration of the composition. Generally, the compositions provided herein exhibit a range of viscosities, depending on the temperature. At lower temperatures, the hydrogen bonding between water molecules and polymer become unfavorable, while at increasing temperatures it is favored. Thus, polymer compositions provided herein are generally aqueous and exhbit a low viscosity at ambient temperatures (e.g. at temperatures of from or from about 21° C. to 27° C., such as at least or about or no more than 25° C.). The viscosity of the composition increases with increased in temperature, generally forming a semi-solid or solid gel at physiologic or body temperature. In addition to temperature, the particular viscosity of the composition can be affected by the particular SELP in the composition, the weight percentage of SELP, the amount or concentration of virus contained therein, and/or the presence of agents that increase or decrease the rate at which the liquid form acquires a non-liquid form (e.g. urea and other agents described herein or known in the art). Exemplary compositions provided herein can have a viscosity of from between or about between 1 centipoise (cp) to 2,000,000 cp, for example, 50 to 100 cp (e.g. like Mazola corn oil), 150 to 200 cp (e.g. like maple syrup), 250 to 500 cp (e.g. like castor oil); 1,000 to 2,000 cp (e.g. like glycerin), 2,000 to 3,000 cp (e.g. like honey), 5,000 to 10,000 cp (e.g. like molasses), 10,000 to 25,000 cp (e.g. like hershey chocolate syrup), 50,000 to 70,000 cp (e.g. like mustard), 150,000 to 250,000 cp (e.g. like peanut butter), 1,000,000 to 2,000,000 cp (e.g. like lard).

For example, when the composition is formulated as a liquid composition for administration as a hydrogel precursor for direct injection using a device having a narrow channel (e.g. a needle, cannula, or piece of flexible or narrow bore tubing), the composition should be in a liquid or easily-deformable form. As noted above, it is within the level of one of skill in the art to prepare a liquid composition to retain aqueous or fluid properties for a sufficient period of time depending on the particular application. Typically, liquid compositions provided herein retain aqueous or fluid properties for from at least or about at least 30 seconds to 1 hour, and generally 1 minute to 40 minutes or 5 minutes to 30 minutes at ambient temperatures.

In another example, when the hydrogel precursor mixture is prepared to use in generating gels, including semi-solid and solid forms (e.g. to prepare or coat devices), the mixture can be prepared to have a higher viscosity to retain its geometerical form after generation of the matrix but prior to formation of the hydrogel matrix. The viscosity of the mixture can even be sufficiently high that the mixtrure retains its geometrical form regardless of whether it is suspended in an aqueous liquid. Hence, the hydrogel matrix precursor or hydrogel can be a viscoscity that is like glycerine or is substantially rigid. Generally, the viscosity is such that the composition can be easily spread, coated or applied on the surface of the body or on the surface of a device or other material. It is within the level of one of skill in the art to prepare a composition having any desired viscosity for the desired application, in particular by varying the particular protein polymer (e.g. SELP) or its weight percentage. Upon exposure to physiologic temperatures or body temperatures, the viscosity of the composition can increase further to form a semi-solid or solid gel form.

4. Combinations

Provided herein are combinations of a VV-protein polymer composition, such as a VV-SELP (e.g. LIVP-SELP), and a second agent. The second agent can be a second virus or virus in polymer composition or other therapeutic or diagnostic agent. For example, the second agent can be a therapeutic compound, a therapeutic or diagnostic virus, an antiviral or chemotherapeutic agent or an agent or compound for modulation of gene expression of endogenous or heterologous genes encoded by to virus.

Combinations provided herein can contain a VV-protein polymer (e.g. VV-SELP or LIVP-SELP) composition provided herein and a therapeutic compound. Therapeutic compounds for the combinations provided herein can be, for example, an anti-cancer or chemotherapeutic compound. Exemplary therapeutic compounds include, for example, cytokines, growth factors, photosensitizing agents, radionuclides, toxins, siRNA molecules, enzyme/pro E drug pairs, anti-metabolites, signaling modulators, anti-cancer antibiotics, anti-cancer antibodies, angiogenesis inhibitors, chemotherapeutic compounds, antimetastatic compounds or a combination of any thereof. Viruses provided herein can be combined with an anti-cancer compound, such as a platinum coordination complex. Exemplary platinum coordination complexes include, for example, cisplatin, carboplatin, oxaliplatin, DWA2114R, NK121, IS 3 295, and 254-S. Exemplary chemotherapeutic agents also include, but are not limited to, methotrexate, vincristine, adriamycin, non-sugar containing chloroethylnitrosoureas, 5-fluorouracil, mitomycin C, bleomycin, doxorubicin, dacarbazine, taxol, fragyline, Meglamine GLA, valrubicin, carmustine, polifeprosan, MM 1270, BAY 12-9566, RAS farnesyl transferase inhibitor, farnesyl transferase inhibitor, MMP, MTA/LY231514, lometrexol/LY264618, Glamolec, CI-994, TNP-470, Hycamtin/topotecan, PKC412, Valspodar/PSC833, Novantrone/mitoxantrone, Metaret/suramin, BB-94/batimastat, E7070, BCH-4556, CS-682, 9-AC, AG3340, AG3433, Incel/VX-710, VX-853, ZD0101, IS1641, ODN 698, TA 2516/marimastat, BB2516/marimastat, CDP 845, D2163, PD183805, DX8951f, Lemonal DP 2202, FK 317, picibaniil/OK-432, valrubicin/AD 32, strontium-89/Metastron, Temodal/temozolomide, Yewtaxan/paclitaxel, Taxol/paclitaxel, Paxex/paclitaxel, Cyclopax/oral paclitaxel, Xeloda/capecitabine, Furtulon/doxifluridine, oral taxoids, SPU-077/cisplatin, HMR 1275/flavopiridol, CP-358 (774)/EGFR, CP-609 (754)/RAS oncogene inhibitor, BMS-182751/oral platinum, UFT (Tegafur/Uracil), Ergamisol/levamisole, Campto/levamisole, Eniluracil/776C85/5FU enhancer, Camptosar/irinotecan, Tomudex/raltitrexed, Leustatin/cladribine, Caelyx/liposomal doxorubicin, Myocet/liposomal doxorubicin, Doxil/liposomal doxorubicin, Evacet/liposomal doxorubicin, Fludara/fludarabine, Pharmorubicin/epirubicin, DepoCyt, ZD 1839, LU 79553/Bis-Naphthalimide, LU 103793/Dolastain, Gemzar/gemcitabine, ZD 0473/Anormed, YM 116, Iodine seeds, CDK4 and CDK2 inhibitors, PARP inhibitors, D4809/dexifosfamide, Ifex/Mesnex/ifosfamide, Vumon/teniposide, Paraplatin/carboplatin, Platinol/cisplatin, VePesid/Eposin/Etopophos/etoposide, ZD 9331, Taxotere/docetaxel, prodrugs of guanine arabinoside, taxane analogs, nitrosoureas, alkylating agents such as melphalan and cyclophosphamide, aminoglutethimide, asparaginase, busulfan, carboplatin, chlorambucil, cytarabine HCl, dactinomycin, daunorubicin HCl, estramustine phosphate sodium, etoposide (VP16-213), floxuridine, fluorouracil (5-FU), flutamide, hydroxyurea (hydroxycarbamide), ifosfamide, interferon alfa-2a, interferon alfa-2b, leuprolide acetate (LHRH-releasing factor analogue), lomustine (CCNU), mechlorethamine HCl (nitrogen mustard), mercaptopurine, mesna, mitotane (o,p′-DDD), mitoxantrone HCl, octreotide, plicamycin, procarbazine HCl, streptozocin, tamoxifen citrate, thioguanine, thiotepa, vinblastine sulfate, amsacrine (m-AMSA), azacitidine, erythropoietin, hexamethylmelamine (HMM), interleukin 2, mitoguazone (methyl-GAG; methyl glyoxal bis-guanylhydrazone; MGBG), pentostatin (2′ deoxycoformycin), semustine (methyl-CCNU), teniposide (VM-26) and vindesine sulfate. Additional exemplary therapeutic compounds for the use in pharmaceutical compositions and combinations provided herein can be found elsewhere herein (see e.g., Section H.3 for exemplary cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, and chemotherapeutic compounds).

Other exemplary therapeutic compounds include, for example, compounds that are substrates for enzymes encoded and expressed by the virus, or other therapeutic compounds provided herein or known in the art to act in concert with a virus. For example, the virus can express an enzyme that converts a prodrug into an active chemotherapy drug for killing the cancer cell. Hence, combinations provided herein can contain a therapeutic compound, such as a prodrug. An exemplary virus/therapeutic compound combination can include a virus encoding Herpes simplex virus thymidine kinase with the prodrug ganciclovir. Additional exemplary enzyme/pro-drug pairs, for the use in combinations provided include, but are not limited to, varicella zoster thymidine kinase/ganciclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid, cytochrome P450/acetominophen, horseradish peroxidase/indole-3-acetic acid, nitroreductase/CB 1954, rabbit carboxylesterase/7-ethyl-10-[4-(1-piperidino)-1-piperidino]carbonyloxycamptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin-glucuronide, thymidine phosphorylase/5′-deoxy-5-fluorouridine, deoxycytidine kinase/cytosine arabinoside, beta-lactamase and linamerase/linamarin. Additional exemplary prodrugs, for the use in combinations can also be found elsewhere herein (see e.g., Section H.3). Any of a variety of known combinations provided herein or otherwise known in the art can be included in the combinations provided herein.

In some examples, the combination can include compounds that can kill or inhibit viral growth or toxicity. Such compounds can be used to alleviate one or more adverse side effects that can result from viral infection (see, e.g. U.S. Patent Pub. No. US 2009-016228-A1). Combinations provided herein can contain antibiotic, antifungal, anti-parasitic or antiviral compounds for treatment of infections. In some examples, the antiviral compound is a chemotherapeutic agent that inhibits viral growth or toxicity. Exemplary antibiotics which can be included in a combination with a virus provided herein include, but are not limited to, ceftazidime, cefepime, imipenem, aminoglycoside, vancomycin and antipseudomonal β-lactam. Exemplary antifungal agents which can be included in a combination with a virus provided herein include, but are not limited to, amphotericin B, dapsone, fluconazole, flucytosine, griseofulvin, itraconazole, ketoconazole, miconazole, clotrimazole, nystatin, and combinations thereof. Exemplary antiviral agents can be included in a combination with a virus provided herein include, but are not limited to, cidofovir, alkoxyalkyl esters of cidofovir (CDV), cyclic CDV, and (S)-9-(3-hydroxy-2 phosphonylmethoxypropyl)adenine, 5-(dimethoxymethyl)-2′-deoxyuridine, isatin-beta-thiosemicarbazone, N-methanocarbathymidine, brivudine, 7-deazaneplanocin A, ST-246, Gleevec, 2′-beta-fluoro-2′,3′-dideoxyadenosine, indinavir, nelfinavir, ritonavir, nevirapine, AZT, ddI, ddC, and combinations thereof. Typically, combinations with an antiviral agent contain an antiviral agent known to be effective against the virus of the combination. Exemplary antiviral compounds include, for example, cidofovir, alkoxyalkyl esters of cidofovir, ganciclovir, acyclovir, ST-246, Gleevec, and derivatives thereof.

In some examples, the combination can include a detectable compound. A detectable compound can include, for example, a ligand, substrate or other compound that can interact with and/or bind specifically to a protein or RNA encoded and expressed by the virus, and can provide a detectable signal, such as a signal detectable by tomographic, spectroscopic, magnetic resonance, or other known techniques. In some examples, the protein or RNA is an exogenous protein or RNA. In some examples, the protein or RNA expressed by the virus modifies the detectable compound where the modified compound emits a detectable signal. Exemplary detectable compounds can be, or can contain, an imaging agent such as a magnetic resonance, ultrasound or tomographic imaging agent, including a radionuclide. The detectable compound can include any of a variety of compounds as provided elsewhere herein or are otherwise known in the art. Exemplary proteins that can be expressed by the virus and a detectable compound combinations employed for detection include, but are not limited to luciferase and luciferin, β-galactosidase and (4,7,10-tri(acetic acid)-1-(2-β-galactopyranosylethoxy)-1,4,7,10-tetraazacyclododecane) gadolinium (Egad), and other combinations known in the art.

In some examples, the combination can include a gene expression modulating compound that regulates expression of one or more genes encoded by the virus. Compounds that modulate gene expression are known in the art, and include, but are not limited to, transcriptional activators, inducers, transcriptional suppressors, RNA polymerase inhibitors and RNA binding compounds such as siRNA or ribozymes. Any of a variety of gene expression modulating compounds known in the art can be included in the combinations provided herein. Typically, the gene expression modulating compound included with a virus in the combinations provided herein will be a compound that can bind, inhibit or react with one or more compounds, active in gene expression such as a transcription factor or RNA of the virus of the combination. An exemplary virus/expression modulator combinations can be a virus encoding a chimeric transcription factor complex having a mutant human progesterone receptor fused to a yeast GALA DNA-binding domain an activation domain of the herpes simplex virus protein VP16 and also containing a synthetic promoter containing a series of GAL4 recognition sequences upstream of the adenovirus major late E1B TATA box, where the compound can be RU486 (see, e.g., Yu et al., (2002) Mol Genet Genomics 268:169-178). A variety of other virus/expression modulator combinations known in the art also can be included in the combinations provided herein.

In some examples, the combination can contain one or more additional therapeutic and/or diagnostic viruses or other therapeutic and/or diagnostic microorganism (e.g. therapeutic and/or diagnostic bacteria) for diagnosis or treatment. Exemplary therapeutic and/or diagnostic viruses are known in the art and include, but are not limited to, therapeutic and/or diagnostic poxviruses, herpesviruses, adenoviruses, adeno-associated viruses, and reoviruses.

5. Kits

The VV-protein polymer (e.g. a VV-SELP or LIVP-SELP) compositions, or components thereof, can be packaged as kits. For example, provided herein are kits for making or generating the compositions provided herein. The kits can include a polymer protein (e.g. a SELP protein), a vaccinia virus and optionally instructions for preparing the polymer compositions herein by mixture of the polymer protein and vaccinia virus. In the provided kit, the polymer protein and/or vaccinia virus are provided as separate compositions for mixture together. The polymer protein and vaccinia virus can be provided in a dried or concentrated suspension or in a suspension containing the compounds at the concentration for use.

Kits can optionally include one or more components such as instructions for use, devices and additional reagents, and components, such as tubes, containers, syringes and other devices for practice of the methods. For example, optionally, the kit also can contain an aqueous solution such as a solvent or buffer for supending or dissolving the agents, a device for detecting a virus in a subject, a device for administering the virus to a subject, or a device for administering an additional agent or compound to a subject. Exemplary devices include, but are not limited to, a hypodermic needle, an intravenous needle, a catheter, a needle-less injection device, an inhaler and a liquid dispenser, such as an eyedropper. For example, a kit containing a composition, or components to generate a composition, to be delivered systemically, for example, by intravenous injection, can be included in a kit with a hypodermic needle and syringe. In another example, a kit containing a composition, or components to generate a composition, to be delivered topically can contain a device (e.g. a bandage or dressing) for applying or coating the composition, an applicator for applying the composition (e.g. disposable or reusable) The kit optionally can include a pharmaceutical carrier for combination with the composition prior to administering the composition to an animal subject and/or an additional therapeutic agent for administering in combination with the prepared composition. In addition to instructional materials for preparing the hydrogel mixture, the kit also can optionally include instructions for methods of using or administering the composition or device to an animal subject.

In one example, a kit can contain instructions. Instructions typically include a tangible expression describing the virus, describing the polymer and/or describing the generation of the compositions herein and, optionally, other components included in the kit, and methods for administration, including methods for determining the proper state of the subject, the proper dosage amount, and the proper administration method, for administering the virus. Instructions can also include guidance for monitoring the subject over the duration of the treatment time.

### F. DEVICES AND ARTICLES OF MANUFACTURE

The compositions provided herein can be used to make, prepare or to coat a device that is to be applied to a surface of the body of a animal (e.g. human) or that is to be inserted with the body of an animal (e.g. a human). Hence, provided herein are devices prepared from any of the protein polymer hydrogel compositions containing a vaccinia virus (e.g. an LIVP) provided herein in Section E. In other examples, provided herein are devices having a surface coated with any of the protein polymer hydrogel compositions containing a vaccinia virus (e.g. an LIVP virus) provided herein in Section E. In particular, the hydrogel composition is a SELP hydrogel. The device can be an implantable device or other device that is amenable to providing the VV-SELP hydrogel composition to physiologic environment of an animal (e.g. human). Exemplary of such devices include, but are not limited to, suture, dressing, bandage, film, mesh, suture, shunt or other implantable device.

The devices are prepared by forming the device from or coating the device with a composition provided herein. For example, a thin slab of a liquid composition can be coated onto a bandage, wrap or other dressing. Thereafter, the device (e.g. bandage, wrap or other dressing) can be exposed to physiologic temperature where the hydrogel will form. This can be achieved by applying the bandage or other device to the site of a wound or other skin lesion. This also can be achieved prior to applying the bandage or other device to a subject by heating or warming the device ex vivo.

In particular examples, the compositions provided herein can be used to coat virtually any medical device. The coated devices provide a convenient means for local administration of the vaccinia virus in polymer composition. For example, the compositions can be used to coat degradable and non-degradable sutures, orthopedic prostheses such as supporting rod implants, joint prostheses, pins for stabilizing fractures, bone cements and ceramics, tendon reconstruction implants, ligament reconstruction implants, cartilage substitutes, prosthetic implants, cardiovascular implants such as heart valve prostheses, pacemaker components, defibrillator components, angioplasty devices, intravascular stents, acute and in-dwelling catheters, ductus arteriosus closure devices, implants deliverable by cardiac catheters such as atrial and ventricular septal defect closure devices, urologic implants such as urinary catheters and stents, neurosurgical implants such as neurosurgical shunts, ophthalmologic implants such as lens prosthesis, thin ophthalmic sutures, and corneal implants, dental prostheses, tissue scaffolds (particularly soft tissue scaffolds), internal and external wound dressings such as bandages and hernia repair meshes, and other devices and implants known to one of skill in the art.

For example, the device having a surface coated with a composition provided herein is a suture. Typically, these devices are coated with a hydrogel matrix containing a vaccina virus (e.g. an LIVP). Sutures that can be coated include any suture of natural or synthetic origin. Typical suture materials include, by way of example and not limitation, silk, cotton, linen, polyolefins such as polyethylene and polypropylene, polyesters such as polyethylene terephthalate, homopolymers and copolymers of hydroxycarboxylic acid esters, plain or chromicized collagen, plain or chromicized catgut, and suture substitutes such as cyanoacrylates. The sutures can take any convenient form such as braids or twists, and can have a wide range of sizes, such as are commonly employed in the art.

In particular examples, the material or device is a patch, such as a dressing, bandage, wrap, dressing a film, a mesh. Bandages, films, dressing, meshes and other similar device can be include any device known in the art or capable of being used as a wound dressing.

### G. ASSAYS TO ASSESS VIRUS ACTIVITY OR COMPOSITION PROPERTIES

The compositions provided herein as a hydrogel matrix or a liquid hydrogel precursor matrix can be tested to determine the viral integrity, stability or infectivity of the vaccinia virus contained therein. Assays also can be performed to assess the diffusion or release of the virus therefrom. Assays also can be performed to assess the therapeutic efficacy of the virus, and in particular its anti-tumorigenicity or toxicity/safety. Such assays are well known in the art, and exemplary of such assays are described herein.

1. Characterization of Hydrogel Compositions

The gelling behavior, swelling, mechanical strength and other properties of virus contained in protein polymer compositions provided herein can be assessed. Methods to assess or evaluate the gelling behavior of protein polymer matrix compositions, in particular SELP compositions, are known in the art (see e.g. Megeed et al. (2002) Advanced Drug Delivery Reviews, 54:1075-1091; Cappello et al. (1998) Journal of Controlled Release, 53:105-117). Such methods include, but are not limited to, differential scanning calorimetry (DSC; e.g. modulated DSC, for example, model MDSC 2920, TA, Instruments, DE) and rotational viscometry (e.g. using a cone and plate viscometer, for example, Brookfield Syncro-Lectric rotational viscometer; Brookfield Engineering Laboratories). Rheology studies, for example using a rheometer, also can be performed to assess changes in viscosity upon matrix formation.

In addition, the degree of swelling in water also can be determined, which is a property of the hydrogels (Sudipto et al. (2002) Journal of Microelctromechanical Systems, 11:544; Dinerman et al. (2002) Biomaterials, 23:4203-4210; Gutafson et al. (2010) Advanced Drug Delivery Reviews, 62:1509-1523)). Swelling of the hydrogels can be influenced by the interaction and cross-linking of the polymer side chains, such that reduced or decreased interaction results in a higher degree of swelling. The hydrogel swelling ratio (q) is a measure of the weight of a hydrated hydrogel divided by its dry weight. Generally, hydrogel compositions exhibit a swelling ratio that is less than 16, 15, 14, 13, 12, 11, 10, 9, 8, 7, 6, 5 or less. The temperature dependence of the swelling ratio can also be assessed, since some protein polymer compositions can exhibit temperature-dependent swelling.

The mechanical strength of the protein polymer hydrogels also can be evaluated. Methods to assess or evaluate the mechanical strength of protein polymer matrix compositions, in particular SELP compositions, are known in the art (see e.g. Gutafson et al. (2010) Advanced Drug Delivery Reviews, 62:1509-1523). Such methods include measurements of the shear modulus (i.e. the ability of a material to resist shear strain under exposure to shear stress, for example, using cone and plate rheology); measurement of the storage modulus using dynamic mechanic analysis (DMA) at various strain rates and frequencies; and small-angle neutron scattering (SANS).

The rate of gelation or hydrogel formation, swelling or mechanical strength of the compositions herein can be evaluated based on factors such as the particular protein polymer (e.g. SELP) in the composition, including the number of protein polymers (e.g. the number of silk-liked blocks contained in the polymer), molecular weight and weight percentage of the composition; the presence and effect of virus in the composition, including the amount of virus in the composition; the effect of the temperature on gelation; the time to gelation (i.e. cure time); the pH; ionic strength and/or the effect of any additives or excipients on gelation. It is within the level of one of skill in the art to empirically adjust various parameters to optimize the particular virus in protein polymer composition depending on its particular application or use.

2. Evaluation of Virus Diffusion or Release

The release or release rate of virus contained in protein polymers also can be assessed. Such methods also are known in the art (see e.g. Hatefi et al. (2006) Molecular Therapy, 13:S205). For example, hydrogel forms of virus in protein polymer compositions can be incubated in a solution (e.g. a physiological buffer, such as phosphate buffered saline) for a period of time and at a temperature (e.g. 37° C.), and virus release therefrom can be monitored. In particular examples, virus in protein polymer hydrogel samples are already formed, and gel discs of a known volume (e.g. 0.02 to 0.1 cm³ or 20 μL to 100 μL) can be excised and plated in an elution tube with a physiologic buffer and incubated at 37° C. Samples from the incubated solutions can be taken at various intervals or predetermined time points. The amount of virus in the sample can be assessed by various methods known to one of skill in the art, for example, by standard plaque assay or extraction of DNA and quantification using RT-PCR. In some examples, viruses containing or expressing a detectable moiety or a moiety capable of detection also can be used to measure the release or release rate of virus. For example, a fluorometer can be used to measure fluorescence emission from the sample if the virus is one that expresses a fluorescent protein. The amount of virus remaining in the gel at termination of the analysis also can be determined by dissolving the gel. For example, the gel can be dissolved in 88% formic acid, neutralized to pH 7.4 with sodium hydroxide.

The results can be compared to a known standard curve to determine the number of virus particles released over time. Also, the rate of release can be monitored by comparing the amount of virus in the sample as a function of time. Viral integrity of the released virus also can be assessed to ensure that it remains bioactive after release from the hydrogel (discussed below).

It is within the level of one of skill in the art to empirically adjust various parameters to optimize the release or release rate of virus in protein polymer composition depending on its particular application or use. For example, a protein polymer composition can be chosen that permits a time-dependent rate of release of virus such that virus is steadily released over the course of several days or up to a week or more. In one example, a protein polymer composition is chosen that effects release of greater than 50%, 60%, 70%, 80%, 90% or 100% of the loaded virus within 7 days, 8 days, 9 days, 10 days, 11 days, 12 days, 13 days or 14 days. Generally, 100% of the loaded virus is not released over the first few hours or days, but is steadily released over the course of one day, two days, three days, four days, five days, six days seven days or more. For example, protein polymer compositions can be chosen that release no more than 50%, 40%, 30%, 20%, 10%, 5% or less of virus within the first 24 hours.

3. Evaluation of Viral Integrity

The virus integrity of virus contained in protein polymers can be assessed.

Any assay that is capable of assessing infectivity of a virus can be used. Exemplary of such assays for assessing viral integrity or infectivity is assessment of viral titer by a standard plaque assay or the expression of a viral gene expression or replication after infection of a tumor cell line or other permissive cell. Such assays are known to one of skill in the art, and exemplary of such assays are described below. In particular, as described below, various tumor cell lines or other permissive cell lines are known to one of skill in the art and can be used in such assays to assess viral integrity over time in hydrogel compositions herein. For purposes of assessing viral integrity, the hydrogel forms of the virus in protein polymer compositions are incubated over time and at physiologic temperatures to determine if virus contained therein remains viable. Typically, virus in protein polymer compositions provided herein, for example VV-SELP such as LIVP-SELP compositions, are stable at physiologic temperatures (e.g. 34° C. to 37° C.) for at least one week and up to four weeks or more, and are stable at room temperature for even longer. For example, the viral integrity of vaccinia virus, such as an LIVP, in a protein polymer composition provided herein is at least or greater than or about or 10%, 11%, 12%, 13%, 14%, 15%, 16%, 17%, 18%, 19%, 20%, 25%, 30%, 35%, 40%, 45%, 50%, 55%, 65%, 70%, 80%, 90%, 95% of the viral integrity of the starting virus stock prior to incubation at 37° C.

In one example, for testing a virus that is in a hydrogel matrix, the matrix can be degraded prior to testing of the virus therein. For example, the matrix can be degraded either by providing an enzyme having matrix-degrading activity (e.g. elastase) or by physically degrading or dissolving the matrix by crushing, grinding, or by incubation with acid (e.g. 88% formic acid, neutralized to pH 7.4 with sodium hydroxide). The non-matrix virus-containing portion of the sample can be separated therefrom, and the sample can be assessed and used to transfect or infect permissive cells or tumor cells. A standard plaque assay can be performed to assess viral titer as a measure of viral integrity. Alternatively, a marker gene expressed by the virus (e.g. a fluorescent protein) can be quantitated as an indication of virus infection and replication.

In another example, the virus that is assessed is one that is released from a hydrogel composition. For example, virus-containing hydrogel compositions can be incubated at or about 37° C. for varying periods of time by incubation of a gel disk in a physiologic buffer. The gels can be agitated, for example, in a shaking incubator. The release media can be removed and used to transfect or infect permissive cells or tumor cells. A standard plaque assay can be performed to assess viral titer as a measure of viral integrity. Alternatively, a marker gene expressed by the virus (e.g. a fluorescent protein) can be quantitated as an indication of virus infection and replication.

Viral integrity also can be assessed in vivo. For example, viral gene expression can be monitored over time following injection into a subject or animal model. In such examples, the VV-protein polymer composition, such as a VV-SELP composition (e.g. LIVP-SELP) is one that expresses a detectable gene product or a product capable of detection. After administration of the virus to a subject (e.g. a tumor bearing animal model, such as any generated as described below), the expression of a marker or other gene product can be monitored over time to assess if virus released from the composition is capable of infecting tumor cells. To specifically assess tumor-specific gene expression, tumors of animals can be imaged to detect the marker without sacrificing the subject or animals. In other examples, animals can be sacrificed over various time points, and the amount of marker present in tumor tissue can be determined.

4. Anti-Tumorigenicity and Efficacy

Viruses in protein polymer compositions provided herein can be tested for parameters indicative of its anti-tumorigenic property. Generally, the parameters selected for are desirable for the treatment of proliferative diseases and disorders, including the treatment of a tumor or metastasis. For example, a virus can destroy tumor cells by replicating such that continual amplification of the virus results in infection of adjacent cells and their subsequent destruction. Oncolytic viruses also exhibit anti-tumorigenicity by expression of proteins that are cytotoxic to cancer cells. In further examples, viruses can exhibit anti-tumorigenicity by initiating specific and nonspecific anti-tumor immune responses, for example, the initiation of cytokine expression from infected cells (e.g. TNF) or through a specific response (e.g. CTL response). Hence, any of the above parameters can be assessed as indicative of anti-tumorigenicity of a virus.

For example, the isolated virus is tested in one or more in vitro and/or in vivo assays that assess infectivity, viral nucleic acid replication, virus production, viral gene expression from tumor cells, effects on the host cell, cytotoxicity of tumor cells, tumor cell selectivity, tumor cell type selectivity, specific and nonspecific immune response, and therapeutic efficacy. Parameters indicative of anti-tumorigenicity can be assessed in vitro or in vivo. In particular examples, anti-tumorigenicity is assessed in vivo. In vivo parameters of anti-tumorigenicity include, but are not limited to, a desirable therapeutic index in an animal model of cancer, release of tumor antigens and preferential accumulation of the virus in tumor tissues following administration. Exemplary of assays or methods to assess such parameters are described below.

a. Tumor-Associated Replication Indicator

Viruses in protein polymer compositions provided herein can be tested for replication and/or infectivity in tumor cells. The replication indicator that is measured is any parameter from which the level or amount or relative amount of viral replication, typically within a day of administration to the tumor cells, can be assessed or inferred. In some examples, replication can be assessed by measurement of a viral replication indicator, such as, for example, viral titer (i.e. as assessed by the number of plaques produced in a plaque assay) or the changes in viral gene expression or host gene expression (see, e.g. U.S. Patent Pub. No. 2009-0136917). For example, replication can be determined by infecting or introducing the test virus into a tumor cell and assessing a replication indicator at a particular time or as a function of time. This can be compared to a predetermined standard, for example the parental virus preparation or mixture or other reference strain (e.g. recombinant virus), or compared to other test strains or controls. Viruses can be tested to assess selective replication in tumor cells compared to normal cells.

Assays to assess replication can be performed on cell lysates of cells infected in vitro with any of the vaccinia virus in protein compositions provided herein (e.g. VV-SELP, such as LIVP-SELP compositions), for example, various tumor cell lines, primary tissues or cells as well as tumor cells such as from a biopsy. For example, a tissue or cell sample can be obtained (e.g., biopsy) from a subject (e.g., human or non-human animal subject), and the sample can be infected with one or more types of virus compositions. In other examples, tumor cell lines can be used. Tumor cell lines are known and available to one of skill in the art, for example, from the American Type Culture Collection (ATTC; Manassas, Va.) or from the European Collection of Cell Cultures (ECACC). Tumor cell lines also are available from the Division of Cancer Treatment and Diagnosis (DCTD) Tumor Repository (National Cancer Institute/National Institute of Health; dtp.nih.gov/index.html.) Exemplary of tumor cell lines include human and other animal cells lines and include, but are not limited to, DU145 human prostate carcinoma cells, LNCaP human prostate cancer cells, MCF-7 human breast cancer cells, MRC-5 human lung fibroblast cells, MDA-MB-438 human breast cancer cells, MDA-MB-231 human breast carcinoma cells, PC3 human prostate cancer cells, T47D human breast cancer cells, THP-1 human acute myeloid leukemia cells, U87 human glioblastoma cells, SH-SY5Y human neuroblastoma cells, Saos-2 human cells, A549 human lung carcinoma cells, A2780 human ovarian carcinoma cells, HCT 116 human colon cells, HT-29 human colon cells, SW260 human colon cells, HT-180 human fibrosarcoma, MIA PaCa-2 human pancreatic carcinoma cells, PANC-1 human pancreatic cells, CMT 64 C57BL/6 mouse cell, JC mouse mammary cells, TIB-75 mouse hepatic cells, CT26 WT mouse colon carcinoma cells, MC-38 mouse adenocarcinoma cells, B 16-F10 mouse melanoma cells, 4T1 murine mammary carcinoma cells and hamster pancreatic tumor HP-1 cells.

For example, cells or cell lines can be seeded onto wells of a plate. Virus compositions can then be added and allowed to infect the cells. At the end of the infection, the media can be changed to remove any residual virus and the cells further incubated. Then, the cells can be scraped into the media and collected. Cells can be lysed, for example, by freeze-thaw and/or sonication, to obtain virus-containing lysates. The extent of replication can be measured, such as by determination of viral titer or expression of genes as described further below. It is understood that the extent and degree of replication and/or infectivity efficiency of a virus will differ between various tumor cell types.

Assays to assess replication also can be performed on tumor-harvested virus propagated in vivo upon infection of tumor-bearing animals. Such an assay is a measure of the accumulation of the virus in tumor tissues. As discussed below, tumors can be established in animals by implantation of different tumor cell types. For example, tumor-bearing animals can be administered (e.g. topically or via intravenous administration or other route of administration) with a vaccinia virus in protein compositions provided herein (e.g. VV-SELP, such as LIVP-SELP compositions), virus propagated in tumors and virus or tumor extracted therefrom. The extent of replication can be measured, such as by determination of viral titer or expression of genes, as described further below.

In one example, cell culture supernatants or cell lysates from the infected cells or tumor cell extracts can be obtained following infection and subjected to assays to measure viral titer. For example, a standard plaque assay can be used. The plaque assay can indicate the biological activity in different cell types, including different tumor cell types. Titration of virus by plaque assay is known to one of skill in the art. In one example of a plaque assay, supernatants or cells lysates of tumors or cells infected with the virus is harvested and plaque assays can be performed. Typically, serial dilutions of the virus supernatant or lysate is made in the range of 10⁻² (1:100) to 10⁻¹⁰, and in particular from 10⁻⁵ to 10⁻¹⁰. Diluted virus is added to a monolayer of cells, for example, monolayers of permissive cell line, such as, for example, CV-1, Vero, BHK, RK13 or HEK-293 cell line, and incubated with virus. In some examples, the plaque assay can be performed directly on a cell monolayer of a tumor cells provided that the tumor cells can form a monolayer. Following incubation, an agarose overlay is added to the monolayer of cells without dislodging the cells, and the plate is further incubated until plaques become visible. A dye or color stain solution that is taken up by healthy cells but not dead cells, such as neutral red, is added to each of the wells or plate. After incubation, the dye or stain is removed such that the plaques are observed to be clear, while non-lysed cells remain stained. Titer (pfu/mL) is calculated by counting the number of plaques in the well and dividing by the dilution factor (d) and the volume (V) of diluted virus added to the well (# plaques/d×V). The virus yield can be converted to pfu/cell by dividing the total amount of virus present in the sample by the number of cells originally infected in the sample.

Other indicators of replication also can be assessed. For example, expression of viral genes, tumor proteins and/or housekeeping genes that are correlated with viral replication and/or infectivity in tumor cells can be assessed (see e.g. U.S. Patent Pub. No. 2009-0136917). For example, expression of housekeeping genes or other genes in tumor cells associated with virus replication and infectivity can be assessed (U.S. Patent Pub. No. 2009-0136917). For example, expression of a plurality of such genes, such as housekeeping genes, whose expression increases in tumor cells upon infection with virus are assessed. Exemplary of such genes that can be assessed include expression of one or more genes encoding a protein selected from among IL-18 (Interleukin-18), MCP-5 (Monocyte Chemoattractant Protein-5; CCL12), IL-11 (Interleukin-11), MCP-1 (Monocyte Chemoattractant Protein-1), MPO (Myeloperoxidase), Apo A1 (Apolipoprotein A1), TIMP-1 (Tissue Inhibitor of Metalloproteinase Type-1), CRP(C Reactive Protein), Fibrinogen, MMP-9 (Matrix Metalloproteinase-9), Eotaxin (CCL11), GCP-2 (Granulocyte Chemotactic Protein-2; CXCL6), IL-6 (Interleukin-6), Tissue Factor (TF), SAP (Serum Amyloid P), FGF-basic (Fibroblast Growth Factor-basic), MCP-3 (Monocyte Chemoattractant Protein-3; CCL7), IP-10 (CXCL 10), MIP-2, Thrombopoetin, Cancer antigen 125, CD40, CD40 ligand, ENA-78, Ferritin, IL-12p40, IL-12p70, IL-16, MMP-2, PAI-1, TNF RII, TNF-beta and VCAM-1. In another example, expression of a plurality of genes, such as housekeeping genes, whose expression decreases in tumor cells upon infection with virus are assessed. Exemplary of such genes include one or more genes encoding a protein selected from among MIP-1beta (Macrophage Inflammatory Protein-1beta), MDC (Macrophage-Derived Chemokine; CCL22), MIP-1alpha (Macrophage Inflammatory Protein-1alpha; CCL3), KC/GROalpha (Melanoma Growth Stimulatory Activity Protein), VEGF (Vascular Endothelial Cell Growth Factor), Endothelin-1, MIP-3 beta (Macrophage Inflammatory Protein-3 beta; Exodus-3 or ELC), Beta-2 microglobulin, IL-5 (Interleukin-5), IL-1 alpha (Interleukin-1 alpha), EGF (Epidermal Growth Factor), Lymphotactin (XCL1), GM-CSF (Granulocyte Macrophage-Colony Stimulating Factor), MIP-lgamma (Macrophage Inflammatory Protein-1 gamma; CCL4), IL-1beta (Interleukin-1 beta), BDNF (Brain-derived neutrophic factor), Cancer antigen 19-9, Carcinoembryonic antigen, C reactive protein, EGF, Fatty acid binding protein, Factor VII, Growth hormone, IL-1 alpha, IL-1 beta, IL-1 ra, IL-7, IL-8, MDC, Prostatic acid phosphatase, Prostate specific antigen, free, Stem cell factor, Tissue factor, TNF-alpha, VEGF and Von Willebrand factor.

Gene expression can be assayed after contacting a tumor sample with the virus for a period of time in vitro or in vivo and measuring the level of expression of one or more housekeeping genes or other genes. Any method known in the art can be used for assessing the expression of genes in a tumor can be employed. For example, methods for measuring protein expression levels which can be used include, but are not limited to, microarray analysis, ELISA assays, Western blotting, or any other technique for the quantitation of specific proteins. For RNA levels, examples of techniques which can be used include microarray analysis, quantitative PCR, Northern hybridization, or any other technique for the quantitation of specific nucleic acids. In some examples, a difference in expression of the same marker between the contacted and non-contacted biological samples of about less than 2-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold or greater than about 100-fold is indicative of specific replication and/or infectivity of a tumor cell.

For in vitro tests, varying doses/multiplicity of infection (MOI; ratio of virus to cell) of the virus can be assessed in order to assess the rate of viral infection and virus production at different infection levels. Viruses that exhibit a high rate of replication at a lower MOI are generally desirable for therapy of a proliferative disorder or disease. For example, cells can be infected at an MOI of at or between 0.1 to 10, such as 0.5 to 5, for example, 0.5 to 2, for example, an MOI of at or at least 0.25, 0.5, 1, 1.5, 2 or more.

In any of the examples herein of assessing replication or infectivity of a virus, tumor cell selectivity of the virus also can be assessed. For example, normal cells and tumor cells can be infected with the vaccinia virus in protein composition (e.g. VV-SELP, such as LIVP-SELP compositions) followed by assessment of replication and or infectivity using any of the assays described herein or known to one of skill in the art. For example, measurement of viral titer by plaque assay or by expression of genes as described can be determined in virally-infected tumor cells versus virally-infected normal cells. Normal or non-transformed cells include, but are not limited to, MRC-5 lung fibroblast cells, Beas-2B bronchial epithelial cells, normal human bronchial epithelial (NHBE), small airway bronchial epitherlial (SAEC). Tumor cells include any described herein or known to one of skill in the art and include, but are not limited to, A2780, A549, HCT 116, HT 1080, LNCaP or SW620 cells. In some examples, paired tumor and non-tumor cell lines can be infected with virus and compared. Exemplary corresponding or paired tumor and non-tumor cell lines are known to one of skill in the art (see e.g., Gazdar et al. (1998) Int. J. Cancer, 78:766-774, Theodore et al. (2010) Int. J. Oncology, 37:1477-1482; Niedbala et al. (2001) Radiation Research, 155:297-303). In other examples, tumors infected in vivo can be harvested and can be compared to normal cells or tissues that also are extracted from the same infected animal. Infection and replication of virus in normal cells and tumor cells can be assessed and compared. The therapeutic index of the virus can be determined by the ratio of replication in the tumor cell compared to the normal cell (e.g. virus produced per cell; pfu/cell).

b. Cytotoxicity

Viruses in protein polymer compositions provided herein (e.g. VV-SELP, for example LIVP-SELP) can be tested to determine if they are cytotoxic or kill tumor cells. For example, viruses can eliminate tumor cells via induction of cell death and/or lysis of the tumor cell (i.e. oncolysis). The cell killing activity of the virus can be assessed by a variety of techniques known in the art including, but not limited to, cytotoxicity/cell viability assays that can be employed to measure cell necrosis and/or apoptosis following virus infection, such as MTT (3-(4,5-dimethylthiazol-2-yl)-2,5-diphenyltetrazolium bromide) assays and other related tetrazolium salt based assays (e.g. XTT, MTS or WST), ATP assays, apoptosis assays, such as TUNEL staining of infected cells, DNA fragmentation assays, DNA laddering assays, and cytochrome C release assays. Such assays are well known to one of skill in the art.

For example, viability of virally-infected cells can be assessed. Various tumor cell lines, for example any described above or known to one of skill in the art, can be seeded in a 96-well plate (e.g. at or about 5,000 cells/well) or other size well-plate and grown overnight, and then can be infected with serial dilution of virus or the VV-protein polymer compositions provided herein (e.g. VV-SELP, for example LIVP-SELP). For example, various MOI of the virus can be tested. MOI can range from, for example, 1000 to 0.0001, such as 100 to 0.001 or 10 to 0.01. It is within the level of one of skill in the art to empirically select or determine an appropriate MOI range in which to use. Once infected, the cells can be incubated for a period of time before assessment of cytotoxicity. For example, samples for assessment of cytotoxicity are typically obtained at selected time points following virus infection of the cells, such as, for example, 1 hour, 2 hours, 4 hours, 8 hours, 16 hours, 24 hours, 1.5 days. 2 days, 2.5 days, 3 days, 4 days, 5 days, 6 days or more. One of skill in the art can select appropriate time points for assessment of viral replication based on the relative infectivity of the virus compared to other known virus strains. Generally, infection is allowed to proceed at least 12 hours, 24 hours, 36 hours, 48 hours, 72 hours, 84 hours, 96 hours or more.

Following infection for the designated period, media is replaced and viability of the cells is determined based on any assay or procedure known to one of skill in the art. Exemplary of assays to assess viability are colorimetric assays that permit visualization of cells based on metabolic activity and measure the reducing potential of the tetrazolium salt to a colored formazan product (e.g. MTT assay, MTS assay or XTT assay). Other redox assays include assays that measure the ability of cells to convert a redox dye resazurin to a fluorescent end product resorufin (McMillian et al. (2002) Cell Biol. Toxicology, 18:157-173; CellTiter-Blue™ Cell Viability Assay, Promega). In other examples, viability can be assessed using a CASY cell counting technology, which is an electric field multi-channel cell counting system based on existence of a transmitted electric field through injured or dead cells as compared to normal cells (e.g. CASY® Model TT; Roche Innovatis AG). Additional examples include, but are not limited to, trypan blue or propidium iodide dye exclusion assay, measurement of lactate dehydrogenase (LDH; see e.g., LDH Cytotoxicity Detection Kit, Clontech, Cat. #630117), sulforhodamine B (SRB) assay (e.g. CytoScan™ SRB Cytotoxicity Assay, GBiosciences, Cat. No. 786-213, WST assay (e.g. Cytoscan™ WST-1 Cell Proliferation Assay, GBiosciences, Cat No. 786-212), clonogenic assay and luciferase-based ATP-based assays (e.g., CelTiter-Glo™ Luminexcent Cell Viability Assay; Promega).

Generally, the assays are performed using various controls. For example, any assay to assess viability generally is performed with untreated wells containing cells only (e.g. 100% viable) as well as cell-free wells (0% viable). Also, in addition to the testing vaccinia virus in protein polymer compositions, other control viruses can be tested. For example, a reference virus strain, for example, a known attenuated recombinant strain can be tested that is a non-matrix virus composition. Exemplary of such as strain is GLV-1h68 or a derivative thereof containing inserted heterologous genes. In examples where virus is added as a control, the MOI range of virus that is used is the same as the tested VV-protein polymer composition.

A virus exhibits a cytopathic effect if it is determined to exhibit a reduction in cell viability relative to an untreated well containing cells only (100% viable). In other examples, a virus exhibits a cytopathic effect if it is determined to exhibit a reduction in cell viability relative to the viability of cells in a well treated with a control or reference virus that is not oncolytic. In a further example, a virus exhibits a cytopathic effect if it is determined to exhibit a similar or greater effect on cell viability relative to the viability of cells in a well treated with a known reference attenuated virus strain, such as an attenuated recombinant virus (e.g. GLV-1h68 or derivative thereof).

c. Tumor Growth

Viruses in protein polymer compositions provided herein (e.g. VV-SELP, for example LIVP-SELP) can be tested to determine if it causes shrinkage of tumor size and/or delays tumor progression. Tumor size can be assessed in vivo in tumor-bearing human or animal models treated with virus. Tumor shrinkage or tumor size can be assessed by various assays known in art, such as, by weight, volume or physical measurement.

Tumor-bearing animal models can be generated. In vivo tumors can be generated by any known method, including xenograft tumors generated by inoculating or implanting tumor cells (e.g. by subcutaneous injection) into an immunodeficient rodent, syngeneic tumors models generated by inoculating (e.g. by subcutaneous injection) a mouse or rat tumor cell line into the corresponding immunocompetent mouse or rat strain, metastatic tumors generated by metastasis of a primary tumor implanted in the animal model, allograft tumors generated by the implantation of tumor cells into the same species as the origin of the tumor cells, and spontaneous tumors generated by genetic manipulation of the animal. The tumor models can be generated orthotopically by injection of the tumor cells into the tissue or organ of their origin, for example, implantation of breast tumor cells into a mouse mammary fat pad. Any of the above models provide a consistent and reproducible tool for evaluating tumor cell growth, as well as permitting easy access to assess the mass of the tumor.

In particular examples, xenograft models or syngenic models are used. For example, tumors can be established by subcutaneous injection at the right armpit with a cell suspension (e.g. 1×10⁶ to 5×10⁶ cells/animal) of different tumor cell types into immunocompetent hosts (syngeneic) or immunodeficient hosts (e.g. nude or SCID mice; xenograft). Exemplary human tumor xenograft models in mice, such as nude or SCID mice, include, but are not limited to, human lung carcinoma (A549 cells, ATCC No. CCL-185); human breast tumor (G1-101A cells, Rathinavelu et al., Cancer Biochem. Biophys., 17:133-146 (1999)); human ovarian carcinoma (OVCAR-3 cells, ATCC No. HTB-161); human pancreatic carcinoma (PANC-lcells, ATCC No. CRL-1469 and MIA PaCa-2 cells, ATCC No. CRL-1420); DU145 cells (human prostate cancer cells, ATCC No. HTB-81); human prostate cancer (PC-3 cells, ATCC# CRL-1435); colon carcinoma (HT-29 cells); human melanoma (888-MEL cells, 1858-MEL cells or 1936-MEL cells; see e.g. Wang et al., (2006) J. Invest. Dermatol. 126:1372-1377); and human fibrosarcoma (HT-1080 cells, ATCC No. CCL-121,) and human mesothelioma (MSTO-211H cells). Exemplary rat tumor xenograft models in mice include, but are not limited to, glioma tumor (C6 cells; ATCC No. CCL-107). Exemplary mouse tumor homograft models include, but are not limited to, mouse melanoma (B 16-F10 cells; ATCC No. CRL-6475). Exemplary cat tumor xenograft models in mice include, but are not limited to, feline fibrosarcoma (FC77.T cells; ATCC No. CRL-6105). Exemplary dog tumor xenograft models in mice include, but are not limited to, canine osteosarcoma (D 17 cells; ATCC No. CCL-183). Non-limiting examples of human xenograft models and syngeneic tumor models are set forth in the Tables 7 and 8 below.

TABLE 7
Human Tumor Xenograft Models

| | Cell Line | | |
|---|---|---|---|
| Tumor Type | Name | Tumor Type | Cell Line |
| Adenoid cystic | ACC-2 | Leukemia | HL-60 |
| carcinoma | | | |
| Bladder carcinoma | EJ | Liver carcinoma | Bel-7402 |
| Bladder carcinoma | T24 | Liver carcinoma | HepG-2 |
| Breast carcinoma | BCaP-37 | Liver carcinoma | QGY-7701 |
| Breast carcinoma | MX-1 | Liver carcinoma | SMMC7721 |
| Cervical carcinoma | SiHa | Lung carcinoma | A549 |
| Cervical carcinoma | Hela | Lung carcinoma | NCI-H460 |
| Colon carcinoma | Ls-174-T | Melanoma | A375 |
| Colon carcinoma | CL187 | Melanoma | M14 |
| Colon carcinoma | HCT-116 | Melanoma | MV3 |
| Colon carcinoma | SW116 | Ovary carcinoma | A2780 |
| Gastric carcinoma | MGC-803 | Pancreatic carcinoma | BXPC-3 |
| Gastric carcinoma | SGC-7901 | Prostate carcinoma | PC-3M |
| Gastric carcinoma | BGC-823 | Tongue carcinoma | Tca-8113 |
| Kidney carcinoma | Ketr-3 | | |

TABLE 8
Syngeneic Mouse Tumor Model

| Tumor Type | Cell Line Name | Strain of Mice |
|---|---|---|
| Cervical carcinoma | U14 | ICR |
| Liver carcinoma | H22 | ICR |
| Lung carcinoma | Lewis | C57BL6 |
| Melanoma | B16F1, B16F10, B16BL6 | C57BL6 |
| Sarcoma | S180 | ICR |

Tumor size and volume can be monitored based on techniques known to one of skill in the art. For example, tumor size and volume can be monitored by radiography, ultrasound imaging, necropsy, by use of calipers, by microCT or by ¹⁸F-FDG-PET. Tumor size also can be assessed visually. In particular examples, tumor size (diameter) is measured directly using calipers. In other examples, tumor volume can be measured using an average of measurements of tumor diameter (D) obtained by caliper or ultrasound assessments. The volume can be determined from the formula V=D³×π/6 (for diameter measured using calipers) or V=D²×d×π/6 (for diameter measured using ultrasound where d is the depth or thickness). For example, caliper measurements can be made of the tumor length (1) and width (w) and tumor volume calculated as length×width²×0.52. In another example, microCT scans can be used to measure tumor volume (see e.g. Huang et al. (2009) PNAS, 106:3426-3430). In such an example, mice can be injected with Optiray Pharmacy loversol injection 74% contrast medium (e.g. 741 mg of loversol/mL), mice anesthetized, and CT scanning done using a MicroCat 1A scanner or other similar scanner (e.g. IMTek) (40 kV, 600 μA, 196 rotation steps, total angle or rotation=196). The images can be reconstructed using software (e.g. RVA3 software program; ImTek). Tumor volumes can be determined by using available software (e.g. Amira 3.1 software; Mercury Computer Systems).

Once the implanted tumors reach a predetermined size or volume, the models can be used for treatment with virus. The exact final tumor volume can be empirically determined and is a function of the particular type of tumor as well as the end-point of the analysis. Generally, mice are sacrificed if the tumor volume is greater than 3 cm³.

Tumor-bearing animals are infected with virus. The route of administration for infection can be any desired route of administration, for example, intravenous or topical. Other routes also can be employed, for example, intraperitoneal, such as subcutaneous, or can be intratumoral. The vaccinia virus in protein polymer compositions provided herein (e.g. VV-SELP, for example LIVP-SELP) can be administered at varying dosages. For example, the virus can be administered to tumor-bearing animals at or between about 1×10⁴ to 1×10⁸ pfu, such as 1×10⁵ to 1×10⁷ pfu, for example at least or about or 1×10⁶, 2×10⁶, 3×10⁶, 4×10⁶ or 5×10⁶ pfu. Progressing tumors are visualized and tumor size and tumor volume can be measured using any technique known to one of skill in the art. For example, tumor volume or tumor size can be measured using any of the techniques described herein. Tumor volume and size can be assessed or measured at periodic intervals over a period of time following virus infections, such as, for example, every hour, every 6 hours, every 12 hours, every 24 hours, every 36 hours, every 2 days, every 3 days, every 4 days, every 5 days, every 6 days, every 7-days, every week, every 3 weeks, every month or more post-infection. A graph of the median change in tumor volume over time can be made and the total area under the curve (AUC) can be calculated. A therapeutic index also can be calculated using the formula AUC_{untreated animals}−AUC_{virus-treated animals}/AUC_{untreated}×100.

In additional examples, tumors can be harvested from the animals and weighed. In further examples, the harvested tumors can be lysed. For example, lysis of tumors can be by freeze thaw of the harvested tumor several times (e.g. at least 2 times, 3 times or 4 times) shortly after removal of the tumor from the animal. For example, the tumor is lysed by 3 freeze thaw cycles within 2 hours of removal. The virus in the tumor lysates can be titered as described above and the amount of virus in each tumor sample determined. In some examples, the virus titer can be expressed as tissue culture infectious dose normalized to the tissue weight (TCID₅₀/mg tissue). In particular examples, the effect of the virus on other organs or tissues in the animal can be assessed. For example, other organs can be harvested from the animals, weighed and/or lysed for viral titer determination.

Generally, tumor-bearing animals generated in the same manner, at the same time and with the same type of tumor cells are used as controls. Such control tumor-bearing animals include those that remain untreated (not infected with virus). Additional controls animals can include those infected with a reference virus strain, such as a non-matrix virus composition or solution. Exemplary of such a strain is GLV-1h68 or a derivative thereof containing inserted heterologous genes. Comparison of tumor size or volume can be made at any predetermined time post-infection, and can be empirically determined by one of skill in the art. In some examples, a comparison can be made at the day in which the untreated control is sacrificed. In other examples, analysis of the total AUC can be made, and AUC values compared as an indicator of the size and volume of the tumor over the time period of infection. A decrease in tumor size, volume or weight compared to control treated or untreated tumor-bearing animals means that the virus itself is mediating tumor regression or shrinkage or that the virus is mediating delayed tumor progression compared to control treated or untreated tumor-bearing animals. Tumor shrinkage or delay in tumor progression are parameters indicative of anti-tumorigenicity.

5. Toxicity/Safety

Virus in protein polymer compositions provided herein can be tested for parameters indicative of its toxicity/safety property. Viruses can be toxic to their hosts by manufacturing one or more compounds that worsen the health condition of the host. In addition, the biocompatibility of the protein polymer also can be assessed by evaluating toxicity. Toxicity to the host can be manifested in any of a variety of manners, including septic shock, neurological effects, or muscular effects. Typically, vaccinia virus exhibits minimal to no toxicity to a host, such that the host does not die or become severely ill from the toxic effects of the virus. For example, the viruses are not toxic or exhibit minimal toxicity if a host typically has no significant long-term effect from the presence of the viruses in the host, beyond any effect on tumorous, metastatic or necrotic organs or tissues. For example, minimal toxicity can be a minor fever or minor infection, which lasts for less than about a month, and following the fever or infection, the host experiences no adverse effects resultant from the fever or infection. In another example, the minimal toxicity can be measured as an unintentional decline in body weight of about 5% or less for the host after administration of the virus. In other examples, the virus has no toxicity to the host.

Parameters indicative of toxicity or safety of a virus can be tested in vitro or in vivo. Typically, assessment is in vivo. Exemplary methods include administration of the virus to a subject (e.g. animal model) and assessment of one or more properties associated with toxicity including, but not limited to, survival of the subject, decrease in body weight, existence of side effects such as fever, rash or other allergy, fatigue or abdominal pain, induction of an immune response in the subject, tissue distribution of the virus, amount of tumor antigens that are released and decreased rate of pock formation. Hence, any of the above parameters can be assessed as indicative of toxicity/safety of a virus.

As above, subjects (e.g. animals such as tumor-bearing animal models) are infected with virus. The route of administration for infection can be any desired route of administration, for example, intravenous or topical. Other routes also can be employed, for example, intraperitoneal, such as subcutaneous, or can be intratumoral. The virus can be administered at varying dosages. For example, the virus can be administered to tumor-bearing animals at or between about 1×10⁴ to 1×10⁸ pfu, such as 1×10⁵ to 1×10⁷ pfu, for example at least or about or 1×10⁶, 2×10⁶, 3×10⁶, 4×10⁶ or 5×10⁶ pfu. For humans, the virus can be administered at or between about 1×10⁷ to 1×10¹⁴ pfu, such as 1×10⁷ to 1×10¹⁰ pfu or 1×10⁹ to 1×10¹⁰ pfu, for example at least or about 1×10⁹, 2×10⁹, 3×10⁹, 4×10⁹, or 5×10⁹ pfu. Parameters indicative of toxicity such as the survival and weight of the subject can be monitored over time. For example, survival and weight can be monitored at periodic intervals over a period of time following virus infections, such as, for example, every hour, every 6 hours, every 12 hours, every 24 hours, every 36 hours, every 2 days, every 3 days, every 4 days, every 5 days, every 6 days, every 7-days, every week, every 3 weeks, every month or more post-infection.

Generally, control subjects (e.g. animal models such as tumor-bearing animal models) are similarly monitored. Such control subjects include those that remain untreated (not infected with virus). Additional controls animals can include those infected with a reference virus strain, such as a non-matrix virus composition or solution. Exemplary of such a strain is GLV-1h68 or a derivative thereof containing inserted heterologous genes.

### H. THERAPEUTIC, DIAGNOSTIC AND MONITORING METHODS

Vaccinia virus (e.g. LIVP) in protein polymer (e.g. SELP) compositions (e.g. VV-SELP or LIVP-SELP) provided herein can be used in diagnostic, monitoring and therapeutic methods. The compositions provided herein are particularly suitable for treatment of hyperproliferative diseases or conditions, such as in the treatment of tumors or cancers. The compositions are suitable for such treatments and therapies because they 1) form a hydrogel matrix that is safe and biodegradable; 2) protect viral integrity within the gel at body temperature; 3) allow for sustained viral release in vivo; and 4) facilitate effective viral infection of adjacent tumor cells.

For example, in therapeutic methods the VV-protein polymer, such as VV-SELP and in particular an LIVP-SELP composition provided herein, can be used for the treatment of proliferative disorders or conditions, including the treatment of cancerous cells, neoplasms, tumors, metastases and other immunoprivileged cells or tissues, such as wounds or wounded or inflamed tissues. The VV-protein polymer, such as VV-SELP and in particular LIVP-SELP, compositions provided herein can be used in diagnostic methods for detecting and imaging of cancerous cells, tumors and metastases monitoring treatment. The diagnostic and therapeutic methods provided herein include, but are not limited to, delivering a composition provided herein to a subject containing a tumor and/or metastases or wound. In one example, in examples of treatments or methods provided herein, delivery of the composition can be effected by systemic administration, for example intravenous administration of the composition to the subject. In another example of treatment or methods provided herein, delivery of the composition can be effected by topical application of the composition to a surface of the subject.

The compositions provided herein, and in particular the LIVP in protein polymer compositions (e.g. LIVP-SELP) provided herein, can be used or modified for use in any known methods (or uses) in which LIVP viruses have been employed or can be employed (see e.g. see e.g. U.S. Pub. Nos. US2003-0059400, US2003-0228261, US2009-0117034, US2009-0098529, US2009-0053244, US2009-0081639 and US2009-0136917; U.S. Pat. Nos. 7,588,767 and 7,763,420; and International Pub. No. WO 2009/139921). Any LIVP virus, including the GLV-1h68 virus and derivatives thereof, can be used for the compositions herein for use in therapeutic and diagnostic methods described below and discussed throughout the disclosure herein.

The subject can be any subject, such as a animal subject, including mammal or avian species. For example, the animal subject can be a human or non-human animal including, but not limited to, a goat, sheep, horse, cat, or dog. In particular examples, the animal subject is a human subject.

The combinations provided herein also can be used in combination with other treatments. For example, treatment also can be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

1. Therapeutic Methods

The compositions provided herein can be used for the treatment of disease or conditions associated with immunoprivileged cells or tissues, including proliferative disorders or conditions, including the treatment (such as inhibition) of cancerous cells, neoplasms, tumors, metastases, cancer stem cells, and other immunoprivileged cells or tissues, such as wounds and wounded or inflamed tissues.

In particular, provided herein are methods of treating cancerous cells, neoplasms, tumors, metastases and cancer stem cells. The viruses in the compositions provided herein preferentially accumulate in tumors or metastases. In some examples, the administration of a virus provided herein results in a slowing of tumor growth, and in some cases an inhibition in tumor growth. In other examples, the administration of a virus provided herein results in a decrease in tumor volume, including elimination or eradication of the tumor.

Methods of reducing or inhibiting tumor growth, inhibiting metastasis growth and/or formation, decreasing the size of a tumor or metastasis, eliminating a tumor or metastasis and/or cancer stem cell or other tumor therapeutic methods provided herein include causing or enhancing an anti-tumor immune response in the host. The immune response of the host, being anti-tumor in nature, can be mounted against tumors and/or metastases in which viruses have accumulated, and can also be mounted against tumors and/or metastases in which viruses have not accumulated, including tumors and/or metastases that form after administration of the virus to the subject. Hence, the virus compositions provided herein can be used in methods to inhibit or prevent recurrence of a neoplastic disease or new tumor growth, where the methods include administering to a subject a composition provided herein, whereby a virus that can accumulate in a tumor and/or metastasis, and can cause or enhance an anti-tumor immune response, and the anti-tumor immune response can inhibit or prevent recurrence of a neoplastic disease or inhibit or prevent new tumor growth.

For example, viruses in the compositions provided herein, when administered or delivered to a subject, can be used to stimulate humoral and/or cellular immune response, induce strong cytotoxic T lymphocytes responses in subjects who can benefit from such responses. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

Methods of administering a composition containing a virus also can cause tumor cell lysis or tumor cell death. For example viruses, such as the viruses in compositions provided herein, can cause cell lysis or tumor cell death as a result of expression of an endogenous gene or as a result of an exogenous gene. Endogenous or exogenous genes can cause tumor cell lysis or inhibit cell growth as a result of direct or indirect actions, as is known in the art, including lytic channel formation or activation of an apoptotic pathway. Gene products, such as exogenous gene products can function to activate a prodrug to an active, cytotoxic form, resulting in cell death where such genes are expressed.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005-0031643, now U.S. Pat. Nos. 7,588,767, 7,588,771, 7,662,398), which provide and exemplify the GLV-1h68 virus and derivatives thereof. Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production.

In one example, the tumor treated is a cancer such as pancreatic cancer, non-small cell lung cancer, multiple myeloma or leukemia, although the cancer is not limited in this respect, and other metastatic diseases can be treated by the combinations provided herein. For example, the tumor treated can be a solid tumor, such as of the lung and bronchus, breast, colon and rectum, kidney, stomach, esophagus, liver and intrahepatic bile duct, urinary bladder, brain and other nervous system, head and neck, oral cavity and pharynx, cervix, uterine corpus, thyroid, ovary, testes, prostate, malignant melanoma, cholangiocarcinoma, thymoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS. Exemplary tumors include, for example, pancreatic tumors, ovarian tumors, lung tumors, colon tumors, prostate tumors, cervical tumors and breast tumors. In one example, the tumor is a carcinoma such as, for example, an ovarian tumor or a pancreatic tumor.

Any mode of administration of a virus to a subject can be used, provided the mode of administration permits the virus to enter a tumor or metastasis. Modes of administration can include, but are not limited to, systemic, parenteral, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, endoscopic, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, ocular or topical administration. In some examples, a diagnostic or therapeutic agent as described elsewhere herein also can be similarly administered.

One skilled in the art can select any mode of administration compatible with the subject and the virus, and that also is likely to result in the virus reaching tumors and/or metastases. The route of administration can be selected by one skilled in the art according to any of a variety of factors, including the nature of the disease, the kind of tumor, and the particular virus contained in the pharmaceutical composition. Exemplary of modes of administration of the vaccinia virus in protein polymer compositions provided herein are systemic administration (e.g. intravenous administration) or topical administration directly to the surface of a wound or lesion or other surface of a subject. For example, administration to the target site can be performed, for example, by systemic administration by injection into an artery or by topical administration by direct application onto a surface or by surface application of a coated device (e.g. bandage).

a. Systemic Delivery to Treat or Detect Proliferative or Inflammatory Cells or Tissues (e.g. Tumors)

Provided herein are methods of systemically administering a vaccinia virus in protein polymer composition (e.g. VV-SELP or LIVP-SELP), such as any provided herein, to treat a proliferative or inflammatory disease or condition. In particular, the condition is associated with immunoprivileged cells or tissues. A disease or condition associated with immunoprivileged cells or tissues includes, for example, proliferative disorders or conditions, including the treatment (such as inhibition) of cancerous cells, neoplasms, tumors, metastases, cancer stem cells, and other immunoprivileged cells or tissues, such as wounds and wounded or inflamed tissues. In particular examples of such methods, compositions provided herein are administered by intravenous administration.

Vaccinia virus, in particular Lister strain, such as LIVP viruses, can be administered systemically, for example by intravenous administration, because it exhibits little to no host toxicity. Although administration of a bolus of virus directly into the bloodstream can result in rapid dissemination of the virus throughout the organism, the virus compositions herein are efficiently delivered and infect immunoprivileged cells and tissues, for example, tumors. Systemic administration, such as intravenous administration, of the virus compositions is possible because the virus is able to accumulate in immunoprivileged cells and tissues (e.g. tumors), yet is efficiently cleared from the subject and does not significantly accumulate in non-tumor tissues. This can result in decreased toxicity.

In addition; when delivered systemically (e.g. intravenously), the vaccinia virus in protein polymer (e.g. SELP) compositions provided herein also can demonstrate an increased infectivity of tumors than non-polymer containing virus compositions. This increased infectivity or delivery of virus to tumors can be the result of an increased accumulation of the polymer composition to tumors than virus alone based on the high molecular weight of the hydrogels. For example, the vasculature of tumors is generally more leaky than healthy tissues or associated vasculature, which permits access of the hydrogel compositions to diseased sites. In addition, increased infectivity of tumors also can be associated with an increased viral integrity of the virus in vivo, and the concomitant increase in tumor exposure and amount of virus that is available for accumulation into the tumor.

By intravenous administration, the VV-polymer compositions provided herein, for example VV-SELP compositions such as LIVP-SELP compositions, can be used to treat any neoplastic disease, such as carcinoma, sarcoma, lymphoma or leukemia. In particular, intravenous administration of compositions provided herein can effect treatment of solid tumors (e.g. sarcomas, arcinomas or epithelial tumors or lymphomas) and cancers other than solid tumors, such as leukemia and metastastic disease. For example, exemplary of such tumors, cancers or neoplastic diseases include, for example, carcinoma of the tongue, mouth, throat, stomach, cecum, colon, rectum, breast, ovary, uterus, thyroid, adrenal cortex, lung, kidney, prostate, pancreas, a melanoma, a basal cell carcinoma of the skin, a leukemia, a lymphoma, or an osteosarcoma.

In particular, examples of solid tumors include, but are not limited to, sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cellcarcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Cancers for treatment by systemic administration (e.g. intravenous administration) of a composition herein also includes cancers that metastasize. It is understood by those in the art that metastasis is the spread of cells from a primary tumor to a noncontiguous site, usually via the bloodstream or lymphatics, which results in the establishment of a secondary tumor growth. Examples of cancers contemplated for treatment include, but are not limited to melanoma, bladder, non-small cell lung, small cell lung, lung, hepatocarcinoma, retinoblastoma, astrocytoma, glioblastoma, neuroblastoma, head, neck, breast, pancreatic, gum, tongue, prostate, renal, bone, testicular, ovarian, mesothelioma, cervical, gastrointestinal lymphoma, brain, or colon cancer and any other tumors or neoplasms that are metastasized or at risk of metastasis.

The compositions provided herein can be administered by a single injection, by multiple injections, or continuously. For example, the compositions can be administered by slow infusion including using an intravenous pump, syringe pump, intravenous drip or slow injection. If administered continuously, the compositions are generally administered while in liquid form prior to hydrogel formation. For example, continuous administration of the compositions can occur over the course of minutes to hours, such as between or about between 1 minutes to 1 hours, such as between 20 and 60 minutes.

b. Delivery to Treat or Detect Wounds or Hyperproliferative Surface Lesions

Provided herein are methods using a VV-polymer compositions, for example VV-SELP or LIVP-SELP compositions such as any provided herein, for the treatment of a surface wound or hyperproliferative surface lesion. The compositions provided herein also can be used for detection or a surface wound or hyperproliferative surface lesion. The compositions provided herein are particularly suitable for such topical applications because they are able to be applied as gels or rapidly form a highly viscous gel that can adhere to a body surface (e.g. a tumor) for prolonged viral contact with the surface (e.g. tumor). In particular, the topical application or delivery of virus in protein polymers (e.g. SELP) is advantageous over topical delivery of non-matrix viral solutions, because non-matrix viral solutions are hindered by inadequate or only brief contact with the wound or lesion surface, runoff of the viral solution away from the wound or lesion surface, and rapid viral degradation at body temperature. Delivery of virus in a matrix as provided in the VV-protein polymer (e.g. VV-SELP, such as LIVP-SELP) compositions herein permits the composition to adhere to the wound or lesion surface to permit prolonged viral-contact, protects viral integrity within the gel at body temperature, allows for sustained viral release in vivo and facilitates effective viral infection of cells. In addition, the matrix compositions are safe and biodegradable.

The surface of a lesion can be any lesion, whether benign, premalignant or malignant. The disease can be a precancerous lesion, such as leukoplakia of the oral cavity or actinic keratosis of the skin, or a wound that is a traumatic wound or a post-surgical wound. Hence, methods provided herein include methods of detecting, ameliorating or treating disease in a subject that involves applying to a body surface of the subject any of the compositions provided herein. In such examples, the compositions provided herein are formulated for application to a surface of a subject. The compositions provided herein permit sustained release of the virus to the affected wound or lesion.

In some examples, the compositions can be applied directly to a surface in liquid form as a hydrogel matrix precursor, which upon exposure to the surface, will eventually stiffen or harden to a hydrogel form. In other examples, the composition can be applied as a more gel-like or semi-solid composition with a higher viscosity than a liquid composition so that it retains its geometrical form as a matrix but prior to becoming a more solid hydrogel matrix. In examples where the composition is topically applied as a composition to a body surface of the subject, an applicator can be used for application of a gel, such as using a cotton-tipped applicator or spatula. If desired a device or material can be applied to the surface (e.g. to the skin) after application of the compositions onto the surface. For example, the surface-applied compositions can be covered by a suitable patch, bandage, wrap, dressing, mesh or other similar material or device for prevention of movement and dehydration of the composition. In other examples, the surface area in which the compositions herein are applied can be closed or covered by sutures, staples, or stiches to close the area, thereby preventing movement and dehydration of the composition. In these examples, the composition is exposed directly to the site of the wond or hyperproliferative lesion, thereby allowing sustained delivery of the virus from the matrix to the affected surface.

In other examples, the compositions can be coated on a device for application to the surface of a subject, such as any of the devices described above. In particular examples, the compositions provided herein are applied or coated onto the surface of a patch, a bandage, wrap, dressing, mesh or other similar material or device for application to the surface of a subject. Upon exposure to a physiologic or high temperature, such as can occur upon exposure of the coated device to the surface of a subject, the hydrogel will form. The coated device or material can be applied or used to directly cover a wound or hyperproliferative lesion. Since the composition is contained on the device or material, the composition is exposed directly to the wound or lesion, thereby allowing sustained delivery of the virus from the matrix to the affected surface.

The surface to be treated can be any area outside the of the body of a subject. For example, the surface can be a skin surface, a mucosal surface, the surface of a lesion, the surface of a wound, or the surface of a hollow viscus. The surface can be the skin or can be an internal organ such as the surface of the gastrointestinal tract, surface of the bladder, vagina, cervix or the uterus. The surface can be pretreated, such as abraded, as discussed below, to allow for more efficient transfer to underlying tissue.

For example, the compositions provided herein can be used to treat a skin lesion. In particular examples, the lesion is a hyperproliferative skin lesion, such as skin cancer. Exemplary hyperproliferative lesions include the following: squamous cell carcinoma, basal cell carcinoma, adenoma, adenocarcinoma, linitis plastica, insulinoma, glucagonoma, gastrinoma, vipoma, cholangiocarcinoma, hepatocellular carcinoma, adenoid cystic carcinoma, carcinoid tumor, prolactinoma, oncocytoma, hurthle cell adenoma, renal cell carcinoma, endometrioid adenoma, cystadenoma, pseudomyxoma peritonei, Warthin's tumor, thymoma, thecoma, granulosa cell tumor, arrhenoblastoma, Sertoli-Leydig cell tumor, paraganglioma, pheochromocytoma, glomus tumor, melanoma, soft tissue sarcoma, desmoplastic small round cell tumor, fibroma, fibrosarcoma, myxoma, lipoma, liposarcoma, leiomyoma, leiomyosarcoma, myoma, myosarcoma, rhabdomyoma, rhabdomyosarcoma, pleomorphic adenoma, nephroblastoma, brenner tumor, synovial sarcoma, mesothelioma, dysgerminoma, germ cell tumors, embryonal carcinoma, yolk sac tumor, teratomas, dermoid cysts, choriocarcinoma, mesonephromas, hemangioma, angioma, hemangiosarcoma, angiosarcoma, hemangioendothelioma, hemangioendothelioma, Kaposi's sarcoma, hemangiopericytoma, lymphangioma, cystic lymphangioma, osteoma, osteosarcoma, osteochondroma, cartilaginous exostosis, chondroma, chondrosarcoma, giant cell tumors, Ewing's sarcoma, odontogenic tumors, cementoblastoma, ameloblastoma, craniopharyngioma gliomas mixed oligoastrocytomas, ependymoma, astrocytomas, glioblastomas, oligodendrogliomas, neuroepitheliomatous neoplasms, neuroblastoma, retinoblastoma, meningiomas, neurofibroma, neurofibromatosis, schwannoma, neurinoma, neuromas, granular cell tumors, alveolar soft part sarcomas, lymphomas, non-Hodgkin's lymphoma, lymphosarcoma, Hodgkin's disease, small lymphocytic lymphoma, lymphoplasmacytic lymphoma, mantle cell lymphoma, primary effusion lymphoma, mediastinal (thymic) large cell lymphoma, diffuse large B-cell lymphoma, intravascular large B-cell lymphoma, Burkitt lymphoma, splenic marginal zone lymphoma, follicular lymphoma, extranodal marginal zone B-cell lymphoma of mucosa-associated lymphoid tissue (MALT-lymphoma), nodal marginal zone B-cell lymphoma, mycosis fungoides, Sezary syndrome, peripheral T-cell lymphoma, angioimmunoblastic T-cell lymphoma, subcutaneous panniculitis-like T-cell lymphoma, anaplastic large cell lymphoma, hepatosplenic T-cell lymphoma, enteropathy type T-cell lymphoma, lymphomatoid papulosis, primary cutaneous anaplastic large cell lymphoma, extranodal NK/T cell lymphoma, blastic NK cell lymphoma, plasmacytoma, multiple myeloma, mastocytoma, mast cell sarcoma, mastocytosis, mast cell leukemia, langerhans cell histiocytosis, histiocytic sarcoma, langerhans cell sarcoma dendritic cell sarcoma, follicular dendritic cell sarcoma, Waldenstrom macroglobulinemia, lymphomatoid granulomatosis, acute leukemia, lymphocytic leukemia, acute lymphoblastic leukemia, acute lymphocytic leukemia, chronic lymphocytic leukemia, adult T-cell leukemia/lymphoma, plasma cell leukemia, T-cell large granular lymphocytic leukemia, B-cell prolymphocytic leukemia, T-cell prolymphocytic leukemia, precursor B lymphoblastic leukemia, precursor T lymphoblastic leukemia, acute erythroid leukemia, lymphosarcoma cell leukemia, myeloid leukemia, myelogenous leukemia, acute myelogenous leukemia, chronic myelogenous leukemia, acute promyelocytic leukemia, acute promyelocytic leukemia, acute myelomonocytic leukemia, basophilic leukemia, eosinophilic leukemia, acute basophilic leukemia, acute myeloid leukemia, chronic myelogenous leukemia, monocytic leukemia, acute monoblastic and monocytic leukemia, acute megakaryoblastic leukemia, acute myeloid leukemia and myelodysplastic syndrome, chloroma or myeloid sarcoma, acute panmyelosis with myelofibrosis, hairy cell leukemia, juvenile myelomonocytic leukemia, aggressive NK cell leukemia, polycythemia vera, myeloproliferative disease, chronic idiopathic myelofibrosis, essential thrombocytemia, chronic neutrophilic leukemia, chronic eosinophilic leukemia/hypereosinophilic syndrome, post-transplant lymphoproliferative disorder, chronic myeloproliferative disease, myelodysplastic/myeloproliferative diseases, chronic myelomonocytic leukemia and myelodysplastic syndrome. Exemplary of such lesions or cancers include, for example, basal cell carcinoma or squamous cell carcinoma.

In particular examples, the hyperproliferative lesion is a disease that can affect the skin of a subject. Examples include squamous cell carcinoma, basal cell carcinoma, melanoma, papillomas (warts), and psoriasis. The lesion can include cells such as keratinocytes, epithelial cells, skin cells, and mucosal cells.

The hyperproliferative lesion also can be a disease that affects the mouth of a subject. For example, such diseases include, but are not limited to, leukoplakia, squamous cell hyperplastic lesions, premalignant epithelial lesions, oral dysplasia, intraepithelial neoplastic lesions, focal epithelial hyperplasia and squamous carcinoma lesion. In another example, the compositions provided herein can be used to treat any mucosal surface of the body, such as the surface of the oral cavity, the surface of the esophagus, lung mucosal surface, stomach, duodenum, small intestine, large intestine, colon, rectum, vagina or bladder. The mucosal surface can be the surface of a lesion of the mucosa, such as a leukoplakia of the mouth, colon polyp or tumor. In other examples, the compositions provided herein can be used to treat a wound surface. The wound can be a traumatic wound, such as a burn, scrape, cut or other surface wound. The wound can be a post-surgical wound such as following surgical resection of a tumor.

Surgically Resected Tumor

Provided herein are methods using a VV-polymer compositions, for example VV-SELP or LIVP-SELP compositions such as any provided herein, for the treatment of tumors by delivering the oncolytic virus composition directly to a resected tumor. Delivery of the compositions herein to tumors following surgical resection permits penetration of the virus into tumors. This is advantageous over other methods of viral delivery, such as intratumoral injection, where virus is administered into three dimensional tumors and is not able to adequately penetrate or distribute throughout the tumor mass (see e.g. Nemunaitis et al. (2001) J. Clin. Oncol., 19:289-298). In addition, the application of virus compositions to a tumor bed immediately following surgical resection also can provide an effective treatment to a patient that harbors residual disease in a surgical field associated with an incomplete surgical resection. Tumor resection is the primary therapeutic option for the majority of patients diagnosed with a solid tumor. In some clinical situations, however, surgical resection can remove the majority of disease, but residual disease can be left on critical anatomic structures that are considered unresectable. In these scenarios, direct application of virus to a surface of residual disease can optimize viral delivery and tumor penetration.

Further, the application or delivery of virus in protein polymers (e.g. SELP) is advantageous over topical delivery of non-matrix viral solutions that can be hindered by inadequate or only brief contact with the tumor surface, runoff of the viral solution away from the tumor surface to the surgical cavity, rapid viral degradation at body temperature and immunologic clearance of virus that fails to infect cancer cells. Delivery of virus in a matrix as provided in the VV-protein polymer (e.g. VV-SELP, such as LIVP-SELP) compositions herein permits the composition to adhere to the tumor surface to permit prolonged viral-tumor contact, protects viral integrity within the gel at body temperature, allows for sustained viral release in vivo and facilitates effective viral infection of adjacent tumor cells. In addition, the matrix compositions are safe and biodegradable.

For example, in the methods provided herein the compositions are applied to the surface of a resected tumor, whereby at least part of a tumor has been physically removed. In the methods herein of delivering a vaccinia virus in polymer composition to a resected tumor, any solid tumor can be surgically resected. For example, for purpose of the methods herein, among the solid tumors that can be resected include those that are otherwise determined to be unresectable, for example, because it cannot be surgically resected with clear margins. For example, patients with anaplastic thyroid cancer typically present with a large tumor that cannot be surgically resected with clear margins. Clinically, physicians often consider such a tumor to be unresectable or marginally resectable, since resection does not remove all disease. Nevertheless, in the methods herein, virus compositions are applied to the surface of a resected tumor, including those that cannot be surgically resected with clear margins, thereby effecting exposure and penetration of the oncolytic virus to residual tumor cells. It is within the level of a skilled physician to determine if a tumor is resectable for purposes of delivery of a virus composition herein. Factors to be considered in determining if a tumor is resectable include, but are not limited to, its size and location, whether it has spread to other parts of the body, and if the person is healthy enough for surgery.

Methods of surgically resecting tumors are well known to one of skill in the art and can be practiced by a skilled physician. Accepted types of resection depend on the particular tumor types. The tumor can be completely resected or can be partially removed such that residual tumor remains. In the methods herein, the extent of resection of the tumor is within the level of the skilled physician and depends on factors such as the type of tumor, the size of the tumor, the state or severity of disease and the particular patient being treated. In the methods herein, compositions can be administered to resected tumors in which from or from about 10 mm³ to 300 m³ of residual tumor remains, for example, from or from about 10 mm³ to 100 mm³, 25 mm³ to 100 mm³, 50 mm³ to 100 mm³, 50 mm³ to 250 mm³ or 100 mm³ to 200 mm³. Generally, the compositions provided herein are applied to low volume residual disease, wherein equal to or less than 100 mm³, such as equal to or less than 50 mm³ of the tumor remains. For example, in the methods herein the surgical resection should remove the large bulk of the tumor and leave behind only low volume or microscopic disease, likely shaped as a sheet or a thin layer. Typically, surgery is effected to generate a flat surface or sheet of residual disease within a surgical cavity so that the compositions herein can be topically applied to the flat surface or sheet to facilitate immediate and direct contact of virus with cancer cells.

In some examples of the method herein, the compositions provided herein can be delivered by postoperative application to a resected tumor. In such examples, at the time of the treatment, the surgery should be fairly recent, and yet the patient should be fully recovered from the surgery. Typically, patients receive treatment within 24 hours to 12 weeks after surgery, such as 24 hours to 96 hours, 2 days to 7 days, 1 week to 6 weeks, and generally no more than 8-12 weeks after surgery. In other examples of the methods herein, the compositions provided herein can be delivered by intraoperative application to a resected tumor.

Upon excision of part of all of cancerous cells, tissue, or tumor, a cavity can be formed in the body. A VV-protein polymer composition provided herein, such as an VV-SELP or LIVP-SELP composition provided herein, can be topically applied to the cavity, and generally to a flat surface within the surgical cavity. The composition can be applied as a liquid composition, which then will solidify to a gel upon contact with the body surface resulting in a continuous contact with that surface to permit controlled viral release over time. In other examples, the composition can be applied at a higher viscosity, such as a gelatin-like or semi-solid gel, which then also will further solidify upon contact with the body surface resulting in a continuous contact with that surface to permit controlled viral release over time. In further examples, the compositions can be applied by exposure of the surface to a device coated with the composition, such as a bandage or wrap or other device described herein or known in the art that is capable of being coated with a virus hydrogel composition as described herein.

2. Dosages and Dosage Regime

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art.

In the present methods, appropriate minimum dosage levels and dosage regimes of viruses in the compositions herein can be levels sufficient for the virus to survive, grow and replicate in a tumor, metastasis or other wound or lesion. Generally, the virus is administered in an amount that is at least or about or 1×10⁵ pfu. Exemplary minimum levels for administering a virus to a 65 kg human can include at least about 1×10⁵ plaque forming units (pfu), at least about 5×10⁵ pfu, at least about 1×10⁶ pfu, at least about 5×10⁶ pfu, at least about 1×10⁷ pfu, at least about 1×10⁸ pfu, at least about 1×10⁹ pfu, or at least about 1×10¹⁰ pfu. For example, the virus is administered in an amount that is at least or about or is 1×10⁵ pfu, 1×10⁶ pfu, 1×10⁷ pfu, 1×10⁸ pfu, 1×10⁹ pfu, 1×10¹⁰ pfu, 1×10¹¹ pfu, 1×10¹² pfu, 1×10¹³ pfu, or 1×10¹⁴ pfu at least one time over a cycle of administration.

In the dosage regime, the amount of virus can be delivered as a single administration or multiple times over a cycle of administration. Hence, the methods provided herein can include a single administration of a virus to a subject or multiple administrations of a virus to a subject. In some examples, a single administration is sufficient to establish a virus in a tumor, where the virus can proliferate and can cause or enhance an anti-tumor response in the subject; such methods do not require additional administrations of a virus in order to cause or enhance an anti-tumor response in a subject, which can result, for example in-inhibition of tumor growth, inhibition of metastasis growth or formation, reduction in tumor or size, elimination of a tumor or metastasis, inhibition or prevention of recurrence of a neoplastic disease or new tumor formation, or other cancer therapeutic effects.

In other examples, the virus in protein polymer (e.g. SELP) compositions can be administered on different occasions, separated in time typically by at least one day. For example, the compositions can be administered two times, three time, four times, five times, or six times or more, with one day or more, two days or more, one week or more, or one month or more time between administrations. Separate administrations can increase the likelihood of delivering a virus to a tumor or metastasis, where a previous administration has been ineffective in delivering a virus to a tumor or metastasis. Separate administrations can increase the locations on a tumor or metastasis where virus proliferation can occur or can otherwise increase the titer of virus accumulated in the tumor, which can increase the scale of release of antigens or other compounds from the tumor in eliciting or enhancing a host's anti-tumor immune response, and also can, optionally, increase the level of virus-based tumor lysis or tumor cell death. Separate administrations of a virus can further extend a subject's immune response against viral antigens, which can extend the host's immune response to tumors or metastases in which viruses have accumulated, and can increase the likelihood of a host mounting an anti-tumor immune response.

When separate administrations are performed, each administration can be a dosage amount that is the same or different relative to other administration dosage amounts. In one example, all administration dosage amounts are the same. In other examples, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same, smaller amount relative to the first administration.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a virus, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject's anti-virus antibody titer, the subject's anti-tumor antibody titer, the overall health of the subject, the weight of the subject, the presence of virus solely in tumor and/or metastases, the presence of virus in normal tissues or organs.

The time period between administrations can be any of a variety of time periods. The time period between administrations can be a function of any of a variety of factors, including monitoring steps, as described in relation to the number of administrations, the time period for a subject to mount an immune response, the time period for a subject to clear the virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

For example, an amount of virus is administered two times, three times, four times, five times, six times or seven times over a cycle of administration. The amount of virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. Generally, the cycle of administration is 7 days, 14 days, 21 days or 28 days. Depending on the responsiveness or prognosis of the patient the cycle of administration is repeated over the course of several months or years.

Generally, appropriate maximum dosage levels or dosage regimes of viruses are levels that are not toxic to the host, levels that do not cause splenomegaly of 3 times or more, levels that do not result in colonies or plaques in normal tissues or organs after about 1 day or after about 3 days or after about 7 days.

3. Combination Therapy

The subject also can be undergoing secondary treatment for a tumor, cancer, wound or hyperproliferative surface lesion. For example, the methods herein include combination therapy with a secondary anti-cancer therapy. Examples of such therapy include, but are not limited to, surgical therapy, chemotherapy, radiation therapy, immunotherapy, treatment with another therapeutic substance or agent and/or administration with another therapeutic virus. These can be administered simultaneously, sequentially or intermittently with the compositions provided herein.

a. Oncolytic or Therapeutic Virus

Methods are provided for administering to a subject in combination with a composition provided herein another oncolytic or therapeutic virus. The additional virus can be administered as a matrix composition as provided herein or as a non-matrix composition. The virus can be any virus that is capable of effecting treatment of diseases or conditions associated with immunoprivileged cells or tissues, including proliferative disorders or conditions, including the treatment (such as inhibition) of cancerous cells, neoplasms, tumors, metastases, cancer stem cells, and other immunoprivileged cells or tissues, such as wounds and wounded or inflamed tissues. For example, the virus is an oncolytic virus. The virus can contain a heterologous gene product that encodes a therapeutic protein or that is detectable or capable of being detected. For example, the virus can be a vaccinia virus (e.g. Lister strain or LIVP), an adenovirus, an adeno-associated virus, a retrovirus, a herpes simplex virus, a reovirus, a mumps virus, a foamy virus, an influenza virus, a myxoma virus, a vesicular stomatitis virus, or any other virus described herein or known in the art, or derivatives or modified forms thereof.

The virus can be provided as combinations of compositions and/or as kits that include the virus and compositions provided herein packaged for administration and optionally including instructions therefore. The additional virus compositions can contain the viruses formulated for single dosage administration (i.e., for direct administration) and can require dilution or other additions.

Administration can be effected simultaneously, sequentially or intermittently. The time period between administrations can be any time period that achieves the desired effects, as can be determined by one skilled in the art. Selection of a time period between administrations of different viruses can be determined according to parameters similar to those for selecting the time period between administrations of the same virus, including results from monitoring steps, the time period for a subject to mount an immune response, the time period for a subject to clear virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

b. Therapeutic Compounds

Any therapeutic or anti-cancer agent can be used as the second, therapeutic or anti-cancer agent in the combined cancer treatment methods provided herein. The methods can include administering one or more therapeutic compounds to the subject in addition to administering the compositions provided herein to a subject. Therapeutic compounds can act independently, or in conjunction with the virus, for tumor therapeutic effects. Therapeutic compounds or agents also include those that are immunotherapeutic compounds. Therapeutic compounds to be administered can be any of those provided herein or in the art.

Therapeutic compounds that can act independently include any of a variety of known chemotherapeutic compounds that can inhibit tumor growth, inhibit metastasis growth and/or formation, decrease the size of a tumor or metastasis, eliminate a tumor or metastasis, without reducing the ability of a virus to accumulate in a tumor, replicate in the tumor, and cause or enhance an anti-tumor immune response in the subject.

Therapeutic compounds that act in conjunction with the viruses include, for example, compounds that alter the expression of the viruses or compounds that can interact with a virally-expressed gene, or compounds that can inhibit virus proliferation, including compounds toxic to the virus. Therapeutic compounds that can act in conjunction with the virus include, for example, therapeutic compounds that increase the proliferation, toxicity, tumor cell killing or immune response eliciting properties of a virus, and also can include, for example, therapeutic compounds that decrease the proliferation, toxicity or cell killing properties of a virus. Optionally, the therapeutic agent can exhibit or manifest additional properties, such as, properties that permit its use as an imaging agent, as described elsewhere herein.

For example, tumors, cancers and metastasis can be a monotherapy-resistant tumor such as, for example, one that does not respond to therapy with virus alone or other therapeutic agent (e.g. anti-cancer agent alone), but that does respond to therapy with a combination of virus and other therapeutic agent (e.g. anti-cancer agent). Typically, a therapeutically effective amount of a virus composition provided herein is administered to the subject and the virus localizes and accumulates in the tumor. Subsequent to administering the virus, the subject is administered a therapeutically effective amount of another therapeutic agent, for example an anti-cancer agent, such as a chemotherapeutic agent (e.g. cisplatin). In one example, the other therapeutic agent is administered once-daily for five consecutive days. One of skill in the art could determine when to administer the therapeutic agent subsequent to the virus using, for example, in vivo animal models. Using the methods provided herein, administration of a virus composition provided herein and other therapeutic agent can cause a reduction in tumor volume, can cause tumor growth to stop or be delayed or can cause the tumor to be eliminated from the subject. The status of tumors, cancers and metastasis following treatment can be monitored using any of the methods provided herein and known in the art.

Therapeutic compounds or agents include, but are not limited to, chemotherapeutic agents, nanoparticles, radiation therapy, siRNA molecules, enzyme/pro-drug pairs, photosensitizing agents, toxins, microwaves, a radionuclide, an angiogenesis inhibitor, a mitosis inhibitor protein (e.g., cdc6), an antitumor oligopeptide (e.g., antimitotic oligopeptides, high affinity tumor-selective binding peptides), a signaling modulator, anti-cancer antibiotics, or a combination thereof.

Exemplary photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl)chlorin or tin(IV) chlorin e6, purpurins such as tin ethyl etiopurpurin, purpurinimides, bacteriochlorins, pheophorbides, pyropheophorbides or cationic dyes. In one example, a vaccinia virus, such as a vaccinia virus provided herein, is administered to a subject having a tumor, cancer or metastasis in combination with a photosensitizing agent.

Radionuclides, which depending up the radionuclide, amount and application can be used for diagnosis and/or for treatment. They include, but are not limited to, for example, a compound or molecule containing ³²Phosphorus, ⁶⁰Cobalt, ⁹⁰Yttrium, ⁹⁹Technitium, ¹⁰³Palladium, ¹⁰⁶Ruthenium, ¹¹¹Indium, ¹¹⁷Lutetium, ¹²⁵Iodine, ¹³¹Iodine, ¹³⁷Cesium, ¹⁵³Samarium, ¹⁸⁶Rhenium, ¹⁸⁸Rhenium, ¹⁹²Iridium, ¹⁹⁸Gold, ²¹¹Astatine, ²¹²Bismuth or ²¹³Bismuth. In one example, a vaccinia virus, such as a vaccinia virus provided herein, is administered to a subject having a tumor, cancer or metastasis in combination with a radionuclide.

Toxins include, but are not limited to, chemotherapeutic compounds such as, but not limited to, 5-fluorouridine, calicheamicin and maytansine. Signaling modulators include, but are not limited to, for example, inhibitors of macrophage inhibitory factor, toll-like receptor agonists and stat3 inhibitors. In one example, a vaccinia virus, such as a vaccinia virus provided herein, is administered to a subject having a tumor, cancer or metastasis in combination with a toxin or a signaling modulator.

Chemotherapeutic compounds include, but are not limited to, alkylating agents such as thiotepa and cyclophosphamide; alkyl sulfonates such as busulfan, improsulfan and piposulfan; aziridines such as benzodepa, carboquone, meturedepa and uredepa; ethylenimine and methylmelamines, including altretamine, triethylenemelamine, triethylenephosphoramide, triethylenethiophosphoramide and trimethylmelamine nitrogen mustards such as chlorambucil, chlornaphazine, chlorophosphamide, estramustine, ifosfamide, mechlorethamine, mechlorethamine oxide hydrochloride, melphalan, novobiocin, phenesterine, prednimustine, trofosfamide, uracil mustard; nitrosoureas such as carmustine, chlorozotocin, fotemustine, lomustine, nimustine, ranimustine; antibiotics such as aclacinomycins, actinomycin, anthramycin, azaserine, bleomycins, cactinomycin, calicheamicin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defosfamide; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, e.g., paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; Navelbine; Novantrone; teniposide; daunomycin; aminopterin; Xeloda; ibandronate; CPT11; topoisomerase inhibitor RFS 2000; difluoromethylornithine (DMFO); retinoic acid; esperamycins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston); and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. Chemotherapeutic agents also include new classes of targeted chemotherapeutic agents such as, for example, imatinib (sold by Novartis under the trade name Gleevec in the United States), gefitinib (developed by AstraZeneca under the trade name Iressa) and erlotinib. Particular chemotherapeutic agents include, but are not limited to, cisplatin, carboplatin, oxaliplatin, DWA2114R, NK121, IS 3 295, and 254-S vincristine, prednisone, doxorubicin and L-asparaginase; mechlorethamine, vincristine, procarbazine and prednisone (MOPP), cyclophosphamide, vincristine, procarbazine and prednisone (C-MOPP), bleomycin, vinblastine, gemcitabine and 5-fluorouracil. Exemplary chemotherapeutic agents are, for example, cisplatin, carboplatin, oxaliplatin, DWA2114R, NK121, IS 3 295, and 254-S.

Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and pirarubicin hydrochloride, phleomycins such as phleomycin and peplomycin sulfate, mitomycins such as mitomycin C, actinomycins such as actinomycin D, zinostatinstimalamer and polypeptides such as neocarzinostatin.

Anti-cancer antibodies include, but are not limited to, Rituximab (RITUXAN), ADEPT, Trastuzumab (HERCEPTIN), Tositumomab (BEXXAR), Cetuximab (ERBITUX), Ibritumomab (ZEVALIN), Alemtuzumab (Campath-1H), Epratuzumab (Lymphocide), Gemtuzumab ozogamicin (MYLOTARG), Bevacizumab (AVASTIN), and Edrecolomab (PANOREX).

Cancer growth inhibitors use cell-signaling molecules which control the growth and multiplication of cells, such as cancer cells. Drugs that block these signaling molecules can stop cancers from growing and dividing. Cancer growth inhibitors include drugs that block tyrosine kinases (i.e. tyrosine kinase inhibitors; TKIs) or that inhibit the proteasome inhibitors. Examples of TKIs include, but are not limited to, Erlotinib (Tarceva, OSI-774), Iressa (Gefitinib, ZD 1839), Imatinib (Glivec, STI 571) and Bortezomib (Velcade).

In one example, nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen. In one example, a vaccinia virus in protein polymer composition, such as any provided herein, is administered to a subject having a tumor, cancer or metastasis in combination with a nanoparticle carrying any of the therapeutic agents provided herein.

Radiation therapy has become a foremost choice of treatment for a majority of cancer patients. The wide use of radiation treatment stems from the ability of gamma-irradiation to induce irreversible damage in targeted cells with the preservation of normal tissue function. Ionizing radiation triggers apoptosis, the intrinsic cellular death machinery in cancer cells, and the activation of apoptosis seems to be the principal mode by which cancer cells die following exposure to ionizing radiation. In one example, a vaccinia virus in protein polymer composition, such as any provided herein, is administered to a subject having a tumor, cancer or metastasis in combination with radiation therapy.

Therapeutic compounds also include those that can act in conjunction with the virus to increase the proliferation, toxicity, tumor cell killing or immune response eliciting properties of a virus are compounds that can alter gene expression, where the altered gene expression can result in an increased killing of tumor cells or an increased anti-tumor immune response in the subject. A gene expression-altering compound can, for example, cause an increase or decrease in expression of one or more viral genes, including endogenous viral genes and/or exogenous viral genes. For example, a gene expression-altering compound can induce or increase transcription of a gene in a virus such as an exogenous gene that can cause cell lysis or cell death, that can provoke an immune response, that can catalyze conversion of a prodrug-like compound, or that can inhibit expression of a tumor cell gene. Any of a wide variety of compounds that can alter gene expression are known in the art, including IPTG and RU486. Exemplary genes whose expression can be up-regulated include proteins and RNA molecules, including toxins, enzymes that can convert a prodrug to an anti-tumor drug, cytokines, transcription regulating proteins, siRNA and ribozymes. In another example, a gene expression-altering compound can inhibit or decrease transcription of a gene in a virus such as a heterologous gene that can reduce viral toxicity or reduces viral proliferation. Any of a variety of compounds that can reduce or inhibit gene expression can be used in the methods provided herein, including siRNA compounds, transcriptional inhibitors or inhibitors of transcriptional activators. Exemplary genes whose expression can be down-regulated include proteins and RNA molecules, including viral proteins or RNA that suppress lysis, nucleotide synthesis or proliferation, and cellular proteins or RNA molecules that suppress cell death, immunoreactivity, lysis, or viral replication.

In another example, therapeutic compounds that can act in conjunction with the virus to increase the proliferation, toxicity, tumor cell killing, or immune response eliciting properties of a virus are compounds that can interact with a virally expressed gene product, and such interaction can result in an increased killing of tumor cells or an increased anti-tumor immune response in the subject. A therapeutic compound that can interact with a virally-expressed gene product can include, for example a prodrug or other compound that has little or no toxicity or other biological activity in its subject-administered form, but after interaction with a virally expressed gene product, the compound can develop a property that results in tumor cell death, including but not limited to, cytotoxicity, ability to induce apoptosis, or ability to trigger an immune response. In one non-limiting example, the virus carries an enzyme into the cancer cells. Once the enzyme is introduced into the cancer cells, an inactive form of a chemotherapy drug (i.e., a prodrug) is administered. When the inactive prodrug reaches the cancer cells, the enzyme converts the prodrug into the active chemotherapy drug, so that it can kill the cancer cell. Thus, the treatment is targeted only to cancer cells and does not affect normal cells. The prodrug can be administered concurrently with, or sequentially to, the virus. A variety of prodrug-like substances are known in the art and an exemplary set of such compounds are disclosed elsewhere herein, where such compounds can include gancyclovir, 5-fluorouracil, 6-methylpurine deoxyriboside, cephalosporin-doxorubicin, 4-[(2-chloroethyl)(2-mesyloxyethyl)amino]benzoyl-L-glutamic acid, acetaminophen, indole-3-acetic acid, CB1954, 7-ethyl-10-[4-(1-piperidino)-1-piperidino]carbonyloxycamptothecin, bis-(2-chloroethyl)amino-4-hydroxyphenyl-aminomethanone 28, 1-chloromethyl-5-hydroxy-1,2-dihydro-3H-benz[e]indole, epirubicin-glucuronide, 5′-deoxy-5-fluorouridine, cytosine arabinoside, linamarin, and a nucleoside analogue (e.g., fluorouridine, fluorodeoxyuridine, fluorouridine arabinoside, cytosine arabinoside, adenine arabinoside, guanine arabinoside, hypoxanthine arabinoside, 6-mercaptopurineriboside, theoguanosine riboside, nebularine, 5-iodouridine, 5-iododeoxyuridine, 5-bromodeoxyuridine, 5-vinyldeoxyuridine, 9-[(2-hydroxy)ethoxy]methylguanine (acyclovir), 9-[(2-hydroxy-1-hydroxymethyl)-ethoxy]methylguanine (DHPG), azauridien, azacytidine, azidothymidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, 3′-deoxythymidine).

In another example, therapeutic compounds that can act in conjunction with the virus to decrease the proliferation, toxicity or cell killing properties of a virus are compounds that can inhibit viral replication, inhibit viral toxins or cause viral death. A therapeutic compound that can inhibit viral replication, inhibit viral toxins, or cause viral death can generally include a compound that can block one or more steps in the viral life cycle, including, but not limited to, compounds that can inhibit viral DNA replication, viral RNA transcription, viral coat protein assembly, outer membrane or polysaccharide assembly. Any of a variety of compounds that can block one or more steps in a viral life cycle are known in the art, including any known antiviral compound (e.g., cidofovir), viral DNA polymerase inhibitors, viral RNA polymerase inhibitors, inhibitors of proteins that regulate viral DNA replication or RNA transcription. In another example, a virus can contain a gene encoding a viral life cycle protein, such as DNA polymerase or RNA polymerase that can be inhibited by a compound that is, optionally, non-toxic to the host organism.

Therapeutic compounds also include, but are not limited to, compounds that exert an immunotherapeutic effect, stimulate or suppress the immune system, carry a therapeutic compound, or a combination thereof. Such therapeutic compounds include, but are not limited to, anti-cancer antibodies, radiation therapy, siRNA molecules and compounds that suppress the immune system (i.e. immunosuppressors, immunosuppressive agents). In some cases, it is desirable to administer an immunosuppressive agent to a subject to suppress the immune system prior to the administration of the virus in order to minimize any adverse reactions to the virus. Exemplary immunosuppressive agents include, but are not limited to, glucocorticoids, alkylating agents, antimetabolites, cytokines and growth factors (e.g. interferons) and immunosuppressive antibodies (e.g., anti-CD3 and anti-IL2 receptor antibodies). For example, immunosuppresisive agents include biological response modifiers, such as monoclonal antibodies (mAbs), cancer vaccines, growth factors for blood cells, cancer growth inhibitors, anti-angiogenic factors, interferon alpha, interleukin-2 (IL-2), gene therapy and BCG vaccine for bladder cancer

Cytokines and growth factors include, but are not limited to, interleukins, such as, for example, interleukins (e.g. interleukin-1, interleukin-2, interleukin-6 and interleukin-12), tumor necrosis factors, such as tumor necrosis factor alpha (TNF-α), interferons such as interferon gamma (IFN-γ) or interferon alpha (IFN-α), Granulocyte Colony Stimulating Factor (G-CSF; also called filgrastim (Neupogen) or lenograstim (Granocyte)), Granulocyte and Macrophage Colony Stimulating Factor (GM-CSF; also called molgramostim), angiogenins, erythropoietin (EPO) and tissue factors.

Cancer vaccines include, for example, antigen vaccines, whole cell vaccines, dendritic cell vaccines, DNA vaccines and anti-idiotype vaccines. Antigen vaccines are vaccines made from tumor-associated antigens in, or produced by, cancer cells. Antigen vaccines stimulate a subject's immune system to attack the cancer. Whole cell vaccines are vaccines that use the whole cancer cell, not just a specific antigen from it, to make the vaccine. The vaccine is made from a subject's own cancer cells, another subject's cancer cells or cancer cells grown in a laboratory. The cells are treated in the laboratory, usually with radiation, so that they can't grow, and are administered to the subject via injection or through an intravenous drip into the bloodstream so they can stimulate the immune system to attack the cancer. One type of whole cell vaccine is a dendritic cell vaccine, which help the immune system to recognize and attack abnormal cells, such as cancer cells. Dendritic cell vaccines are made by growing dendritic cells alongside the cancer cells in the lab. The vaccine is administered to stimulate the immune system to attack the cancer. Anti-idiotype vaccines are vaccines that stimulate the body to make antibodies against cancer cells. Cancer cells make some tumor-associated antigens that the immune system recognizes as foreign. But because cancer cells are similar to non-cancer cells, the immune system can respond weakly. DNA vaccines boost the immune response. DNA vaccines are made from DNA from cancer cells that carry the genes for the tumor-associated antigens. When a DNA vaccine is injected, it enables the cells of the immune system to recognize the tumor-associated antigens, and activates the cells in the immune system (i.e., breaking tolerance).

The dose scheme of the combination therapy administered is such that the combination of the two or more therapeutic modalities is therapeutically effective. Dosages will vary in accordance with such factors as the age, health, sex, size and weight of the patient, the route of administration, the toxicity of the drugs, frequency of treatment and the relative susceptibilities of the cancer to each of the therapeutic modalities. For combination therapies with additional therapeutic agents provided herein (e.g. chemotherapeutic compounds), dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, for example, tumor size, and other viruses, treatments, or compounds, such as other chemotherapeutic drugs, being administered concurrently). As will be understood by one of skill in the art, the optimal treatment regimen will vary and it is within the scope of the treatment methods to evaluate the status of the disease under treatment and the general health of the patient prior to, and following one or more cycles of combination therapy in order to determine the optimal therapeutic combination.

4. Monitoring

The methods provided herein can further include one or more steps of monitoring the subject, monitoring the tumor, and/or monitoring the virus administered to the subject. Any of a variety of monitoring steps can be included in the methods provided herein, including, but not limited to, monitoring tumor size, monitoring anti-(tumor antigen) antibody titer, monitoring the presence and/or size of metastases, monitoring the subject's lymph nodes, monitoring the subject's weight or other health indicators including blood or urine markers, monitoring anti-(viral antigen) antibody titer, monitoring viral expression of a detectable gene product, and directly monitoring viral titer in a tumor, tissue or organ of a subject.

The purpose of the monitoring can be for assessing the health state of the subject or the progress of therapeutic treatment of the subject, or can be for determining whether or not further administration of the same or a different virus is warranted, or for determining when or whether or not to administer a compound to the subject where the compound can act to increase the efficacy of the therapeutic method, or the compound can act to decrease the pathogenicity of the virus administered to the subject.

a. Monitoring Viral Gene Expression

In some examples, the methods provided herein can include monitoring one or more virally expressed genes. Viruses can express one or more detectable gene products, including but not limited to, detectable proteins (e.g. luminescent or fluorescent proteins) or proteins that induce a detectable signal (e.g. proteins that bind or transport detectable compounds or modify substrates to produce a signal). The infected cells/tissue can thus be imaged by one more optical or non-optical imaging methods.

Measurement of a detectable gene product expressed by a virus can provide an accurate determination of the level of virus present in the subject. The detectable gene product can be measured by methods, such as for example, by imaging methods including, but not limited to, magnetic resonance, fluorescence, and tomographic methods, can determine the localization of the virus in the subject. Accordingly, the methods provided herein that include monitoring a detectable viral gene product can be used to determine the presence or absence of the virus in one or more organs or tissues of a subject, and/or the presence or absence of the virus in a tumor or metastases of a subject. Further, the methods provided herein that include monitoring a detectable viral gene product can be used to determine the titer of virus present in one or more organs, tissues, tumors or metastases.

Since methods provided herein can be used to monitor the amount of viruses at any particular location in a subject, the methods that include monitoring the localization and/or titer of viruses in a subject can be performed at multiple time points, and, accordingly can determine the rate of viral replication in a subject, including the rate of viral replication in one or more organs or tissues of a subject; accordingly, the methods of monitoring a viral gene product can be used for determining the replication competence of a virus. The methods provided herein also can be used to quantitate the amount of virus present in a variety of organs or tissues, and tumors or metastases, and can thereby indicate the degree of preferential accumulation of the virus in a subject; accordingly, the viral gene product monitoring methods provided herein can be used in methods of determining the ability of a virus to accumulate in tumor or metastases in preference to normal tissues or organs. Since the viruses used in the methods provided herein can accumulate in an entire tumor or can accumulate at multiple sites in a tumor, and can also accumulate in metastases, the methods provided herein for monitoring a viral gene product can be used to determine the size of a tumor or the number of metastases that are present in a subject. Monitoring such presence of viral gene product in tumor or metastasis over a range of time can be used to assess changes in the tumor or metastasis, including growth or shrinking of a tumor, or development of new metastases or disappearance of metastases, and also can be used to determine the rate of growth or shrinking of a tumor, or development of new metastases or disappearance of metastases, or the change in the rate of growth or shrinking of a tumor, or development of new metastases or disappearance of metastases. Accordingly, the methods of monitoring a viral gene product can be used for monitoring a neoplastic disease in a subject, or for determining the efficacy of treatment of a neoplastic disease, by determining rate of growth or shrinking of a tumor, or development of new metastases or disappearance of metastases, or the change in the rate of growth or shrinking of a tumor, or development of new metastases or disappearance of metastases.

Any of a variety of detectable proteins can be detected in the monitoring methods provided herein; an exemplary, non-limiting list of such detectable proteins includes any of a variety of fluorescent proteins (e.g., green or red fluorescent proteins), any of a variety of luciferases, transferrin or other iron binding proteins; or receptors, binding proteins, and antibodies, where a compound that specifically binds the receptor, binding protein or antibody can be a detectable agent or can be labeled with a detectable substance (e.g., a radionuclide or imaging agent); or transporter proteins (e.g. hNET or hNIS) that can bind to and transport detectable molecules into the cell. Viruses expressing a detectable protein can be detected by a combination of the method provided herein and know in the art. Viruses expressing more than one detectable protein or two or more viruses expressing various detectable protein can be detected and distinguished by dual imaging methods. For example, a virus expressing a fluorescent protein and an iron binding protein can be detected in vitro or in vivo by low light fluorescence imaging and magnetic resonance, respectively. In another example, a virus expressing two or more fluorescent proteins can be detected by fluorescence imaging at different wavelength. In vivo dual imaging can be performed on a subject that has been administered a virus expressing two or more detectable gene products or two or more viruses each expressing one or more detectable gene products.

b. Monitoring Tumor Size

Also provided herein are methods of monitoring tumor and/or metastasis size and location. Tumor and or metastasis size can be monitored by any of a variety of methods known in the art, including external assessment methods or tomographic or magnetic imaging methods. In addition to the methods known in the art, methods provided herein, for example, monitoring viral gene expression, can be used for monitoring tumor and/or metastasis size.

Monitoring size over several time points can provide information regarding the increase or decrease in size of a tumor or metastasis, and can also provide information regarding the presence of additional tumors and/or metastases in the subject. Monitoring tumor size over several time points can provide information regarding the development of a neoplastic disease in a subject, including the efficacy of treatment of a neoplastic disease in a subject.

c. Monitoring Antibody Titer

The methods provided herein also can include monitoring the antibody titer in a subject, including antibodies produced in response to administration of a virus to a subject. The viruses administered in the methods provided herein can elicit an immune response to endogenous viral antigens. The viruses administered in the methods provided herein also can elicit an immune response to exogenous genes expressed by a virus. The viruses administered in the methods provided herein also can elicit an immune response to tumor antigens. Monitoring antibody titer against viral antigens, viral expressed exogenous gene products, or tumor antigens can be used in methods of monitoring the toxicity of a virus, monitoring the efficacy of treatment methods, or monitoring the level of gene product or antibodies for production and/or harvesting.

In one example, monitoring antibody titer can be used to monitor the toxicity of a virus. Antibody titer against a virus can vary over the time period after administration of the virus to the subject, where at some particular time points, a low anti-(viral antigen) antibody titer can indicate a higher toxicity, while at other time points a high anti-(viral antigen) antibody titer can indicate a higher toxicity. The viruses used in the methods provided herein can be immunogenic, and can, therefore, elicit an immune response soon after administering the virus to the subject. Generally, a virus against which a subject's immune system can quickly mount a strong immune response can be a virus that has low toxicity when the subject's immune system can remove the virus from all normal organs or tissues. Thus, in some examples, a high antibody titer against viral antigens soon after administering the virus to a subject can indicate low toxicity of a virus. In contrast, a virus that is not highly immunogenic can infect a host organism without eliciting a strong immune response, which can result in a higher toxicity of the virus to the host. Accordingly, in some examples, a high antibody titer against viral antigens soon after administering the virus to a subject can indicate low toxicity of a virus.

In other examples, monitoring antibody titer can be used to monitor the efficacy of treatment methods. In the methods provided herein, antibody titer, such as anti-(tumor antigen) antibody titer, can indicate the efficacy of a therapeutic method such as a therapeutic method to treat neoplastic disease. Therapeutic methods provided herein can include causing or enhancing an immune response against a tumor and/or metastasis. Thus, by monitoring the anti-(tumor antigen) antibody titer, it is possible to monitor the efficacy of a therapeutic method in causing or enhancing an immune response against a tumor and/or metastasis. The therapeutic methods provided herein also can include administering to a subject a virus that can accumulate in a tumor and can cause or enhance an anti-tumor immune response. Accordingly, it is possible to monitor the ability of a host to mount an immune response against viruses accumulated in a tumor or metastasis, which can indicate that a subject has also mounted an anti-tumor immune response, or can indicate that a subject is likely to mount an anti-tumor immune response, or can indicate that a subject is capable of mounting an anti-tumor immune response.

In other examples, monitoring antibody titer can be used for monitoring the level of gene product or antibodies for production and/or harvesting. As provided herein, methods can be used for producing proteins, RNA molecules or other compounds by expressing an exogenous gene in a virus that has accumulated in a tumor. Further provided herein are methods for producing antibodies against a protein, RNA molecule or other compound produced by exogenous gene expression of a virus that has accumulated in a tumor. Monitoring antibody titer against the protein, RNA molecule or other compound can indicate the level of production of the protein, RNA molecule or other compound by the tumor-accumulated virus, and also can directly indicate the level of antibodies specific for such a protein, RNA molecule or other compound.

d. Monitoring General Health Diagnostics

The methods provided herein also can include methods of monitoring the health of a subject. Some of the methods provided herein are therapeutic methods, including neoplastic disease therapeutic methods. Monitoring the health of a subject can be used to determine the efficacy of the therapeutic method, as is known in the art. The methods provided herein also can include a step of administering to a subject a virus. Monitoring the health of a subject can be used to determine the pathogenicity of a virus administered to a subject. Any of a variety of health diagnostic methods for monitoring disease such as neoplastic disease, infectious disease, or immune-related disease can be monitored, as is known in the art. For example, the weight, blood pressure, pulse, breathing, color, temperature or other observable state of a subject can indicate the health of a subject. In addition, the presence or absence or level of one or more components in a sample from a subject can indicate the health of a subject. Typical samples can include blood and urine samples, where the presence or absence or level of one or more components can be determined by performing, for example, a blood panel or a urine panel diagnostic test. Exemplary components indicative of a subject's health include, but are not limited to, white blood cell count, hematocrit, or reactive protein concentration.

e. Monitoring Coordinated with Treatment

Also provided herein are methods of monitoring a therapy, where therapeutic decisions can be based on the results of the monitoring. Therapeutic methods provided herein can include administering to a subject a virus, where the virus can preferentially accumulate in a tumor and/or metastasis, and where the virus can cause or enhance an anti-tumor immune response. Such therapeutic methods can include a variety of steps including multiple administrations of a particular virus, administration of a second virus, or administration of a therapeutic compound. Determination of the amount, timing or type of virus or compound to administer to the subject can be based on one or more results from monitoring the subject. For example, the antibody titer in a subject can be used to determine whether or not it is desirable to administer a virus or compound, the quantity of virus or compound to administer, and the type of virus or compound to administer, where, for example, a low antibody titer can indicate the desirability of administering additional virus, a different virus, or a therapeutic compound such as a compound that induces viral gene expression. In another example, the overall health state of a subject can be used to determine whether or not it is desirable to administer a virus or compound, the quantity of virus or compound to administer, and the type of virus or compound to administer, where, for example, determining that the subject is healthy can indicate the desirability of administering additional virus, a different virus, or a therapeutic compound such as a compound that induces viral gene expression. In another example, monitoring a detectable virally expressed gene product can be used to determine whether or not it is desirable to administer a virus or compound, the quantity of virus or compound to administer, and the type of virus or compound to administer. Such monitoring methods can be used to determine whether or not the therapeutic method is effective, whether or not the therapeutic method is pathogenic to the subject, whether or not the virus has accumulated in a tumor or metastasis, and whether or not the virus has accumulated in normal tissues or organs. Based on such determinations, the desirability and form of further therapeutic methods can be derived.

In one example, determination of whether or not a therapeutic method is effective can be used to derive further therapeutic methods. Any of a variety of methods of monitoring can be used to determine whether or not a therapeutic method is effective, as provided herein or otherwise known in the art. If monitoring methods indicate that the therapeutic method is effective, a decision can be made to maintain the current course of therapy, which can include further administrations of a virus or compound, or a decision can be made that no further administrations are required. If monitoring methods indicate that the therapeutic method is ineffective, the monitoring results can indicate whether or not a course of treatment should be discontinued (e.g., when a virus is pathogenic to the subject), or changed (e.g., when a virus accumulates in a tumor without harming the host organism, but without eliciting an anti-tumor immune response), or increased in frequency or amount (e.g., when little or no virus accumulates in tumor).

In one example, monitoring can indicate that a virus is pathogenic to a subject. In such instances, a decision can be made to terminate administration of the virus to the subject, to administer lower levels of the virus to the subject, to administer a different virus to a subject, or to administer to a subject a compound that reduces the pathogenicity of the virus. In one example, administration of a virus that is determined to be pathogenic can be terminated. In another example, the dosage amount of a virus that is determined to be pathogenic can be decreased for subsequent administration; in one version of such an example, the subject can be pre-treated with another virus that can increase the ability of the pathogenic virus to accumulate in tumor, prior to re-administering the pathogenic virus to the subject. In another example, a subject can have administered thereto a virus that is pathogenic to the subject; administration of such a pathogenic virus can be accompanied by administration of, for example, an antiviral compound (e.g., cidofovir), pathogenicity attenuating compound (e.g., a compound that down-regulates the expression of a lytic or apoptotic gene product), or other compound that can decrease the proliferation, toxicity, or cell killing properties of a virus, as described herein elsewhere. In one variation of such an example, the localization of the virus can be monitored, and, upon determination that the virus is accumulated in tumor and/or metastases but not in normal tissues or organs, administration of the antiviral compound or pathogenicity attenuating compound can be terminated, and the pathogenic activity of the virus can be activated or increased, but limited to the tumor and/or metastasis. In another variation of such an example, after terminating administration of the antiviral compound or pathogenicity attenuating compound, the presence of the virus and/or pathogenicity of the virus can be further monitored, and administration of such a compound can be reinitiated if the virus is determined to pose a threat to the host by, for example, spreading to normal organs or tissues, releasing a toxin into the vasculature, or otherwise having pathogenic effects reaching beyond the tumor or metastasis.

In another example, monitoring can determine whether or not a virus has accumulated in a tumor or metastasis of a subject. Upon such a determination, a decision can be made to further administer additional virus, a different virus or a compound to the subject. In another example, monitoring the presence of a virus in a tumor can be used in deciding to administer to the subject a compound, where the compound can increase the pathogenicity, proliferation, or immunogenicity of a virus or the compound can otherwise act in conjunction with the virus to increase the proliferation, toxicity, tumor cell killing, or immune response eliciting properties of a virus; in one variation of such an example, the virus can, for example, have little or no lytic or cell killing capability in the absence of such a compound; in a further variation of such an example, monitoring of the presence of the virus in a tumor or metastasis can be coupled with monitoring the absence of the virus in normal tissues or organs, where the compound is administered if the virus is present in tumor or metastasis and not at all present or substantially not present in normal organs or tissues; in a further variation of such an example, the amount of virus in a tumor or metastasis can be monitored, where the compound is administered if the virus is present in tumor or metastasis at sufficient levels.

### I. EXAMPLES

The following examples are included for illustrative purposes only and are not intended to limit the scope of the invention.

### Example 1

### LIVP Viruses

A. GLV-1h68

GLV-1h68 is a recombinant, replication-competent vaccinia virus derived from the vaccinia virus LIVP strain (Lister strain from the Institute of Viral Preparations, Moscow, Russia). GLV-1h68 contains an expression cassette containing a Ruc-GFP cDNA molecule (a fusion of DNA encoding Renilla luciferase and DNA encoding GFP) under the control of a vaccinia synthetic early/late promoter P_{SEL} ((P_{sEL})Ruc-GFP) inserted into the F14.5L gene locus; an expression cassette containing a DNA molecule encoding beta-galactosidase under the control of the vaccinia early/late promoter P_{7.5k} ((P_{7.5k})LacZ), DNA encoding a rat transferrin receptor positioned in the reverse orientation for transcription relative to the vaccinia synthetic early/late promoter P_{SEL} ((P_{SEL})ᵣTrfR) inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) inserted into the HA gene locus. The genome of GLV-1h68 has the sequence of nucleotides set forth in SEQ ID NO:9.

B. GLV-1h189

GLV-1h189 was generated by insertion of an expression cassette encoding TurboFP635 (set forth in SEQ ID NO:24) under the control of the vaccinia P_{SEL} promoter into the HA locus of starting strain GLV-1h68 thereby deleting the gusA expression cassette at the HA locus of starting GLV-1h68. Thus, in strain GLV-1h189, the vaccinia HA gene is interrupted within the coding sequence by a DNA fragment containing DNA encoding TurboFP635 operably linked to the vaccinia synthetic early/late promoter. The genome of GLV-1h189 has the sequence of nucleotides set forth in SEQ ID NO:20.

C. GLV-2b372

GLV-2b372 is a recombinant, replication-competent vaccinia virus generated from the LIVP clonal isolate designated LIVP 1.1.1 (SEQ ID NO:2; see e.g. International PCT Application No. PCT/US 12/033,684). GLV-2b372 contains TurboFP635 (Far-red fluorescent protein “katushka”; set forth in SEQ ID NO:24) under the control of the vaccinia synthetic early/late promoter at the TK locus. The genome of GLV-1b372 has the sequence of nucleotides set forth in SEQ ID NO:25.

### Example 2

### Effect of Silk-Elastinlike Protein Polymer (SELP) on the Infectivity of Recombinant LIVP Vaccinia Virus

GLV-1h189-SELP47K was generated by mixing a 1.29×10⁹ pfu/mL stock of GLV-1h189 virus (described in Example 1) with an equal volume of 8% SELP-47K. SELP-47K (SEQ ID NO:62) was prepared as a 12% by weight solution as previously described (U.S. Pat. No. 6,380,154; Megeed et al. (2002) Advanced Drug Delivery Reviews, 54:1075-1091; Cappello et al. (1998) Journal of Controlled Release, 53:105-117; Gustafson et al. (2010) Advanced Drug Delivery Reviews, 62:1509-1523; Haider et al. (2004) Molecular Pharmaceutics, 2:139-150) and diluted to 8% concentration in 1 mM Tris, pH 9). The mixture was incubated at room temperature for 1 hour. As a control, virus also was mixed with an equal volume of 1 mM Tris, pH9 (GLV-1h189-Tris) and also incubated for 1 hour.

To determine infectivity of the virus compositions, a viral titer assay was performed in a standard plaque assay using African green monkey kidney fibroblast CV-1 cells (ATCC No. CCL-70; American Type Culture Collection, Manassas, Va.). CV-1 cells were plated in a 24-well plate at 2.5×10⁵ cells per well and grown until near confluency. Wells containing a cell monolayer were infected with serial dilutions of the virus compositions. The cells in each well were overlayed with virus overlay medium (DMEM+5% FBS+1% Antibiotic-Antimycotic Solution+1.5% carboxymethylcellulose), and the cells were further incubated until plaques were visible. After addition of color dye to visualize the plaques, viral titer (pfu/mL) was calculated by counting the number of plaques in the well and dividing by the dilution factor (d) and volume (V) of diluted virus added to the well. The results show that the viral titer for both virus compositions were approximately the same at about 5.0×10⁸ pfu/mL. Thus, the results show that SELP does not affect vaccinia virus infectivity in cell cultures.

### Example 3

### In Vitro Infection of 8505c Anaplastic Thyroid Cancer Cells with GLV-1h68 in SELP

A. Infection of 8505c Cells with GLV-1h68

Human anaplastic thyroid carcinoma cell line 8505c (Japanese Collection of Research Bioresources Cell Bank, Shinjuku, Japan) were infected with varying concentrations of GLV-1h68 virus (described in Example 1). The 8505c cells were maintained in minimal essential medium (MEM) with 10% fetal calf serum and penicillin and streptomycin at 37° C. and 5% carbon dioxide. For infection, 8505c cells were grown overnight in 12-well plates (plated at 2×10⁴ cells per well) in 1 mL of MEM media. GLV-1h68 in MEM was added to each well at a multiplicity of infection (MOI) of 0, 0.01, 0.1, and 1.

Cell viability was assessed daily for seven days by measuring the release of intracellular lactate dehydrogenase (LDH). Cells were washed with PBS and lysed with Triton X-100 (1.35%, Sigma-Aldrich, St. Louis, Mo.) to release intracellular LDH. The supernatants were analyzed for released LDH using CytoTox 96® Non-Radioactive Cytotoxicity Assay kit (Catalog #G1780, Promega, Madison, Wis.). The CytoTox 96® Assay is a 30 minute coupled enzymatic assay in which lactate dehydrogenase catalyzes the conversion of a tetrazolium salt into a red formazan product. The product formation was monitored by measuring the absorbance at 450 nm using an EL321E spectrophotometer (BioTek Instruments). Results were expressed as the percentage of surviving cells, determined by comparing the measured LDH of each infected sample relative to control cells that were not infected. All samples were analyzed in triplicate. The results showed that GLV-1h68 dose-dependently infects and lyses 8505c cells, since at an MOI of 1, 0.1 and 0.01 there was just 1%, 5% and 23% cell viability remaining, respectively, at day 7.

Viral replication was assessed in 8505c cells that were infected with GLV-1h68 at an MOI of 0.1 as described above. Supernatants were collected daily for seven days and stored at −80° C. After thawing, standard plaque assays were performed on confluent CV-1 cells infected with serial dilutions of the thawed supernatants. All samples were measured in triplicate. The results of the assay by viral titer demonstrated viral replication in infected cells with a logarithmic viral growth from days 1 through 6. For example, at day 1, viral titer was approximately 5.0×10² pfu/mL, which steadily increased until day 6 to approximately 1×10⁶ pfu/mL. By day 7, the viral titers plateaued and were substantially the same as observed at day 6.

Thus, the results show that 8505c cells support GLV-1h68 infection and effective viral replication.

B. Infection of 8505c cells with GLV-1h68-SELP or GLV-1h68

SELP-47K (described in Example 2) that was stored frozen at −80° C. as a 12% by weight solution was thawed at room temperature and diluted with PBS or MEM to a 4% concentration. GLV-1h68-SELP47K (GLV-1h68 in 4% SELP gel) was generated by mixing a stock solution of GLV-1h68 with 4% SELP-47K to the appropriate concentrations. As a control, the same concentration of GLV-1h68 was mixed with media (MEM or PBS).

To assess infectivity, 8505c cells, plated overnight at 2×10⁴ cells per well in 24-well plates, were infected with either GLV-1h68-media, or GLV-1h68-SELP at an MOI of 1.0. Infectivity was assessed by measuring GFP expression by microscopy at 12, 24, 36 and 48 hours using a fluorescence inverted microscope (Nikon Eclipse TS100, Nikon, Japan). Cells infected with GLV-1h68-SELP and GLV-1h68-MEM exhibited identical intensity of GFP expression by microscopy at 12, 24, 36, and 48 hours. For both groups, GFP expression was not substantially detectable at 12 hours, but was measurable at 24 hours. There also was a decreased GFP expression observed at 48 hours, which reflects expected decreased cell viability from viral cytotoxic effects.

The ability of GLV-1h68 to infect and replicate in cells under a solidified layer of SELP also was assessed. 8505c cells were plated overnight at 2×10⁴ cells per well in 24-well plates as described above. A 4% SELP was layered over the cells and allowed to solidify for 10 minutes. GLV-1h68 in 50 μL PBS was injected at an MOI of 1.0 by micropipette under the solidified layer of SELP. When compared to the other two groups of GLV-1h68-media and GLV-1h68-SELP47K, cells infected with the identical amount of GLV-1h68 injected under a solidified layer of SELP showed diminished GFP expression.

Cell lysis by each of the virus-treated or control groups also was assessed. A confluent monolayer of 8505c cells in wells of a 24-well plate were infected with GLV-1h68-MEM or GLV-1h68-SELP47K at an MOI of 1.0 as described above or were infected with GLV-1h68 in 50 μL PBS under a layer of 4% SELP. As controls, cells also were infected with MEM or 4% SELP diluted in MEM. To visualize cell viability, at days 1, 2, 3, 4 and 5 cells were fixed with 20% ethanol, stained with 0.1% crystal violet for 10 minutes, washed with water and room air dried. Photographs were taken with an inverted microscope (Nikon Eclipse TS100). All samples were assessed in triplicate. The results showed that 8505c cells in media or SELP gel that were not treated with virus showed progressive 8505c growth to confluence by day 5. GLV-1h68-SELP47K and GLV-1h68-media both caused similar patterns of cell lysis by microscopy over a 5 day period. GLV-1h68 injected under a layer of SELP resulted in slightly diminished cell lysis as compared with the other two virally treated groups.

### Example 4

### Infection of CV-1 Cells with GLV-1h189-SELP or GLV-1h189

GLV-1h189-SELP47K and GLV-1h189-Tris were prepared as described in Example 2. CV-1 cells (ATCC No. CCL-70) were plated at 2×10⁴ cells per well and grown overnight in 96-well plates in 100 μL of DMEM-10. When the cells were confluent, GLV-1h189-SELP47K and GLV-1h189-Tris were added to each well at a multiplicity of infection (MOI) of 0.1, 0.01, or 0.001 for 1 hour at 37° C. Then, additional medium was added to cells infected at an MOI 0.1, 0.01 or 0.001, or inoculum was removed and fresh medium added for cells infected at an MOI of 0.01 or 0.001. For all groups, cells were allowed to grow for up to 192 hours. At various times after infection, TurboFP635 fluorescence intensity was measured using a SpectraMax M5 with an excitation/emission/cutoff of 588/635/630 nm.

For cells infected with virus at an MOI of 0.1, there was a steady increase in measured fluorescence in cells up to about 72 hours, which steadily decreased at later time points. The decrease in fluorescence was less for cells infected with GLV-1h189-Tris than for cells infected with GLV-1h189-SELP47K. For cells infected with virus at an MOI of 0.01, including cells in which the inoculum was removed, there was a steady increase in fluorescence in cells up to about 72 hours after infection, which then plateaued with similar observable fluorescence measured at the later time points up to 192 hours. The measured fluorescence was similar at all time points in cells infected with GLV-1h189-Tris and cells infected with GLV-1h189-SELP47K. At an MOI of 0.001, the fluorescence steadily increased up to 120 to 140 hours after infection and then plateaued at 192 hours, with similar levels of observed fluorescence in cells infected with GLV-1h189-Tris and cells infected with GLV-1h189-SELP47K and in cells where the inoculum was removed or was not removed. Thus, the results show that SELP-47K does not affect vaccinia virus replication in cell cultures.

### Example 5

### Effect of SELP on GLV-1h68 Tumor Cell Infection Following Intratumoral Injection into Flank Tumors

Tumors were established by injecting 5×10⁶ 8505c cells into the subcutaneous flanks of 6 week old female nude athymic mice (NCl, Bethesda, Md.) under inhalational anesthesia with isoflurane (Baxter, Deerfield, Ill.). Tumor volumes were calculated as the shape of an ellipsoid: (4/3*π)*(a/2)*(b/2)². At a mean tumor volume of 100 mm³, mice (n=5 per group) were given a single intratumoral injection with 50 μL volume of either (1) 1×10⁷ pfu of GLV-1h68-PBS, (2) 1×10⁷ pfu of GLV-1h68-SELP47K, or (3) PBS. GLV-1h68-SELP47K was prepared as described in Example 3B. Viral transgene expression, tumor volumes and body weights were measured.

A. Viral Transgene Expression Virus infection of tumors was assessed by measuring GFP expression in the tumors of mice. Mice (n=3 per group) were imaged with a Maestro In Vivo Fluoroescence Imaging System (Cambridge Research & Instrumentation, Woburn, Mass.). GFP signals were quantified by using Maestro 2.4 software. The results showed that at day 1, GFP expression in mice treated with GLV-1h68-SELP47K was barely detectable, whereas GFP expression in mice treated with GLV-1h68-PBS could be seen as early as day 1. At day 10 and later, GFP expression was slightly higher in mice treated with GLV-1h68-SELP47K compared to mice treated with GLV-1h68-PBS reflecting a continuous slow viral release from the SELP matrix and subsequent cancer cell infection. By day 25, GFP expression was barely detectable in mice treated with GLV-1h68-PBS, yet GFP expression was still slightly detectable in mice treated with GLV-1h68-SELP47K

Virus infection of tumors also was assessed by monitoring luciferase activity. At varying times post-injection of virus, 5 μL coelenterazine (0.5 μL; Biotium, Inc., Hayward, Calif.) in 95 μL PBS was injected via the retro-orbital sinus (n=5 per group) of mice under inhalational anesthesia. Luciferase activity was detected with a cooled CCD camera (Xenogen IVIS; Xenogen Corp., Caliper Life Sciences, Hopkinton, Mass.). Emitted photons were measured for 60 seconds. Images were analyzed using Living Image Software (Xenogen, Caliper Life Sciences). The results showed that mice treated with GLV-1h68-PBS or GLV-1h68-SELP47K showed peak luciferase expression at day 4, with those mice treated with GLV-1h68-PBS having slightly higher peak expression compared to those treated with GLV-1h68-SELP47K. After day 10, mice treated with GLV-1h68-SELP47K had higher levels of luciferase expression compared to those mice treated with GLV-1h68-PBS. Luciferase expression was still detectable in mice treated with GLV-1h68-SELP47K at day 20, whereas luciferase expression was barely detectable in those mice treated with GLV-1h68-PBS. As a control, mice treated only with PBS showed no detectable luciferase expression at any time-point tested.

### B. Tumor Size

The effect of intratumoral injection of GLV-1h68-PBS or GLV-1h68-SELP47K on tumor regression also was assessed by monitoring tumor volumes at various times post injection of virus. The results showed that mice treated with GLV-1h68-PBS or GLV-1h68-SELP47K showed an equivalent and complete regression of tumor volume. In both groups, tumor volume steadily decreased from a maximal volume of about 180 mm³ at day 5 to less than about 50 mm³ by days 20 to 25, and even further regression up to day 45. Thus, the results demonstrate that intratumoral injection of GLV-1h68-SELP47K provides no advantage in facilitating tumor regression in mice compared to intratumoral injection of virus alone.

### Example 6

### Effect of SELP on GLV-1h189 Infection and Replication in Tumor Cells Following Intravenous Injection

The effect of SELP on virus infection and replication following intravenous injection was evaluated using a mouse model of human prostate cancer. Male nude mice (Hsd:Athymic Nude-Foxn1^{nu}; Harlan, Indianapolis, Ind.; 4-5 weeks old) were injected subcutaneously (s.c. on the right hind leg 1.0×10⁷ cells in 100 μL PBS) with DU145 cells (ATCC No. HTB-81; American Type Culture Collection (Manassas, Va.)) to establish tumors. Four weeks following tumor cell implantation, mice were injected with 5×10⁶ pfu of GLV-1h189-SELP47K (3 mice) or GLV-1h189-PBS (2 mice) via a tail vein injection. Tumors were imaged for TurboFP635 and GFP expression with a stereo fluorescence macroimaging system (Lightools Research), at 3 and 5 days after treatment. All mice were sacrificed on day 6 after treatment. At day 6, each tumor was cut into 2 halves: one half was used for imaging and the other half was used for virus titration in CV-1 cells using a standard plaque assay.

The results show that at day 3 after treatment there was expression of viral genes in the tumors of all treated mice. Tumors of mice that were treated with GLV-1h189-SELP47K exhibited greater intensity of the TurboFP635 and GFP transgenes, demonstrating that SELP enhanced vaccinia virus replication in tumors by 3 days post-injection. Similar results were observed 5 days after injection of virus, although the overall intensity of expressed genes was higher than at day 3. In excised tumors at day 6, the transgene expression in tumors of mice treated with GLV-1h189-SELP47K remained enhanced compared to tumors of mice treated with GLV-1h189-PBS, in particular when TurboFP635 expression was imaged.

The results from assessing viral titration by standard plaque assay at day 6 confirmed the enhanced replication efficiency of SELP-coated virus in tumors. The viral titer was normalized to the half tumor size, and was depicted as pfu/gram tumor weight (pfu/gm). In excised tumors at day 6, the viral titer in tumors of mice treated with GLV-1h189-SELP47K was about 5.17×10⁸ pfu/gm, while in tumors of mice treated with GLV-1h189-PBS, the viral titer was about 2.37×10⁸ pfu/gm.

### Example 7

### Assessment of Viral Integrity and Stability with and without SELP

GLV-1h68 viral integrity was studied in SELP (GLV-1h68-SELP47K) as compared to in PBS (GLV-1h68-PBS) following incubation at 37° C. over a four week period in the absence of any cells. GLV-1h68-SELP47K was formed as a cylindrical gel of GLV-1h68 (1×10⁶ pfu) in 50 μL, of 4% SELP in PBS using insulin syringes. A solution of GLV-1h68 (1×10⁶ pfu) in 50 μL PBS was used as a control. Samples were placed in 2 mL cryogenic tubes in 1.5 mL PBS, and incubated at 37° C. in a shaking incubator to facilitate viral elution. Triplicate samples were removed at varying time points and stored at −80° C. To disrupt the SELP gels for viral titer studies, SELP samples were shaken in a TissueLyser (Qiagen, Valencia, Calif.) at 30 Hz for 2 minutes to create tiny gel particles. The method of using shaking to disrupt the gel enhanced viral recovery from SELP gel compared to other methods using vortexing or homogenizer beads. It is unlikely, however, that all viral particles in the SELP gel specimens were eluted from the solid gel matrix. Viral integrity was measured by measuring infectious GLV-1h68 viral titers using a standard plaque assay.

The results show that incubation of GLV-1h68-PBS at 37° C. exhibits a rapid decline of infectious viral plaque forming units over time, dropping off from over 8×10⁵ pfu to below 2.5×10⁵ pfu by 12 hours. GLV-1h68-SELP47K resulted in more stable infectious particle retention when incubated from 2 hours to 7 days at 37° C., with over 3.2×10⁵ pfu infectious particles retained at 7 days compared to only 2.9×10² pfu for the GLV-1h68-PBS group when incubated over the same 7 day period. Infectious viral plaque forming units were not detected in the GLV-1h68-PBS sample past one week, while infectious viral plaque forming units could be detected in the GLV-1h68-SELP47K sample for up to four weeks. Thus, the results show that SELP gel confers a protective effect against normal viral degradation that occurs at room temperature.

### Example 8

### Effect of SELP on GLV-1h68 Infection of a Partially Resected High Volume Tumor Following Topical Application

Flank tumors were established in mice as described in Example 5. To mimic an intraoperative scenario of an incomplete surgical resection with high volume residual disease, animals with 8505c flank tumors underwent general anesthesia and surgical resection, leaving approximately 120 mm³ of the deep portion of the tumor as residual disease. Tumors (n=7 per group) were then treated topically over the residual tumor with a direct application of 300 μL volume of: (1) PBS, (2) 4% SELP47K, (3) 1×10⁷ pfu of GLV-1h68-PBS, or (4) 1×10⁷ pfu of GLV-1h68-SELP47K. GLV-1h68-SELP47K was prepared as described in Example 3B. After application of the treatment to the surface of the residual tumor, the skin flap was closed with staples. Tumors were measured every other day, beginning 7 days after the surgery to allow for resolution of tissue edema and fluid/gel volume. Animals underwent in vivo luciferase imaging and quantification as described in Example 5. Tumor volumes (mm³) also were measured.

At day 12, partially resected tumors of mice topically treated with GLV-1h68-SELP47K had more than double peak expression levels of luciferase expression when compared to partially resected tumors of mice treated with GLV-1h68-PBS. This increased gene expression correlated with tumor regression. In control mice not treated with virus (PBS and 4% SELP), tumor volume steady increased from 120 mm³ at day 0 to about 200 mm³ at day 6, about 400 mm³ at day 14 and about 600 mm³ at day 19 when animals were sacrificed. For example, at day 14, the mean volume of control tumors treated with topical PBS alone was 398±142 mm³, and SELP alone was 424±86 mm³. For virus-treated groups, the tumor volume declined over the same time period for both GLV-1h68-SELP47K and GLV-1h68-PBS treated tumors. After day 12, however, the results showed significantly smaller tumor volumes for GLV-1h68-SELP47K treated tumors as compared to GLV-1h68-PBS treated tumors. For example, at day 14, the mean volume of tumors treated with topical virus was 104±5 mm³ for GLV-1h68-PBS, and 30±14 mm³ for GLV-1h68-SELP47K (p<0.05, t-test, 2-tailed).

### Example 9

### Effect of SELP on GLV-1h68 Infection of a Partially Resected Low Volume Tumor Following Topical Application

Flank tumors were established in mice as described in Example 5. The flank model described in Example 8 was modified to leave a very thin layer of residual tumor to mimic a low volume residual disease. Briefly, animals with 8505c flank tumors underwent surgical resection of the superficial tumor, leaving approximately 50 mm³ of tumor remaining as a flat surface. Tumors (n=7-8 per group) were then treated with a direct application of 50 μL volume topically over the residual tumor of: (1) PBS, (2) 1×10⁷ pfu of GLV-1h68-PBS, (3) 1×10⁷ pfu of GLV-1h68-SELP47K in 4% SELP, or (4) 1×10⁷ pfu of GLV-1h68 in tiny 4% SELP particles. GLV-1h68-SELP47K (GLV-1h68 in SELP gel) was prepared as described in Example 3B. The 4% SELP particles were created by shaking the SELP gel at 30 Hz in a TissueLyser (Qiagen, Valencia, Calif.) for two minutes to create tiny gel particles. After application to the surface of the residual tumor, the skin flap was closed with staples. Tumor volumes were measured, and animals underwent in vivo luciferase imaging as described in Example 3.

### A. Luciferase Expression

In vivo imaging demonstrated no luciferase expression in tumors of mice treated with the no virus PBS control. The results also demonstrated that for all groups where tumors were topically treated with virus, there was a steady increase in luciferase expression up until day 10 (GLV-1h68-PBS) or up until day 12 (GLV-1h68-SELP47K or GLV-1h68 in 4% SELP particles), which then decreased close to control levels at day 14. The luciferase expression was the greatest in tumors that were topically treated with GLV-1h68 in 4% SELP particles, followed by tumors treated with GLV-1h68-SELP47K and then GLV-1h68-PBS. For example, at day 12, there was more than a 2.5-fold increase in peak luciferase expression for GLV-1h68-SELP47K as compared with GLV-1h68-PBS. Also, GLV-1h68 in the SELP particles resulted in 50% higher luciferase expression at day 12 as compared to GLV-1h68-SELP47K.

### B. β-Galactosidase Expression

β-galactosidase histochemical staining was performed on excised, low volume, post-resection tumor specimens treated with topical GLV-1h68 in PBS, SELP, or SELP particles to assess lacZ expression as a measure of viral infection. Mice (n=2 per group) were sacrificed at varying time points after treatment up to 30 days post-treatment (t=12 h, day 1, 2, 4, 8, 12, 16, 24 and 30 post-treatment), and residual tumors excised, frozen in Tissue Tek (Sakura Finetek USA, Torrance, Calif.) and sectioned. Slides were fixed with 1% glutaraldehyde and stained with X-Gal (bromo-chloro-indolyl-galactopyranoside) at 1 mg/mL in 5 mM K₄Fe(CN)₆ and 2 mM MgCl₂, and counterstained with nuclear fast red. Sections were digitally photographed using an inverted microscope (Nikon Eclipse TS 100).

Tumors of mice treated with GLV-1h68-PBS exhibited maximal lacZ expression by day 8, with subsequent gradual loss of expression to nearly complete loss of expression by day 30. GLV-1h68-SELP47K or GLV-1h68 in SELP particles resulted in similar early expression of lacZ expression with maximal expression by day 8. The lacZ expression in tumors treated with GLV-1h68-SELP47K or GLV-1h68 in SELP particles was sustained over the next 22 days with intense expression even at day 30, and was consistently greater than lacZ expression in tumors treated with GLV-1h68-PBS. Thus, the results show that there was more potent and sustained viral expression in cancer cells treated with GLV-1h68 in SELP or SELP particles as compared with PBS.

### C. Tumor Regression

The increased gene expression correlated with tumor regression. In control mice not treated with virus (PBS control), tumor volume steadily increased from 50 mm³ to about 100 mm³ at day 25 post-topical application of virus. As assessed by tumor volume, the time course demonstrated greater tumor regression for tumors treated with GLV-1h68 in SELP particles, followed by GLV-1h68-SELP47K, and GLV-1h68-PBS. For example, at day 13, tumor volumes of tumors treated with GLV-1h68 in SELP particles was 8.0±3.0 mm³, while tumor volumes of tumors treated with GLV-1h68-PBS was 31.5±10.8 mm³, demonstrating that the GLV-1h68 in SELP particles groups had a lower tumor volume (p=0.05, t-test, 2-tailed). At day 13, tumor volumes of tumors treated with GLV-1h68-SELP47K was slightly higher than tumors treated with GLV-1h68 in SELP particles, but remained substantially less than tumors treated with GLV-1h68-PBS.

Topical treatment with GLV-1h68 in the SELP particles or GLV-1h68-SELP47K also led to a higher number of animals being rendered free of disease as compared with GLV-1h68 in PBS. For example, 10-12 days post-treatment about 25% of animals were determined to be free of disease when treated with GLV-1h68 in SELP particles or GLV-1h68-SELP47K, which was about 50% greater than animals treated with GLV-1h68-PBS. At day 25 days post-treatment about 50% of animals treated with GLV-1h68 in SELP particles or GLV-1h68-SELP47K were determined to be free of disease, while only about 30% of animals treated with GLV-1h68-PBS were determined to be free.

### Example 10

### Effect of Various SELP Polymers on Viral Delivery and Replication Efficiency in Tumors in Both Immune-Compromised and Immune-Competent Mouse Models

The effect of various SELP polymers on virus infection and replication following intravenous injection was evaluated in immune-compromised and immune-competent mouse models of melanoma. L1VP virus GLV-2b372 (described in Example 1) in various SELP polymers were generated by mixing a stock of GLV-2b372 (3.42×109 pfu/mL) with an equal volume of 8% SELP stock solution (dissolved in 1 mM Tris, pH 9). The mixture was incubated at room temperature for 1 hour. As a control, GLV-2b372 was mixed with an equal volume of 1 mM Tris, pH 9 and also incubated at room temperature for 1 hour. Coated virus or control was diluted into PBS to achieve a concentration of 1×10⁸ pfu/mL.

Tumors were established in C57BL/6 mice (4-5 weeks old) or nude mice (4-5 weeks old) by injecting B16-F10 mouse melanoma cells (ATCC No. CRL-6475) subcutaneously (s.c. on the right hind leg 2.0×10⁵ cells in 100 μL PBS). Fifteen (15) days following tumor cell implantation, mice were injected via a tail vein injection with 1×10⁷ pfu of GLV-2b372 (as described in Example 1) or GLV-2b372 in various SELP polymers as set forth in Table 9. Mice were sacrificed on day 6 after treatment. At day 6, tumors were excised and virus content was determined by viral titration in CV-1 cells using a standard plaque assay, and is depicted as pfu/gram tumor weight (pfu/gm).

TABLE 9
Treatment Groups

| Group | mouse strain | Number of mice | Treatment |
|---|---|---|---|
| Group 1 | nude | 10 | GL-2b372-Tris |
| Group 2 | nude | 10 | GL-2b372-SELP27CK |
| Group 3 | nude | 10 | GL-2b372-SELP47K |
| Group 4 | nude | 10 | GL-2b372-SELP815K |
| Group 5 | C57B1/6 | 10 | GL-2b372 |
| Group 6 | C57B1/6 | 10 | GL-2b372-SELP27CK |
| Group 7 | C57B1/6 | 10 | GL-2b372-SELP47K |
| Group 8 | C57B1/6 | 10 | GL-2b372-SELP815K |

The results are set forth in Table 10. The results show that the virus titer in tumors from nude mice was generally greater than from tumors from C57BL16 mice. For both nude mice and immune-competent mice, GLV-2b372-SELP-27CK and GLV-2b372-SELP-815 exhibited a higher viral titer than the GLV-2b372-Tris treated group. The viral titer of GLV-2b372-SELP47K in tumors from immune-competent mice was slightly increased compared to GLV-2b372 treated mice. In this tumor model and using the GLV-2b372 virus strain, these results show that SELP-27CK and SELP-815 enhance virus replication in tumors in both nude mice and immune-competent C57BL/6 mice.

TABLE 10
Viral Replication in Immune-Compromised and Immune-
Competent Tumor Models

| | |
|---|---|
| Mouse | Viral Titer (pfu/g) |

| Type | Treatment | Average | STDEV |
|---|---|---|---|
| Nude | GLV-2b372 | 9.16E+06 | 1.14E+07 |
| | GLV-2b372/27CK | 1.55E+07 | 3.49E+07 |
| | GLV-2b372/47K | 4.80E+06 | 6.73E+06 |
| | GLV-2b372/815K | 1.93E+07 | 4.27E+07 |
| C57BL/6 | GLV-2b372 | 1.85E+06 | 1.94E+06 |
| | GLV-2b372/27CK | 2.22E+07 | 3.64E+07 |
| | GLV-2b372/47K | 3.02E+06 | 6.57E+05 |
| | GLV-2b372/815K | 9.21E+06 | 1.31E+07 |

Since modifications will be apparent to those of skill in this art, it is intended that this invention be limited only by the scope of the appended claims.

1. A composition, comprising:
a vaccinia virus;
a silk-elastin like protein polymer (SELP), wherein the SELP is capable of transitioning from a liquid to a hydrogel to form a hydrogel composition; and
a pharmaceutically acceptable carrier.

2. The composition of claim 1, wherein the SELP comprises alternating blocks of at least two units each of a silk-like sequence of amino acids and an elastin-like sequence of amino acids set forth by the formula {[S]ₘ[E]ₙ}_{O}, wherein:
S is the silk-like sequence of amino acids;
m is the number of silk-like amino acid units;
E is the elastin-like polypeptide amino acid sequence;
n is the number of elastin-like amino acid units; and
o is the number of monomer repeats.

3. The composition of claim 1 that is a liquid.

4. The composition of claim 1 that is a hydrogel.

5. The composition of claim 1, wherein the SELP has a molecular weight of at least 15 kD.

6. The composition of claim 1, wherein:
m is 2 to 16, 2 to 10, 2 to 8, 4 to 16, 4 to 10 or 4 to 8;
n is 1 to 40, 1 to 16, 2 to 12 or 4 to 8; and/or
o is 2 to 100, 4 to 50, 6 to 25 or 2 to 20.

7. The composition of claim 1, wherein:
m is 2 to 16 or 2 to 8;
n is 1 to 16; and
o is chosen so that the SELP has a molecular weight of 15,000 to 100,000 Da.

8. The composition of claim 1, wherein:
the sequence of amino acids of the silk-like polypeptide is selected from among GAGAGS (SEQ ID NO:26) or SGAGAG (SEQ ID NO:27), or is a variant thereof that is capable of effecting formation of hydrogen bonds; and/or
the sequence of amino acids of the elastin-like polypeptide is selected from among VPGG (SEQ ID NO:30), APGVGV (SEQ ID NO:31), VPGVG (SEQ ID NO:32), or GVGVP (SEQ ID NO:29), or is a variant thereof that confers aqueous solubility.

9. The composition of claim 8, wherein the elastin-like amino acid sequence is a variant that has the amino acid sequence GXGVP (SEQ ID NO:35) or VPGXG (SEQ ID NO:36), wherein X is valine, lysine, histidine, glutamic acid, arginine, aspartic acid, serine, tryptophan, tyrosine, phenylalanine, leucine, glutamine, asparagine, cysteine or methionine.

10. The composition of claim 9, wherein the elastin-like sequence is VPGKG (SEQ ID NO:37) or GKGVP (SEQ ID NO:38).

11. The composition of claim 1, wherein the SELP comprises the sequence of amino acids selected from among [(VPGVG)₈(GAGAGS)₂]₁₈ (SEQ ID NO:39); [(GVGVP)₄(GAGAGS)₉]₁₃ (SEQ ID NO:40); [(VPGVG)₈(GAGAGS)₄]₁₂ (SEQ ID NO:41); [(VPGVG)₈(GAGAGS)₆]₁₂ (SEQ ID NO:42); [(VPGVG)₈(GAGAGS)₈]₁₁ (SEQ ID NO:43); [(VPGVG)₁₂(GAGAGS)₈]₈ (SEQ ID NO:44); [(VPGVG)₁₆(GAGAGS)₈]₇ (SEQ ID NO:45); [(VPGVG)₃₂ (GAGAGS)₈]₅ (SEQ ID NO:46); (GAGAGS)₁₂ GAAVTGRGDSPASAAGY (GAGAGS)₅(GVGVGP)₈]₆ (SEQ ID NO:47); [(GAGAGS)₂ (GVGVP)₄ GKGVP (GVGVP)₃]₆ (SEQ ID NO:48); [(GAGAGS)₂(GVGVP)₄GKGVP (GVGVP)₃]₁₂ (SEQ ID NO:49); [(GAGAGS)₂(GVGVP)₄GKGVP (GVGVP)₃]₁₈ (SEQ ID NO:50); [(GAGAGS)₂ (GVGVP)₄ GKGVP (GVGVP)₃]₁₇GAGAGS)₂(SEQ ID NO:51); [(GAGAGS)₂-(GVGVP)₄-(GKGVP)-(GVGVP)₃-(GAGAGS)₂]₁₃ (SEQ ID NO:52); [GAGAGS (GVGVP)₄ GKGVP (GVGVP)₃(GAGAGS)₂]₁₂(SEQ ID NO:53); [(GVGVP)₄GKGVP(GVGVP)₁₁(GAGAGS)₄]₅(GVGVP)₄GKGVP(GVGVP)₁₁(GAG AGS)₂(SEQ ID NO:54); [(GVGVP)₄(GKGVP)(GVGVP)₁₁(GAGAGS)₄]₇(GVGVP)₄GKGVP(GVGVP)₁₁(GA GAGS)₂ (SEQ ID NO:55); [(GVGVP)₄GKGVP(GVGVP)₁₁(GAGAGS)₄]₉(GVGVP)₄GKGVP(GVGVP)₁₁(GAG AGS)₂(SEQ ID NO:56); [GAGS(GAGAGS)₂(GVGVP)₄GKGVP(GVGVP)₁₁(GAGAGS)₅GA]₆ (SEQ ID NO:57); [(GAGAGS)₂ (GVGVP)₁ LGPLGP (GVGVP)₃ GKGVP (GVGVP)₃]₁₅ (GAGAGS)₂ (SEQ ID NO:73); and [(GAGAGS)₂ (GVGVP)₁ GFFVRARR (GVGVP)₃ GKGVP (GVGVP)₃)₁₅(GAGAGS)₂ (SEQ ID NO:74).

12. The composition of claim 1, wherein the SELP comprises the sequence of amino acids selected from among [(GAGAGS)₂(GVGVP)₄GKGVP (GVGVP)₃]₁₇ GAGAGS)₂ (SEQ ID NO:51); [(GAGAGS)₂-(GVGVP)₄-(GKGVP)-(GVGVP)₃-(GAGAGS)₂]₁₃ (SEQ ID NO:52); and [GAGS(GAGAGS)₂(GVGVP)₄GKGVP(GVGVP)₁₁(GAGAGS)₅GA]₆(SEQ ID NO:57).

13. The composition of claim 1, wherein the SELP comprises the sequence of amino acids selected from among SELP-27K (SEQ ID NO: 61), SELP-47K (SEQ ID NO:62) and SELP-815K (SEQ ID NO:63).

14. The composition of claim 1, wherein the concentration of the SELP protein polymer is at a weight percentage (wt %) of the composition of from or from about 2% (w/w) to about to 50% (w/w) or 2% (w/w) to about 20% (w/w), each inclusive.

15. The composition of claim 1, wherein the strain of vaccinia virus is selected from among Lister, Western Reserve (WR), Copenhagen (Cop), Bern, Paris, Tashkent, Tian Tan, Wyeth (DRYVAX), IHD-J, IHD-W, Brighton, Ankara, CVA382, Modified Vaccinia Ankara (MVA), Dairen I, LC16 m8, LC16M0, LIVP, ACAM2000, WR 65-16, Connaught, New York City Board of Health (NYCBH), EM-63 and NYVAC strain.

16. The composition of claim 15, wherein the vaccinia virus is a Lister strain virus.

17. The composition of claim 1, wherein the vaccinia virus is an LIVP virus or a clonal strain of an LIVP virus.

18. The composition of claim 17, wherein the virus is a modified form containing nucleic acid encoding a heterologous gene product.

19. The composition of claim 18, wherein the heterologous gene product is a therapeutic or reporter gene product.

20. The composition of claim 19, wherein the heterologous gene product is selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging.

21. The composition of claim 19, wherein the heterologous gene product is a therapeutic agent selected from among a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor.

22. The composition of claim 1, wherein the vaccinia virus is present in the composition in an amount that is from or from about 1×10⁵ to 1×10¹² pfu, inclusive.

23. The composition of claim 1 that is formulated for direct administration.

24. The composition of claim 23, wherein the volume of the composition is from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.5 mL to 50 mL or 0.5 mL to 5 mL, each inclusive.

25. The composition of claim 1, further comprising an agent that inhibits or decreases hydrogen bonding in an amount effective to decrease the hydrogen bonding.

26. The composition of claim 24, wherein the agent is selected from among urea, guanidine hydrochloride, dimethyl formamide, colloidal gold sol, aqueous lithium bromide and formic acid.

27. The composition of claim 1 that is formulated for local or systemic injection.

28. The composition of claim 27 that is formulated for intravenous administration.

29. The composition of claim 27 that is formulated for topical administration.

30. A combination, comprising a composition of claim 1 and an anti-cancer agent.

31. The combination of claim 30, wherein the anticancer agent is selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent and a combination of any of the preceding thereof.

32. A virus delivery device, comprising:
the composition of claim 1; and
a device for administration of the composition.

33. The virus delivery device of claim 32, wherein the device is for local administration of the vaccinia virus in polymer composition.

34. The virus delivery device of claim 32, wherein the composition is in the form of a hydrogel.

35. The virus delivery device of claim 34, wherein the composition is coated on a surface of the device.

36. The virus delivery device of claim 32, wherein the device can be applied to a surface of the body of a subject.

37. The virus delivery device of claim 36 that is a patch, bandage, wrap, dressing, suture, film or mesh.

38. The virus delivery device of claim 37 that is a wound dressing or bandage.

39. A method of treating a disease or condition in a subject, comprising administering a composition of claim 1 to a subject, wherein the disease or condition is one that is treated by a administering a therapeutic vaccinia virus.

40. The method of claim 39, wherein the disease or condition is a proliferative disorder.

41. The method of claim 39, wherein the composition is administered locally or systemically.

42. The method of claim 41, wherein the composition is administered intravenously.

43. The method of claim 41, wherein the composition is administered locally inside a body cavity.

44. The method of claim 40, wherein the proliferative disease is a cancer, tumor or metastasis.

45. The method of claim 44, wherein the cancer is a carcinoma, sarcoma, lymphoma or leukemia.

46. The method of claim 44, wherein the tumor is a solid tumor.

47. The method of claim 46, wherein the tumor is a surgically resected tumor.

48. The method of claim 47, wherein the composition is administered topically.

49. The method of claim 40, wherein the proliferative disease is a skin cancer.

50. The method of claim 49, wherein the skin cancer is a melanoma, a basal cell carcinoma of the skin or a squamous cell carcinoma.

51. The method of claim 50, wherein the composition is administered topically.

52. The method of claim 39, wherein the subject is a human or non-human animal.

53. The method of claim 52, wherein the non-human animal is selected from among a horse, cat, dog, cow, pig, sheep, goat, mouse, rabbit, chicken, rat, and guinea pig

54. The method of claim 39, wherein the composition as administered delivers at least or about 1×10⁵ pfu of virus.

55. The method of claim 39, further comprising administering a second therapeutic agent or treatment for the treatment of the proliferative disorder.

56. The method of claim 55, wherein a second therapeutic agent is administered, and the therapeutic agent is an anti-cancer agent.

57. The method of claim 55, wherein a second treatment is administered and is selected from among surgery, radiation therapy and immunosuppressive therapy.

58. The method of claim 56, wherein the composition and the anticancer agent are administered sequentially, simultaneously, or intermittently.

59. A method of treating a skin lesion in a subject, comprising applying a virus delivery device of claim 32 to the surface of the skin of a subject to cover the skin lesion, thereby delivering virus to the skin lesion to treat the skin lesion.

60. The method of claim 59, wherein the skin lesion is a wound or proliferative skin lesion.

61. The method of claim 59, wherein the proliferative skin lesion is benign, premalignant or malignant.

62. The method of claim 60, wherein the proliferative skin lesion is a skin cancer.

63. The method of claim 62, wherein the skin cancer is a melanoma, a basal cell carcinoma of the skin or a squamous cell carcinoma.

64. The method of claim 60, wherein the wound is a traumatic wound or a post-surgical wound.

65. The method of claim 64, wherein the wound is a post-surgical wound that is a surgically resected tumor.

66. The method of claim 59, wherein the subject is a human or non-human animal.
